(19) United States
(12) Patent Application Publication
(10) Pub. No.: US 2014/0359899 A1
(43) Pub. Date: Dec. 4, 2014

(54) Sucrose Transporters and Methods of Generating Pathogen-Resistant Plants

(71) Applicant: Carnegie Institution of Washington, Washington, DC (US)

(72) Inventor: Wolf B. Frommer, Washington, DC (US)

(73) Assignee: Carnegie Institution of Washington, Washington, DC (US)

(21) Appl. No.: 14/363,480

(22) PCT Filed: Dec. 10, 2012
(86) PCT No.: PCT/US2012/068746

§ 371 Date: Jun. 6, 2014

Related U.S. Application Data
(60) Provisional application No. 61/568,493, filed on Dec. 8, 2011.

(51) Int. Cl. C12N 15/82

(52) U.S. Cl. CPC C12N 15/8281; C12N 15/8245; C12N 15/8282
USPC 800/279; 435/419; 435/468; 800/301

(57) ABSTRACT

The present invention relates to genetically modified plant cells that have altered expression or activity of at least one sucrose efflux transporter compared to levels of expression or activity of the at least one sucrose efflux transporter in an unmodified plant cell.

### STATEMENT REGARDING FEDERALLY SPONSORED RESEARCH OR DEVELOPMENT

Part of the work performed during development of this invention utilized U.S. Government funds under Department of Energy Contract No. DE-FG02-04ER1554. The U.S. Government has certain rights in this invention.

### BACKGROUND OF THE INVENTION

1. Field of the Invention

The present invention relates to genetically modified plant cells that have altered expression or activity of at least one sugar efflux transporter compared to levels of expression or activity of the at least one sugar efflux transporter in an unmodified plant cell.

2. Background of the Invention

Microbes and higher organisms depend on an adequate supply of nutrients in order to sustain a basal level of vitality. These nutrients range from inorganic or organic compounds, they include metals, ions, minerals, amino acids, nitrogenous bases, sugars and vitamins. In the need for the vast array of nutrients, there is also a need for absorption and distribution of the nutrients throughout an organism.

Many organisms obtain the necessary nutrients by consuming other organisms and using their own metabolism to digest and process the consumed organism and extract the necessary components. Other organisms, such as pathogens, can parasitically thrive on a host organism and make the host provide the necessary fuels needed to survive.

As described in U.S. Published Application No. 20110209248, plant pathogens can affect the transport of nutrients, such as sugar, in order to manipulate a plant into providing a pathogen with sugars. Thus, a need to inhibit these mechanisms is ever present.

### SUMMARY OF THE INVENTION

The present invention relates to genetically modified plant cells that have increased or decreased expression or activity of at least one sucrose efflux uniporter compared to levels of expression or activity of the at least sucrose efflux transporter in an unmodified plant cell.

The present invention also relates to methods of producing pathogen-resistant or pathogen-tolerant plant cells, with the methods comprising identifying at least one sugar efflux uniporter wherein the levels of expression or activity of the at least one sugar efflux uniporter are altered in the plant cell in response to an infection of the pathogen as compared to an uninfected plant cell, and subsequently modifying the plant cell to either increase or decrease the activity or the expression of the at least one identified sugar efflux uniporter, whereby increasering or decreasing the activity or the expression of the at least one identified sugar efflux uniporter produces the pathogen-resistant plant cell.

### BRIEF DESCRIPTION OF THE DRAWINGS

FIG. 1 depicts the identification of sucrose transporters. (A) HEK293T cell/FRET sensor uptake assay: Out of ^{˜}50 membrane protein genes tested, AtSWEET10 to 15 showed sucrose influx as measured with the sucrose sensor FLIPsuc90μΔ1V; HEK293T cells transfected with sensor only (control) or the sensors and the H+/sucrose cotransporter StSUT1 served as controls (±SEM, n≧11). (B) HEK293T cell/FRET sensor uptake assay: The rice transporters OsSWEET11 and 14 mediate sucrose transport in HEK293T cells (±SEM, n≧11). (C) Oocyte uptake assay: OsSWEET11 and 14, and AtSWEET11 and 12 mediate [¹⁴C]-sucrose uptake (1 mM sucrose; ±SEM, n≧7). (D) Oocyte efflux assay: [¹⁴C]-sucrose efflux by OsSWEET11 in Xenopus oocytes injected with 50 nL of a solution containing 50 mM [¹⁴C]-sucrose; the truncated version OsSWEET11_F205* served as control (±SEM, n≧7). (E) HEK293T cell/FRET sensor transport assay: Reversible accumulation of sucrose in HEK293T cells by AtSWEET11±SEM, n≧10). (F) Oocyte uptake assay: Kinetics of AtSWEET12 for sucrose uptake in Xenopus oocytes (±SEM, n≧14).

FIG. 2 depicts the phenotypic characterization of AtSWEET11 and 12 mutants. (A) Reduced growth of AtSWEEET11;12 double mutant compared to Col-0 wild type and isogenic wild type (control). (B, C) Elevated starch accumulation in AtSWEEET11;12 single and double mutants at the end of the dark period (high light conditions). (D) Sugar levels in mature leaves at the end of light period and end of dark period (±SEM, n≧6; identical letters indicate significance between pairs (day time) according to T-test p≦0.001; c: indicates control; 11;12 indicates atsweet11;12)(high light conditions). (E) Cumulative exudation of [¹⁴C]-derived assimilates from cut petioles of leaves fed with [¹⁴CO₂] (¹⁴C in exudate shown as the percent in exudate plus exudate from the previous exudation period for each time point; ±SEM, n≧5; *t significant at p<0.05; **t significant at p<0.01) (low light conditions). (F, G) Impaired root growth of atsweet11;12 seedlings grown on sugar-free media and media supplemented with sucrose (±SEM, n≧60); two way ANOVA indicates a significant (p<0.0001) between genotype and sucrose treatment).

FIG. 3 depicts GUS and eGFP localization of AtSWEET11 and 12 promoter-reporter fusions. (A-D) GUS histochemistry analysis in rosette leaves of transgenic Arabidopsis plants expressing translational GUS fusions of AtSWEET11 (A, C, D) or 12 (B) with their native promoters. (A, B) GUS staining was detected in leaf vein network; (C) High resolution images of expression in one cell file of an individual vein; (D) Cross section of Arabidopsis leaf showing cell specific localization of AtSWEET11. (E, F) Confocal images of eGFP fluorescence in sepal vein cell files of transgenic Arabidopsis plants expressing translational AtSWEET11-eGFP fusions under control of its native promoter. Insets in (F) show eGFP channel in black and white; red dotted line indicates position of z-scan shown in inset below. eGFP accumulation is observed in static puncta, which may be caused by accumulation of AtSWEET11 in membranes in cell wall ingrowths, which are a feature of phloem parenchyma cells. The presence of cell wall ingrowth was confirmed by electron microscopy.

FIG. 4 depicts the functional characterization of AtSWEET12 and AtSWEET11 in Xenopus oocytes. (A) AtSWEET12 mediates sucrose but not maltose uptake. The truncated mutant AtSWEET12_L203* served as a control (mean±SEM, n≧7). (B) Uptake of radiolabelled sucrose or glucose into Xenopus oocytes expressing AtSWEET11 or 12. Oocytes injected with cRNA for the truncated mutants AtSWEET11_F201* and AtSWEET12_L203* and oocytes injected with RNase-free water (instead of cRNA) served as controls (mean±SEM, n≧3). (C) Time-dependent sucrose uptake was mediated by AtSWEET12 in Xenopus oocytes. Water-injected oocytes served as controls (mean±SEM, n≧6). (D) Time-dependent sucrose efflux was measured in Xenopus oocytes expressing AtSWEET12. Maltose efflux was undetectable. The truncated mutant AtSWEET12_L203* served as a control (mean±SEM, n≧7).

FIG. 5 depicts the functional characterization of AtSWEET12 using a sucrose sensor in HEK293T cells. HEK293T cells were transfected with the sensor FLIPsuc90μΔ1V alone (A) or cotransfected with the sensor and AtSWEET12 (B). Cells were perfused with HBSS buffer, followed by square pulses of 0.1, 0.5, 10 and 20 mM sucrose (0 mM indicated intermittent perfusion with Hank's buffer).

FIG. 6 depicts the affinity and pH dependence of the transport activity of OsSWEET11, OsSWEET14 or AtSWEET12 expressed in Xenopus oocytes. (A) Uptake of radiolabelled sucrose into Xenopus oocytes expressing OsSWEET11 or 14. The truncated mutant OsSWEET11_F205* or water-injected oocytes served as controls. A five-fold increase in the sucrose concentration led to an approximately five-fold increase in the sucrose uptake rate when using low millimolar concentrations, consistent with a high Km of the transporters for sucrose (mean±SEM, n≧6). (B, C) Concentration- and time-dependent sucrose export mediated by AtSWEET12 in Xenopus oocytes injected with radiolabeled sucrose. The truncated mutant AtSWEET12_L203* served as a control to monitor for potential leakage caused by injection. The concentration of sucrose in the oocyte was estimated assuming a cell volume of X pL. The efflux rate increased with increasing sucrose concentration between 1 and 50 mM sucrose; consistent with the data from uptake studies and supporting that AtSWEET12 functions as a low affinity transporter (mean±SEM, n≧7; note that not all error bars are visible, because they are small). (D) Sucrose uptake mediated by AtSWEET12 or OsSWEET11 shows low pH dependence. This pH independence is consistent with a uniport mechanism, as already suggested for the glucose transport activity of the SWEETs (mean±SEM, n≧9)(4).

FIG. 7 depicts the expression of AtSWEET11 and 12 in leaves and coexpression analysis. (A) Organ-specific expression of Arabidopsis SWEET genes derived from publicly available microarray data (www.genevestigator.com/gv/). Among the sucrose-transporting clade III AtSWEET genes (AtSWEET10-15), AtSWEET11 and 12 appear to be most highly expressed (white spots indicate low levels of expression, darker spots mean higher levels of expression). (B, C) Coexpression analysis based on microarray data for AtSWEET11. Some of the most highly coexpressed genes are involved in sucrose biosynthesis and transport (SUC2, the H⁺/sucrose cotransporter; AHA3, a corresponding H⁺/ATPase potentially involved in phloem loading; KAT1, a guard cell potassium channel; the sucrose transporter AtSWEET12 and AtSPS4F, a sucrose phosphate synthase gene encoding a key enzyme for sucrose biosynthesis).

FIG. 8 depicts Translatome data for AtSWEET11 and 12 and the companion cell-expressed H⁺/sucrose cotransporter gene AtSUC2. Data are derived from microarray studies of RNA bound to polysomes.

FIG. 9 depicts molecular characterization of atsweet11, atsweet12 and atsweet11;12 double mutants. (A) Schematic representation of the AtSWEET11 and 12 loci and the respective T-DNA insertion sites. (B) RT-PCR testing AtSWEET11 and AtSWEET12 gene expression levels relative to AtACTIN2 in single and double mutants. Col-0 and a segregating wild type from the double mutant atsweet11;12 (control) served as controls. (C) Schematic drawing of the approximate position of primers, which are specific pairs for amplifying fragments upstream or downstream of the T-DNA insertion sites. (D) Verification of the presence of low levels of a partial transcript for AtSWEET11 and AtSWEET12 genes by qPCR (mean±SEM, n=4).

FIG. 10 depicts significantly reduced rosette diameter of atsweet11;12 double mutants observed under low and high light conditions. (A) Plants were grown under low light (LL) (90-110 μE m⁻² s⁻¹ with 8 hour photoperiod) conditions. The rosette diameter of atsweet11;12 was ˜20% smaller compared to controls, i.e. plants which segregated from the same population as the double mutant. (B) Plants were initially grown under low light (LL) (90-110 μE m⁻² s⁻¹ with 8 hr photoperiod) conditions for two weeks and then transferred to high light (HL) (400-450 μE m⁻² s⁻¹ with 16 hr photoperiod) for 10 days. The rosette diameter of AtSWEET11;12 was ˜35% smaller compared to controls.

FIG. 11 depicts the complementation of the starch accumulation phenotype of the atsweet11;12 double mutant by AtSWEET11 or 12 genes. AtSWEET11 or 12 genes were expressed individually under control of their native promoters in the atsweet11;12 double mutants. (A) RT-PCR analysis of two individual complementation lines transformed with either pAtSWEET11:AtSWEET11 or pAtSWEET12:AtSWEET12. (B) Starch accumulation was analyzed at the end of the darkness in T2 generation complementation lines. Either of the complementation constructs provides partial complementation of the starch accumulation phenotype.

FIG. 12 depicts the low expression of AtSWEET13 in wild type and induction in the atsweet11;12 double mutant. (A) Translatome data indicate that the close paralogs of AtSWEET11 and 12, namely AtSWEET13 and 14 under standard conditions are only lowly expressed in the leaf. (B) Analysis of the expression of AtSWEET13 in atsweet11;12 double mutants shows a ^{˜}15-fold induction of AtSWEET13 in the mutant compared to controls.

FIG. 13 depicts the localization of AtSWEET11 by GUS histochemistry. (A) Cross sections of veins in rosette leaves of transgenic plants expressing AtSWEET11 fused with GUS and driven by the AtSWEET11 promoter. In each vein up to four cells show GUS activity. Bottom panels depict consecutive sections with a comparable staining pattern. The number of cells that express AtSWEET11 is consistent with a phloem parenchyma identity (B) GUS histochemistry showing that AtSWEET12 can be found in two cell files in a rosette leaf vein.

FIG. 14 depicts data supporting localization of AtSWEET11 and AtSWEET12 proteins to the plasma membrane in transgenic lines. Stable transformants of Arabidopsis expressing translational fusions of AtSWEET11 or 12 to eYFP and driven by the CaMV 35S promoter were generated. (A) Confocal image showing a z-section through the root tip of a transgenic line stably expression 35S:AtSWEET11-eYFP. Cells in the root tip of Arabidopsis, in contrast to roots cells above the elongation zone, are characterized by smaller vacuoles and dense cytoplasm (bright field image for orientation; confocal image of the corresponding z-section). The peripheral localization of the fusions indicates plasma membrane localization and is not compatible with vacuolar localization. (B) Confocal image showing a z-section through the root of a transgenic line stably expression 35S:AtSWEET12-eYFP. Analysis of eYFP localization shows peripheral eYFP localization, consistent with a plasma membrane localization as also shown for plants expressing eGFP fusions under the native promoter in phloem cells. Merged image shows that the YFP fluorescence follows the outer contour of the nuclei (see arrows, marked n), indicating that AtSWEET11-eYFP does not localize to the vacuolar membrane. (C) AtSWEET11-eYFP samples were plasmolyzed in 4% NaCl. Hechtian strands, marked with asterisks between plasmolyzed cells, were observed, further supporting AtSWEET11 plasma membrane localization.

FIG. 15 depicts transmission electron microscopic image of a small vein in a sepal from Arabidopsis. Cell wall ingrowth was observed in phloem parenchyma (PP). Blue arrows indicate cell wall ingrowths (SE sieve element; CC companion cell).

FIG. 16 depicts model of sucrose transport in leaves. SWEET sucrose efflux transporters secrete sucrose into the cell wall. H⁺/sucrose cotransporters (SUT1/SUC2) concentrate sucrose in the SE/CC. The H⁺ gradient is provided by the H⁺/ATPase. Membrane potential is maintained by K⁺ channels. Osmotically driven water influx is mediated by aquaporins.

FIG. 17 depicts the expression of SWEETs in response to infection of Arabidopsis wild type plants with C. higginsianum as measured by qPCR.

FIG. 18 depicts resistance to C. higginsianum in plants with SWETT11 and/or SWEET 12 mutants. FIG. 18 B depicts the formation of infection structures is significantly delayed in the SWEET11/SWEET12 double mutant

FIG. 19 depicts the presence of C. higginsianum pathogen genomic DNA in infected plants.

FIG. 20 depicts that osSWEET13 also functions as a weak glucose and as a highly efficient efficient sucrose transporter as shown by coexpressing the rice gene with either a FRET glucose sensor (FLIPGLU600Δ13) in A; or with a sucrose FRET sensor FLIPSUC90μ in B In HEK293T cells.

FIG. 21 depicts that ZmSWEET11 is induced during Ustilago maydis infection. (A) Controls (smaller bar) show base level expression, the taller bar shows about 5-fold induction as measured by qPCR. (B) shows function of ZmSweet11 as a sucrose transporter by coexpression of the maize gene with a sucrose FRET sensor FLIPsuc90μ in HEK293T cells. (C) shows that ZmSweet11 does not transport glucose.

FIG. 22 depicts a Weblogo representation of the alignment of members of the clade III family of SWEETs from Arabidopsis, rice, Medicago, maize and wheat. Weblogo (available on the world wide web at weblogo.berkeley.edu/) illustrates the probability of finding amino acids in corresponding positions in the SWEET genes, e.g. if only a single large letter is visible, this indicates the presence of the respective amino acid in >95% of all cases. If two amino acids are shown with equal height of the letters, this indicates that ^{˜}50% of the proteins have either the one or the other amino acid in that position.

FIG. 23 depicts a phylogenetic tree showing members of the Clade III family of SWEETs from Arabidopsis, Medicago, rice, selected members from maize and wheat and highlights some of the genes that are induced in response to pathogen infection. Pathogens also induce expression of other SWEET clade members and different pathovars and different pathogens induce or activate different SWEET members.

FIG. 24 depicts the assay used for identifying sucrose transporters with the help of FRET sensors in mammalian cells. The Y axis shows the fluorescence emission ratio of the yellow versus cyan proteins normalized to the starting ratio. The top bar indicates the perfusion of the HEK293T cells on an inverted microscope transfected with a construct carrying the FRET sensor FLIPsuc90μΔ1V. Under A, cells perfused first with medium containing no sucrose, then with 2 mM sucrose and then with 20 mM sucrose. The control cells do not show any change in ratio at external concentrations of 2 and 20 mM sucrose, and thus no accumulation of sucrose in the cytosol of the HEK293T cells. In B, a negative ratio change indicated accumulation of sucrose in the HEK293T cells that coexpress the Arabidopsis sucrose proton cotransporters AtSUC1 after addition of 20 mM sucrose. In C, the potato sucrose proton cotransporter mediates uptake of sucrose detectable upon addition of 2 or 20 mM sucrose. StSUT1 is more active in this assay compared to AtSUC1 since a FRET change is detectable already with addition of 2 mM sucrose.

FIG. 25 depicts a chart showing that the activity of various SWEET proteins is induced by different plant pathogens.

FIG. 26 depicts the sugar uptake and efflux activity of AtSWEET9 in an oocyte system. (A) Oocyte uptake assay: AtSWEET9 and NaNEC1 mediate [¹⁴C]-glucose, fructose and sucrose uptake (1 mM glucose, fructose and sucrose); (B, C and D) [¹⁴C]-sucrose (B), -glucose (C) and -fructose (D) efflux by AtSWEET9 in Xenopus oocytes injected with 50 nL of a solution containing 10 mM [¹⁴C]-sucrose, -glucose and -fructose.

FIG. 27 depicts GUS and eGFP localization of AtSWEET11 and 12 promoter-reporter fusions. (A-D) GUS histochemistry analysis in flowers of transgenic Arabidopsis plants expressing translational GUS fusions of AtSWEET9 with its native promoters. GUS staining was detected in lateral nectary (A) and median nectaries (B); (C and D) Transverse (C) and vertical (D) section of Arabidopsis flower showing cell specific localization of AtSWEET9. The plant cell walls were stained with safranin-O. (E and F) Confocal images of eGFP fluorescence in lateral (E) and median (F) nectaries in transgenic Arabidopsis plants expressing translational AtSWEET9-eGFP fusions under control of its native promoter. Auto-fluorescence of chloroplasts is shown. (G) The subcellular localization of eGFP accumulation is observed in the plasma membrane, Golgi and vesicles in the lateral nectaries.

FIG. 28 depicts nectar production in wild-type and sweet9 mutant transgenic flowers. (A) The nectar droplet was clinging to the inside of a sepal of a wild-type flower. (B and C) No nectar was secreted from the nectaries of both sweet9-1 and sweet9-2 mutant lines. (D) More nectar was secreted from the nectaries of the wild-type flowers which containing more one copy of SWEET9-eGFP. (E and F) The nectar was secreted from the nectaries of the complemented sweet9 mutants containing native promoter and the AtSWEET9 (E) or AtSWEET9-eGFP (F). (G, H and I) The nectar production phenotype was complemented by expression of AtSWEET1 (G), AtSWEET11 (H) and 12 (I) under AtSWEET9 promoter in the sweet9 mutant plants.

FIG. 29 depicts accumulation of starch grains stained with Lugol's iodine solution in the floral stalks and the nectaries in sweet9 mutant lines at anthesis. (A) The flowers of wild-type and sweet9-1 mutant stained with Lugol's iodine solution. The starch accumulated in the floral stalk of sweet9-1 mutant lines. The flowers were sampled at 10 a.m. (B) Close-up of the flower stalks in wild-type and sweet9-1 mutant lines. (C) Close-up of nectaries in wild-type and sweet9-1 mutant lines. The starch grains accumulated in the guard cells of the nectaries in wild-type flowers; the starch grains accumulated in the whole nectary parenchyma in the sweet9-1 flowers. The flowers were sampled at the end of the dark. (D) LR White resin sections of Arabidopsis nectaries in wild-type and sweet9-1 mutant lines stained with Lugol's iodine solution. The rectangle indicates the position of nectaries. The starch grains accumulate in the whole section in sweet9-1 mutant lines. The starch grains showed as dark red spots. The plant cell walls were stained with safranin-O.

FIG. 30 depicts AtSWEETs expression in the different seed development stages. Abbreviations are as follows. A: Absent, INS: inconsistent detection, M: marginal, P: present, PGLOB: pre-globular stage, GLOB: globular stage, HRT: heart stage, LCOT: linear cotyledon stage, MG: maturation green stage, CZE: chalazal endosperm, CZSC: chalazal seed coat, EP: embryo proper, GSC: general seed coat, MCE: micropylar endosperm, PEN: peripheral endosperm, S: suspensor, WS: whole seed.

FIG. 31 depicts the localization of AtSWEET11 and AtSWEET15 in seed.

FIG. 32 depicts response of HEK cells transfected with various SWEETS from corn (Zm), rice (Os) and citrus (Cs). The graphs show influx of sucrose into the transfected cells.

FIG. 33 depicts response of HEK cells transfected with various SWEETS from corn (Zm), rice (Os) and citrus (Cs). The graphs show influx of glucose into the transfected cells.

FIG. 34 depicts amino acid sequences from various SWEET transporters from various species. At: arabidopsis thaliana (arabidopsis), Os: oryza sativa (rice), Zm: zea mays (corn), Cs: citrus sinensis (orange), Mt: medicago trunculata (barrel medic), Ta: triticum aestivum (wheat), Gm: glycine max (soybean), Ph: Petunia hybrida (petunia), Pt: populus trichocarpa (poplar), Vv: vitis vinifera (grape), Bd: brachypodium distachyon, Hv: hordeum vulgare (barley), Sb: sorghum bicolor (sorghum), Ps: picea sitchensis (spruce), Lj: lotus japonicus, Na: nicotiana alata (tobacco), Sl: solanum lycopersicum (tomato).

FIG. 35 depicts the identification of sucrose transport activity for soybean SWEET11 (GmSweet11) by co-expression with cytosolic FRET sucrose sensor FLIPsuc90mΔ1V in HEK293T cells. Individual cells were analyzed by quantitative ratio imaging of CFP and Venus emission (acquisition interval 10s). HEK293T/FLIPsuc90mΔ1V cells were perfused with medium, followed by a pulse of 10 mM sucrose. HEK293T cells transfected with sensor only (top trace) or the sensor and the Arabidopsis Sweet12 (bottom trace) served as controls. GmSweet11 shows sucrose influx (middle trace) as measured with the sucrose sensor.

### DETAILED DESCRIPTION OF THE INVENTION

The present invention relates to genetically modified plant cells that have altered expression or activity of at least one sugar efflux uniporter compared to levels of expression or activity of the at least one sucrose efflux transporter in an unmodified plant cell. The present invention also relates to genetically modified plant cells that have altered expression or activity of at least one sugar influx transporter compared to levels of expression or activity of the at least sucrose influx transporter in an unmodified plant cell.

As described herein, the genetically modified plant cell may be a plant cell from a dicot or monocot or gymnosperm. The plant may be crops, such as a food crops, feed crops or biofuels crops. Exemplary important crops may include corn, wheat, soybean, cotton and rice. Crops also include corn, wheat, barley, triticale, soybean, cotton, millet, sorghum, sugarcane, sugar beet, potato, tomato, grapevine, citrus (orange, lemon, grapefruit, etc), lettuce, alfalfa, common bean, fava bean and strawberries, sunflowers and rapeseed, cassava, miscanthus and switchgrass. Other examples of plants include but are not limited to an African daisy, African violet, alfalfa, almond, anemone, apple, apricot, asparagus, avocado, azalea, banana and plantain, beet, bellflower, black walnut, bleeding heart, butterfly flower, cacao, caneberries, canola, carnation, carrot, cassava, diseases, chickpea, cineraria, citrus, coconut palm, coffee, common bean, maize, cotton, crucifers, cucurbit, cyclamen, dahlia, date palm, douglas-fir, elm, English walnut, flax, Acanthaceae, Agavaceae, Araceae, Araliaceae, Araucariacea, Asclepiadaceae, Bignoniaceae, Bromeliaceae, Cactaceae, Commelinaceae, Euphobiaceae, Gentianaceae, Gesneriaceae, Maranthaceae, Moraceae, Palmae, Piperaceae, Polypodiaceae, Urticaceae, Vitaceae, fuchsia, geranium, grape, hazelnut, hemp, holiday cacti, hop, hydrangea, impatiens, Jerusalem cherry, kalanchoe, lettuce, lentil, lisianthus, mango, mimulus, monkey-flower, mint, mustar, oats, papaya, pea, peach and nectarine, peanut, pear, pearl millet, pecan, pepper, Persian violet, pigeonpea, pineapple, pistachio, pocketbook plant, poinsettia, potato, primula, red clover, rhododendron, rice, rose, rye, safflower, sapphire flower, spinach, strawberry, sugarcane, sunflower, sweetgum, sweet potato, sycamore, tea, tobacco, tomato, verbena, and wild rice.

The plant cell can be from any part or tissue of a plant including but not limited to the root, stem, leaf, seed, seedcoat, flower, fruit, anther, nectary, ovary, petal, tapetum, xylem, or phloem. If the genetically modified plant cell is comprised within a whole plant, the entire plant need not contain or express the genetic modification.

A Clade III transporter can be identified through a highly conserved domain. The present invention provides for a Clade III transporter comprising the domain V-M/F-Y/V-A-G-S/A-S/P/L-S-M/X/I-V-A/M-I-L-V/X/X/V/I-V/K-X/T-S/K-R-E/S/V-A/E-K-Q-A/Y-F/M/P/F/X/L-M/S. The conserved domain may be between the fifth and sixth transmembrane domains of a seven transmembrane transporter. The present invention provides for Clade III transporters that comprise seven Trans-membrane Domains (TMd), and the consensus Sequence. Clade III transporters may further comprise a combination of two or more of the following sequences: the sequence K-R-A/K-N-S/K/S-T/T-S-I-A/E-K-Q-G/G-S-C/F-Y/Q-S-E-H/S-A/I-L-V-T/P/Y/X/V-S-T-C/A-S-T/L/F-L-A/S/A-C-S-T/M-T-G-L/L/W-F-L/I-L-M-V/Y-F-L/Y/A-G/X/K-R-Q-S-T between the second transmembrane domain (TMd); the sequence V-M/F/V-A/A-S/P/L/S-A-F-M-T/I-V/I-M-V/X/X/V/I-V-M/K-R-Q/T-S/K-R/S/V/E-A/Y-F/M-L/P/F-I/X/L/S between the fifth and the sixth TMd, and the sequence P/N/V-I-G-T/L-G-V-I/G/F-L-A/X/F-L/G-S/X/X/Q/M/X/X/Y-F/X/X/Y-F in the seventh TMd.

Examples of Clade III sucrose efflux transporters that cane be used in the present invention include but are not limited to sucrose transporters terms SWEET9, SWEET10, SWEET11, SWEET12, SWEET13, SWEET14, SWEET15 NaNEC1 and PhNEC1. The invention provides sucrose efflux transporters that are utilized, modified and/or altered in the plant cells that belong to the Clade III family of efflux transporters. The Clade III sucrose efflux transporter proteins generally posses a highly conserved region between the fifth and sixth transmembrane domains.

In another embodiment, the sugar uniporter is a sucrose transporter from one of the other clades, e.g., the citrus SWEET1 belonging to Clade I is induced by citrus canker (Xanthomonas ssp.) infection and functions as a sucrose transporter.

In one aspect, the invention provides deletion variants wherein one or more amino acid residues in the transporter proteins. Deletions can be effected at one or both termini of the transporter protein.

The proteins of the present invention may also comprise substitution variants of an efflux transporter protein. Substitution variants include those polypeptides wherein one or more amino acid residues of the efflux transporters are removed and replaced with alternative residues. In general, the substitutions are conservative in nature. Conservative substitutions for this purpose may be defined as set out in the tables below. Amino acids can be classified according to physical properties and contribution to secondary and tertiary protein structure. A conservative substitution is recognized in the art as a substitution of one amino acid for another amino acid that has similar properties. Exemplary conservative substitutions are set out in below.

TABLE I
Conservative Substitutions

| Side Chain Characteristic | Amino Acid |
|---|---|
| Aliphatic | |
| Non-polar | Gly, Ala, Pro, Iso, Leu, Val |
| Polar-uncharged | Cys, Ser, Thr, Met, Asn, Gln |
| Polar-charged | Asp, Glu, Lys, Arg |
| Aromatic | His, Phe, Trp, Tyr |
| Other | Asn, Gln, Asp, Glu |

Alternatively, conservative amino acids can be grouped as described in Lehninger (1975) Biochemistry, Second Edition; Worth Publishers, pp. 71-77, as set forth below.

TABLE II
Conservative Substitutions

| Side Chain Characteristic | Amino Acid |
|---|---|
| Non-polar (hydrophobic) | |
| Aliphatic: | Ala, Leu, Iso, Val, Pro |
| Aromatic: | Phe, Trp |
| Sulfur-containing: | Met |
| Borderline: | Gly |
| Uncharged-polar | |
| Hydroxyl: | Ser, Thr, Tyr |
| Amides: | Asn, Gln |
| Sulfhydryl: | Cys |
| Borderline: | Gly |
| Positively Charged (Basic): | Lys, Arg, His |
| Negatively Charged (Acidic) | Asp, Glu |

And still other alternative, exemplary conservative substitutions are set out below.

TABLE III
Conservative Substitutions

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe |
| Leu (L) | Ile, Val, Met, Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr |
| Tyr (Y) | Trp, Phe, Thr, Ser |
| Val (V) | Ile, Leu, Met, Phe, Ala |

The invention thus also provides isolated peptides, with the peptides comprising an amino acid sequence at least about 75%, 80%, 85%, 86%, 87%, 88%, 89%, 90%, 91%, 92%, 93%, 94%, 95%, 96%, 97%, 98%, 99% or 100% identical to the amino acid sequences of the sucrose efflux transporters or disclosed or incorporated by reference herein. For example, the invention provides for polypeptides comprising or consist of amino acid sequences that are 75%, 80%, 85%, 86%, 87%, 88%, 89%, 90%, 91%, 92%, 93%, 94%, 95%, 96%, 97%, 98%, 99% or 100% identical to the amino acid sequences of any of the efflux transport proteins disclosed or incorporated by reference herein.

A polypeptide having an amino acid sequence at least, for example, about 95% “identical” to a reference an amino acid sequence is understood to mean that the amino acid sequence of the polypeptide is identical to the reference sequence except that the amino acid sequence may include up to about five modifications per each 100 amino acids of the reference amino acid sequence. In other words, to obtain a peptide having an amino acid sequence at least about 95% identical to a reference amino acid sequence, up to about 5% of the amino acid residues of the reference sequence may be deleted or substituted with another amino acid or a number of amino acids up to about 5% of the total amino acids in the reference sequence may be inserted into the reference sequence. These modifications of the reference sequence may occur at the N-terminus or C-terminus positions of the reference amino acid sequence or anywhere between those terminal positions, interspersed either individually among amino acids in the reference sequence or in one or more contiguous groups within the reference sequence.

As used herein, “identity” is a measure of the identity of nucleotide sequences or amino acid sequences compared to a reference nucleotide or amino acid sequence. In general, the sequences are aligned so that the highest order match is obtained. “Identity” per se has an art-recognized meaning and can be calculated using well known techniques. While there are several methods to measure identity between two polynucleotide or polypeptide sequences, the term “identity” is well known to skilled artisans (Carillo (1988) J. Applied Math. 48, 1073). Examples of computer program methods to determine identity and similarity between two sequences include, but are not limited to, GCG program package (Devereux (1984) Nucleic Acids Research 12, 387), BLASTP, ExPASy, BLASTN, FASTA (Atschul (1990) J. Mol. Biol. 215, 403) and FASTDB. Examples of methods to determine identity and similarity are discussed in Michaels (2011) Current Protocols in Protein Science, Vol. 1, John Wiley & Sons.

In one embodiment of the present invention, the algorithm used to determine identity between two or more polypeptides is BLASTP. In another embodiment of the present invention, the algorithm used to determine identity between two or more polypeptides is FASTDB, which is based upon the algorithm of Brutlag (1990) Comp. App. Biosci. 6, 237-245). In a FASTDB sequence alignment, the query and reference sequences are amino sequences. The result of sequence alignment is in percent identity. In one embodiment, parameters that may be used in a FASTDB alignment of amino acid sequences to calculate percent identity include, but are not limited to: Matrix=PAM, k-tuple=2, Mismatch Penalty=1, Joining Penalty=20, Randomization Group Length=0, Cutoff Score=1, Gap Penalty=5, Gap Size Penalty 0.05, Window Size=500 or the length of the subject amino sequence, whichever is shorter.

If the reference sequence is shorter or longer than the query sequence because of N-terminus or C-terminus additions or deletions, but not because of internal additions or deletions, a manual correction can be made, because the FASTDB program does not account for N-terminus and C-terminus truncations or additions of the reference sequence when calculating percent identity. For query sequences truncated at the N- or C-termini, relative to the reference sequence, the percent identity is corrected by calculating the number of residues of the query sequence that are N- and C-terminus to the reference sequence that are not matched/aligned, as a percent of the total bases of the query sequence. The results of the FASTDB sequence alignment determine matching/alignment. The alignment percentage is then subtracted from the percent identity, calculated by the above FASTDB program using the specified parameters, to arrive at a final percent identity score. This corrected score can be used for the purposes of determining how alignments “correspond” to each other, as well as percentage identity. Residues of the reference sequence that extend past the N- or C-termini of the query sequence may be considered for the purposes of manually adjusting the percent identity score. That is, residues that are not matched/aligned with the N- or C-termini of the comparison sequence may be counted when manually adjusting the percent identity score or alignment numbering.

For example, a 90 amino acid residue query sequence is aligned with a 100 residue reference sequence to determine percent identity. The deletion occurs at the N-terminus of the query sequence and therefore, the FASTDB alignment does not show a match/alignment of the first 10 residues at the N-terminus. The 10 unpaired residues represent 10% of the reference sequence (number of residues at the N- and C-termini not matched/total number of residues in the reference sequence) so 10% is subtracted from the percent identity score calculated by the FASTDB program. If the remaining 90 residues were perfectly matched (100% alignment) the final percent identity would be 90% (100% alignment−10% unmatched overhang). In another example, a 90 residue query sequence is compared with a 100 reference sequence, except that the deletions are internal deletions. In this case the percent identity calculated by FASTDB is not manually corrected, since there are no residues at the N- or C-termini of the subject sequence that are not matched/aligned with the query. In still another example, a 110 amino acid query sequence is aligned with a 100 residue reference sequence to determine percent identity. The addition in the query occurs at the N-terminus of the query sequence and therefore, the FASTDB alignment may not show a match/alignment of the first 10 residues at the N-terminus. If the remaining 100 amino acid residues of the query sequence have 95% identity to the entire length of the reference sequence, the N-terminal addition of the query would be ignored and the percent identity of the query to the reference sequence would be 95%.

As used herein, the terms “correspond(s) to” and “corresponding to,” as they relate to sequence alignment, are intended to mean enumerated positions within a reference protein, e.g., wild-type SWEET9, and those positions in a modified SWEET9 that align with the positions on the reference protein. Thus, when the amino acid sequence of a subject protein is aligned with the amino acid sequence of a reference protein, the amino acids in the subject sequence that “correspond to” certain enumerated positions of the reference sequence are those that align with these positions of the reference sequence, but are not necessarily in these exact numerical positions of the reference sequence. Methods for aligning sequences for determining corresponding amino acids between sequences are described herein.

The invention also provides isolated nucleic acids, with the nucleic acids comprising polynucleotide sequence at least about 75%, 80%, 85%, 86%, 87%, 88%, 89%, 90%, 91%, 92%, 93%, 94%, 95%, 96%, 97%, 98%, 99% or 100% identical to the polynucleotide sequences disclosed herein.

As a practical matter, whether any particular nucleic acid molecule is at least 80%, 85%, 90%, 92%, 95%, 96%, 97%, 98% or 99% identical to a disclosed nucleic acid can be determined conventionally using known computer programs a discussed herein. For example, percent identity can be determined using the Bestfit program (Wisconsin Sequence Analysis Package, Version 8 for Unix, Genetics Computer Group, University Research Park, 575 Science Drive, Madison, Wis. 53711. Bestfit uses the local homology algorithm of Smith and Waterman, Advances in Applied Mathematics 2: 482-489 (1981), to find the best segment of homology between two sequences. When using Bestfit or any other sequence alignment program to determine whether a particular sequence is, for instance, 95% identical to a reference sequence according to the present invention, the parameters are set, of course, such that the percentage of identity is calculated over the full length of the reference nucleotide sequence and that gaps in homology of up to 5% of the total number of nucleotides in the reference sequence are allowed. Methods for correcting percent identity of polynucleotides are the same as those described and disclosed herein with respect to polypeptides.

The engineered proteins of the present invention may or may not contain additional elements that, for example, may include but are not limited to regions to facilitate purification. For example, “histidine tags” (“his tags”) or “lysine tags” may be appended to the engineered protein. Examples of histidine tags include, but are not limited to hexaH, heptaH and hexaHN. Examples of lysine tags include, but are not limited to pentaL, heptaL and FLAG. Such regions may be removed prior to final preparation of the engineered protein. Other examples of a fusion partner for the engineered proteins of the present invention include, but are not limited to, glutathione S-transferase (GST) and alkaline phosphatase (AP), or fluorescent proteins such as the green fluorescent protein (GFP).

The addition of peptide moieties to engineered proteins, whether to engender secretion or excretion, to improve stability and to facilitate purification or translocation, among others, is a familiar and routine technique in the art and may include modifying amino acids at the terminus to accommodate the tags. For example the N-terminus amino acid may be modified to, for example, arginine and/or serine to accommodate a tag. Of course, the amino acid residues of the C-terminus may also be modified to accommodate tags. One particularly useful fusion protein comprises a heterologous region from immunoglobulin that can be used solubilize proteins.

Other types of fusion proteins provided by the present invention include but are not limited to, fusions with secretion signals and other heterologous functional regions. Thus, for instance, a region of additional amino acids, particularly charged amino acids, may be added to the N-terminus of the engineered protein to improve stability and persistence in the host cell, during purification or during subsequent handling and storage.

The engineered proteins of the current invention may be recovered and purified from recombinant cell cultures by well-known methods including, but not limited to, ammonium sulfate or ethanol precipitation, acid extraction, anion or cation exchange chromatography, phosphocellulose chromatography, hydrophobic interaction chromatography, affinity chromatography, e.g., immobilized metal affinity chromatography (IMAC), hydroxylapatite chromatography and lectin chromatography. High performance liquid chromatography (“HPLC”) may also be employed for purification. Well-known techniques for refolding protein may be employed to regenerate active conformation when the fusion protein is denatured during isolation and/or purification.

Engineered proteins of the present invention include, but are not limited to, products of chemical synthetic procedures and products produced by recombinant techniques from a prokaryotic or eukaryotic host, including, for example, bacterial, yeast, higher plant, insect and mammalian cells. Depending upon the host employed in a recombinant production procedure, the engineered proteins of the present invention may be glycosylated or may be non-glycosylated. In addition, engineered proteins of the invention may also include an initial modified methionine residue, in some cases as a result of host-mediated processes.

The present invention also provides for nucleic acids encoding some of the engineered proteins of the present invention.

The invention also relates to isolated nucleic acids and to constructs comprising these nucleic acids. The nucleic acids of the invention can be DNA or RNA, for example, mRNA. The nucleic acid molecules can be double-stranded or single-stranded; single stranded RNA or DNA can be the coding, or sense, strand or the non-coding, or antisense, strand. In particular, the nucleic acids may encode any engineered protein of the invention. For example, the nucleic acids of the invention include polynucleotide sequences that encode the engineered proteins that contain or comprise glutathione-S-transferase (GST) fusion protein, poly-histidine (e.g., His₆), poly-HN, poly-lysine, etc. If desired, the nucleotide sequence of the isolated nucleic acid can include additional non-coding sequences such as non-coding 3′ and 5′ sequences (including regulatory sequences, for example).

The nucleic acid molecules of the invention can be “isolated.” As used herein, an “isolated” nucleic acid molecule or nucleotide sequence is intended to mean a nucleic acid molecule or nucleotide sequence that is not flanked by nucleotide sequences normally flanking the gene or nucleotide sequence (as in genomic sequences) and/or has been completely or partially removed from its native environment (e.g., a cell, tissue). For example, nucleic acid molecules that have been removed or purified from cells are considered isolated. In some instances, the isolated material will form part of a composition (for example, a crude extract containing other substances), buffer system or reagent mix. In other circumstances, the material may be purified to near homogeneity, for example as determined by PAGE or column chromatography such as HPLC. Thus, an isolated nucleic acid molecule or nucleotide sequence can includes a nucleic acid molecule or nucleotide sequence which is synthesized chemically, using recombinant DNA technology or using any other suitable method. To be clear, a nucleic acid contained in a vector would be included in the definition of “isolated” as used herein. Also, isolated nucleotide sequences include recombinant nucleic acid molecules (e.g., DNA, RNA) in heterologous organisms, as well as partially or substantially purified nucleic acids in solution. “Purified,” on the other hand is well understood in the art and generally means that the nucleic acid molecules are substantially free of cellular material, cellular components, chemical precursors or other chemicals beyond, perhaps, buffer or solvent. “Substantially free” is not intended to mean that other components beyond the novel nucleic acid molecules are undetectable. The nucleic acid molecules of the present invention may be isolated or purified. Both in vivo and in vitro RNA transcripts of a DNA molecule of the present invention are also encompassed by “isolated” nucleotide sequences.

The invention also provides nucleic acid molecules that hybridize under high stringency hybridization conditions, such as for selective hybridization, to the nucleotide sequences described herein (e.g., nucleic acid molecules which specifically hybridize to a nucleotide sequence encoding engineered proteins described herein). Hybridization probes include synthetic oligonucleotides which bind in a base-specific manner to a complementary strand of nucleic acid.

Such nucleic acid molecules can be detected and/or isolated by specific hybridization e.g., under high stringency conditions. “Stringency conditions” for hybridization is a term of art that refers to the incubation and wash conditions, e.g., conditions of temperature and buffer concentration, which permit hybridization of a particular nucleic acid to a second nucleic acid; the first nucleic acid may be perfectly complementary, i.e., 100%, to the second, or the first and second may share some degree of complementarity, which is less than perfect, e.g., 60%, 75%, 85%, 95% or more. For example, certain high stringency conditions can be used which distinguish perfectly complementary nucleic acids from those of less complementarity.

“High stringency conditions”, “moderate stringency conditions” and “low stringency conditions” for nucleic acid hybridizations are explained in Current Protocols in Molecular Biology, John Wiley & Sons). The exact conditions which determine the stringency of hybridization depend not only on ionic strength, e.g., 0.2×SSC, 0.1×SSC of the wash buffers, temperature, e.g., room temperature, 42° C., 68° C., etc., and the concentration of destabilizing agents such as formamide or denaturing agents such as SDS, but also on factors such as the length of the nucleic acid sequence, base composition, percent mismatch between hybridizing sequences and the frequency of occurrence of subsets of that sequence within other non-identical sequences. Thus, high, moderate or low stringency conditions may be determined empirically.

By varying hybridization conditions from a level of stringency at which no hybridization occurs to a level at which hybridization is first observed, conditions which will allow a given sequence to hybridize with the most similar sequences in the sample can be determined. Exemplary conditions are described in Krause (1991) Methods in Enzymology, 200:546-556. Washing is the step in which conditions are usually set so as to determine a minimum level of complementarity of the hybrids. Generally, starting from the lowest temperature at which only homologous hybridization occurs, each degree (° C.) by which the final wash temperature is reduced, while holding SSC concentration constant, allows an increase by 1% in the maximum extent of mismatching among the sequences that hybridize. Generally, doubling the concentration of SSC results in an increase in Tm. Using these guidelines, the washing temperature can be determined empirically for high, moderate or low stringency, depending on the level of mismatch sought. Exemplary high stringency conditions include, but are not limited to, hybridization in 50% formamide, 1 M NaCl, 1% SDS at 37° C., and a wash in 0.1×SSC at 60° C. Example of progressively higher stringency conditions include, after hybridization, washing with 0.2×SSC and 0.1% SDS at about room temperature (low stringency conditions); washing with 0.2×SSC, and 0.1% SDS at about 42° C. (moderate stringency conditions); and washing with 0.1×SSC at about 68° C. (high stringency conditions). Washing can be carried out using only one of these conditions, e.g., high stringency conditions, washing may encompass two or more of the stringency conditions in order of increasing stringency. Optimal conditions will vary, depending on the particular hybridization reaction involved, and can be determined empirically.

Equivalent conditions can be determined by varying one or more of the parameters given as an example, as known in the art, while maintaining a similar degree of identity or similarity between the target nucleic acid molecule and the primer or probe used. Hybridizable nucleotide sequences are useful as probes and primers for identification of organisms comprising a nucleic acid of the invention and/or to isolate a nucleic acid of the invention, for example. The term “primer” is used herein as it is in the art and refers to a single-stranded oligonucleotide, which acts as a point of initiation of template-directed DNA synthesis under appropriate conditions in an appropriate buffer and at a suitable temperature. The appropriate length of a primer depends on the intended use of the primer, but typically ranges from about 15 to about 30 nucleotides. Short primer molecules generally require cooler temperatures to form sufficiently stable hybrid complexes with the template. A primer need not reflect the exact sequence of the template, but must be sufficiently complementary to hybridize with a template. The term “primer site” refers to the area of the target DNA to which a primer hybridizes. The term “primer pair” refers to a set of primers including a 5′ (upstream) primer that hybridizes with the 5′ end of the DNA sequence to be amplified and a 3′ (downstream) primer that hybridizes with the complement of the 3′ end of the sequence to be amplified.

Although the gene nomenclature herein often refers to genes and proteins identified in The Arabidopsis Information Resource (TAIR) database, which is available on the worldwide web at www.arabidopsis.org, it is understood that the invention is not limited to genes in Arabidposis or any other species. The invention also encompasses orthologs of genes and proteins in other species. For example, it is understood that methods and plant cells utilizing the transporter encoded by the gene AT2G39060 (SWEET9) in Arabidopsis can be applied to the orthologous gene in another species. As used herein, orthologous genes are genes from different species that perform the same or similar function and are believed to descend from a common ancestral gene. Proteins from orthologous genes, in turn, are the proteins encoded by the orthologs. As such the term “ortholog” may be to refer to a gene or a protein. Often, proteins encoded by orthologous genes have similar or nearly identical amino acid sequence identities to one another, and the orthologous genes themselves have similar nucleotide sequences, particularly when the redundancy of the genetic code is taken into account. Thus, by way of example, the ortholog of an efflux sucrose transporter in Arabidopsis would be an efflux sucrose efflux transporter in another species of plant, regardless of the amino acid sequence of the two proteins. For example, Table IV below shows the name of the sugar transporter protein and the corresponding TAIR accession database number for various SWEET proteins in arabidopsis. Each of the records and all information contained therein, including but not limited to information embedded in hyperlinks, from the TAIR database is incorporated by reference in its entirety.

TABLE IV
Arabidopsis SWEET Genes

| Name | Gene Record ID |
|---|---|
| SWEET1 | AT1G21460 |
| SWEET2 | AT3G14770 |
| SWEET3 | AT5G53190 |
| SWEET4 | AT3G28007 |
| SWEET5 | AT5G62850 |
| SWEET6 | AT1G66770 |
| SWEET7 | AT4G10850 |
| SWEET8 | AT5G40260 |
| SWEET9 | AT2G39060 |
| SWEET10 | AT5G50790 |
| SWEET11 | AT3G48740 |
| SWEET12 | AT5G23660 |
| SWEET13 | AT5G50800 |
| SWEET14 | AT4G25010 |
| SWEET15 | AT5G13170 |
| SWEET16 | AT3G16690 |
| SWEET17 | AT4G15920 |

Other databases include but are not limited to the greenphyl, which is located on the world wide web at greenphyl.org. A rice database is available on the internet at: mips.helmholtz-muenchen.de/plant/rice/searchjsp/index.jsp. For example, Table V below shows the name of the sugar transporter protein and the corresponding greenphyl accession database number for various SWEET proteins in rice (oryza sativa). Each of the records and all information contained therein, including but not limited to information embedded in hyperlinks, from the greenphyl database is incorporated by reference in its entirety.

TABLE V
Oryza Sativa SWEET Genes

| Name | Gene Record ID |
|---|---|
| OsSWEET1a | Os01g65880 |
| OsSWEET1b | Os05g35140 |
| OsSWEET2a | Os01g36070 |
| OsSWEET2b | Os01g50460 |
| OsSWEET3a | Os05g12320 |
| OsSWEET3b | Os01g12130 |
| OsSWEET4 | Os02g19820 |
| OsSWEET5 | Os05g51090 |
| OsSWEET6a | Os01g42110 |
| OsSWEET6b | Os01g42090 |
| OsSWEET7a | Os09g08030 |
| OsSWEET7b | Os09g08440 |
| OsSWEET7c | Os12g07860 |
| OsSWEET7d | Os09g08490 |
| OsSWEET7e | Os09g08270 |
| OsSWEET11/Os8N3 | Os08g42350 |
| OsSWEET12 | Os03g22590 |
| OsSWEET13 | Os12g29220 |
| OsSWEET14/Os11N3 | Os11g31190 |
| OsSWEET15 | Os02g30910 |
| OsSWEET16 | Os03g22200 |

The present invention provides for plant cells that are resistant to pathogens. In one embodiment, the plant cells comprise at least one copy of a gene encoding a sucrose efflux transporter that is modified or mutated such that the overall activity of expression of sucrose transporter is decreased as compared to unmodified plants. In another embodiment, the plant cells comprise a genetic such that the overall activity of expression of the sucrose efflux transporter is increased as compared to unmodified plants. In certain specific embodiments, the genetic mutation to increase the overall activity of expression of sucrose efflux transporter comprises one or more additional copies of the efflux transporter gene inserted into the plant cells.

As used herein, the term “gene” means a stretch of nucleotides that encode a polypeptide. The “gene,” for the purposes of the present invention, need not have introns and regulatory regions associated with the coating region. Accordingly, a cDNA that encodes a polypeptide is considered a “gene” for the purposes of the present invention. Of course, the term “gene” also includes the full length polynucleotide, or any portion thereof, that encodes a polypeptide and may or may not include introns, promoters, enhancers, UTRs, etc.

The modification may be a mutation to a regulatory domain such as a promoter or other 5′ or 3′ untranslated domain. The modification may be to a promoter, a coding region, an intron of the gene, a splice site of the gene or an exon of the gene. The modification may be a point mutation, a silent mutation, an insertion or a deletion. An insertion or a deletion may be any number of nucleic acids, and the invention is not limited by the number of additions or deletions that effectuate the genetic modification. In one embodiment, the modification to the efflux transporter should decrease or reduce the ability of the efflux transporter to transport or sense a nutrient. Accordingly, the modification may occur at the biogenesis of the efflux transporter transporter at the genetic level from promoter to posttranslational modification, as well as at the level of affecting turnover and inactivation, e.g., by phosphorylation or ubiquitination (see, e.g., Niittylae et al. Mol Cell Proteomics, 6(10):1711-26 (2007)). For example, disruption of a site for post-translational modification, such as a site for phosphorylation or ubiquitination, may provide a suitable modification to disrupt the functioning of the transporter.

In one embodiment, the present invention provides methods of regulating a sucrose efflux transporter expression by modifying a sucrose efflux transporter gene. In one embodiment, inserting or introducing one ineffective (or less effective) copy of an efflux transporter may be sufficient to inhibit or reduce the function of an efflux transporter, if the efflux transporter normally exists as a multimer. One can also express only a domain of the transporter, wild type or mutated, to block activity of the intact versions in the plant. In another embodiment, inserting one additional copy of an efflux transporter may be sufficient to increase the expression or function of an efflux transporter, if the efflux transporter normally exists as a multimer. The gene encoding the sucrose efflux transporter may be modified upstream of the coding region, such as in a transcription factor binding site, such as a TAL effector. The binding site may be modified by mutating a repeat sequence upstream of the coding region. As discussed herein, mutations may include insertion or deletion of one or several nucleic acids. Mutations may also include the replacement of a region with that of another region, such as a promoter for a tissue specific promoter or a transcription binding factor domain with that of a second transcription factor binding domain. Data from Li et al., Nat. Biotechnol. 30(5):390-392 (2012) demonstrate that site directed genomic mutagenesis with artificial TALENs can be used successfully to engineer rice blight resistance.

The present invention provides for affecting the transport of nutrients that interact with sucrose efflux transporters. The interacting nutrient may be a ligand, which may refer to a molecule or a substance that can bind to a protein such as a periplasmic binding protein to form a complex with that protein. The binding of the ligand to the protein may distort or change the shape of the protein, particularly the tertiary and quaternary structures.

In one embodiment, the present invention provides for introducing exogenous nucleic acids encoding a sucrose efflux transporter protein into a plant cell. The introduced exogenous nucleic acids may be intended to be expressed as a mutant protein or wild-type protein. As used herein, an exogenous nucleic acid is a polynucleotide that normally does not exist or occur in the genome of the plant cell. For example, an extra copy of polynucleotide encoding a wild-type efflux transporter would be an exogenous nucleic acid. Of course copies of polynucleotides encoding mutant efflux transporters would also be considered an exogenous nucleic acid. As used herein with respect to proteins and polypeptides, the term “recombinant” may include proteins and/or polypeptides and/or peptides that are produced or derived by genetic engineering, for example by translation in a cell of non-native nucleic acid or that are assembled by artificial means or mechanisms.

The present invention provides for sucrose efflux transporters operably linked with other nucleic acids encoding peptides intended to alter the expression, activity or location of the efflux transporter, such as targeting sequences. As used herein, fusion may refer to nucleic acids and polypeptides that comprise sequences that are not found naturally associated with each other in the order or context in which they are placed according to the present invention. A fusion nucleic acid or polypeptide does not necessarily comprise the natural sequence of the nucleic acid or polypeptide in its entirety. In general, fusion proteins have the two or more segments joined together through normal peptide bonds. Fusion nucleic acids have the two or more segments joined together through normal phosphodiester bonds.

In one embodiment, the present invention provides for decreasing expression of a sucrose efflux transporter post-transcriptionally. In certain embodiments embodiment, antisense technology or RNAi technology can be used to reduce expression of an efflux or influx transporter protein. These techniques are well known. For example, a single-stranded RNA that can hybridize to an mRNA transcript transcribed from an endogenous efflux transporter gene can be introduced into the cell to interfere with translation. Alternatively, dsRNA containing a region of perfect or significant nucleotide sequence identity with an mRNA transcript transcribed from an endogenous efflux transporter gene, and containing the complement thereto, can be introduced into the cell to interfere with translation by inducing RNAi through well-known principles. Alternatively, the plant cell may be contacted with an antibody or fragment directed against the efflux transporter. As used herein, the term dsRNA refers to double-stranded RNA, wherein the dsRNA may be two separate strands or may be a single strand that folds back on itself in a self-complementary fashion to form a hairpin loop. The dsRNA used in the methods and plant cells of the present invention may comprise a nucleotide sequence identical or nearly identical to the nucleotide of a target gene such that expression of the target gene is specifically downregulated. dsRNA may be produced by expression vectors (also referred to as RNAi expression vectors) capable of giving rise to transcripts which form self-complementary dsRNAs, such as hairpin RNAs, or dsRNA formed by separate complementary RNA strands in cells, and/or transcripts which can produce siRNAs in vivo. Vectors may include a transcriptional unit comprising an assembly of (1) genetic element(s) having a regulatory role in gene expression, for example, promoters, operators, or enhancers, operatively linked to (2) a “coding” sequence which is transcribed to produce a double-stranded RNA (two RNA moieties that anneal in the cell to form an siRNA, or a single hairpin RNA which can be processed to an siRNA), and (3) appropriate transcription initiation and termination sequences. The choice of promoter and other regulatory elements generally varies according to the intended host cell. In general, expression vectors of utility in recombinant DNA techniques are often in the form of “plasmids” which refer to circular double stranded DNA loops, which in their vector form are not bound to the chromosome. Specifically in this embodiment, expression of the RNAi constrict or addition of the exogenous DNA/RNA in specific cells that do not typically express the genes, but where the gene is induced by pathogen infection can be used to generate resistance without causing loss of yield or other side effects. Data from Li et al., Plant Cell Rep. 31(5):851-862 (2012) using amiRNA expressed from the Rubisco small subunit promoter demonstrate that rice blight resistance can be obtained with this approach.

The genetic modifications used in the methods of the present invention or present in the plant cells of the present invention may comprise more than one modification. For example, the expression or activity of more than one efflux transporter may be modified according to the methods of the present invention. Alternatively, more than one modification may be performed on a single efflux transporter. For example, a genetic construct encoding a hairpin dsRNA, amiRNA or siRNA may be inserted into a plant cell. The hairpin dsRNA might be designed to reduce expression of an endogenous efflux transporter by designing the nucleotide sequence of the dsRNA to correspond to the 3′ UTR of the endogenous efflux transporter mRNA. Additionally, another genetic construct might be inserted into the same plant cell containing the dsRNA construct, and this additional construct might code for a mutant version of the same efflux transporter, where the mutant version of the efflux transporter is designed not to include a 3′ UTR, e.g., a cDNA, such that the dsRNA would not be able to interfere with the expression of the mutant efflux transporter gene. In this manner, the expression of activity of the endogenous (or normal) sucrose efflux transporter would be reduced in the genetically modified plant cell compared to an unmodified plant cell.

Similarly, in one embodiment of the present invention, a genetic construct encoding a hairpin dsRNA may be inserted into a plant cell. The hairpin dsRNA might be designed to reduce expression of an endogenous efflux transporter by designing the nucleotide sequence of the dsRNA to correspond to the 3′-UTR of the endogenous efflux transporter mRNA. Additionally, another genetic construct might be inserted into the same plant cell containg the dsRNA construct, and this additional construct might code for a normal version of the same efflux transporter, except that the promoter driving expression of the exogenous copy of the efflux transporter gene would be replaced with a promoter that the pathogen is not be able to manipulate. The exogenous copy of the efflux transporter gene with the “mismatched” promoter may or may not be designed to exclude a 3′ UTR, e.g., a cDNA, such that the dsRNA would not be able to interfere with the expression of the exogenous efflux transporter gene. In this manner, the expression of activity of the endogenous (or normal) sucrose efflux transporter would be reduced in the genetically modified plant cell compared to an unmodified plant cell.

The present invention provides for methods of altering the expression or functioning of a sucrose efflux transporter, either in the transporter itself or in regulatory element within the gene of the transporter.

A transporter may be isolated. As used herein, the term isolated refers to molecules separated from other cell/tissue constituents (e.g. DNA or RNA) that are present in the natural source of the macromolecule. The term isolated may also refer to a nucleic acid or peptide that is substantially free of cellular material, viral material, and culture medium when produced by recombinant DNA techniques, or that is substantially free of chemical precursors or other chemicals when chemically synthesized. Moreover, an isolated nucleic acid may include nucleic acid fragments, which are not naturally occurring as fragments and would not be found in the natural state.

An expression vector is one into which a desired nucleic acid sequence may be inserted by restriction and ligation such that it is operably joined or operably linked to regulatory sequences and may be expressed as an RNA transcript. Expression refers to the transcription and/or translation of an endogenous gene, transgene or coding region in a cell.

A coding sequence and regulatory sequences are operably joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript might be translated into the desired protein or polypeptide.

Vectors may further contain one or more promoter sequences. A promoter may include an untranslated nucleic acid sequence usually located upstream of the coding region that contains the site for initiating transcription of the nucleic acid. The promoter region may also include other elements that act as regulators of gene expression. In further embodiments of the invention, the expression vector contains an additional region to aid in selection of cells that have the expression vector incorporated. The promoter sequence is often bounded (inclusively) at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains responsible for the binding of RNA polymerase. Eukaryotic promoters will often, but not always, contain “TATA” boxes and “CAT” boxes. Activation of promoters may be specific to certain cells or tissues, for example by transcription factors only expressed in certain tissues, or the promoter may be ubiquitous and capable of expression in most cells or tissues.

A promoter also optionally includes distal enhancer or repressor elements, which can be located as much as several thousand base pairs from the start site of transcription. A constitutive promoter is a promoter that is active under most environmental and developmental conditions. An inducible promoter is a promoter that is active under environmental or developmental regulation. Any inducible promoter can be used, see, e.g., Ward et al. Plant Mol. Biol. 22:361-366, 1993. Exemplary inducible promoters include, but are not limited to, that from the ACEI system (responsive to copper) (Meft et al. Proc. Natl. Acad. Sci. USA 90:4567-4571, 1993; In2 gene from maize (responsive to benzenesulfonamide herbicide safeners) (Hershey et al. Mol. Gen. Genetics 227:229-237, 1991, and Gatz et al. Mol. Gen. Genetics 243:32-38, 1994) or Tet repressor from Tn10 (Gatz et al. Mol. Gen. Genetics 227:229-237, 1991). The inducible promoter may respond to an agent foreign to the host cell, see, e.g., Schena et al. PNAS 88: 10421-10425, 1991.

In one embodiment, the modified sucrose efflux transporters of the present invention may function properly in at least one tissue and may function improperly in at least one tissue. For example, introducing a modified efflux transporter with a tissue specific promoter may provide for modified efflux transporter expression in particular tissues (e.g. leaf), leaving a functioning endogenous copy of an efflux transporter in other tissues (e.g. root).

It is known in the art that expression of a gene can be regulated through the presence of a particular promoter upstream (5′) of the coding nucleotide sequence. Tissue specific promoters for directing expression in plants are known in the art. For example, promoters that direct expression in the roots, seeds, or fruits are known. The promoter may be tissue-specific or tissue-preferred promoters. A tissue specific promoter assists to produce the modified efflux transporter transporter exclusively, or preferentially, in a specific tissue. Any tissue-specific or tissue-preferred promoter can be utilized. In plant cells, for example but not by way of limitation, tissue-specific or tissue-preferred promoters include, a root-preferred promoter such as that from the phaseolin gene (Murai et al. Science 23: 476-482, 1983, and Sengupta-Gopalan et al. PNAS 82: 3320-3324, 1985); a leaf-specific and light-induced promoter such as that from cab or rubisco (Simpson et al. EMBO J. 4(11): 2723-2729, 1985, and Timko et al. Nature 318: 579-582, 1985); an anther-specific promoter such as that from LAT52 (Twell et al. Mol. Gen. Genetics 217: 240-245, 1989); a pollen-specific promoter such as that from Zm13 (Guerrero et al. Mol. Gen. Genetics 244: 161-168, 1993) or a microspore-preferred promoter such as that from apg (Twell et al. Sex. Plant Reprod. 6: 217-224, 1993).

In the alternative, the promoter may or may not be a constitutive promoter. Constitutive promoters include, but are not limited to, promoters from plant viruses such as the 35S promoter from CaMV (Odell et al. Nature 313: 810-812, 1985) and the promoters from such genes as rice actin (McElroy et al. Plant Cell 2: 163-171, 1990); ubiquitin (Christensen et al. Plant Mol. Biol. 12:619-632, 1989, and Christensen et al. Plant Mol. Biol. 18: 675-689, 1992); pEMU (Last et al. Theor. Appl. Genet. 81:581-588, 1991); MAS (Velten et al. EMBO J. 3:2723-2730, 1984) and maize H3 histone (Lepetit et al. Mol. Gen. Genetics 231: 276-285, 1992 and Atanassova et al. Plant Journal 2(3): 291-300, 1992).

Vectors may further contain one or more marker sequences suitable for use in the identification and selection of cells, which have been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase or alkaline phosphatase), and genes which visibly affect the phenotype of transformed or transfected cells, hosts, colonies or plaques. Vectors may be those capable of autonomous replication and expression of the structural gene products present in the DNA segments to which they are operably joined.

The present invention provides for assembling a sucrose efflux transporter with another peptide, typically by fusing different nucleic acids together so that they are operably linked and express a fusion protein or a chimeric protein. As used herein, the term fusion protein or chimeric protein may refer to a polypeptide comprising at least two polypeptides fused together either directly or with the use of spacer amino acids. The fused polypeptides may serve collaborative or opposing roles in the overall function of the fusion protein.

Fusion polypeptides may further possess additional structural modifications not shared with the same organically synthesized peptide, such as adenylation, carboxylation, glycosylation, hydroxylation, methylation, phosphorylation or myristylation. These added structural modifications may be further selected or preferred by the appropriate choice of recombinant expression system. On the other hand, fusion polypeptides may have their sequence extended by the principles and practice of organic synthesis.

The present invention thus provides isolated polypeptides comprising a sucrose efflux transporter fused to additional polypeptides. The additional polypeptides may be fragments of a larger polypeptide. In one embodiment, there are one, two, three, four, or more additional polypeptides fused to the efflux transporter. In some embodiments, the additional polypeptides are fused toward the amino terminus of the efflux transporter protein. In other embodiments, the additional polypeptides are fused toward the carboxyl terminus of the efflux transporter protein. In further embodiments, the additional polypeptides flank the efflux transporter protein. In some embodiments, the nucleic acid molecules encode a fusion protein comprising nucleic acids fused to the nucleic acid encoding the efflux transporter. The fused nucleic acid may encode polypeptides that may aid in purification and/or immunogenicity and/or stability without shifting the codon reading frame of the efflux transporter. In some embodiments, the fused nucleic acid will encode for a polypeptide to aid purification of the efflux transporter. In some embodiments the fused nucleic acid will encode for an epitope and/or an affinity tag. In other embodiments, the fused nucleic acid will encode for a polypeptide that correlates to a site directed for, or prone to, cleavage. In certain embodiments, the fused nucleic acid will encode for polypeptides that are sites of enzymatic cleavage. In further embodiments, the enzymatic cleavage will aid in isolating the efflux transporter protein.

The wild-type or genetically modified sucrose efflux transporters of the present invention may be expressed in any location in the cell, including the cytoplasm, cell surface or subcellular organelles such as the nucleus, vesicles, ER, vacuole, etc. Methods and vector components for targeting the expression of proteins to different cellular compartments are well known in the art, with the choice dependent on the particular cell or organism in which the transporter is expressed. See, for instance, Okumoto et al. PNAS 102: 8740-8745, 2005; Fehr et al. J. Fluoresc. 14: 603-609, 2005. Transport of protein to a subcellular compartment such as the chloroplast, vacuole, peroxisome, glyoxysome, cell wall or mitochondrion or for secretion into the apoplast, may be accomplished by means of operably linking a nucleotide sequence encoding a signal sequence to the 5′ and/or 3′ region of a gene encoding the influx or efflux transporter. Targeting sequences at the 5′ and/or 3′ end of the structural gene may determine during protein synthesis and processing where the encoded protein is ultimately compartmentalized.

The presence of a signal sequence directs a polypeptide to either an intracellular organelle or subcellular compartment or for secretion to the apoplast. The term targeting signal sequence refers to amino acid sequences, the presence of which in an expressed protein targets it to a specific subcellular localization. For example, corresponding targeting signals may lead to the secretion of the expressed sucrose efflux transporter, e.g. from a bacterial host in order to simplify its purification. In one embodiment, targeting of the sucrose efflux transporter may be used to affect the concentration of sucrose in a specific subcellular or extracellular compartment. Appropriate targeting signal sequences useful for different groups of organisms are known to the person skilled in the art and may be retrieved from the literature or sequence data bases.

If targeting to the plastids of plant cells is desired, the following targeting signal peptides can for instance be used: amino acid residues 1 to 124 of Arabidopsis thaliana plastidial RNA polymerase (AtRpoT 3) (Plant Journal 17: 557-561, 1999); the targeting signal peptide of the plastidic Ferredoxin:NADP+ oxidoreductase (FNR) of spinach (Jansen et al. Current Genetics 13: 517-522, 1988) in particular, the amino acid sequence encoded by the nucleotides −171 to 165 of the cDNA sequence disclosed therein; the transit peptide of the waxy protein of maize including or without the first 34 amino acid residues of the mature waxy protein (Klosgen et al. Mol. Gen. Genet. 217: 155-161, 1989); the signal peptides of the ribulose bisphosphate carboxylase small subunit (Wolter et al. PNAS 85: 846-850, 1988; Nawrath et al. PNAS 91: 12760-12764, 1994), of the NADP malat dehydrogenase (Gallardo et al. Planta 197: 324-332, 1995), of the glutathione reductase (Creissen et al. Plant J. 8: 167-175, 1995) or of the R1 protein (Lorberth et al. Nature Biotechnology 16: 473-477, 1998).

Targeting to the mitochondria of plant cells may be accomplished by using the following targeting signal peptides: amino acid residues 1 to 131 of Arabidopsis thaliana mitochondrial RNA polymerase (AtRpoT 1) (Plant Journal 17: 557-561, 1999) or the transit peptide described by Braun (EMBO J. 11: 3219-3227, 1992).

Targeting to the vacuole in plant cells may be achieved by using the following targeting signal peptides: The N-terminal sequence (146 amino acids) of the patatin protein (Sonnewald et al. Plant J. 1: 95-106, 1991) or the signal sequences described by Matsuoka and Neuhaus (Journal of Exp. Botany 50: 165-174, 1999); Chrispeels and Raikhel (Cell 68: 613-616, 1992); Matsuoka and Nakamura (PNAS 88: 834-838, 1991); Bednarek and Raikhel (Plant Cell 3: 1195-1206, 1991) or Nakamura and Matsuoka (Plant Phys. 101: 1-5, 1993).

Targeting to the ER in plant cells may be achieved by using, e.g., the ER targeting peptide HKTMLPLPLIPSLLLSLSSAEF in conjunction with the C-terminal extension HDEL (Haselhoff, PNAS 94: 2122-2127, 1997). Targeting to the nucleus of plant cells may be achieved by using, e.g., the nuclear localization signal (NLS) of the tobacco C2 polypeptide QPSLKRMKIQPSSQP.

Targeting to the extracellular space may be achieved by using e.g. one of the following transit peptides: the signal sequence of the proteinase inhibitor II-gene (Keil et al. Nucleic Acid Res. 14: 5641-5650, 1986; von Schaewen et al. EMBO J. 9: 30-33, 1990), of the levansucrase gene from Erwinia amylovora (Geier and Geider, Phys. Mol. Plant Pathol. 42: 387-404, 1993), of a fragment of the patatin gene B33 from Solanum tuberosum, which encodes the first 33 amino acids (Rosahl et al. Mol Gen. Genet. 203: 214-220, 1986) or of the one described by Oshima et al. (Nucleic Acids Res. 18: 181, 1990).

Additional targeting to the plasma membrane of plant cells may be achieved by fusion to a transporter, preferentially to the sucrose transporter SUT1 (Riesmeier, EMBO J. 11: 4705-4713, 1992). Targeting to different intracellular membranes may be achieved by fusion to membrane proteins present in the specific compartments such as vacuolar water channels (γTIP) (Karlsson, Plant J. 21: 83-90, 2000), MCF proteins in mitochondria (Kuan, Crit. Rev. Biochem. Mol. Biol. 28: 209-233, 1993), triosephosphate translocator in inner envelopes of plastids (Flugge, EMBO J. 8: 39-46, 1989) and photosystems in thylacoids.

Targeting to the golgi apparatus can be accomplished using the C-terminal recognition sequence K(X)KXX where “X” is any amino acid (Garabet, Methods Enzymol. 332: 77-87, 2001

Targeting to the peroxisomes can be done using the peroxisomal targeting sequence PTS I or PTS II (Garabet, Methods Enzymol. 332: 77-87, 2001).

Methods for the introduction of nucleic acid molecules into plants are well-known in the art. For example, plant transformation may be carried out using Agrobacterium-mediated gene transfer, microinjection, electroporation or biolistic methods as it is, e.g., described in Potrykus and Spangenberg (Eds.), Gene Transfer to Plants. Springer Verlag, Berlin, N.Y., 1995. Therein, and in numerous other references available to one of skill in the art, useful plant transformation vectors, selection methods for transformed cells and tissue as well as regeneration techniques are described and can be applied to the methods of the present invention.

The present invention also relates to host cells containing the above-described constructs. The host cell can be a plant cell. The host cell can be stably or transiently transfected with the construct. The polynucleotides may be introduced alone or with other polynucleotides. Such other polynucleotides may be introduced independently, co-introduced or introduced joined to the polynucleotides of the invention. As used herein, a “host cell” is a cell that normally does not contain any of the nucleotides of the present invention and contains at least one copy of the nucleotides of the present invention. Thus, a host cell as used herein can be a cell in a culture setting or the host cell can be in an organism setting where the host cell is part of an organism, organ or tissue.

If a eukaryotic expression vector is employed, then the appropriate host cell would be any eukaryotic cell capable of expressing the cloned sequence. In one embodiment, eukaryotic cells are the host cells.

Introduction of a construct into the host cell can be affected by calcium phosphate transfection, DEAE-dextran mediated transfection, cationic lipid-mediated transfection, electroporation, transduction, infection or other methods.

Other examples of methods of introducing nucleic acids into host organisms take advantage TALEN technology to effectuate site-specific insertion of nucleic actions. TALENs are proteins that have been engineered to cleave nucleic acids at a specific site in the sequence. The cleavage sites of TALENs are extremely customizable and pairs of TALENs can be generated to create double-stranded breaks (DSBs) in nucleic acids at virtually any site in the nucleic acid. See Bogdanove and Voytas, Scienc, 333:1843-1846 (2011), which incorporated by reference herein

Transformants carrying the expression vectors are selected based on the above-mentioned selectable markers. Repeated clonal selection of the transformants using the selectable markers allows selection of stable cell lines expressing the fusion proteins constructs. Increased concentrations in the selection medium allows gene amplification and greater expression of the desired fusion proteins. The host cells containing the recombinant fusion proteins can be produced by cultivating the cells containing the fusion proteins expression vectors constitutively expressing the engineered proteins constructs.

The present invention also relates to methods of producing pathogen-resistant or pathogen-tolerant plant cells. In one embodiment, the methods comprise identifying at least one sucrose efflux transporter wherein the levels of expression or activity of the at least one sucrose efflux transporter are increased in the plant cell in response to an infection of the pathogen as compared to an uninfected plant cell. Subsequently, the plant cell is modified to inhibit the activity or reduce the expression of the at least one identified sucrose efflux transporter, where inhibiting the activity or reducing the expression of the at least one identified sucrose efflux transporter produces the pathogen-resistant or pathogen-tolerant plant cell.

In another embodiment, the methods comprise identifying at least one sucrose efflux transporter wherein the levels of expression or activity of the at least one sucrose efflux transporter are decreased in the plant cell in response to an infection of the pathogen as compared to an uninfected plant cell. Subsequently, the plant cell is modified to increase the activity or the expression of the at least one identified sucrose efflux transporter, where increasing the activity or the expression of the at least one identified sucrose efflux transporter produces the pathogen-resistant plant cell.

Methods of identifying transporters whose expression is decreased or increased in response to exposure to a pathogen are well known in the art. For example, in one embodiment, plant cells are co-cultured with a pathogen and an expression array is performed on RNA isolated from the plant cells. RNA-seq or an expression array can identify the genes that are upregulated and down regulated in response to the pathogen. Of course, different plant cells and different pathogens can be combined in various assays to identify the appropriate efflux and influx transporters. For example, Wang, Y. et al. MPMIm 18(5):385-396 (2005) discloses microarray analysis of gene expression profiles in response to inoculating plant cells with Rhizobacteria.

In another aspect, the invention provides harvestable parts or plants and methods to propagate material of the transgenic plants according to the invention, which contain transgenic plant cells as described above. Harvestable parts can be in principle any useful part of a plant, for example, leaves, stems, fruit, seeds, seedcoats, roots etc. Propagation material includes, for example, seeds, fruits, cuttings, seedlings, tubers, rootstocks etc.

As used herein, pathogen refers to an organism that utilizes plant nutrients to grow and divide. Pathogens may include pests and parasites, e.g., mycoparasites, mycoplasma-like organism (MLO), a Rickettsia-Like Organism (RLO), bacteria, or molds. The pathogen to which the plant cell is modified to become resistant or tolerant includes but is not limited to bacteria or fungi. Pathogens also include organisms that cause infectious diseases, such as but not limited to fungi, oomycetes, bacteria, protozoa, nematodes and parasitic plants.

As used herein, a plant cell that is pathogen resistant is a plant cell that will not support the growth and/or propagation of a pathogen such that a pathogen will not survive in the plant cell or in the environment or vicinity immediately surrounding the genetically modified plant cell. A plant cell that is pathogen tolerant is a plant cell that, while perhaps being infected with a pathogen, cannot or does not supply enough nutrients to the pathogen such that the pathogen can grow and propagate.

A pathogen may be a gram negative bacteria such as: Agrobacterium tumefaciens, Agrobacterium vitis, Burkholderia solanacearum, Burkholderia caryophylli, Erwinia amylovora, Erwinia carotovora, Pseudomonas savastanoi, Pseudomonas syringae, Xanthomonas axonopodis, Xanthomonas campestris, Xanthomonas hortorumpelargonium, Xanthomonas oryzae, and Xanthomonas transluceus.

A pathogen may be a gram positive bacteria, such as: Clavibacter michiganensis, Rhodococcus fascians, and Streptomyces scabies.

A pathogen may be a phytopathogenic mould such as: Aspiognomonia veneta, Cryphonectria parasitica, Diaporthe perniciosa, Leucostoma cincta, Cochliobolus sativus, Cochliobolus victoriae, Didymella aplanata, Leptosphaeria maculans, Mycosphaerella arachidicola, Mycosphaerella graminicola, Mycosphaerella musicola Phaesphaeria nodorum, Pyrenophora chaetomioides, Pyrenophora gramine, Pyrenophora teres, Venturia inequalis, Blumeria graminis, Leveillula tauric, Podosphaera leucotricha, Sphaerotheca fuliginia, Phakopsora pachyrhizi, Uncinula necator, Aspergillus flavus, Penicillium expansum, Claviceps purpurea, Builts black sclerots, Cibberella fujicuroi, Cibberella zeae, Nectria galligena, Diplocarpon rosae, Drepanopeziza ribis, Mollisia acuformis, Pezicula malicortis, Pseudopezicola tracheiphila, Pseudopeziza medicaginis, Magnaporthe grisea, Taphrina deformans, Taphrina pruni, Alternaria solani, Septoria apiicola, Alternaria sp., Aspergillus sp., Aspergillus flavus (which produce aflatoxin B1), Botryodiplodia sp., Botrytis sp., Cercospora musaeis, Cladosporium sp., Colletotrichum sp., Diaporthe sp., Diplodia sp., Fusarium sp., Fusarium oxysporum var. cubense, Geotrichum sp., Gibberella fujikuroi, Gloeosporium sp., Leptosphaeria maculans, Monilia sp., Nigrospora sp., Penicillium sp., Phomopsis sp., Phytophthora sp., Piricularia oryzae, Sclerotinia, Sclerotinia sclerotiorum, Trichoderma sp., and Venturia sp.

The present invention also provides for disease protection, prevention or reducing the likelihood of a plant acquiring a disease by altering the accessibility of a sucrose efflux transporter to a pathogen or a disease caused by a pathogen. By way of example, the present invention may protect a plant cell or plant against anthracnose, scab, canker, leaf spot, end rot, brown rot, rust, club root, smut, gall, damping off, dollar spot, mildew, e.g. downy mildew, or powdery mildew, blight, e.g. early blight, late blight, fire blight, fairy rings, wilt (e.g. Fusarium wilt), mold (e.g. gray mold), leaf curl, scab (such as potato scab), verticillium wilt, Anthracnose of Trees, Apple Scab, Artillery Fungus, Azalea Gall, Bacterial Spot of Peach, Bacterial Wilt of Cucurbits, Bark Splitting, Bentgrass Deadspot, Black Knot, Blossom End Rot, Botrytis Blight, Botrytis Blight of Peony, Botrytis Blight of Tulip, Brown Patch, Cane Diseases of Brambles, Canker Diseases of Poplar, Cedar Apple Rust, Cenangium Canker, Clubroot of Cabbage, Corn smut, Cytospora Canker of Fruit, Cytospora Canker of Ornamentals, Daylily Rust, Dog Urine Damage, Dogwood Crown Canker, Downy Leafspot of Hickory, Drechslera Leafspot, Dutch Elm Disease, Fairy Ring, Filbert Blight, Forsythia Gall, Garlic Diseases, Gladiolus Scab, Gray Leafspot, Gray Snow Mold, Hawthorn Leaf Blight, Hemlock Twig Rust, Hollyhock Rust, Juniper Tip Blight, Late Blight, Leaf Tatter, Lilac Bacterial Blight, Oak Leaf Blister, Oedema, Orange Berry Rust, Pachysandra Leaf Blight, Peach Leaf Curl, Physiological Leaf Scorch, Slime Molds, Sphaeropsis (Diplodia), Tar Spot, Tree Cankers, Turfgrass Anthracnose, Willow Black Canker, Willow Botryosphaeria, Willow Leaf Rust, Willow Leucostoma Canker, Willow Powdery Mildew, Willow Scab or Winter Injury.

The present invention provides for protection, prevention or reducing the likelihood that a plant or plant cell will acquire an infectious agent by decreasing the sequestration of a sucrose efflux transporter by a pathogen, thereby depriving the pathogen of essential nutrition. By way of example infectious agents include: Verticillium fungi, Phragmidium spp., Streptomyces scabies, Taphrina deformans, Phytophthora, Botrytis, Fusarium, Erwinia, Alternaria, Plasmopara, Sclerotinia, Rhizoctonia, Pythium, Agrobacterium, Ustilago, Plasmodiophora, Monilinia, Pseudomonas, Colletotrichum, Puccinia or Tilletia.

By way of example, bacterial pathogens may belong to Erwinia, Pectobacterium, Pantoea, Agrobacterium, Pseudomonas, Ralstonia, Burkholderia, Acidovorax, Xanthomonas, Clavibacter, Streptomyces, Xylella, Spiroplasma, Phytoplasma and Aspergillus. Nematode pathogens may include Root knot (Meloidogyne spp.); Cyst (Heterodera and Globodera spp.); Root lesion (Pratylenchus spp.); Spiral (Helicotylenchus spp.); Burrowing (Radopholus similis); Bulb and stem (Ditylenchus dipsaci); Reniform (Rotylenchulus reniformis); Dagger (Xiphinema spp.); and Bud and leaf (Aphelenchoides spp.). Parasitic plants may include: Striga, Phoradendron, dwarf mistletoe (Ar-ceuthobium spp.) and dodder (Cuscuta spp.). Broomrape (Orobanche spp.). Examples of molds include slime mold on turfgrass such as either the genera Mucilaga or Physarum.

By way of example, the present invention provides for protection from: Stem rust by Puccinia graminis tritici; Leaf rust by Puccinia recondite; Powdery mildew by Erysiphe graminis tritici; Septoria leaf blotch by Stagonospora nodorum or Septoria nodorum, Stagonospora (Septoria) avenae f. sp. triticea, and Septoria tritici; Spot blotch by Cochliobolus sativus or Helminthosporium sativum; Tan spot by Pyrenophora tritici-repentis; Bacterial blight by Xanthomonas translucens pv. translucens or X. campestris pv. Translucens; Bacterial leaf blight by Pseudomonas syringae pv. Syringae; Heat canker; black point by Cochliobolus sativus or Helminthosporium sativum or related fungi; Ergot by Claviceps purpurea; Glume blotch by Stagonospora nodorum or Septoria nodorum; Loose smut by Ustilago tritici; Scab (head blight) by Fusarium sp. (Gibberella zeae); Asian soy rust by Phakopsora pachyrhizi; Stinking smut (bunt) by Tilletia foetida or Tilletia caries; Basal glume rot by Pseudomonas syringae pv. Atrofaciens; Black chaff by Xanthomonas translucens pv. Translucens; Bacterial pink seed by Erwinia rhapontici; Common root rot by Cochliobolus sativus or Helminthosporium sativum; Snow rot and snow mold by Pythium and Fusarium spp.; and Take-all by Gaeumannomyces graminis tritici.

By way of example the crop may be barley. Barley diseases include but are not limited to, Stem rust by Puccinia graminis tritici and Puccinia graminis secalis; Leaf rust by Puccinia hordei; Net blotch by Pyrenophora teres; Powdery mildew by Erysiphe graminis hordei; Scald by Rhynchosporium secalis; Septoria leaf blotch by Stagonospora avenae f. sp. triticea and Septoria passerinii; Spot blotch by Cochliobolus sativus or Helminthosporium sativum; Bacterial blight by Xanthomonas translucens pv. translucens Synonym X. campestris pv. Translucens; Black or semi-loose smut by Ustilago nigra; Covered smut by Ustilago hordei; Black point by Cochliobolus sativus or Helminthosporium sativum or related fungi; Ergot by Claviceps purpurea; Glume blotch by Stagonospora nodorum or Septoria nodorum; Loose smut by Ustilago nuda; Scab (head blight) by Fusarium spp. (Gibberella zeae); Bacterial kernel blights by Pseudomonas syringae pathovars; Black chaff by Xanthomonas translucens pv. Translucens; Common root rot by Cochliobolus sativus or Helminthosporium sativum; and, Take-all by Gaeumannomyces graminis tritici;

By way of example oat diseases include but are not limited to, Stem rust by Puccinia graminis avenae; Crown rust or leaf rust by Puccinia coronate; Bacterial stripe blight by Pseudomonas striafaciens; Black loose smut by Ustilago avenae; Covered smut by Ustilago kolleri; Scab (head blight) by Fusarium spp. (Gibberella zeae); and, Blast by Physiologic disorder;

By way of example, rye diseases include but are not limited to, Stem rust by Puccinia graminis secalis; Leaf rust or brown rust by Puccinia recondita secalis; Tan spot by Pyrenophora tritici-repentis; Ergot by Claviceps purpurea; Scab (head blight) by Fursarium spp. (Gibberella zeae); and, Common root rot and other fungi by Helminthosporium sativum and other fungi.

By way of example, corn disease include but are not limited to, Crazy top by Sclerophthora macrospora; Eyespot by Kabatiella zeae; Northern leaf blight by Helminthosporium turcicum; Rust by Puccinia sorghi; Holcus spot by Pseudomonas syringae; Common Smut by Ustilago maydis; Ear rot by Fusarium moniliforme or Fusarium graminearum; Gibberella stalk rot by Gibberella zeae; Diplodia stalk and ear rot by Diplodia maydis; and, Head smut by Sphacelotheca reiliana.

By way of example, diseases to beans include but are not limited to, Rust by Uromyces appendiculatus var. appendiculatus; White mold (sclerotinia rot) by Sclerotinia sclerotiorum; Alternaria blight by Alternaria sp.; Common blight by Xanthomonas campestris pv. Phaseoli; Halo blight by Pseudomonas syringae pv. Phaseolicola; Brown spot by Pseudomonas syringae pv. Syringae; Common blight by Xanthomonas campestris pv. Phaseoli; Halo blight by Pseudomonas syringae pv. Phaseolicola; Brown spot by Pseudomonas syringae pv. Syringae; and, Root rot by Fusarium spp., Rhizoctonia solani, and other fungi.

By way of example diseases to soybean include, but are not limited to, Sclerotinia stem rot (white mold) by Sclerotinia sclerotiorum; Asian soybean rust (ASR) caused by the fungus Phakopsora pachyrhizi; Stem canker by Diaporthe phaseolorum var. caulivora; Pod and stem blight by Diaporthe phaseolorum var. sojae; Brown stem rot by Phialophora gregata or Cephalosporium gregatum; Brown spot by Septoria glycines; Downy mildew by Peronospora manshurica; Bacterial blight by Pseudomonas syringae pv. Glycinea; Iron chlorosis by Iron deficiency; Pod and stem blight by Diaporthe phaseolorum var. sojae; Purple stain by Cercospora kikuchii; Fusarium root rot by Fusarium spp.; Phytophthora root rot by Phytophthora sojae; Pythium root rot by Pythium spp.; Rhizoctonia root rot by Rhizoctonia solani; and, Soybean cyst nematode by Heterodera glycines.

By way of example canola (rapeseed) and mustard diseases include but are not limited to, Sclerotinia Stem Rot by Sclerotinia sclerotiorum; Alternaria black spot by Alternaria brassicae and A. raphani; White rust by Albugo candida; Blackleg by Leptosphaeria maculans; Downy mildew by Peronospora parasitica; and, Aster yellows by Aster yellows mycoplasm.

By way of example sunflower diseases include but are not limited to, Downy mildew by Plasmopara halstedii; Rust by Puccinia helianthi; Sclerotinia stalk and head rot (white mold) by Sclerotinia sclerotiorum; Verticillium wilt by Verticillium dahlia; Phoma black stem by phoma macdonaldii; Phomopsis stem canker by phomopsis or diaporthe) helianthi; Alternaria leaf and stem spot by Alternaria zinniae and Alternaria helianthi; Septoria leaf spot by Septoria helianthi; Apical chlorosis by Pseudomonas tagetis; Rhizopus head rot by Rhizopus spp.; and, Botrytis head rot by Botrytis cinerea.

By way of example potato diseases include but are not limited to, Soft rot by Erwinia carotovora; RING ROT by Clavibacter sepedonicum; Fusarium dry rot by Fusarium sambucinum or F. sulphureum; Silver scurf by Helminthosporium solani; Blackleg by Erwinia carotovora; Scurf & black canker by Rhizoctonia solani; Early blight by Alternaria solani; Late blight by Phytophthora infestans; Verticillium wilt by Verticillium albo-atrum and V. dahlia; and, Purple top by Aster yellows mycoplasma.

By way of example sugarbeet diseases include, but are not limited to, Bacterial leafspot by Pseudomonas syringae; Cercospora leafspot by Cercospora beticola; sugarbeet powdery mildew by Erysiphe betae; Rhizoctonia root and crown rot by Rhizoctonia solani; and Aphanomyces root rot by Aphonomyces cochlioides.

The present invention also provides methods to prevent accumulation of toxic compounds in a plant cell or plant by controlling pathogen infection. For example inhibiting a pathogen from inducing a host plant to provide a nutrient, specifically a carbohydrate such as sucrose, to the pathogen will prevent accumulation of toxins in crops. By way of further example, Aflatoxin is a term generally used to refer to a group of extremely toxic chemicals produced by two molds, Aspergillus flavus and A. parasiticus. The toxins can be produced when these molds, or fungi, attack and grow on certain plants and plant products.

By way of example, and not as limitation, the pathogen may cause a bacterial disease, which include but are not limited to Bacterial leaf blight (Pseudomonas syringae including subsp. syringae); bacterial mosaic (Clavibacter michiganensis including subsp. tessellarius); Bacterial sheath rot (Pseudomonas fuscovaginae); Basal glume rot (Pseudomonas syringae pv. atrofaciens); Black chaff or bacterial streak (Xanthomonas campestris pv. translucens); Pink seed (Erwinia rhapontici); Spike blight or gummosis (Rathayibacter tritici or Clavibacter tritici, Clavibacter iranicus). The bacterial disease may include Bacterial blight (Pseudomonas amygdali pv. glycinea); Bacterial pustules (Xanthomonas axonopodis pv. glycines or Xanthomonas campestris pv. glycines); Bacterial tan spot (Curtobacterium flaccumfaciens pv. flaccumfaciens or Corynebacterium flaccumfaciens pv. flaccumfaciens); Bacterial wilt (Curtobacterium flaccumfaciens pv. flaccumfaciens); Ralstonia solanacearum or Pseudomonas solanacearum); or Wildfire (Pseudomonas syringae pv. tabaci).

The bacterial diseases include but are not limited to Gumming disease (Xanthomonas campestris pv. vasculorum); Leaf scald (Xanthomonas albilineans); Mottled stripe (Herbaspirillum rubrisubalbicans); Ratoon stunting disease (Leifsonia xyli subsp. xyli); and Red stripe (top rot) (Acidovorax avenae). By further way of example, bacterial pathogens include but are not limited to Bacterial wilt or brown rot (Ralstonia solanacearum or Pseudomonas solanacearum); Blackleg and bacterial soft rot (Pectobacterium carotovorum subsp. Atrosepticum or Erwinia carotovora subsp. Atroseptica or Pectobacterium carotovorum subsp. Carotovorum or E. carotovora subsp. Carotovora or Pectobacterium chrysanthemi or E. chrysanthemi or Dickeya solani); Pink eye (Pseudomonas fluorescens); Ring rot (Clavibacter michiganensis subsp. Sepedonicus or Corynebacterium sepedonicum); Common scab (Streptomyces scabiei or S. scabies or Streptomyces acidiscabies or Streptomyces turgidiscabies); Zebra chip or Psyllid yellows (Candidatus Liberibacter solanacearum); Bacterial streak or black chaff (Xanthomonas campestris pv. Translucens); Halo blight (Pseudomonas coronafaciens pv. Coronafaciens); Bacterial blight (halo blight) (Pseudomonas coronafaciens pv. Coronafaciens); Bacterial stripe blight (Pseudomonas coronafaciens pv. Striafaciens); Black chaff and bacterial streak (stripe) (Xanthomonas campestris pv. Translucens); Bacterial blight (Xanthomonas campestris pv. malvacearum); Crown gall (Agrobacterium tumefaciens); and Lint degradation (Erwinia herbicola or Pantoea agglomerans).

By way of example, and not as limitation, the pathogen may cause a fungal disease, which include but are not limited to Alternaria leaf blight (Alternaria triticina); Anthracnose (Colletotrichum graminicola or Glomerella graminicola [teleomorph]); Ascochyta leaf spot (Ascochyta tritici); Aureobasidium decay (Microdochium bolleyi or Aureobasidium bolleyi); Black head molds or sooty molds (Alternaria spp., Cladosporium spp., Epicoccum spp., Sporobolomyces spp. and Stemphylium spp.); Black point or kernel smudge; Cephalosporium stripe (Hymenula cerealis or Cephalosporium gramineum); Common bunt or stinking smut (Tilletia tritici or Tilletia caries or Tilletia laevis or Tilletia foetida); Common root rot (Cochliobolus sativus [teleomorph], Bipolaris sorokiniana [anamorph], or Helminthosporium sativum); Cottony snow mold (Coprinus psychromorbidus); Crown rot or foot rot, seedling blight, dryland root rot (Fusarium spp., Fusarium pseudograminearum, Gibberella zeae, Fusarium graminearum Group II [anamorph], Gibberella avenacea, Fusarium avenaceum [anamorph], or Fusarium culmorum); Dilophospora leaf spot or twist (Dilophospora alopecuri); Downy mildew or crazy top (Sclerophthora macrospora); Dwarf bunt (Tilletia controversa); Ergot (Claviceps purpurea or Sphacelia segetum [anamorph]); Eyespot or foot rot or strawbreaker (Tapesia yallundae, Ramulispora herpotrichoides [anamorph], or Pseudocercosporella herpotrichoides (W-pathotype), Tapesia acuformis; Ramulispora acuformis [anamorph], or Pseudocercosporella herpotrichoides including var. acuformis R-pathoytpe); False eyespot (Gibellina cerealis); Flag smut (Urocystis agropyri); Foot rot or dryland foot rot (Fusarium spp.); Halo spot (Pseudoseptoria donacis or Selenophoma donacis); Karnal bunt or partial bunt (Tilletia indica or Neovossia indica); Leaf rust or brown rust (Puccinia triticina, Puccinia recondita f. sp. tritici, Puccinia tritici-duri); Leptosphaeria leaf spot (Phaeosphaeria herpotrichoides or Leptosphaeria herpotrichoides or Stagonospora sp. [anamorph]); Loose smut (Ustilago tritici or Ustilago segetum var. tritici, Ustilago segetum var. nuda, Ustilago segetum var. avenae); Microscopica leaf spot (Phaeosphaeria microscopica or Leptosphaeria microscopica); Phoma spot (Phoma spp., Phoma glomerata, Phoma sorghina or Phoma insidiosa); Pink snow mold or Fusarium patch (Microdochium nivale or Fusarium nivale or Monographella nivalis [teleomorph]); Platyspora leaf spot (Clathrospora pentamera or Platyspora pentamera); Powdery mildew (Erysiphe graminis f. sp. tritici, Blumeria graminis, Erysiphe graminis, or Oidium monilioides [anamorph]); Pythium root rot (Pythium aphanidermatum, Pythium arrhenomanes, Pythium graminicola, Pythium myriotylum or Pythium volutum); Rhizoctonia root rot (Rhizoctonia solani); Thanatephorus cucumeris [teleomorph]); Ring spot or Wirrega blotch (Pyrenophora seminiperda, Drechslera campanulata or Drechslera wirreganensis); Scab or head blight (Fusarium spp., Gibberella zeae, Fusarium graminearum Group II [anamorph]; Gibberella avenacea, Fusarium avenaceum [anamorph], Fusarium culmorum, Microdochium nivale, Fusarium nivale, or Monographella nivalis [teleomorph]); Sclerotinia snow mold or snow scald (Myriosclerotinia borealis or Sclerotinia borealis); Sclerotium wilt or Southern blight (Sclerotium rolfsii or Athelia rolfsii [teleomorph]); Septoria blotch (Septoria tritici or Mycosphaerella graminicola [teleomorph]); Sharp eyespot (Rhizoctonia cerealis or Ceratobasidium cereale [teleomorph]); Snow rot (Pythium spp., Pythium aristosporum, Pythium iwayamae or Pythium okanoganense); Southern blight or Sclerotium base rot (Sclerotium rolfsii or Athelia rolfsii [teleomorph]); Speckled snow mold or gray snow mold or Typhula blight (Typhula idahoensis, Typhula incarnata, Typhula ishikariensis or Typhula ishikariensis var. canadensis); Spot blotch (Cochliobolus sativus [teleomorph], Bipolaris sorokiniana [anamorph] or Helminthosporium sativum); Stagonospora blotch (Phaeosphaeria avenaria f. sp. triticae, Stagonospora avenae f. sp. triticae [anamorph], Septoria avenae f. sp. triticea, Phaeosphaeria nodorum, Stagonospora nodorum [anamorph] or Septoria nodorum); Stem rust or black rust (Puccinia graminis, or Puccinia graminis f. sp. tritici (Ug99)); Storage molds (Aspergillus spp. or Penicillium spp.); Stripe rust or yellow rust (Puccinia striiformis or Uredo glumarum [anamorph]); Take-all (Gaeumannomyces graminis var. tritici, Gaeumannomyces graminis var. avenae); Tan spot or yellow leaf spot, red smudge (Pyrenophora tritici-repentis or Drechslera tritici-repentis [anamorph]); Tar spot (Phyllachora graminis or Linochora graminis [anamorph]); or Wheat Blast (Magnaporthe grisea); Zoosporic root rot (Lagena radicicola, Ligniera pilorum, Olpidium brassicae, Rhizophydium graminis). The fungal disease may also include Alternaria leaf spot (Alternaria spp.); Anthracnose (Colletotrichum truncatum, Colletotrichum dematium f. truncatum, Glomerella glycines or Colletotrichum destructivum [anamorph]); Black leaf blight (Arkoola nigra); Black root rot (Thielaviopsis basicola or Chalara elegans [synanamorph]); Brown (Septoria glycines or Mycosphaerella usoenskajae [teleomorph]); Brown stem rot (Phialophora gregata or Cephalosporium gregatum); Charcoal rot (Macrophomina phaseolina); Choanephora leaf blight (Choanephora infundibuliferam or Choanephora trispora); Damping-off (Rhizoctonia solani, Thanatephorus cucumeris [teleomorph], Pythium aphanidermatum, Pythium debaryanum, Pythium irregulare, Pythium myriotylum or Pythium ultimum); Downy mildew (Peronospora manshurica); Drechslera blight (Drechslera glycines); Frogeye leaf spot (Cercospora sojina); Fusarium root rot (Fusarium spp.); Leptosphaerulina leaf spot (Leptosphaerulina trifolii); Mycoleptodiscus root rot (Mycoleptodiscus terrestris); Neocosmospora stem rot (Neocosmospora vasinfecta or Acremonium spp. [anamorph]); Phomopsis seed decay (Phomopsis spp.); Phytophthora root and stem rot (Phytophthora sojae); Phyllosticta leaf spot (Phyllosticta sojaecola); Phymatotrichum root rot or cotton root rot (Phymatotrichopsis omnivora or Phymatotrichum omnivorum); Pod and stem blight (Diaporthe phaseolorum or Phomopsis sojae [anamorph]); Powdery mildew (Microsphaera diffusa); Purple seed stain (Cercospora kikuchii); Pyrenochaeta leaf spot (Pyrenochaeta glycines); Pythium rot (Pythium aphanidermatum or Pythium debaryanum or Pythium irregulare or Pythium myriotylum or Pythium ultimum); Red crown rot (Cylindrocladium crotalariae or Calonectria crotalariae [teleomorph]); Red leaf blotch or Dactuliophora leaf spot (Dactuliochaeta glycines, Pyrenochaeta glycines or Dactuliophora glycines [synanamorph]); Rhizoctonia aerial blight (Rhizoctonia solani or Thanatephorus cucumeris [teleomorph]); Rhizoctonia root and stem rot (Rhizoctonia solani); Rust (Phakopsora pachyrhizi); Scab (Spaceloma glycines); Sclerotinia stem rot (Sclerotinia sclerotiorum); Southern blight (damping-off and stem rot) or Sclerotium blight (Sclerotium rolfsii or Athelia rolfsii [teleomorph]); Stem canker (Diaporthe phaseolorum or Diaporthe phaseolorum var. caulivora or Phomopsis phaseoli [anamorph]); Stemphylium leaf blight (Stemphylium botryosum or Pleospora tarda [teleomorph]); Sudden death syndrome (Fusarium solani f. sp. glycines); Target spot (Corynespora cassiicola); or Yeast spot (Nematospora coryli).

By way of example, fungal diseases also include but are not limited to Anthracnose (Colletotrichum graminicola or Glomerella graminicola [teleomorph]); Blast; Downy mildew (Sclerophthora macrospora); Ergot (Claviceps purpurea or Sphacelia segetum [anamorph]); Fusarium foot rot (Fusarium culmorum); Head blight (Bipolaris sorokiniana or Cochliobolus sativus [teleomorph] or Drechslera avenacea or Fusarium graminearum or Gibberella zeae [teleomorph] or Fusarium spp.); Leaf blotch and crown rot (Helminthosporium leaf blotch) (Drechslera avenacea or Helminthosporium avenaceum or Drechslera avenae or Helminthosporium avenae or Pyrenophora avenae [teleomorph]); Powdery mildew (Erysiphe graminis f. sp. avenae or Erysiphe graminis or Oidium monilioides [anamorph]); Rhizoctonia root rot (Rhizoctonia solani or Thanatephorus cucumeris [teleomorph]); Root rot (Bipolaris sorokiniana or Cochliobolus sativus [teleomorph] or Fusarium spp. or Pythium spp. or Pythium debaryanum or Pythium irregular or Pythium ultimum); Rust, crown (Puccinia coronate); Rust, stem (Puccinia graminis); Seedling blight (Bipolaris sorokiniana or Cochliobolus sativus [teleomorph] or Drechslera avenae or Fusarium culmorum or Pythium spp. or Rhizoctonia solani); Sharp eyespot (Rhizoctonia cerealis or Ceratobasidium cereale [teleomorph]); Smut, covered (Ustilago segetum or Ustilago kolleri); Smut, loose (Ustilago avenae); Snow mold, pink (Fusarium patch) (Microdochium nivale or Fusarium nivale or Monographella nivalis [teleomorph]); Snow mold, speckled or gray (Typhula blight) (Typhula idahoensis or Typhula incarnate or Typhula ishikariensis); Speckled blotch (Septoria blight) (Stagonospora avenae or Septoria avenae or Phaeosphaeria avenaria [teleomorph]); Take-all (white head) (Gaeumannomyces graminis var. avenae or Gaeumannomyces graminis); Victoria blight (Bipolaris victoriae or Cochliobolus victoriae [teleomorph]).

By way of further example, fungal diseases include but are not limited to, Black dot (Colletotrichum coccodes or Colletotrichum atramentarium); Brown spot and Black pit (Alternaria alternate or Alternaria tenuis); Cercospora leaf blotch (Mycovellosiella concors or Cercospora concors or Cercospora solani or Cercospora solani-tuberosi); Charcoal rot (Macrophomina phaseolina or Sclerotium bataticola); Choanephora blight (Choanephora cucurbitarum); Common rust (Puccinia pittieriana); Deforming rust (Aecidium cantensis); Early blight (Alternaria solani); Fusarium dry rot (Fusarium spp. or Gibberella pulicaris or Fusarium solani or Fusarium avenaceum or Fusarium oxysporum or Fusarium culmorum or Fusarium acuminatum or Fusarium equiseti or Fusarium crookwellense); Fusarium wilt (Fusarium spp. or Fusarium avenaceum or Fusarium oxysporum or Fusarium solani f. sp. eumartii); Gangrene (Phoma solanicola f. foveata or Phoma foveata or Phoma exigua var. foveata or Phoma exigua f. sp. Foveata or Phoma exigua var. exigua); Gray mold (Botrytis cinerea); Late blight (Phytophthora infestans); Leak (Pythium spp. or Pythium ultimum var. ultimum or Pythium debaryanum or Pythium aphanidermatum or Pythium deliense); Phoma leaf spot (Phoma andigena var. andina); Pink rot (Phytophthora spp. or Phytophthora cryptogea or Phytophthora drechsleri or Phytophthora erythroseptica or Phytophthora megasperma or Phytophthora nicotianae var. parasitica); Powdery mildew (Erysiphe cichoracearum); Powdery scab (Spongospora subterranea f. sp. subterranean); Rhizoctonia canker and black scurf (Rhizoctonia solani or Thanatephorus cucumeris [teleomorph]); Rosellinia black rot (Rosellinia sp. or Dematophora sp. [anamorph]); Septoria leaf spot (Septoria lycopersici var. malagutii); Silver scurf (Helminthosporium solani); Skin spot (Polyscytalum pustulans); Stem rot (southern blight) (Sclerotium rolfsii or Athelia rolfsii [teleomorph]); Thecaphora smut (Angiosorus solani or Thecaphora solani); Ulocladium blight (Ulocladium atrum); Verticillium wilt (Verticillium albo-atrum or Verticillium dahlia); Wart (Synchytrium endobioticum); and, White mold (Sclerotinia sclerotiorum).

Fungal diseases also include but are not limited to, Anthracnose (Colletotrichum graminicola or Glomerella graminicola [teleomorph]); Black head molds (Alternaria spp. or Cladosporium herbarum or Mycosphaerella tassiana [teleomorph] or Epicoccum spp. or Sporobolomyces spp. or Stemphylium spp.); Black point (Bipolaris sorokiniana or Cochliobolus sativus [teleomorph] or Fusarium spp.); Bunt or stinking smut (Tilletia caries or Tilletia tritici or Tilletia laevis or Tilletia foetida); Cephalosporium stripe (Hymenula cerealis or Cephalosporium gramineum); Common root rot and seedling blight (Bipolaris sorokiniana or Helminthosporium sativum or Cochliobolus sativus [teleomorph]); Cottony snow mold or winter crown rot (Coprinus psychromorbidus); Dilophospora leaf spot (twist) (Dilophospora alopecuri); Dwarf bunt (Tilletia controversa); Ergot (Claviceps purpurea or Sphacelia segetum [anamorph]); Fusarium root rot (Fusarium culmorum); Halo spot (Pseudoseptoria donacis or Selenophoma donacis); Karnal bunt (partial bunt) (Neovossia indica or Tilletia indica); Leaf rust (brown rust) (Puccinia recondite or Aecidium clematidis [anamorph]); Leaf streak (Cercosporidium graminis or Scolicotrichum graminis); Leptosphaeria leaf spot (Phaeosphaeria herpotrichoides or Leptosphaeria herpotrichoides); Loose smut (Ustilago tritici); Pink snow mold (Fusarium patch) (Microdochium nivale or Fusarium nivale or Monographella nivalis [teleomorph]); Powdery mildew (Erysiphe graminis or Pythium root rot or Pythium aphanidermatum or Pythium arrhenomanes or Pythium debaryanum or Pythium graminicola or Pythium ultimum); Scab (Gibberella zeae or Fusarium graminearum [anamorph]); Septoria leaf blotch (Septoria secalis); Septoria tritici blotch (speckled leaf blotch) (Septoria tritici or Mycosphaerella graminicola [teleomorph]); Sharp eyespot and Rhizoctonia root rot (Rhizoctonia cerealis or Ceratobasidium cereale [teleomorph]); Snow scald (Sclerotinia snow mold) (Myriosclerotinia borealis or Sclerotinia borealis); Speckled (or gray) snow mold (Typhula blight) (Typhula idahoensis or Typhula incarnate or Typhula ishikariensis or Typhula ishikariensis var. Canadensis); Spot blotch (Bipolaris sorokiniana); Stagonospora blotch (glume blotch) (Stagonospora nodorum or Septoria nodorum or Phaeosphaeria nodorum [teleomorph] or Leptosphaeria nodorum); Stalk smut (stripe smut) (Urocystis occulta); Stem rust (Puccinia graminis); Storage molds (Alternaria spp. or Aspergillus spp. or Epicoccum spp. or Nigrospora spp. or Penicillium spp. or Rhizopus spp.); Strawbreaker (eyespot or foot rot) (Pseudocercosporella herpotrichoides or Tapesia acuformis [teleomorph]); Stripe rust (yellow rust) (Puccinia striiformis or Uredo glumarum [anamorph]); Take-all (Gaeumannomyces graminis); Tan spot (yellow leaf spot) (Pyrenophora tritici-repentis or Drechslera tritici-repentis [anamorph] or Helminthosporium tritici-repentis).

Fungal diseases also include but are not limited to Alternaria leaf blight (Alternaria tenuissima); Alternaria leaf spot (Alternaria arachidis); Alternaria spot and veinal necrosis (Alternaria alternate); Anthracnose (Colletotrichum arachidis or Colletotrichum dematium or Colletotrichum mangenoti); Aspergillus crown rot (Aspergillus niger); Blackhull (Thielaviopsis basicola or Chalara elegans [synanamorph]); Botrytis blight (Botrytis cinerea or Botryotinia fuckeliana [teleomorph]); Charcoal rot and Macrophomina leaf spot (Macrophomina phaseolina or Rhizoctonia bataticola); Choanephora leaf spot (Choanephora spp.); Collar rot (Lasiodiplodia theobromae or Diplodia gossypina); Colletotrichum leaf spot (Colletotrichum gloeosporioides or Glomerella cingulata [teleomorph]); Cylindrocladium black rot (Cylindrocladium crotalariae or Calonectria crotalariae [teleomorph]); Cylindrocladium leaf spot (Cylindrocladium scoparium or Calonectria kyotensis [teleomorph]); Damping-off, Aspergillus (Aspergillus flavus or Aspergillus niger); Damping-off, Fusarium (Fusarium spp.); Damping-off, Pythium (Pythium spp.); Damping-off, Rhizoctonia (Rhizoctonia spp.); Damping-off, Rhizopus (Rhizopus spp.); Drechslera leaf spot (Bipolaris spicifera or Drechslera spicifera or Cochliobolus spicifer [teleomorph]); Fusarium peg and root rot (Fusarium spp.); Fusarium wilt (Fusarium oxysporum); Leaf spot, early (Cercospora arachidicola or Mycosphaerella arachidis [teleomorph]); Leaf spot, late (Phaeoisariopsis personata or Cercosporidium personatum or Mycosphaerella berkeleyi [teleomorph]); Melanosis (Stemphylium botryosum or Pleospora tarda [teleomorph]); Myrothecium leaf blight (Myrothecium roridum); Olpidium root rot (Olpidium brassicae); Pepper spot and scorch (Leptosphaerulina crassiasca); Pestalotiopsis leaf spot (Pestalotiopsis arachidis); Phoma leaf blight (Phoma microspora); Phomopsis foliar blight (Phomopsis phaseoli or Phomopsis sojae or Diaporthe phaseolorum [teleomorph]); Phomopsis leaf spot (Phomopsis spp.); Phyllosticta leaf spot (Phyllosticta arachidis-hypogaeae or Phyllosticta sojaecola or Pleosphaerulina sojicola [teleomorph]); Phymatotrichum root rot (Phymatotrichopsis omnivore or Phymatotrichum omnivorum); Pod rot (pod breakdown) (Fusarium equiseti or Fusarium scirpi or Gibberella intricans [teleomorph] or Fusarium solani or Nectria haematococca [teleomorph] or Pythium myriotylum or Rhizoctonia solani or Thanatephorus cucumeris [teleomorph]); Powdery mildew (Oidium arachidis); Pythium peg and root rot (Pythium myriotylum or Pythium aphanidermatum or Pythium debaryanum or Pythium irregular or Pythium ultimum); Pythium wilt (Pythium myriotylum); Rhizoctonia foliar blight, peg and root rot (Rhizoctonia solani); Rust (Puccinia arachidis); Scab (Sphaceloma arachidis); Sclerotinia blight (Sclerotinia minor or Sclerotinia sclerotiorum); Stem rot (southern blight) (Sclerotium rolfsii or Athelia rolfsii [teleomorph]); Verticillium wilt (Verticillium albo-atrum or Verticillium dahlia); Web blotch (net blotch) (Phoma arachidicola or Ascochyta adzamethica or Didymosphaeria arachidicola or Mycosphaerella arachidicola); Yellow mold (Aspergillus flavus or Aspergillus parasiticus); Zonate leaf spot (Cristulariella moricola or Sclerotium cinnamomi [syanamorph] or Grovesinia pyramidalis [teleomorph]).

Fungal diseases also include but are not limited to Anthracnose (Glomerella gossypii or Colletotrichum gossypii [anamorph]); Areolate mildew (Ramularia gossypii or Cercosporella gossypii or Mycosphaerella areola [teleomorph]); Ascochyta blight (Ascochyta gossypii); Black root rot (Thielaviopsis basicola or Chalara elegans [synanamorph]); Boll rot (Ascochyta gossypii or Colletotrichum gossypii or Glomerella gossypii [teleomorph] or Fusarium spp. or Lasiodiplodia theobromae or Diplodia gossypina or Botryosphaeria rhodina [teleomorph] or Physalospora rhodina or Phytophthora spp. or Rhizoctonia solani); Charcoal rot (Macrophomina phaseolina); Escobilla (Colletotrichum gossypii or Glomerella gossypii [teleomorph]); Fusarium wilt (Fusarium oxysporum f. sp. vasinfectum); Leaf spot (Alternaria macrospora or Alternaria alternata or Cercospora gossypina or Mycosphaerella gossypina [teleomorph] or Cochliobolus spicifer or Bipolaris spicifera [anamorph] or Curvularia spicifera or Cochliobolus spicifer or Myrothecium roridum or Rhizoctonia solani or Stemphylium solani); Lint contamination (Aspergillus flavus or Nematospora spp. or Nigrospora oryzae); Phymatotrichum root rot or cotton root rot (Phymatotrichopsis omnivora or Phymatotrichum omnivorum); Powdery mildew (Leveillula taurica or Oidiopsis sicula [anamorph] or Oidiopsis gossypii or Salmonia malachrae); Stigmatomycosis (Ashbya gossypii or Eremothecium coryli or Nematospora coryli or Aureobasidium pullulans); Cotton rust (Puccinia schedonnardii); Southwestern cotton rust (Puccinia cacabata); Tropical cotton rust (Phakopsora gossypii); Sclerotium stem and root rot or southern blight (Sclerotium rolfsii or Athelia rolfsii [teleomorph]); Seedling disease complex (Colletotrichum gossypii or Fusarium spp. or Pythium spp. or Rhizoctonia solani or Thanatephorus cucumeris [teleomorph] or Thielaviopsis basicola or Chalara elegans [synanamorph]); Stem canker (Phoma exigua); and Verticillium wilt (Verticillium dahliae).

The fungal disease may also include but are not limited to Banded sclerotial (leaf) disease (Thanatephorus cucumeris or Pellicularia sasakii or Rhizoctonia solani [anamorph]); Black rot (Ceratocystis adiposa or Chalara sp. [anamorph]); Black stripe (Cercospora atrofiliformis); Brown spot (Cercospora longipes); Brown stripe (Cochliobolus stenospilus or Bipolaris stenospila [anamorph]); Downy mildew (Peronosclerospora sacchari or Sclerospora sacchari); Downy mildew, leaf splitting form (Peronosclerospora miscanthi or Sclerospora mischanthi or Mycosphaerella striatiformans); Eye spot (Bipolaris sacchari or Helminthosporium sacchari); Fusarium sett and stem rot (Gibberella fujikuroi or Fusarium moniliforme [anamorph] or Gibberella subglutinans); Iliau (Clypeoporthe iliau or Gnomonia iliau or Phaeocytostroma iliau [anamorph]); Leaf blast (Didymosphaeria taiwanensis); Leaf blight (Leptosphaeria taiwanensis or Stagonospora tainanensis [anamorph]); Leaf scorch (Stagonospora sacchari); Marasmius sheath and shoot blight (Marasmiellus stenophyllus or Marasmius stenophyllus); Myriogenospora leaf binding (tangle top) (Myriogenospora aciculispora); Phyllosticta leaf spot (Phyllosticta hawaiiensis); Phytophthora rot of cuttings (Phytophthora spp. or Phytophthora megasperma); Pineapple disease (Ceratocystis paradoxa or Chalara paradoxa or Thielaviopsis paradoxa [anamorph]); Pokkah boeng (Gibberella fujikuroi or Fusarium moniliforme [anamorph] or Gibberella subglutinans); Red leaf spot (purple spot) (Dimeriella sacchari); Red rot (Glomerella tucumanensis or Physalospora tucumanensis or Colletotrichum falcatum [anamorph]); Red rot of leaf sheath and sprout rot (Athelia rolfsii or Pellicularia rolfsii or Sclerotium rolfsii [anamorph]); Red spot of leaf sheath (Mycovellosiella vaginae or Cercospora vaginae); Rhizoctonia sheath and shoot rot (Rhizoctonia solani); Rind disease (sour rot) (Phaeocytostroma sacchari or Pleocyta sacchari or Melanconium sacchari); Ring spot (Leptosphaeria sacchari or Phyllosticta sp. [anamorph]); Root rot (Marasmius sacchari or Pythium arrhenomanes or Pythium graminicola or Rhizoctonia sp. or Oomycetes); common Rust (Puccinia melanocephala or Puccinia erianthi); Orange Rust (Puccinia kuehnii); Schizophyllum rot (Schizophyllum commune); Sclerophthora disease (Sclerophthora macrospora); Seedling blight (Alternaria alternata or Bipolaris sacchari or Cochliobolus hawaiiensis or Bipolaris hawaiiensis [anamorph] or Cochliobolus lunatus or Curvularia lunata [anamorph] or Curvularia senegalensis or Setosphaeria rostrata or Exserohilum rostratum [anamorph] or Drechslera halodes); Sheath rot (Cytospora sacchari); Smut, culmicolous (Ustilago scitaminea); Target blotch (Helminthosporium sp.); Veneer blotch (Deightoniella papuana); White rash (Elsinoe sacchari or Sphaceloma sacchari [anamorph]); Wilt (Fusarium sacchari or Cephalosporium sacchari); Yellow spot (Mycovellosiella koepkei or Cercospora koepkei); Zonate leaf spot (Gloeocercospora sorghi); Lesion (Pratylenchus spp.); Root-knot (Meloidogyne spp.); Spiral (Helicotylenchus spp. or Rotylenchus spp. or Scutellonema spp.).

The pathogen may be a phytoplasma such as aster yellows phytoplasma, Cowpea mild mottle, Groundnut crinkle, Groundnut eyespot, Groundnut rosette, Groundnut chlorotic rosette, Groundnut green rosette, Groundnut streak, Marginal chlorosis, Peanut clump, Peanut green mosaic, Peanut mottle, Peanut ringspot or bud necrosis, Tomato spotted wilt, Peanut stripe, Peanut stunt, Peanut yellow mottle, Tomato spotted wilt, or Witches' broom.

By way of example nematode pathogens include but are not limited to, Potato cyst nematode, Globodera rostochiensis, Globodera pallid, Lesion nematode, Pratylenchus spp., Pratylenchus brachyurus, Pratylenchus penetrans, Pratylenchus scribneri, Pratylenchus neglectus, Pratylenchus thornei, Pratylenchus crenatus, Pratylenchus andinus, Pratylenchus vulnus, Pratylenchus coffeae, Potato rot nematode, Ditylenchus destructor, Root knot nematode, Meloidogyne spp., Meloidogyne hapla, Meloidogyne incognita, Meloidogyne javanica, Meloidogyne chitwoodi, Sting nematode, Belonolaimus longicaudatus, Stubby-root nematode, Paratrichodorus spp., Trichodorus spp; Heterodera avenae, Ditylenchus dipsaci, Subanguina radicicola, Meloidogyne spp., Anguina tritici, Xiphinema spp., Tylenchorhynchus brevilineatus, Tylenchorhynchus brevicadatus, Criconemella ornate, Macroposthonia ornate, Meloidogyne javanica, Meloidogyne hapla, Meloidogyne arenaria, Pratylenchus brachyurus, Pratylenchus coffeae, Ditylenchus destructor, Scutellonema cavenessi, Belonolaimus glacilis, Belonolaimus longicaudatus, Ditylenchus dipsaci, Heterodera avenae, Heterodera hordecalis, Heterodera latipons, Punctodera chalcoensis, Xiphinema americanum, Pratylenchus spp., Pratylenchus thornei, Pratylenchus spp., Criconemella spp., Nothocriconemella mutabilis, Meloidogyne spp., Meloidogyne chitwoodi, Meloidogyne naasi, Hemicycliophora spp., Helicotylenchus spp., Belonolaimus longicaudatus, Paratrichodorus minor, Quinisulcius capitatus, Tylenchorhynchus spp., and Merlinius spp., Hoplolaimus columbus, Rotylenchulus reniformis, Meloidogyne incognita, Belonolaimus longicaudatus, and Aphelenchoides arachidis.

SWEETs are induced also by beneficial microorganisms such as (but not limited to) mycorrhiza or nitrogen fixing Rhizobia in nodules. Since these organisms depend on adequate supply with energy, regulation of the SWEET activity, up or down, can affect the symbiosis and enhance or reduce flux of nutrients between the two organisms.

SWEETs are critical for phloem loading. Sucrose is transported to phloem parenchyma cells inside the leaf phloem, where it is secreted via a SWEET sucrose transporter. The adjacent sieve element companion cell complex then takes up the sucrose from the extracellular space using a sucrose proton cotransporters of the SUT/SUC family. Because SWEET activity in the leaf can be limiting, upregulation of SWEETs according to any one of the methods disclosed herein can be used to increase flux of sugars towards the other organs, such as but not limited to, seeds. For example, degerulating SWEET promoters, introducing enhancers, replacing the promoter, or introducing an expression vector with a specific promoter can be used to drive the flux of sugars into other organs or portions of the plant, such as but not limited to seeds.

Similar to the leaves, the seed is supplied with sugars by a pair of sugar transporters. In particular, transfer of sugar from the maternal tissue begins with SWEETs on the maternal side vascular endings entering seed coat, release of sugar from seed coat layers, transfer of the sugar through funiculus, uptake of the sugar by SWEETs or SUT/SUCs into endosperm, and subsequent release of the sugar from endosperm and uptake into the developing embryo. SWEETs play critical roles in this process as shown by analysis of expression as well as mutant plants. Because SWEET activity in the leaf can be limiting, upregulation of SWEET expression and/or activity using one the methods of disclosed herein can increase flux of sugars towards the other organs, specifically the seeds.

### EXAMPLES

### Example 1

### Plasmid Constructs—Constructs for Expression in HEK293T Cells

The sucrose sensor FLIPsuc90μΔ1V was excised from the pRSET-B vector using BamHI and HindIII, and ligated into pcDNA3.1(−) (Invitrogen) digested by the same enzymes. (Lager et al. J. Biol. Chem. 281, 30875 (2006)). The potato H+/sucrose transporter StSUT1 gene in the yeast expression vector pDR195 was restricted with NotI and cloned into pcDNA3.1(−), which had been digested with NotI and dephosphorylated by Antarctic phosphatase. (Weise et al. Plant Cell 12, 1345 (2000)). For the screening, candidate ORFs selected from our membrane protein clone collection were transferred into the mammalian expression vector pcDNA3.2/V5-DEST (Invitrogen) using the Gateway™ strategy as described previously. (Lalonde et al. Front. Plant Physiol., 12 (2010), Chen et al. Nature 468, 527 (2010)). All constructs were verified by DNA sequencing.

Constructs for Expression in Xenopus Oocytes

Oocyte expression constructs for OsSWEET11 and 14 and the truncated version of OsSWEET11_F205* have been described previously (Chen et al. Nature 468, 527 (2010)). The ORFs of AtSWEET11 and 12 (with stop codon) in vector pDONR221-f1 were transferred to the oocyte expression vector pOO2-GW as described previously for other SWEETs (Chen et al. Nature 468, 527 (2010)). Non-functional, truncated versions of AtSWEET11-F201* and AtSWEET12-L203* were generated by introducing stop codons in transmembrane helix 7 by site-directed mutagenesis. Primers are listed in the Primer Table. It had previously been shown that mutations that lead to truncation in the 7th transmembrane spanning domain lead to loss of function in plant and human SWEET homologs. (Chen et al. Nature 468, 527 (2010)). The mutants shown here are non-functional, and can be used as controls for transport assays.

Plasmids for Complementation of Mutants

For complementation of the atsweet11;12 (pAtSWEET11:AtSWEET11) double mutant, a 4784 bp genomic sequence consisting of a 2937 bp promoter and 1847 bp of the entire coding region without stop codon from AtSWEET11 was amplified from BAC clone T8P19 (ABRC) using primers AtSWT11attB1 and AtSET11attB2 (cf. primer list below). The genomic AtSWEET11 fragment was cloned into the Gateway donor vector pDONR221-f1 and transferred into the Gateway plant expression vector pGWB1 by LR clonase (Invitrogen). (Chen et al. Nature 468, 527 (2010), Kawai et al. Anal. Chem. 76, 6144 (2004)). A similar strategy was used for generating the AtSWEET12 complementation construct pAtSWEET12:AtSWEET12, which comprises a 1887 bp AtSWEET12 promoter sequence and 1858 bp of the coding region up to but not including the stop codon. The stop codon and 3′-UTR were provided by the binary vector. The proteins produced from these constructs thus contain Gateway sequences at the C-terminus.

GUS and eGFP Fusion Constructs Under Native Promoters

For analyzing the expression of SWEETs via GUS fusions, the same fragments as used for generating the complementation constructs (promoter and gene including introns for AtSWEET11 and 12) were transferred by LR reactions into the plant Gateway vector pMDC163 carrying the GUS gene. (Curtis et al. Plant Physiol. 133, 462 (2003)). The GUS gene was translationally fused to the C-terminus of AtSWEET11 or 12. To generate translational GFP fusion constructs, the pAtSWEET11:AtSWEET11 or pAtSWEET12:AtSWEET12 cassette were re-amplified with the forward primer AtSWT11KpnIF containing a KpnI restriction site and the reverse primer AtSWT11PstIR containing a PstI restriction site and subcloned into the eGFP fusion vector pGTKan3 via KpnI and PstI restriction sites. (Kasaras et al. Plant Biol. 12 Suppl 1, 140 (2010).

eYFP Fusions Under Control of the CaMV 35S Promoter

The ORFs of AtSWEET11 and 12 without stop codon in pDONR221-f1 were cloned into the binary vector pX-YFP-GW by a Gateway LR reaction. (Chen et al. Nature 468, 527 (2010)).

FRET Sucrose Sensor Analysis in HEK293T Cells

The analysis was performed essentially as described using a FRET sucrose sensor instead of a FRET glucose sensor. (Chen et al. Nature 468, 527 (2010), Takanaga et al. FASEB J. 24, 2849 (2010), Hou et al. Nature Protocols 6, in press (2011)). Here, the screening was performed in 96 well plates to increase throughput. Briefly, HEK293T cells were co-transfected with a plasmid carrying the sucrose sensor FLIPsuc90μΔ1V (100 ng) and a plasmid carrying a candidate transporter gene (100 ng) using Lipofectamine 2000 (Invitrogen) in 96-well plates. (Lager et al. J. Biol. Chem. 281, 30875 (2006)). For FRET imaging, the culture medium in each well was replaced with 100 μl Hanks Balanced Saline Salt (HBSS) buffer followed by addition of 100 μl HBSS buffer containing 20 mM sucrose. A Leica inverted fluorescence microscope DM IRE2 with Quant EM camera was used for imaging with SlideBook 4.2 (Intelligent Imaging Innovations) and the following settings: exposure time 200 msec, gain 3, binning 2, and time interval 10 sec. FRET analyses were performed as described. (Hou et al. Nature Protocols 6, in press (2011)).

Tracer Uptake and Tracer Efflux in Xenopus Oocytes

Linearization of the plasmids in pOO2 vector, capped cRNA synthesis, Xenopus oocytes isolation and cRNA injection, [¹⁴C]-labeled sugar uptake and efflux were carried out as described before. (Chen et al. Nature 468, 527 (2010)). For water control, 50 nl RNAse free water instead of any cRNA was injected. For efflux assay, oocytes were injected with 50 nl solution containing 10, 50, 250, 500 or 750 mM sucrose (0.18 μCi μl-1 [¹⁴C(U)] sucrose) or 50 mM maltose (0.18 μCi μl-1 [¹⁴C(U)] maltose).

Plant Material and Growth Conditions

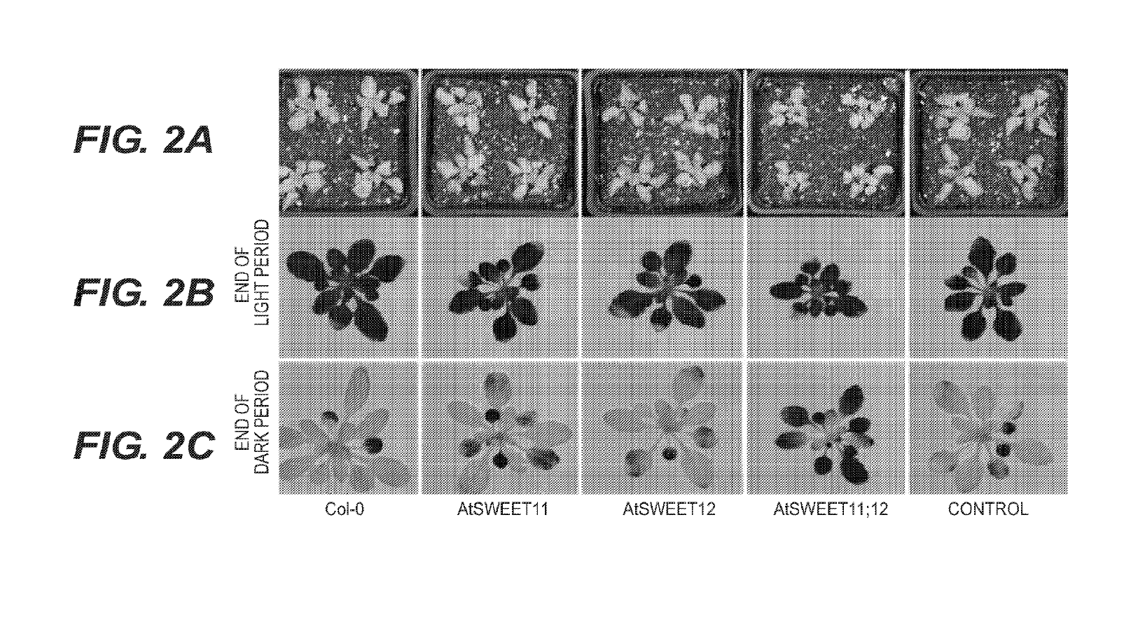

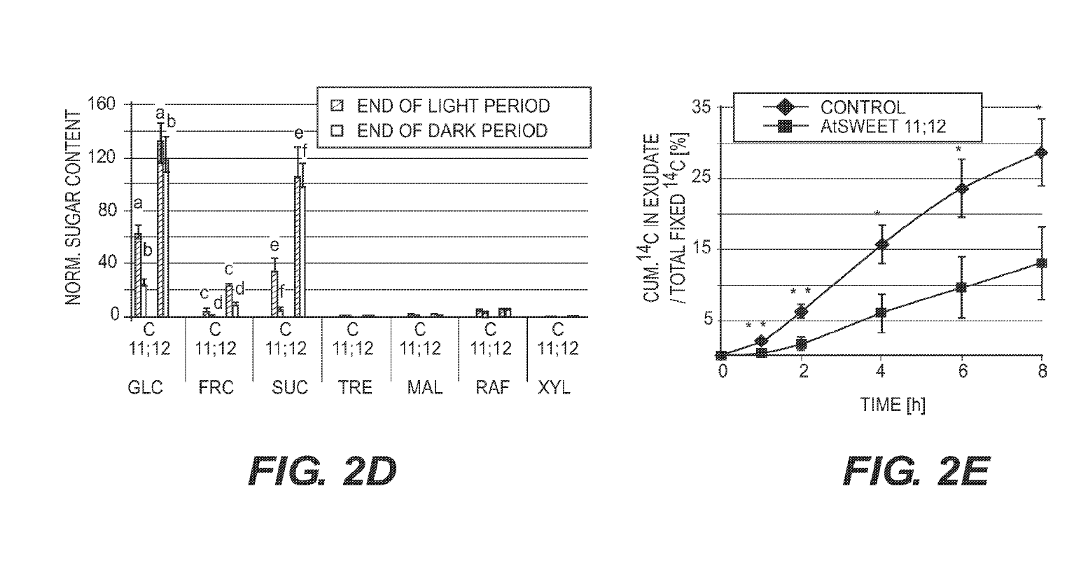

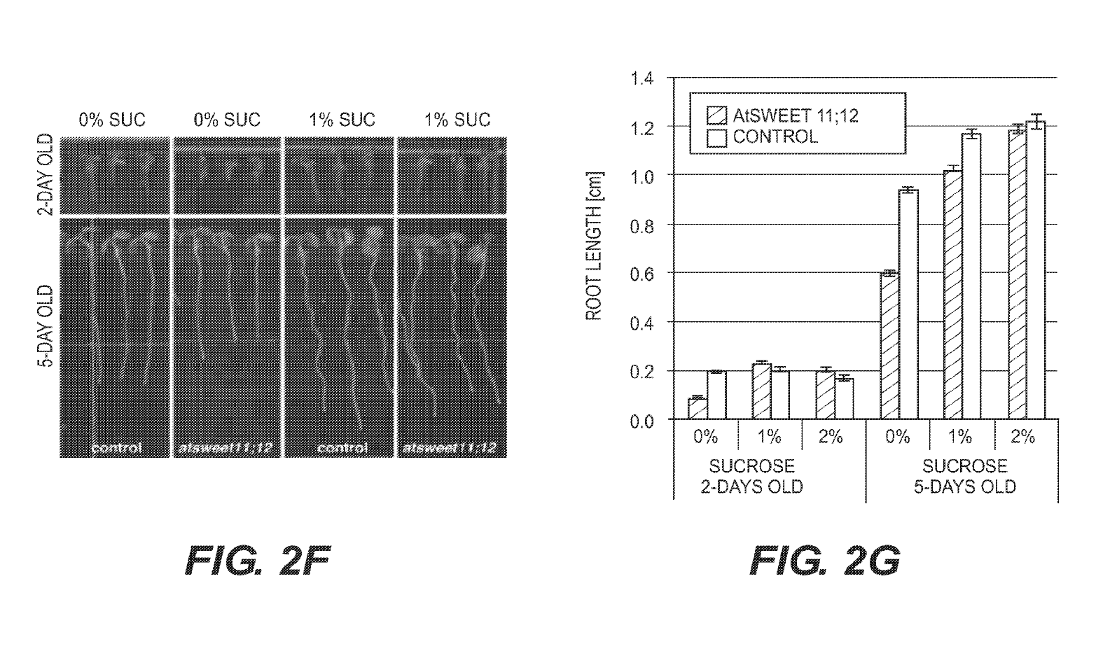

Plants were grown under low light (LL) (90-110 μE m-2 s-1 with 10 hr photoperiod) conditions, or where indicated, transferred to high light (HL) (400-450 μE m-2 s-1 with 16 hr photoperiod). For growth phenotype observation and starch staining, 2-week-old plants were transferred from LL to HL for 1 week (FIGS. 2A, B and C). One day before starch staining or sample collection for metabolomics measurements, three and half week old plants were transferred to HL. Growth chamber temperatures were set at 22° C. during the day and 20° C. during the night. For plastic embedding, GUS transgenic plants were grown in LL conditions.

For seedling growth analysis, seeds were sown on ½ MS medium with or without sucrose (as indicated), then kept at 4° C. for 3 days before transfer to a growth chamber and positioned vertically (16 hr light period). At indicated days post transfer, seedlings were digitally photographed and root length was measured using ImageJ software.

Arabidopsis thaliana wild type Col-0 and AtSWEET11;12 double mutants were transformed by the floral dip method. (Davis et al. Plant Meth 5, 3 (2009)). Transgenic seedlings were selected on media with kanamycin (pAtSWEET11:AtSWEET11-eGFP and pAtSWEET12:AtSWEET12-eGFP), hygromycin (pAtSWEET11:AtSWEET11-GUS, pAtSWEET12:AtSWEET12-GUS, pAtSWEET11:AtSWEET11, and pAtSWEET12:AtSWEET12 in atsweet11;12) or by spraying with glufosinate ammonium (35S:AtSWEET11-eYFP and 35S:AtSWEET11-eYFP).

Genotyping and Transcript Analysis of T-DNA Mutants

Genomic DNA was extracted from Arabidopsis thaliana Col-0, control (wild type lines isogenic to the homozygous double mutant atsweet11;12 (Salk_{—}073269 and Salk_{—}031696 T-DNA insertions)) and the T-DNA insertion lines, and was used as template for PCR amplification of AtSWEET11 or 12 fragments. Primers specific to AtSWEET11 sequences flanking the T-DNA (Salk_{—}073269) insertion site (AtSWT11LP and AtSWT11RP; cf. primer list) and AtSWEET12 sequences flanking the T-DNA (Salk_{—}031696) insertion site (AtSWT12LP and AtSWT12RP) were obtained. The sequence for the left border primer LBb1 was obtained from the SALK Web site (signal.salk.edu/). These primers were used to detect the presence of the T-DNA insert. PCR was performed as described on the SALK Web site.

Total RNA was extracted from leaves of Arabidopsis from Col-0, controls and insertion lines using a Spectrum™ plant total RNA kit (Sigma). First strand cDNA was synthesized using oligo dT and M-MuLV Reverse Transcriptase following the instruction of the supplier (Fermentas). Primers for the full length ORF of AtSWEET11 (AtSWT11FattB1 and AtSWT11attB2) or AtSWEET12 (AtSWT12FattB1 and AtSWT12attB2) were used for RT-PCR to determine the expression levels. AtACTIN2 (Primers: AtACT2F and AtACT2R) served as reference gene. Real-time PCR was carried out as described. (Chen et al. Nature 468, 527 (2010)). To evaluate the possibility of partial transcripts, primers upstream (AtSWT11UPF and AtSWT11UPR) and downstream (AtSWT11DNF and AtSWT11DNR) of the T-DNA inserts were also used for qPCR. The same method was for analyzing AtSWEET12 using primers AtSWT12UPF, AtSWT12UPR, AtSWT12DNF and AtSWT12DNR or AtSWEET13 expression using the primers AtSWT13F and AtSWT13R.

Starch Staining

Whole rosettes of plants were either harvested or covered with black trays in the late afternoon. In the early afternoon of the next day rosettes of covered plants were harvested. Starch staining was performed right after rosette harvest. Samples were cleared in 80% (v/v) ethanol plus 5% (v/v) formic acid at 22 degrees C., stained in KI2 Lugol's iodine solution (43.4 mM KI/5.7 mM) and washed twice in water.

Phloem Exudation

Measurement of phloem exudation from [¹⁴CO₂]-radiolabeled leaves was carried out as described by Srivastava, except for the following modifications. Four to six mature rosette leaves were excised (4 hr into photoperiod) from 4-week-old plants growing in a LL chamber. (Srivastava et al. Plant Physiol. 148, 200 (2008)). The petioles of excised leaves were placed in water in 24-well microtiter plates to keep stomata open and transpiring, and were kept under illumination using a 90 Watt LED light RBO711 (90 Watt UFO LED Grow Light; AIBC International, Ithaca; Red:Blue:Orange 7:1:1) for half an hour before initiating labeling. The distance of the light from the plants was adjusted to obtain a light intensity of 150 μE m-2 s-1. A sealed plastic container was used as the labeling chamber. The 24-well plate was placed in the chamber lied on its one side with a pile water-soaked paper tower to keep high humidity environment. The chamber was covered with two layers of clear plastic wrap bounded with elastic band. A mixture of 30 μl (1 μCi/μl) [¹⁴C]NaHCO₃ (PerkinElmer) and 100 μl 85% lactic acid (EMD Chemicals) in a 1 ml syringe with a 22-gauge needle was send to labeling chamber by pushing the needle into the chamber from side. To make reaction completely, plunger was moved back and forth for several times. Then, 1 ml syringe was replaced with 60 ml syringe, plunger moving was slowly continued until the 20 minute labeling was done. The LED light was turned off right away. Before the leaf were transferred to 24-well plate containing 1 ml 15 mM EDTA each well, the leaf petioles was cut again under the surface of the 15 mM EDTA to prevent sieve plate closed from the new plugs forming. The EDTA solution was collected at the different time points and was replace with fresh EDTA solution. Samples were measured by Scintillation machine after mixed with scintillation cocktail.

GC-MS Metabolite Analysis

Plant materials were prepared for gas chromatography mass spectrometry (GC-MS) and metabolite levels were quantified exactly as described, with the exception that absolute levels were calculated following the calibration method previously described in Roessner-Tunali et al. 2003 (Yeung et al. Science 319, 210 (2008), Oancea et al. Cell Biol. 140, 485 (1998)).

Plastic Embedding and Sectioning

Arabidopsis was grown under LL conditions. Plastic embedding followed the protocol provided with the LR White embedding kit (Sigma). Semi-thin cross sections (3 μm) were cut and stained with 0.1% (w/v) Safranin O, washed three times with distilled water and then mounted with CytoSeal 60 (Electron Microscopy Sciences).

GUS Staining

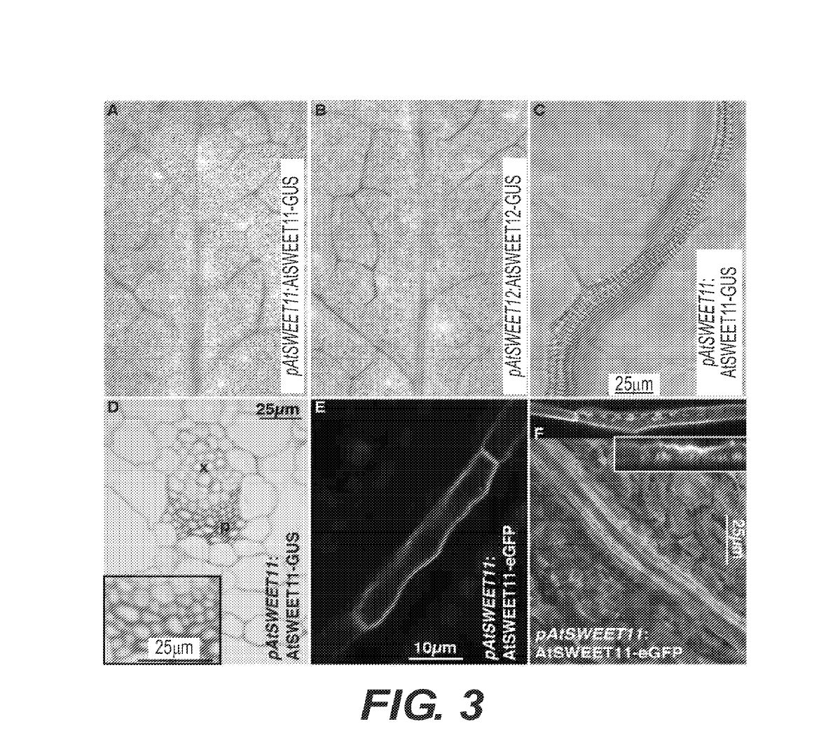

GUS staining was performed following standard procedures with minor changes (Belousov et al. Nat. Methods 3, 281 (2006), Martin et al. in GUS protocols: using the GUS gene as a reporter of gene expression, Gallagher, Ed. (Academic press, San Diego, 1992) pp. 23-43). Samples for GUS staining shown in FIG. 3C were prepared and analyzed using a modified pseudo-Schiff propidium iodide (PS-PI) staining technique. (Truernit et al. Plant Cell 20, 1494 (2008)). Whole seedlings were prefixed in ice-old 90%(v/v) acetone for 20 min on ice and washed three times with 100 mM phosphate buffer (pH 7.2) for 5 min each. Potassium ferrocyanide/ferricyanide were used at a final concentration of 5 mM. Staining intensity and diffusion were checked under a microscope and controlled by modulating incubation time at 37° C. For cross-sections (FIG. 3D), leaves were stained for 1 to 5 hours to reduce diffusion depending on the age of the leaves and expression levels in the individual lines.

Microscopy

Fluorescence imaging of plants was performed on a Leica TCS SP5 microscope. eYFP and eGFP were visualized by standard procedures as described before. (Chen et al. Nature 468, 527 (2010)). GUS staining was recorded under a Leica MZ125 stereomicroscope or Eclipse E600 microscope (Nikon). Image analysis was performed using Fiji software.

Tissue Preparation and Transmission Electron Microscopy

Sepal samples were taken at a flower stage in which the bud had opened, petals were visible, but the long stamens had not extended above stigma. Sepal sections were fixed in 1.5% paraformaldehyde and 1.5% glutaraldehyde in 0.1M sodium cacodylate buffer (0.1 M, pH 6.8, Electron Microscopy Sciences) overnight at 4° C. Specimens were then dehydrated in a graded water/ethanol series and low temperature-embedded in LR White resin modified from as follows: 10% EtOH, 20° C., 10 min; 30% EtOH, 0° C., 1 h; 50% EtOH, −20° C., 1 h; 75% EtOH, −20° C., 1 h; 95% EtOH, −20° C., 1 h; ethanol/resin mixtures of 2:1, 1:1, 1:2, by volume, −20° C., for 1 h each; two baths of pure resin, −20° C., for 4 hours each (VandenBosch, in Electron Microscopy of Plant Cells, Hall et al. Eds. (Academic Press, 1991)). The resin was polymerized at 50° C. in gelatin capsules for 60 hrs. Sections were cut (75 to 90 nm) on a Leica Ultracut S (Leica), picked up on formvar/Carbon coated slot grids or Cu grids. Sections were contrasted with 2% aqueous uranyl acetate (10 min), followed by 0.2% lead citrate (5 min). All sections were examined in the JEOL JEM-1400 TEM at 120 kV and images were taken using a Gatan Orius digital camera.

### Primer List

(The recombination sequences of the “Gateway att” sites are indicated in bold and restriction sites are indicated in italics in the primer sequences)

| | | | Amplicon |
|---|---|---|---|
| | | | size |
| PCR purpose | Primer name | Primer sequence | in bp |
| Truncated version | AtSWEET11- | GCTTTCCCGAATGTGCTTGGTTga | |
| of AtSWEET11-F201* | F201*_F | GCTCTCGGTGCACTCCAAATG | |
| construction in | AtSWEET11- | CATTTGGAGTGCACCGAGAGCtcA | |
| pDONR221f1 | F201*_R | ACCAAGCACATTCGGGAAAGC | |
| Truncated version | AtSWEET13- | GCAGTCCTCTTCCGCAGCAGCTAC | |
| of AtSWEET13-L203* | L203*_F | ATAgCCAGCTTTCTTGTACAAAG | |
| construction in | AtSWEET13- | CTTTGTACAAGAAAGCTGGcTATG | |
| pDONR221f1 | L203*_R | TAGCTGCTGCGGAAGAGGACTGC | |
| Genotyping of | AtSWT11LP | CCGAAGAGTAATGTGACCACG | 1089 |
| atsweet11 mutant | AtSWT11RP | TGAAGTGGGTGCTTTTGTTTC | |
| SALK_073269 | | | |
| Genotyping of | AtSWT12LP | ATGCAGGCCAACGTTCTATAG | 1145 |
| atsweet12 mutant | AtSWT12RP | TCAAAGGCCAAAGCAATATACC | |
| SALK_031696 | | | |
| pAtSWEET11:AtSWEET | AtSWT11attB1 | GGGGACAAGTTTGTACAAAAAAGCA | 4784 |
| 11-GUS fusion and | | GGCTTACACACGCATCGGATCGGAGA | |
| complementation | AtSWT11attB2 | GGGGACCACTTTGTACAAGAAAGCT | |
| constructs | | GGGTATGTAGCTGCTGCGGAAGAGG | |
| pAtSWEET11: | AtSWT11KpnIF | GGGGGGTACCCACACGCATCGGATCGGAGA | 4784 |
| AtSWEET | AtSWT11PstIR | GGGGCTGCAGCTGTAGCTGCTGCGGAAGAGG | |
| 11-eGFP fusion | | | |
| constructs | | | |
| pAtSWEET12: | AtSWT12attB1 | GGGGACAAGTTTGTACAAAAAAGCAG | 3745 |
| AtSWEET | | GCTTCAAATGGTGAACAATCTCGTCG | |
| 12-GUS fusion and | | TTAT | |
| complementation | AtSWT12attB2 | GGGGACCACTTTGTACAAGAAAGCTGG | |
| constructs | | GTAAGTAGTTGCAGCACTGTTTCTA | |
| 35S:AtSWEET11- | AtSWT11FattB1 | GGGGACAAGTTTGTACAAAAAAGCA | 867 |
| eYFP construct | | GGCTTAATGAGTCTCTTCAACACTGAAAAC | |
| or RT-PCR | AtSWT11attB2 | GGGGACCACTTTGTACAAGAAAGCT | |
| | | GGGTATGTAGCTGCTGCGGAAGAGG | |
| 35S:AtSWEET12- | AtSWT12FattB1 | GGGGACAAGTTTGTACAAAAAAGCAGG | 855 |
| eYFP construct | | CTTCAAATGGTGAACAATCTCGTCGTTAT | |
| or RT-PCR | AtSWT12attB2 | GGGGACCACTTTGTACAAGAAAGCTG | |
| | | GGTAAGTAGTTGCAGCACTGTTTCTA | |
| RT-PCR for | AtACT2F | TCCAAGCTGTTCTCTCCTTG | 387 |
| AtACTIN2 | AtACT2R | GAGGGCTGGAACAAGACTTC | |
| qPCR | AtSWT11DNF | GCCAATCTCAGTGGTTCGTCAA | 105 |
| | AtSWT11DNR | GAAGAGGACTGCTTGCCATGT | |
| | AtSWT11UPF | TCCTTCTCCTAACAACTTATATACCATG | 131 |
| | AtSWT11UPR | TCCTATAGAACGTTGGCACAGGA | |
| | AtSWT12DNF | CTCACATCTCCTGAACCAGTAGC | 114 |
| | AtSWT12DNR | TGCAGCACTGTTTCTAACTCCC | |
| | AtSWT12UPF | AAAGCTGATATCTTTCTTACTACTTCGAA | 204 |
| | AtSWT12UPR | CTTACAAATCCTATAGAACGTTGGCAC | |
| | AtSWT13F | CTTCTACGTTGCCCTTCCAAATG | 309 |

Breeding has led to dramatic increases in crop yield. Increased yield potential has mainly been attributed to improvements in allocation efficiency, defined as the amount of total biomass allocated into harvestable organs. (Zhu et al. Annu. Rev. Plant Biol. 61, 235 (2010), Paterson et al. Proc. Natl. Acad. Sci. U.S.A. 108, 10931 (2011)). Despite the critical importance of sucrose translocation in this process, the mechanism of how changes in translocation efficiency elusiveness may have contributed to an increase in harvestable products. Allocation of photoassimilates in plants is conducted by transport of sucrose from the photosynthetic ‘sources’ (predominantly leaves) to the heterotrophic ‘sinks’ (meristems, roots, flowers and seeds). (Lalonde et al. Annu. Rev. Plant Biol. 55, 341 (2004), Giaquinta, Annual Review of Plant Physiology 34, 347 (1983), Ayre, Mol. Plant 4, 377 (2011)). Sucrose, the predominantly transported form of sugars in many plant species (Fu et al. Plant Physiol., (2011)), is produced in leaf mesophyll cells, particularly in the palisade parenchyma of dicots and the bundle sheath of monocots. In apoplasmic loaders, sucrose is loaded into the sieve element/companion cell complex (SE/CC) in the phloem by the sucrose H+/cotransporter SUT1 (named SUC2 in Arabidopsis) from the apoplasm (cell wall space). (Riesmeier et al. The Plant Cell 5, 1591 (1993), Riesmeier et al. EMBO J. 11, 4705 (1992), Riesmeier et al. EMBO J. 13, 1 (1994), Burkle et al. Plant Physiol. 118, 59 (1998), Gottwald et al. Proc. Natl. Acad. Sci. 97, 13979 (2000)). However, sucrose must effuse from inside the cell into the cell wall either directly from mesophyll cells (and then travel to the phloem in the apoplasm), or from cells closer to the site of loading (having traveled cell-to-cell through plasmodesmata). Both the site and the mechanism of sucrose efflux remain to be elucidated, although it has been argued that a site in the vicinity of the site of phloem loading is most probable. (Giaquinta, Annual Review of Plant Physiology 34, 347 (1983), Ayre, Mol. Plant 4, 377 (2011)). The present invention provides methods for identifying proteins that can transport sucrose across the plasma membrane: AtSWEET10-15 in Arabidopsis and OsSWEET11 and 14 in rice. As evidenced herein, AtSWEET11 and 12 are expressed in phloem cells and that inhibition by mutation reduces leaf assimilate exudation and leads to increased sugar accumulation in leaves. Thus apoplasmic phloem loading occurs in a two-step model: sucrose exported by SWEETs from phloem parenchyma cells feeds the secondary active proton-coupled sucrose transporter SUT1 in the SE/CC.

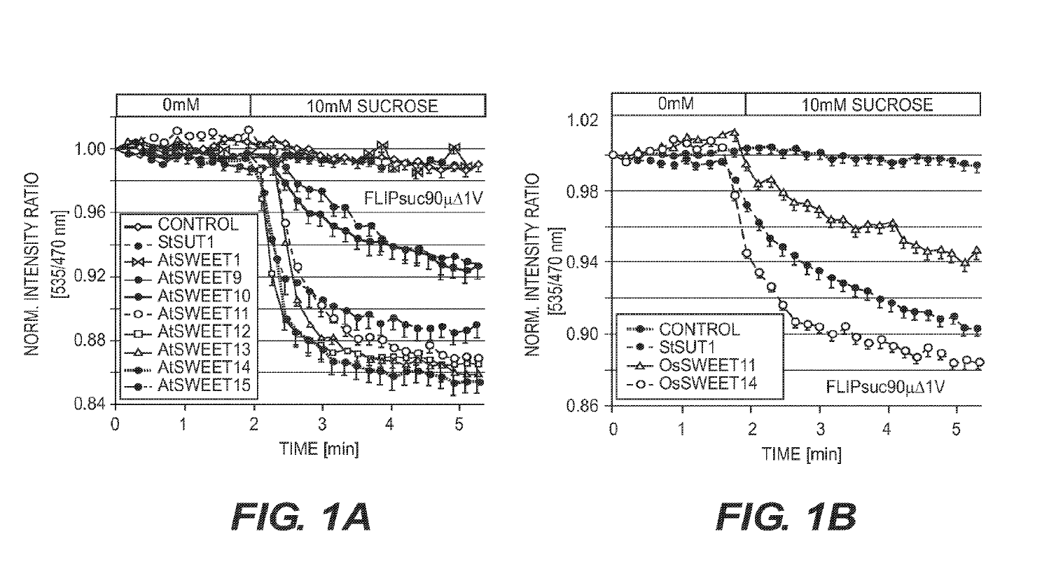

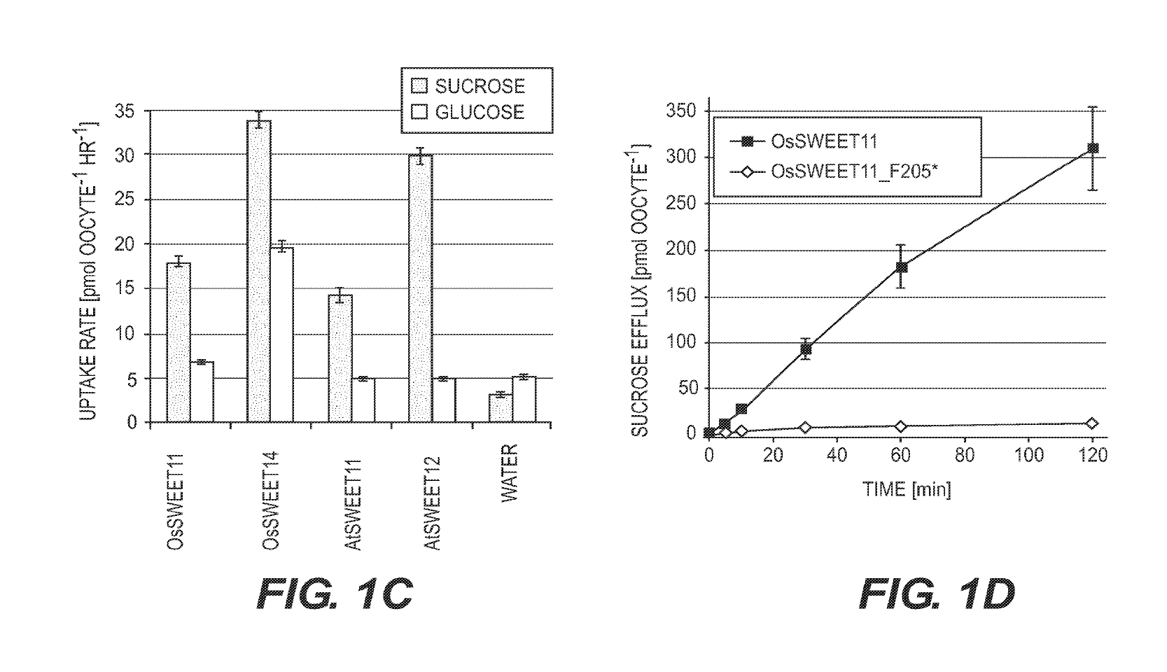

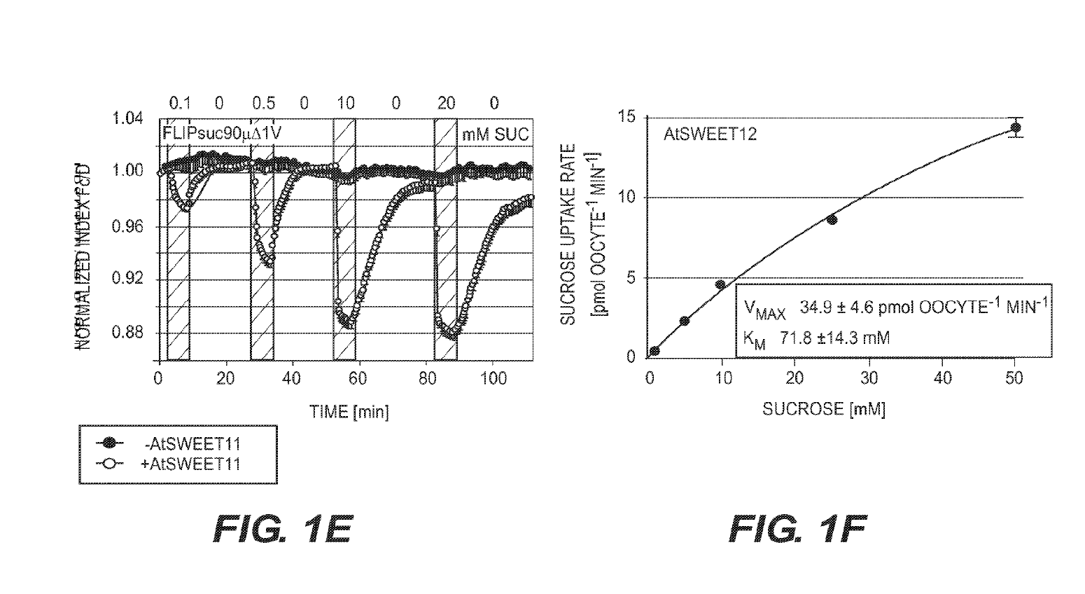

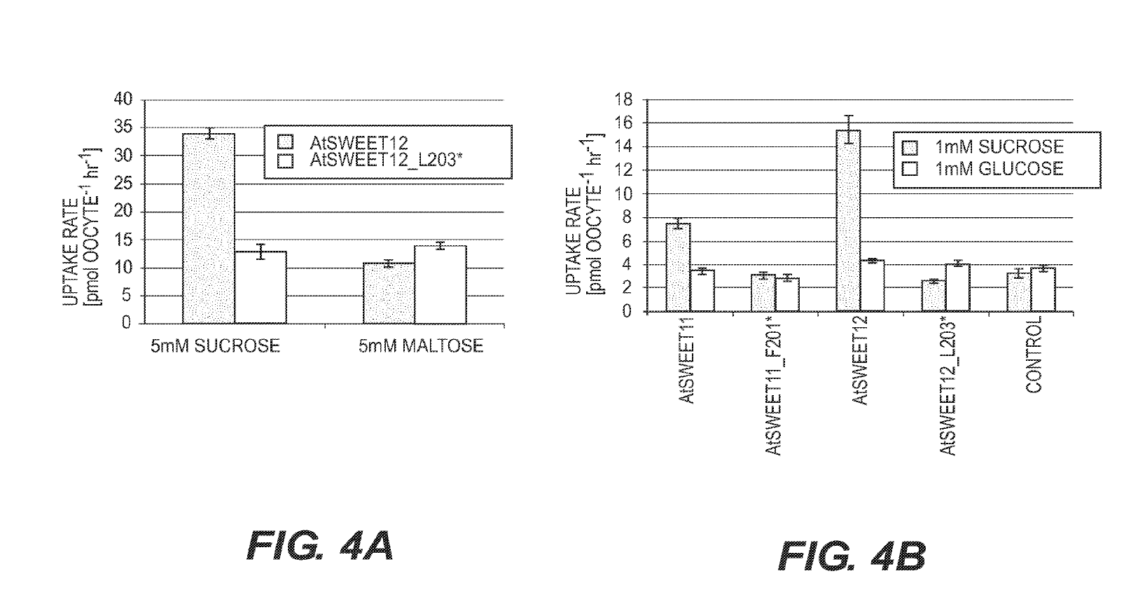

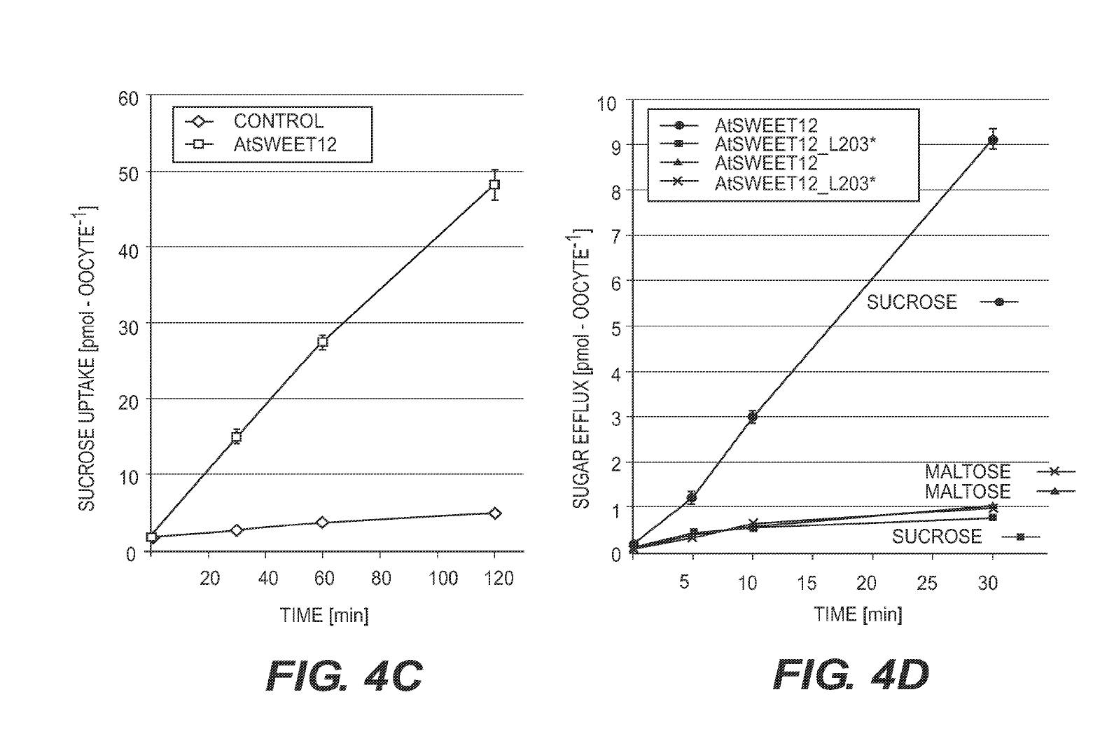

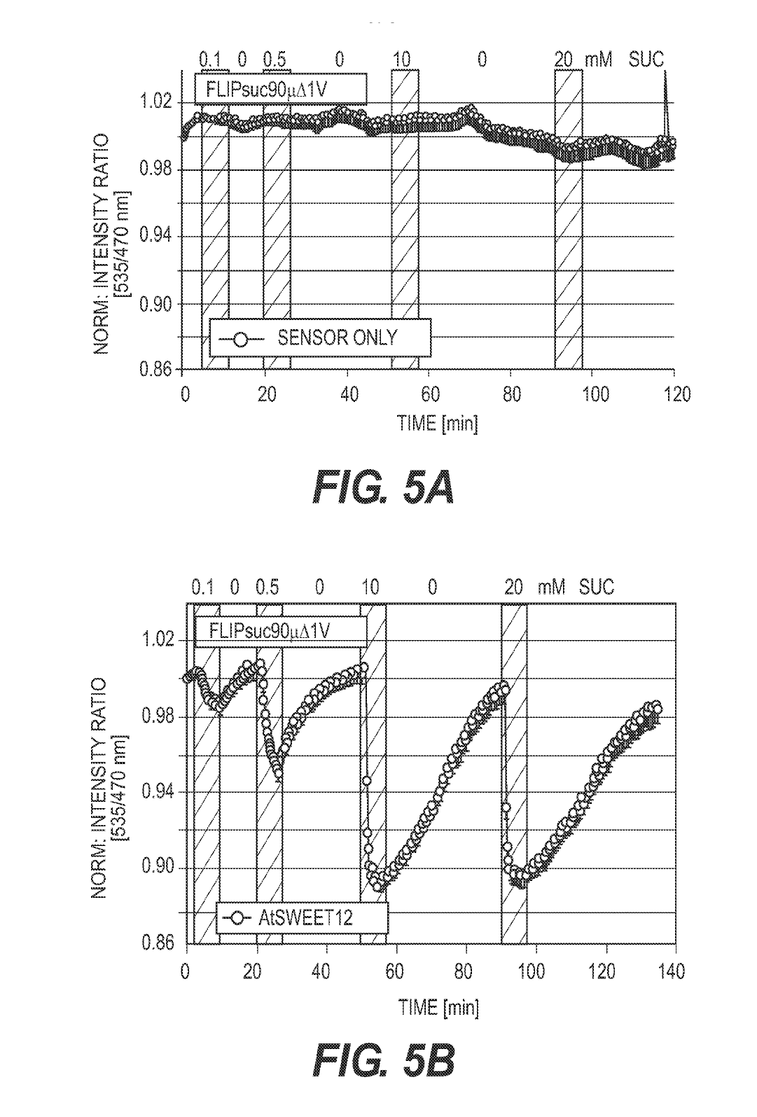

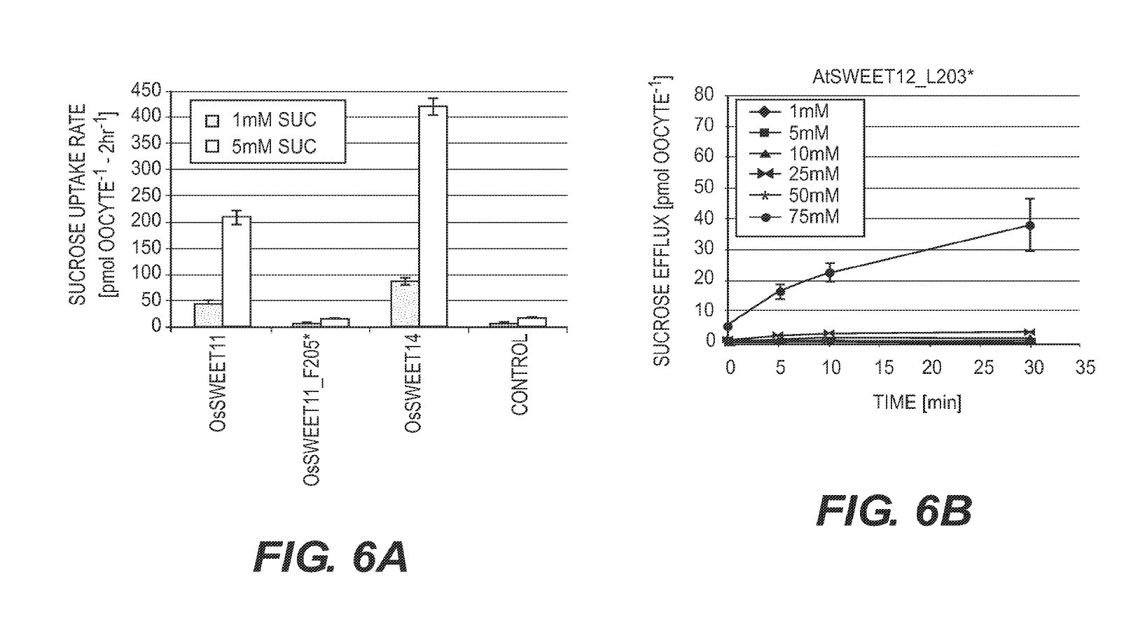

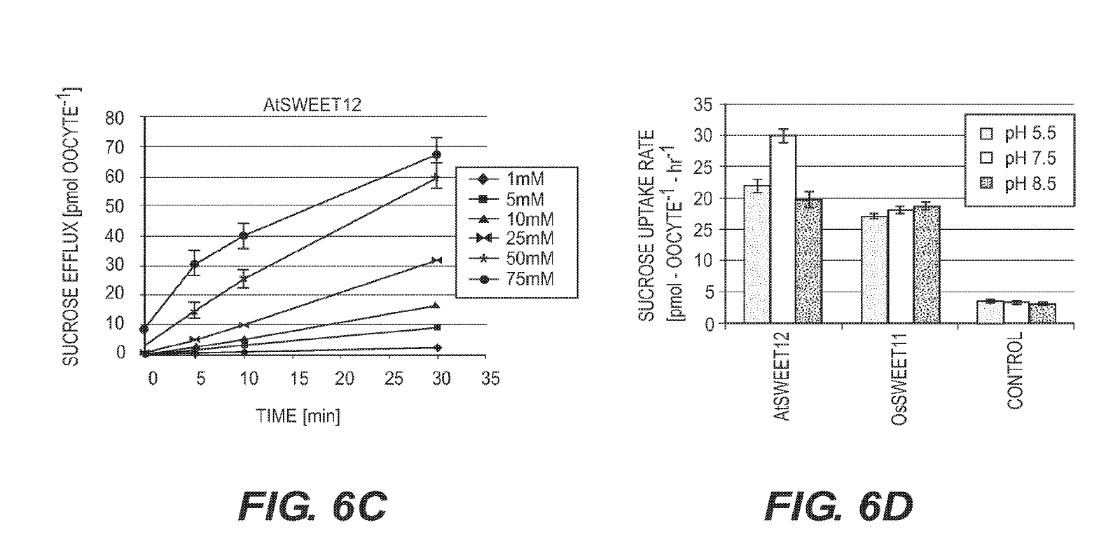

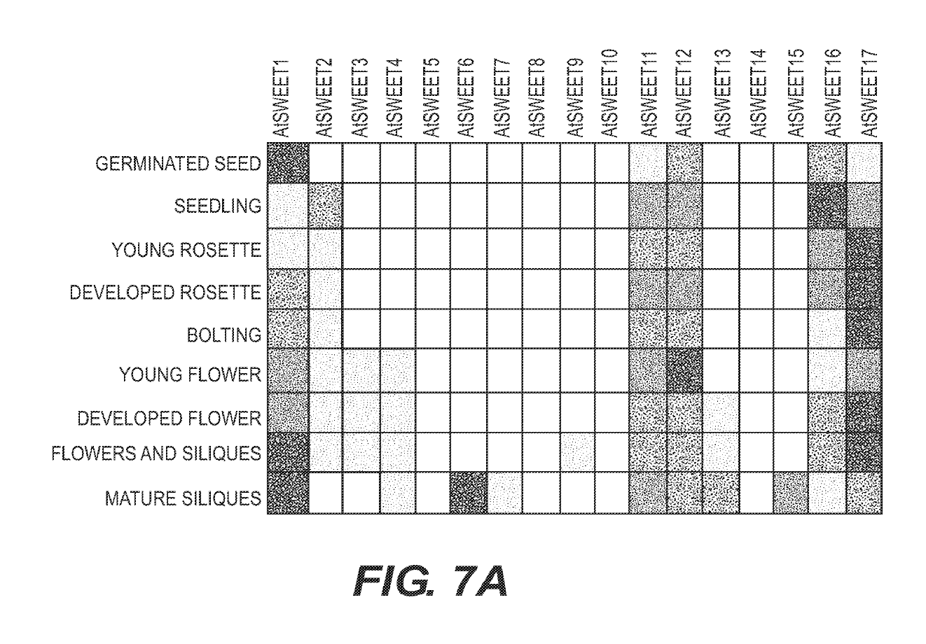

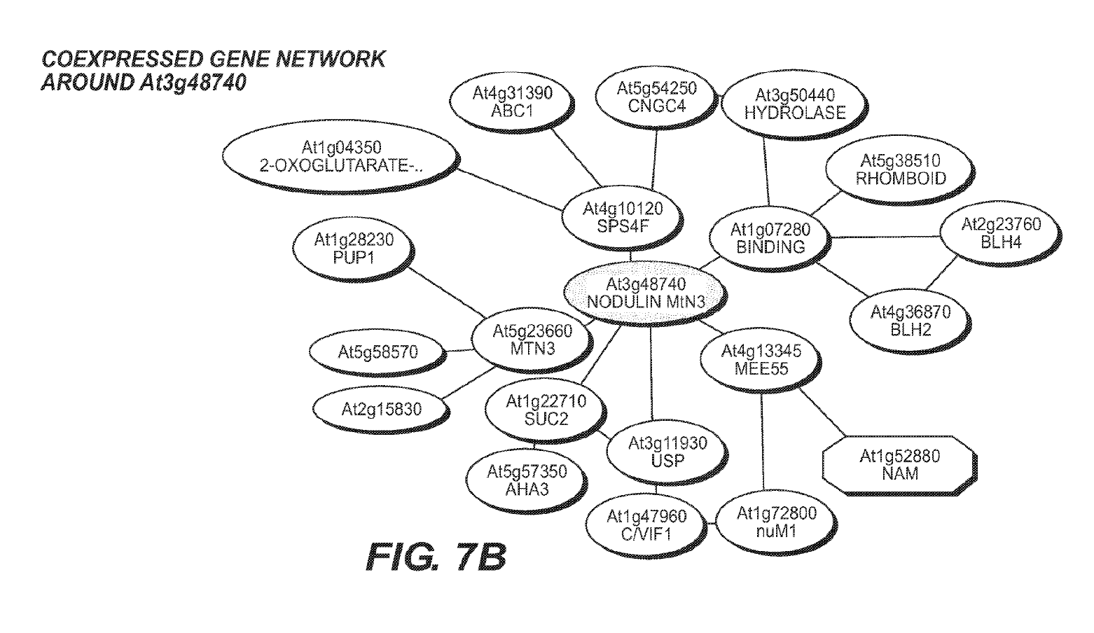

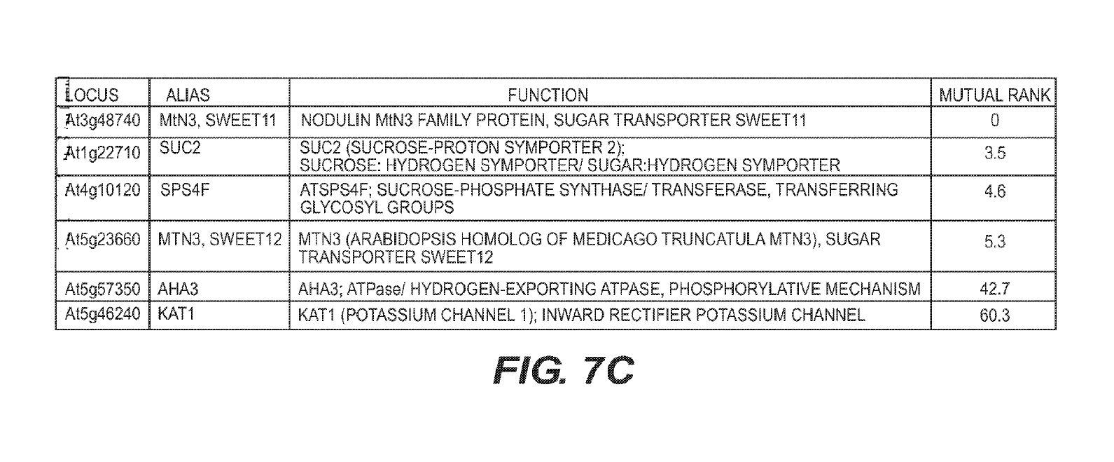

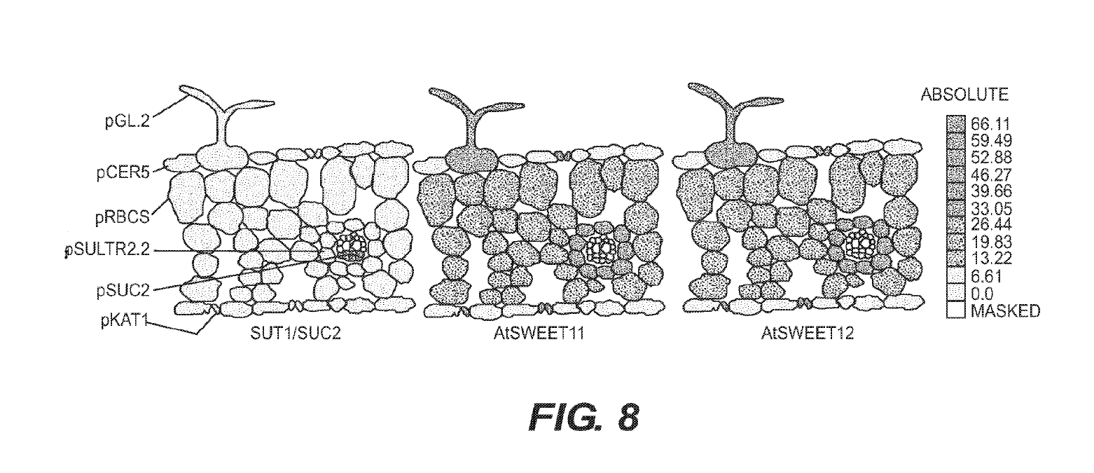

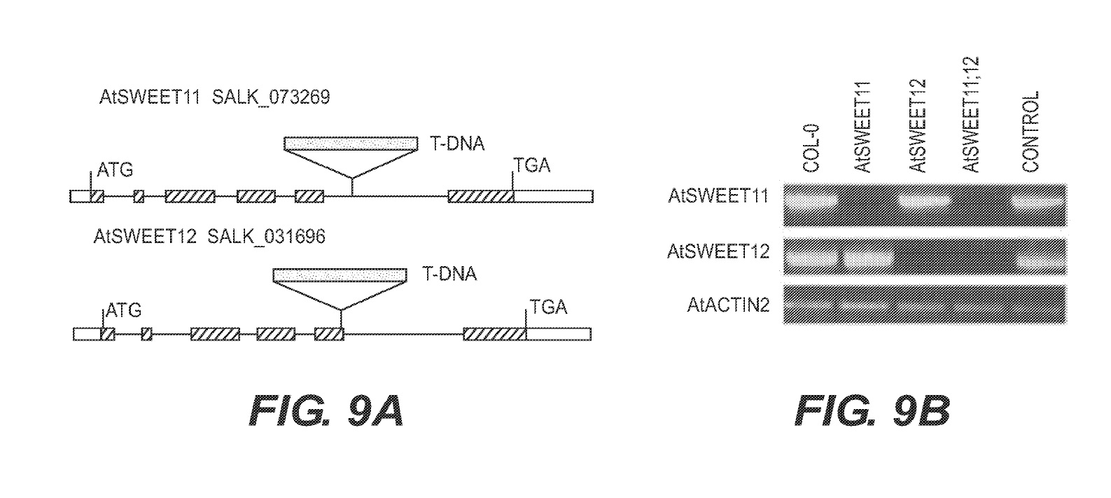

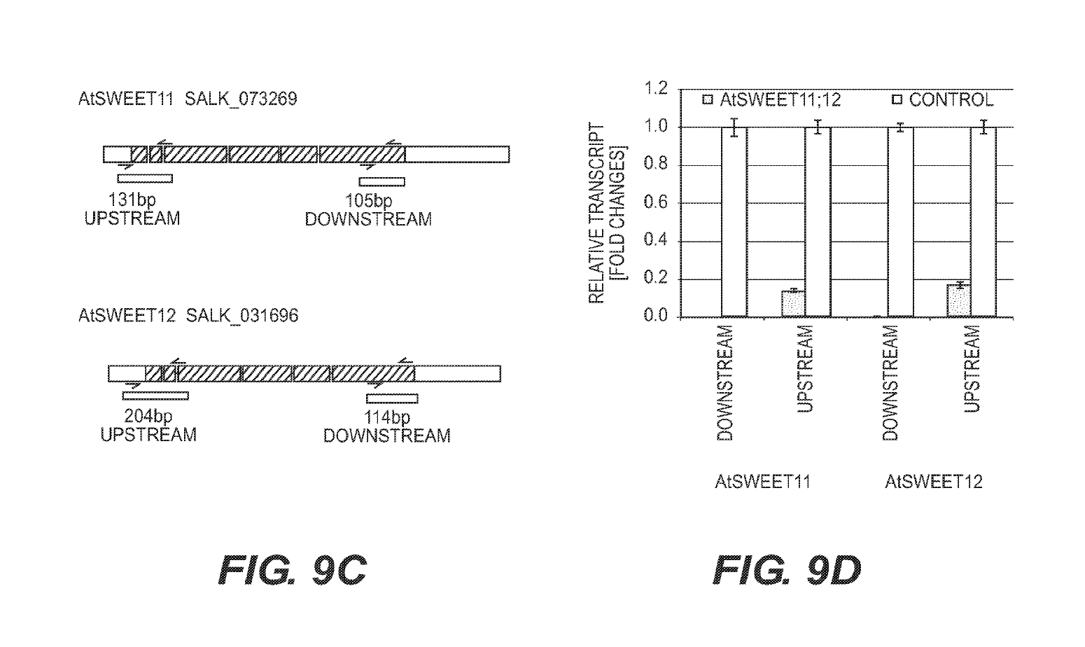

The sucrose efflux transporters were identified using a FRET-based screen. Since humans do not seem to possess sucrose transporters, it was reasoned that human cell lines should lack significant endogenous sucrose transport activity and should thus represent a suitable functional expression system for heterologous sucrose transporters. A preliminary set of ^{˜}50 candidate genes comprising membrane proteins with ‘unknown’ function and members of the recently identified SWEET glucose effluxer family were coexpressed with the FRET sucrose sensor FLIPsuc90μΔ1V in human HEK293T cells. AtSWEET10-15, which all belong to clade III of the AtSWEET family, enabled HEK293T cells to accumulate sucrose as detected by a negative ratio change in sensor output (FIG. 1A). (Chen et al. Nature 468, 527 (2010), Lager et al. J. Biol. Chem. 281, 30875 (2006)). To corroborate these findings, the clade III orthologs OsSWEET11 and 14 from rice (FIG. 1B) were tested and were shown to transport sucrose. By contrast, proteins from the other SWEET clades did not show detectable sucrose uptake into HEK293T cells (FIG. 1A). Clade III SWEETs show preferential transport activity for sucrose over glucose and do not appear to transport maltose (FIG. 1C and FIG. 4). The ability of clade III SWEETs to export sucrose was shown by time-dependent efflux of [¹⁴C]-sucrose injected into oocytes (FIG. 1D and FIG. 4D) and was further supported by the reversibility of sucrose accumulation as measured by optical sensors in mammalian cells (FIG. 1E and FIG. 5). HEK293T cells expressing the sensor alone did not show detectable sucrose accumulation even at the higher levels of sucrose in the perfusing buffer. Cells coexpressing AtSWEET12 with the sensor showed concentration-dependent and reversible accumulation of sucrose. It is reasonable to assume that HEK293T cells do not contain endogenous mechanisms for efficient metabolization of sucrose; the reversibility indicates efflux of sucrose. The asymmetry of uptake rates relative to efflux rates is most probably caused by concentration gradient differences between the two conditions. Before uptake, intracellular sucrose levels are expected to be far below the detection level of the sensor (KD ^{˜}90 μM), and during uptake the inward gradient will be large. However, intracellular levels are limited by the capacity of the transporter and most probably do not reach levels comparable to the extracellular concentration. Thus, during efflux the relative concentration gradient will be lower compared to that generated during uptake. SWEETs function as low affinity sucrose transporters (Km for sucrose uptake by AtSWEET12 was ^{˜}70 mM, Km for efflux was >10 mM; FIG. 1F and FIG. 6A-C). The largely pH-independent transport activity supports a uniport mechanism (FIG. 6D). The observed transport characteristics are compatible with those of the low affinity components for sucrose transport detected in vivo. (R. Lemoine, S. Delrot, FEBS Lett. 248, 129 (1989), Maynard et al. Plant Physiol. 70, 1436 (1982)). AtSWEET11 and 12 are highly expressed in leaves (microarray data and translatome data (Yu et al., Mol. Cell 13, 677 (2004), Santagata et al., Science 292, 2041 (2001)); FIG. 7A and FIG. 8) and were found to be coexpressed with genes involved in sucrose biosynthesis and phloem loading (e.g. sucrose phosphate synthase, SUC2, and AHA3, FIGS. 7B and 7C). Cell-type-specific expression is based on coexpression with any of the six genes whose promoters were used for driving the ribosomal affinity tag: pGL.2 for trichomes, pCER5 for epidermis, pRBCS for mesophyll, pSULTR2.2 for bundle sheath, pSUC2 for companion cells and pKAT1 for guard cells. While the cell-specificity of the pSUC2 promoter is unambiguous in companion cells with leakage into the sieve elements, bundle sheath expression of pSULT2.2 is not as well documented. (Srivastava et al. Plant Physiol. 148, 200 (2008), Rolland et al. Annu. Rev. Plant Biol. 57, 675 (2006)). The representation pattern in the vascular system is crude and does not reflect an anatomically adequate representation of the phloem. The data provide shown here critical information, namely they indicate that the cell-type specific expression site of AtSWEET11 and AtSWEET12 is distinct from that of AtSUC2. The data demonstrate that SWEETs are involved in sugar efflux from either bundle sheath or phloem parenchyma cells, the two cell types adjacent to the SE/CC complex. The GUS and eGFP fusion data shown in FIG. 3 do not support expression in the bundle sheath, indicating at least a significant overlap of the expression of AtSWEET11 and 12 with AtSULTR2.2 in the phloem parenchyma. The tissue-specific expression and cellular localization of AtSWEET11 and 12 and the phenotypes of sweet mutants were analyzed to determine the physiological role of the sucrose transporters.

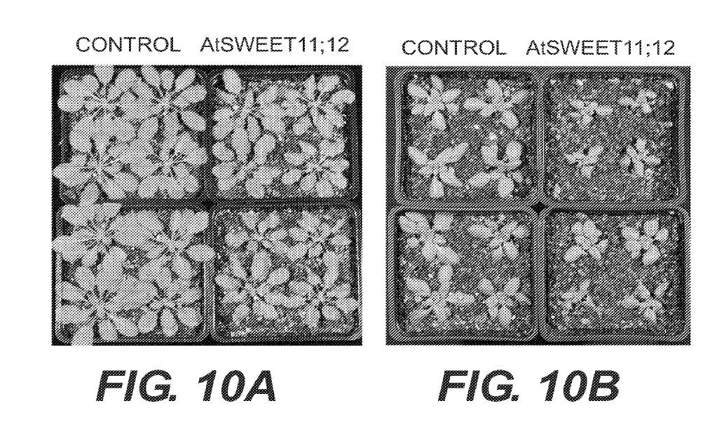

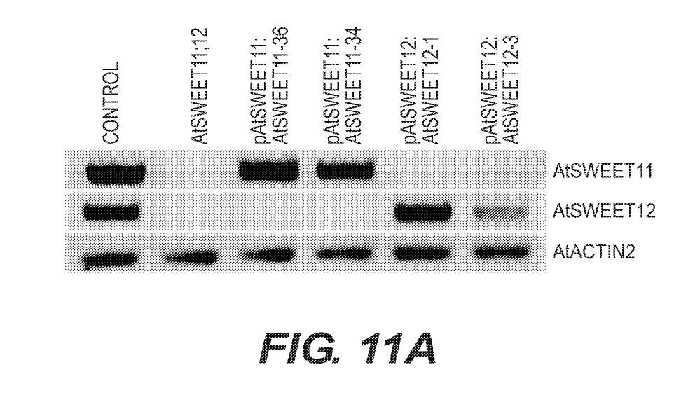

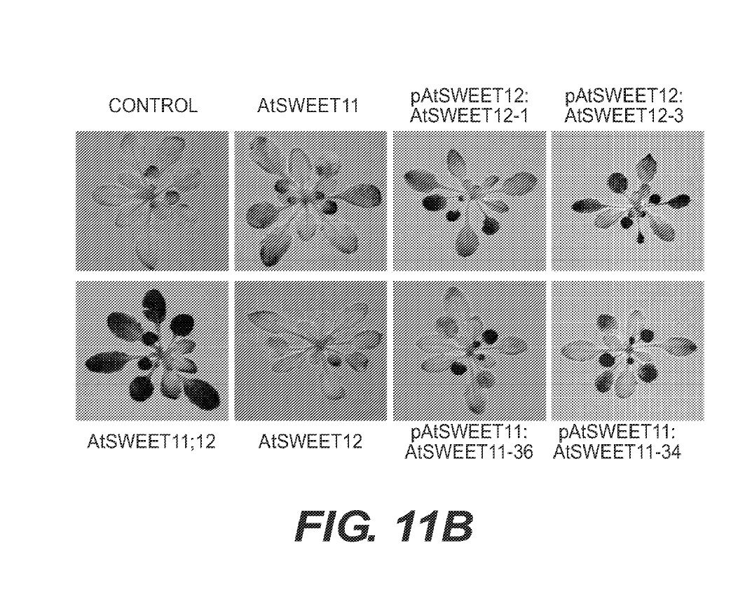

AtSWEET11 and 12 are close paralogs, with 88% similarity at the amino acid level. Lines carrying single T-DNA insertions in the AtSWEET11 and 12 loci did not show any obvious morphological phenotype compared to the wild type Col-0 or wild type siblings segregated from the same mutant populations (FIG. 10). However, at higher light levels the double mutant line was smaller compared to wild type controls (20-35% reduction in rosette diameter depending on light conditions; FIG. 2A and FIG. 11) and contained elevated starch levels at the end of the diurnal dark period (FIGS. 2, B and C). Moreover, mature leaves of the double mutant contained higher sucrose levels both at the end of the light period and the end of dark period (FIG. 2D). Leaves also accumulated higher levels of hexoses, similar as observed in plants exposed to sucrose, or plants in which phloem loading has been blocked. (Osuna et al. Plant J. 49, 463 (2007), Riesmeier et al. EMBO J. 13, 1 (1994), Srivastava et al. Plant Physiol. 148, 200 (2008)). Accumulation of free sugars is expected to lead to downregulation of photosynthesis through sugar signaling networks. (Rolland et al. Annu. Rev. Plant Biol. 57, 675 (2006)). The starch accumulation phenotype was partially complemented by expressing either AtSWEET11 or 12 under their respective promoters in the double mutant (FIG. 11). Together, these data indicate an impaired ability of the mutants to export sucrose from the leaves. Direct [¹⁴CO₂]-labeling experiments indicate that the double mutant exports ^{˜}50% of fixed ¹⁴C relative to control (FIG. 2E). It is noteworthy that the mutant is affected with respect to leaf size, photosynthetic capacity and steady state sugar levels, thus the apparent efflux rates may be compounded by these parameters.

Reduced efflux of sugars from leaves is expected to lead to reduced translocation of photoassimilates to the roots, thus negatively affecting root growth and the ability to acquire mineral nutrients. (Riesmeier et al. EMBO J. 13, 1 (1994), Burkle et al. Plant Physiol. 118, 59 (1998)). When germinated in the light on sugar-free media, atsweet11;12 mutants exhibited reduced root length (FIGS. 2F and 2G). Addition of sucrose to the media rescued the root growth deficiency of atsweet11;12 mutants (FIGS. 2F and 2G). A similar sucrose-dependent root growth deficiency has also been observed for the Arabidopsis sucrose/H+ cotransporter suc2 mutant. (Gottwald et al. Proc. Natl. Acad. Sci. 97, 13979 (2000)). Both the suc2 and the AtSWEET11;12 mutants are apparently able to acquire sucrose or sucrose-derived hexoses from the medium to restore root growth restricted by a carbohydrate deficiency.

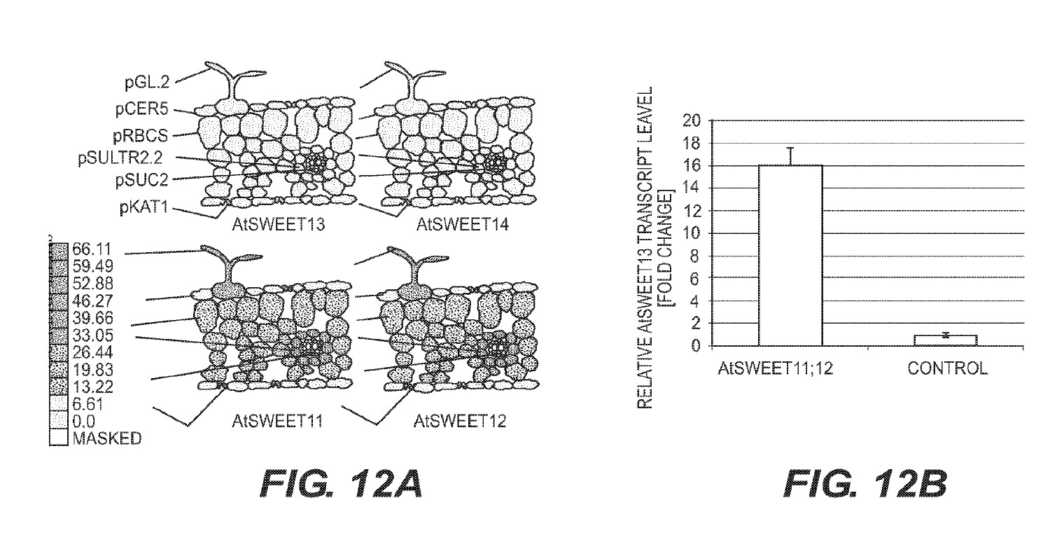

The growth phenotype for AtSWEET11;12 is not as dramatic as described previously for the suc2 mutant. (Riesmeier et al. EMBO J. 13, 1 (1994), Burkle et al. Plant Physiol. 118, 59 (1998), Gottwald et al. Proc. Natl. Acad. Sci. 97, 13979 (2000)). The Arabidopsis genome encodes several SWEET paralogs, including the closely related transporters AtSWEET10, 13, 14 and 15, which were shown to function as sucrose transporters. qPCR analyses showed that AtSWEET13, which is typically expressed at low levels in leaves, is induced ^{˜}16-fold in the AtSWEET11;12 double mutant (FIG. 12B). Thus in contrast to the secondary active SE/CC loaders SUT1/SUC2, SWEETs function as redundant elements of phloem loading. It is noteworthy that ossweet14 rice mutants display stunted growth, possibly a result of reduced sugar efflux from leaves as well. (Antony et al. The Plant Cell 22, 3864 (2010)).

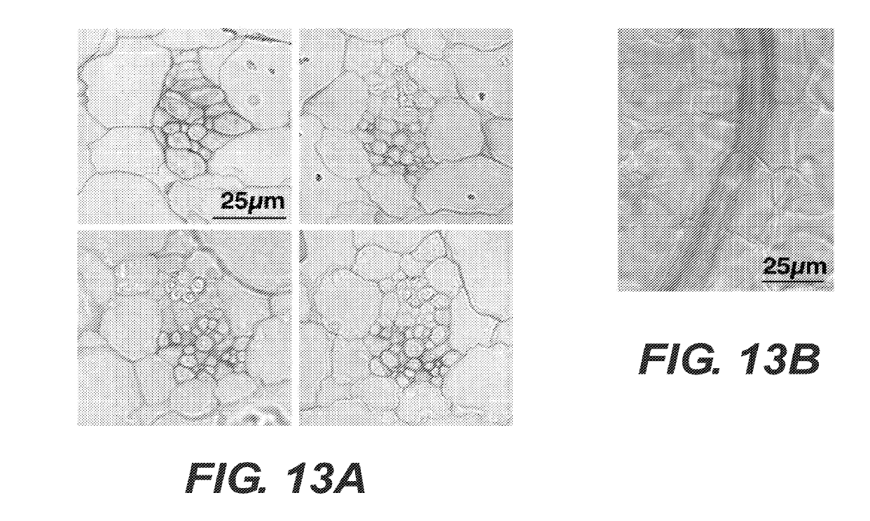

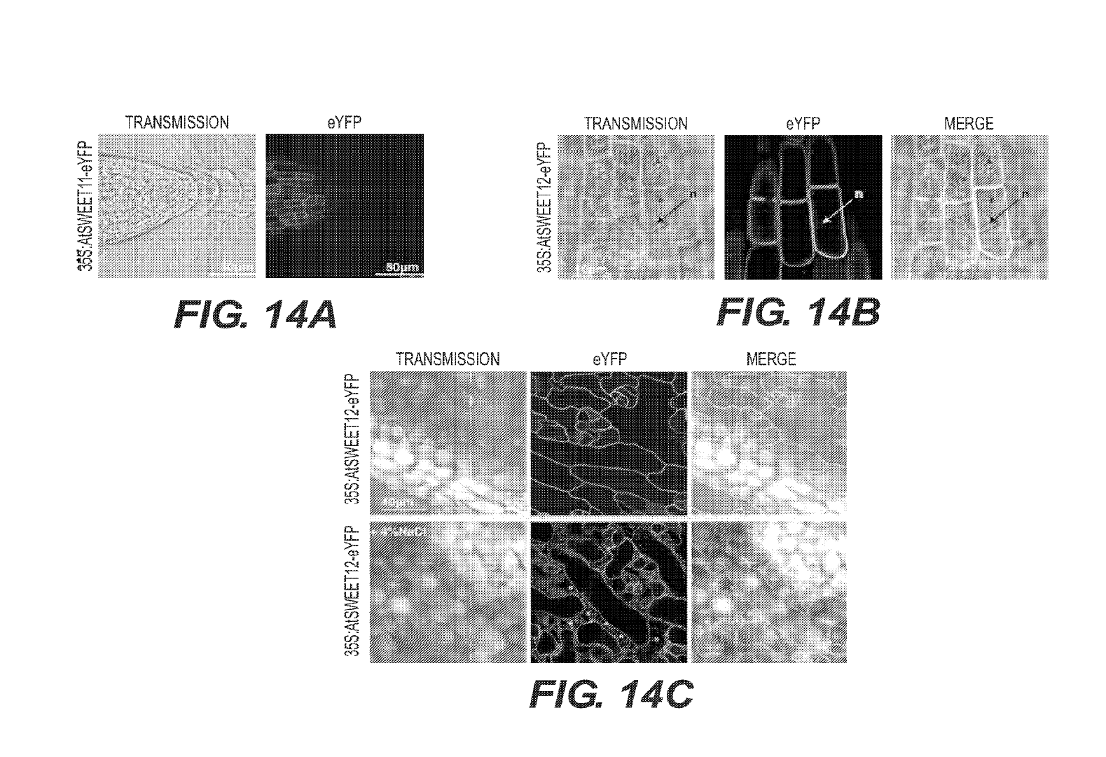

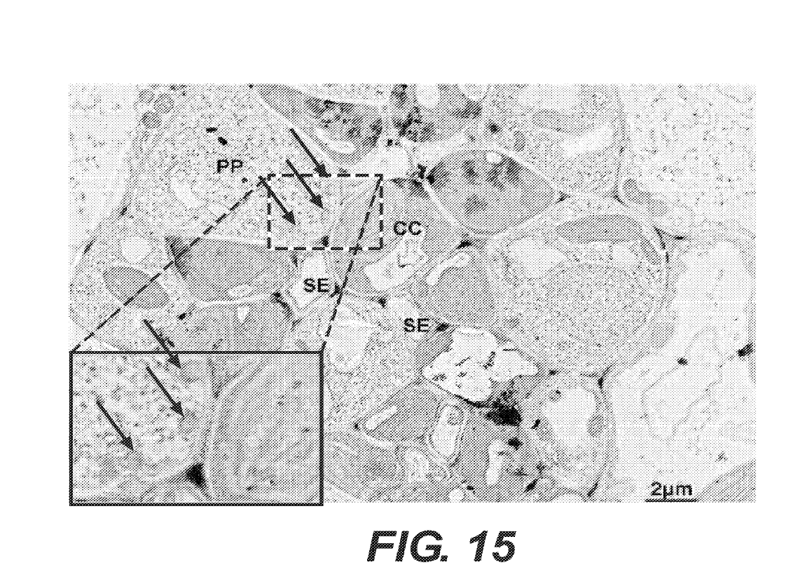

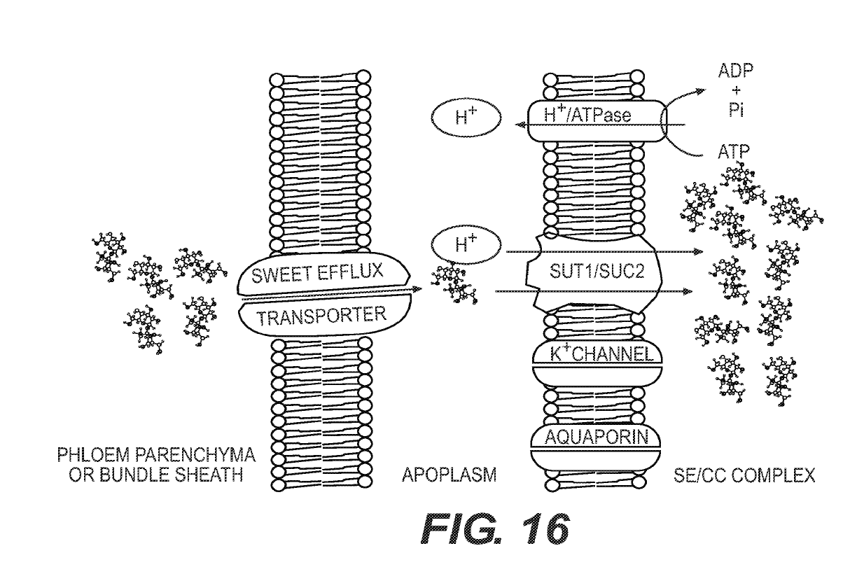

Taken together, the data indicate that clade III SWEETs are involved in export of sucrose and are responsible for the previously undescribed first step in phloem loading. The efflux of sucrose to the apoplasm could theoretically occur directly at the site of production in mesophyll cells, from bundle sheath cells or from phloem parenchyma cells. Localization of AtSWEET11 and 12 driven by their native promoters, as translational GFP or GUS fusions revealed that both proteins are present in the vascular tissue including minor and major veins, which in Arabidopsis are considered to participate in phloem loading (FIG. 3, A-D and FIG. 13). (Haritatos et al. Planta 211, 105 (2000)). The subcellular localization of GFP-tagged AtSWEET11 and 12 was consistent with localization to the plasma membrane (FIGS. 3E and 3F; further supported by data from CaMV 35S-SWEET-YFP plants, FIG. 14). AtSWEET11 and 12 were both expressed in select cells in the phloem, which form cell files along the veins (FIGS. 3C, 3D and 3F and FIG. 13). These cells correspond to phloem parenchyma. Data from cell-specific translatome studies show that AtSWEET11/12-expressing cells have a clearly distinct translatome compared to SUC2-expressing companion cells (FIG. 8). (Santagata et al. Science 292, 2041 (2001)). These data exclude that SWEET11 and 12 are expressed to significant levels in companion cells, supporting a localization in phloem parenchyma cells as the only remaining cell type in the phloem besides the enucleate sieve elements.

Further, OsSWEET11/Xa13 had been found to be expressed in the phloem of uninfected rice leaves, indicating that OsSWEET11 may play a similar role in phloem loading. (Chu et al. Theor. Appl. Genet. 112, 455 (2006)). Co-immunolocalization of SUT1/SUC2 and SWEET11/12 at the TEM level will be required to unambiguously define the cell type in which the SWEETs are functioning.

These findings are compatible with a model in which sucrose moves symplasmically via plasmodesmata towards the phloem and then effluxes close to the site of apoplasmic loading. Communication is needed to coordinate the efflux from phloem parenchyma with the uptake into the SE/CC to prevent spillover and limit the availability of nutrient resource for pathogens in the apoplasm of the leaf. Invertases and glucose/H+ cotransporters that are induced during pathogen infection may serve in retrieval of sugars spilled at the loading site. (Sutton et al. Plant. 129, 787 (2007)). Sugar- and turgor-controlled regulatory mechanisms involved in post-phloem unloading can also apply to sucrose efflux in the phloem loading process. (Patrick et al. J. Exp. Bot. 52, 551 (2001), Zhou et al. J. Exp. Bot. 60, 71 (2009)). The availability of SWEET sucrose transporters, together with FRET sensors, provides valuable tools for studying the regulatory networks coordinating local and long distance transport and metabolism. (Okumoto et al. New Phytol. 180, 271 (2008)).

Clade III SWEETs had previously been implicated as key targets of biotrophic pathogens. OsSWEET11, 13 and 14 are co-opted during infection of rice by Xanthomonas oryzae pv. oryzae (Xoo). (Chen et al. Nature 468, 527 (2010), Antony et al. The Plant Cell 22, 3864 (2010), Yang et al. Proc. Natl. Acad. Sci. 103, 10503 (2006), Yuan et al. Plant Cell Physiol. 50, 947 (2009)); Liu Q, et al. Plant Cell Environ. (2011) 34(11):1958-69).

Pathovar-specific effectors secreted by Xoo activate transcription of clade III SWEET genes and mutations in the effector binding sites in SWEET promoters lead to resistance to Xoo in a wide spectrum of rice lines. (Antony et al. The Plant Cell 22, 3864 (2010), Yang et al. Proc. Natl. Acad. Sci. 103, 10503 (2006), Yuan et al. Plant Cell Physiol. 50, 947 (2009), Chu et al. Genes Dev. 20, 1250 (2006); Liu Q, et al. Plant Cell Environ., 34(11):1958-69(2011); Yu et al., Mol Plant Microbe Interact. 24(9):1102-13 (2011)). The data here, namely that these SWEETs are key elements of the phloem translocation machinery, show that the pathogen retools a critical physiological function (i.e. a cellular sucrose efflux mechanism in the phloem) to gain access to the plant's energy resources at the site of infection. It is interesting to note that this function is redundant in the plant. Such redundancy in both pathogen and host functions has been attributed to increased system robustness and may have evolved to allow the plant to survive mutations in essential functions that create pathogen resistance. (Lundby et al. PLoS One 3, e2514 (2008)). One may speculate that the highly localized transfer of sucrose between phloem parenchyma and SE/CC has evolved to limit sucrose release into the apoplasm to a limited interface of adjacent cells inside the phloem, and thus reduce the availability of sucrose in the apoplasm to pathogens. Pathogens can overcome this first line of defense by targeting exactly this efflux mechanism in order to gain access to sugars in cells surrounding the infection site, for example in the epidermis or mesophyll. Invertase and monosaccharide transporters, which are also typically induced during infection, may then serve as a secondary line of defense to reduce apoplasmic sugar levels at the infection site. (Sutton et al. Physiol. Plant. 129, 787 (2007)).

Plants transport fixed carbon predominantly as sucrose, which is produced in mesophyll cells and imported into phloem cells for translocation throughout the plant. It is not known how sucrose migrates from sites of synthesis in the mesophyll to the phloem or which cells mediate efflux into the apoplasm as a prerequisite for phloem loading by the SUT sucrose/H+ cotransporters. Using optical sucrose sensors, a sub-family of SWEET sucrose efflux transporters was identified. AtSWEET11 and 12 localize to the plasma membrane of the phloem. Mutant plants carrying insertions in AtSWEET11 and 12 are defective in phloem loading, thus revealing a two-step mechanism of SWEET-mediated export from parenchyma cells feeding H+-coupled import into sieve element companion cells. Restriction of intercellular transport to the interface of adjacent phloem cells is therefore an effective mechanism to limit access of pathogens to photosynthetic carbon in the leaf apoplasm.

### Example 2

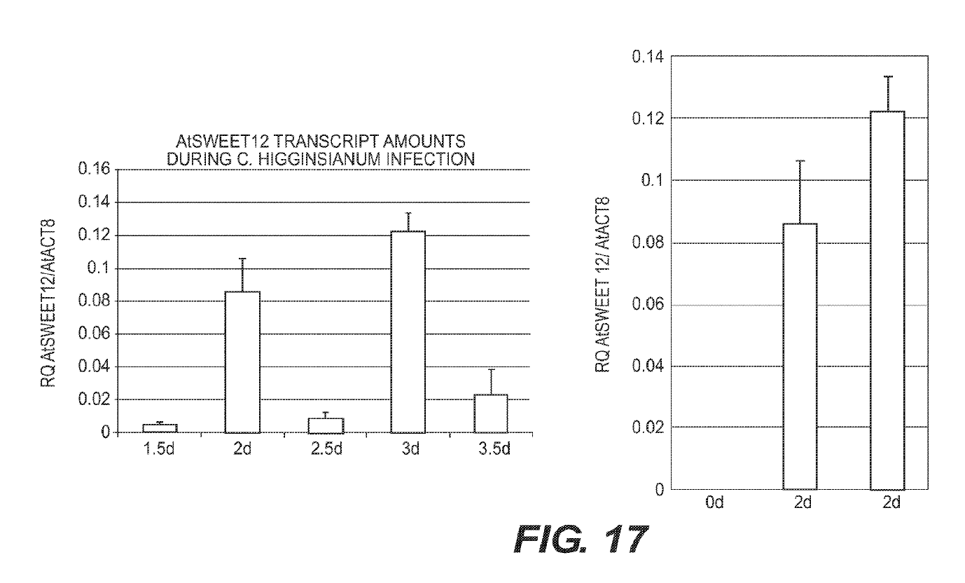

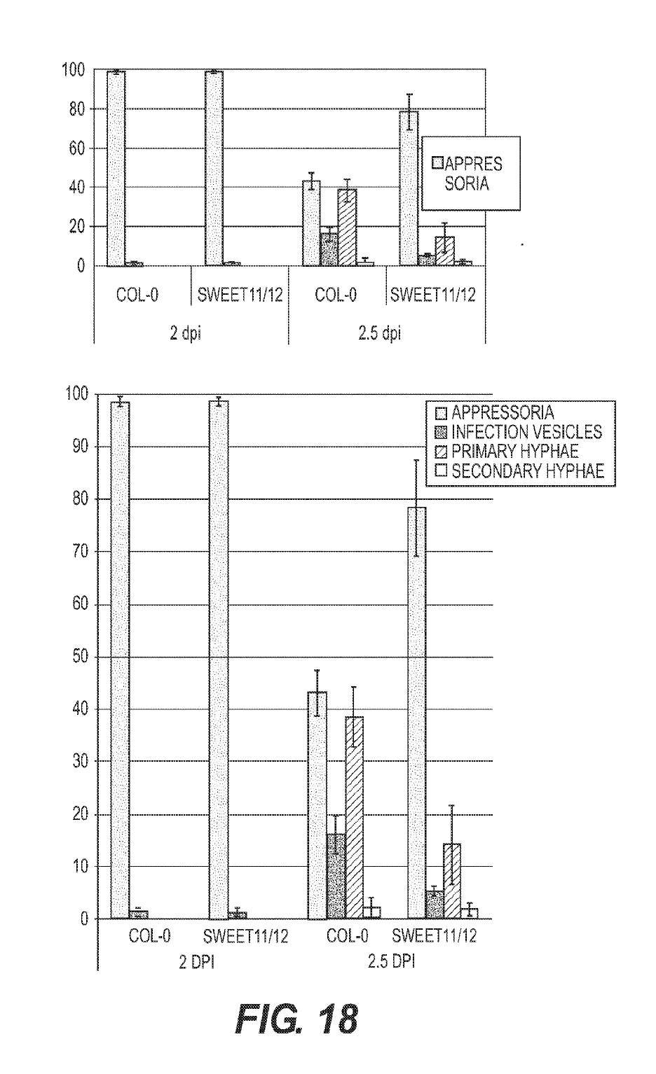

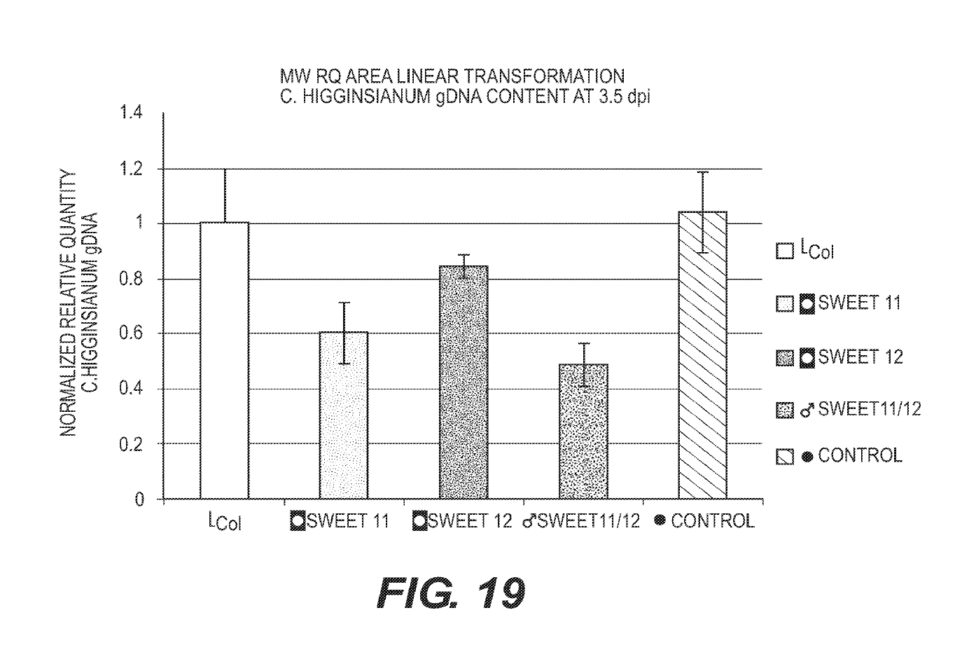

Arabidopsis plants were infected at the end of a light period in a cycle of 12 hr light: 12 hr dark with the fungal hemibiotrophic pathogen Colletotrichum higginsianum. Samples from 2 dpi and 3 dpi were taken 1 h before light was withdrawn and sample from the 2.5 dpi and 3.5 dpi were taken one hour after light was returned. Following the infection of wild type plants with C. higginsianum, quantitative PCR was performed as described. As FIG. 17 demonstrates, the pathogen induced SWEET11 and SWEET 12 expression. Further, as FIGS. 18 and 19 demonstrate, mutants for these SWEET transporters were resistant to the pathogen. These data are significant for two compelling reasons. First, this provides data for a pathogen that is a fungus, which to date are not known to rely on TAL effector molecules to hijack and ectopically induce expression of these genes. This evidences other methods that pathogens may utilize to influence transporter production. Further, this pathogen is a hemibiotroph, which can also grow by destroying cells and living off of the released compounds. As such, the pathogen should not have to rely on transporter induction to survive, but these data show that the fungus absolutely requires the sugar effluxer to survive.

### Example 3

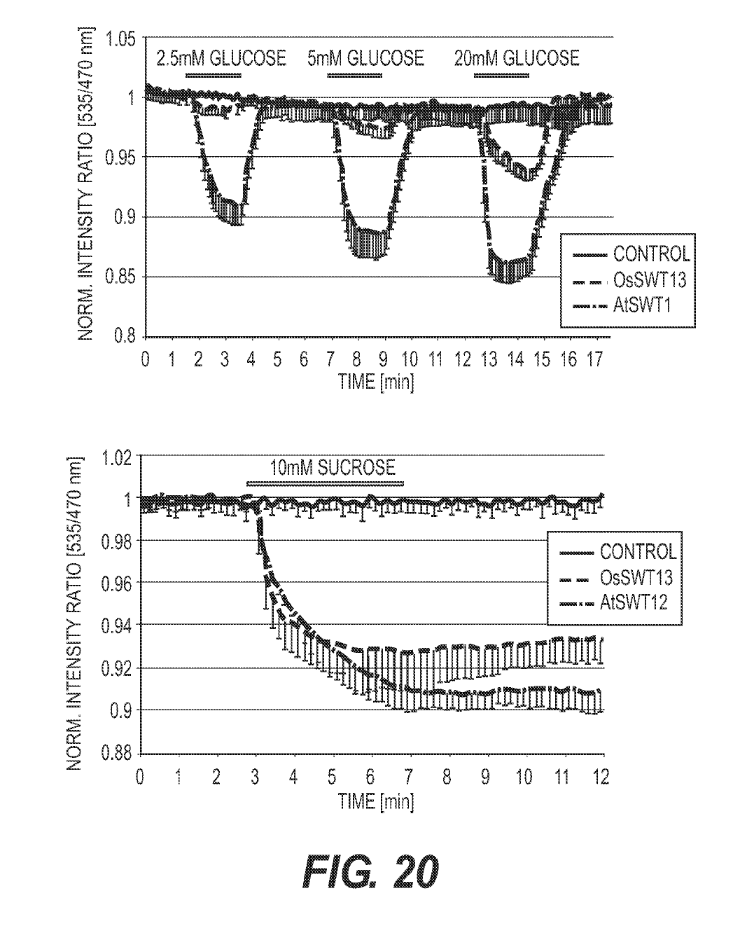

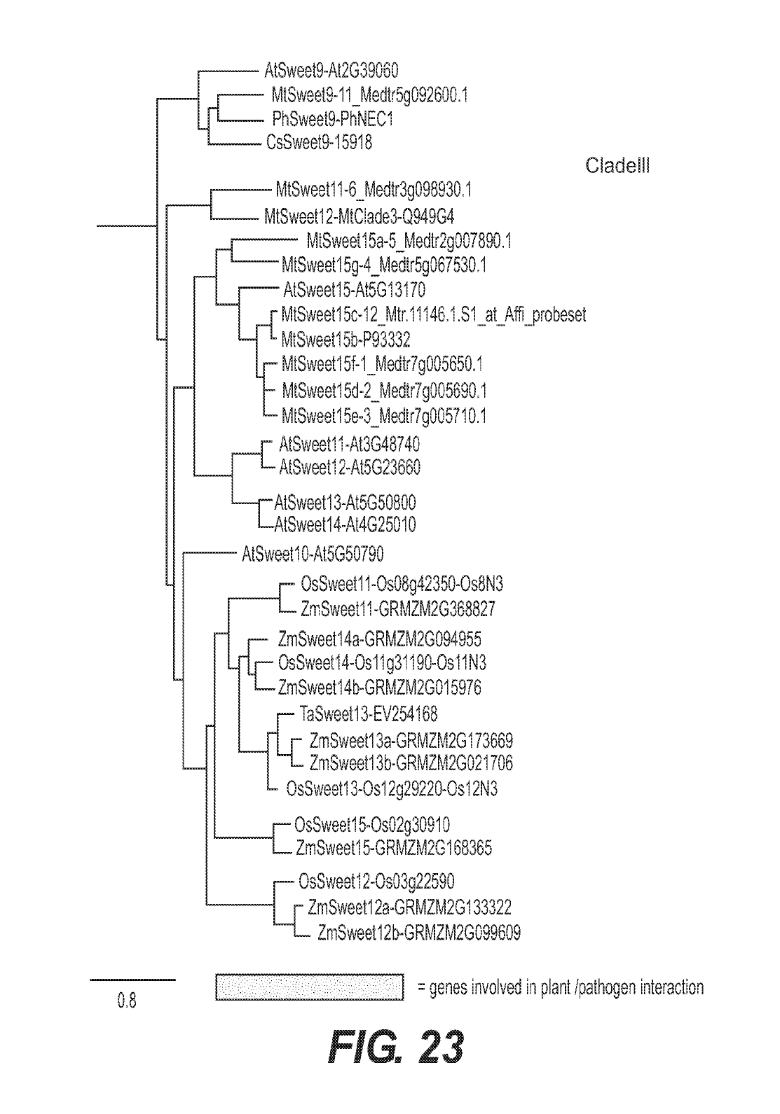

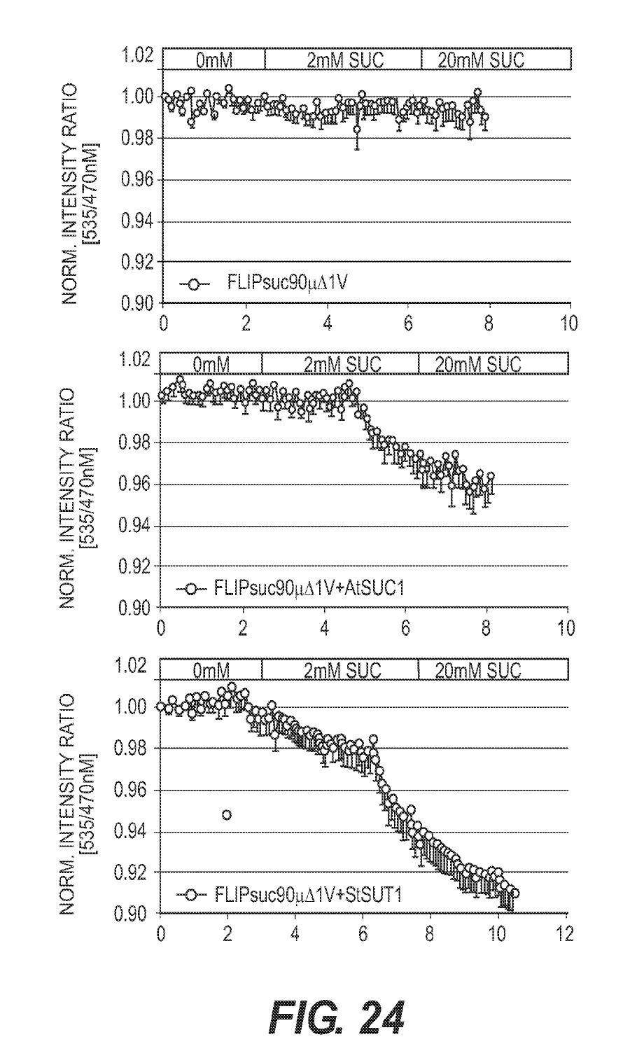

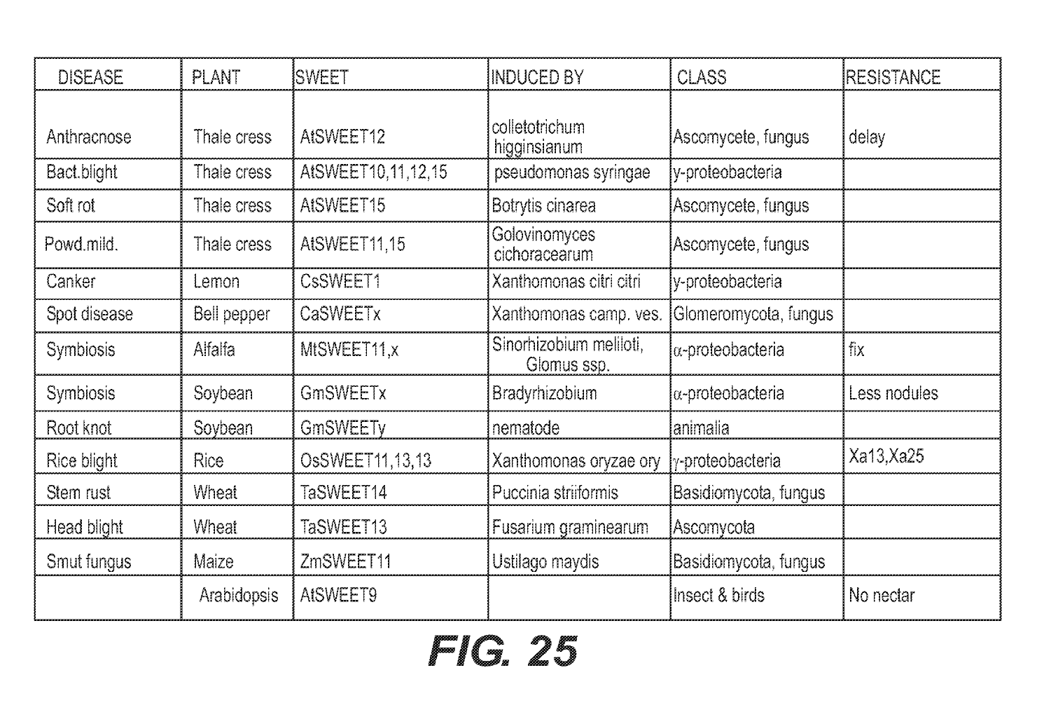

The role of sucrose transporters was also assessed in for the rice clade III transporter, OSSWEET13 (also referred to as OS12G29220; OS12N3) (see FIG. 23). As FIG. 20 demonstrates, when coexpressed in HEK 293 cells with the FRET sucrose and FRET glucose sensors as described above demonstrate that this gene functions as a weak glucose and as a highly efficient sucrose transporter. The experiments were carried out as described above and by Chen et al. (Nature 468, 527 (2010)).

### Example 4

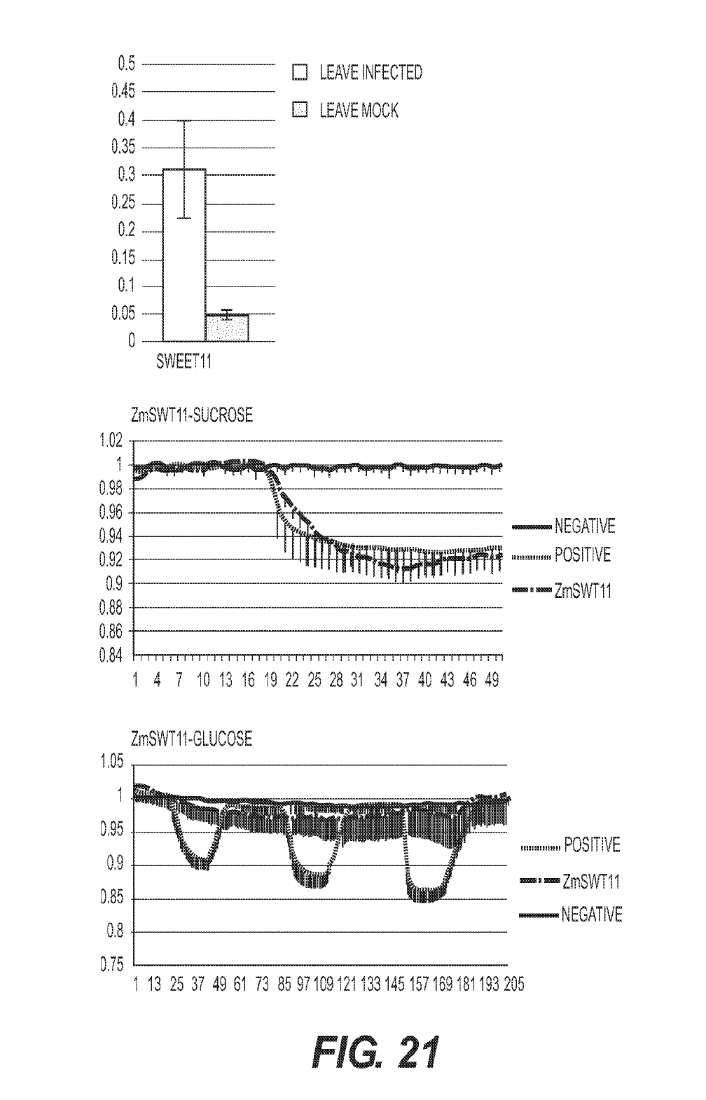

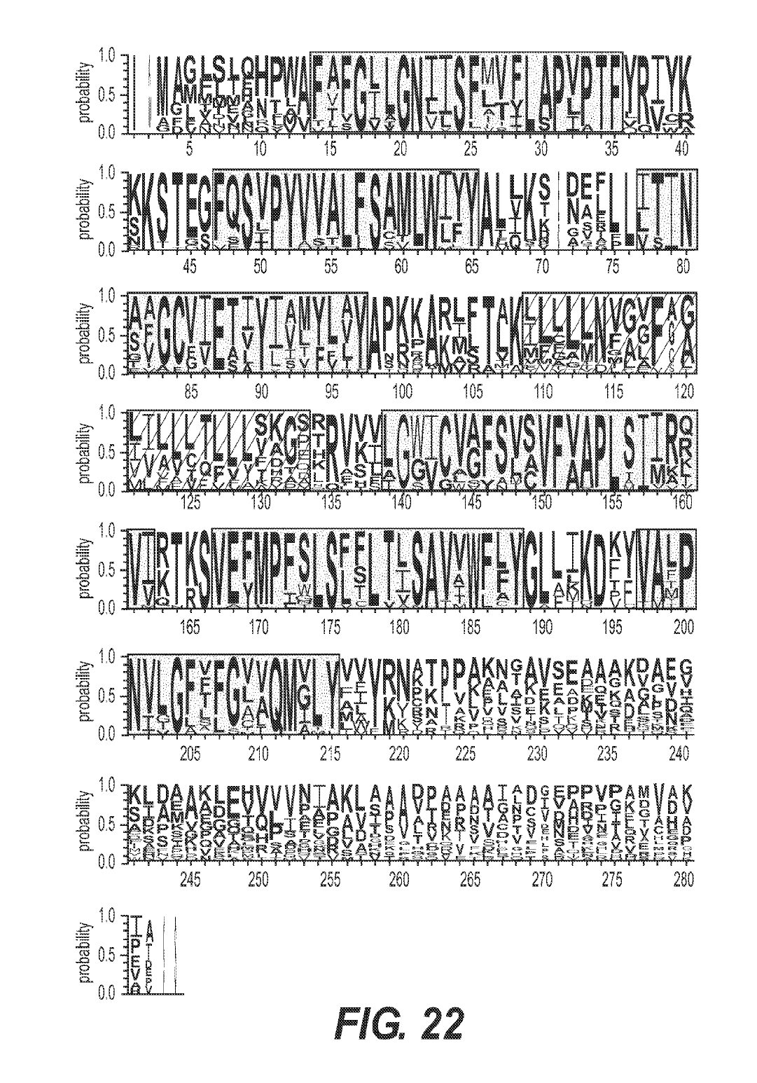

The role of sucrose transporters was also assessed in maize. ZmSWEET11, a further clade III transporter (see FIG. 21) is induced during Ustilago maydis infection. As FIG. 21 demonstrates, based on a comparison with the controls, there was about a 5-fold induction as measured by qPCR (FIG. 21, top panel). The second panel shows function of ZmSweet11 as a sucrose transporter by coexpression of the maize gene with a sucrose FRET sensor FLIPsuc90μ in HEK293T cells. The experiments were carried out as described above and by Chen et al. (Nature 468, 527 (2010)).

Hemibiotrophic fungi can grow either biographic or nectrotrophic. Although initial data only indicated that SWEETs are critical for pathogen infection in rice by a bacterial pathogen, Xanthomonas and although it was highly unlikely that this would be a general mechanism that applies to the specific interaction between Xanthomonas and rice, a domesticated monocot. It was an extreme situation that was tested where a hemibiotrophic fungus Colletotrichum, responsible for massive damage to many different crops, may also require SWEET transporters in a totally different host, namely the dicot weed Arabidopsis. Collectively with the group of Sonnewald and Voll (University Erlangen), it was found that AtSWEET11 and 12 were induced during Colletotrichum infection of Arabidopsis. While it could be potentially viewed as a side effect, when single or double mutants of Arabidopsis in AtSWEET11 or 12 genes were tested for resistance to Colletotrichum infection, it was surprisingly found that the development of the fungal infection was delayed and that the growth of the fungus, as evidenced by the amount of gDNA (genomic DNA specific to fungus) was significantly reduced. These data unambiguously demonstrate that the nutrient efflux mechanism is hijacked by pathogens, including diverse organisms, such as hemibiotrophic fungi and bacteria, such as Xanthomonas, in very diverse plant species, i.e., both monocots and dicots, thus providing proof of concept for the possibility to create not only crops resistant plants for specific pathogens in a binary fashion by the vaccination strategies outlined herein, but that it is possible to use the same mechanism to create stable, broad resistance to bacterial infections from a wide spectrum of bacteria as well as at the same time resistance to a wide spectrum of fungi. Since SWEETs are induced by nematodes, the resistance mechanisms can be much broader and will apply to also other pests and pathogens such as but not limited to nematodes.

The SWEETs are involved in cell-to-cell transport of sugars and thus can contribute to improved local supply of host cells with carbon and energy. Thus the optimization of energy transfer to cells surrounding infections can improve host resistance not only to bacteria, fungi and nematodes, but also to help defend against virus.

### Example 5

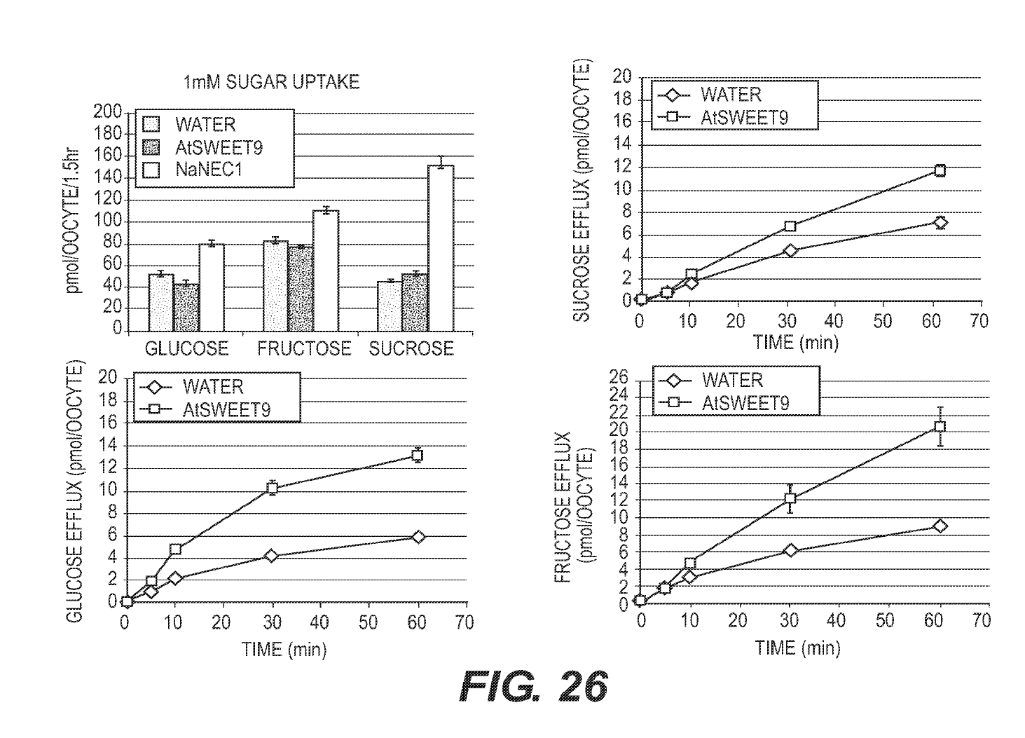

To test if AtSWEET9, like AtSWEET11 and AtSWEET12, can uptake or efflux sugars, Xenopus oocyte uptake and efflux assay were performed. The results showed that AtSWEET9 did not mediate significant uptake of glucose, fructose or sucrose; the AtSWEET9 homolog in Nicotiana attenuate, NaNEC1 showed uptake activity of glucose, fructose and sucrose (FIG. 26). The sucrose uptake activity of AtSWEET9 was also performed in human embryonic kidney cells by coexpressing AtSWEET9 with the FRET sucrose sensor FLIPsuc90μΔ1V. AtSWEET9 did not enable HEK293T cells to accumulate sucrose, as detected by a negative ratio change in sensor output. On the other hand, AtSWEET9 has efflux activity for glucose, fructose and sucrose (FIG. 26). Thus the results suggest that AtSWEET9 is an efflux transporter but shows low sugar uptake activity in oocyte system.

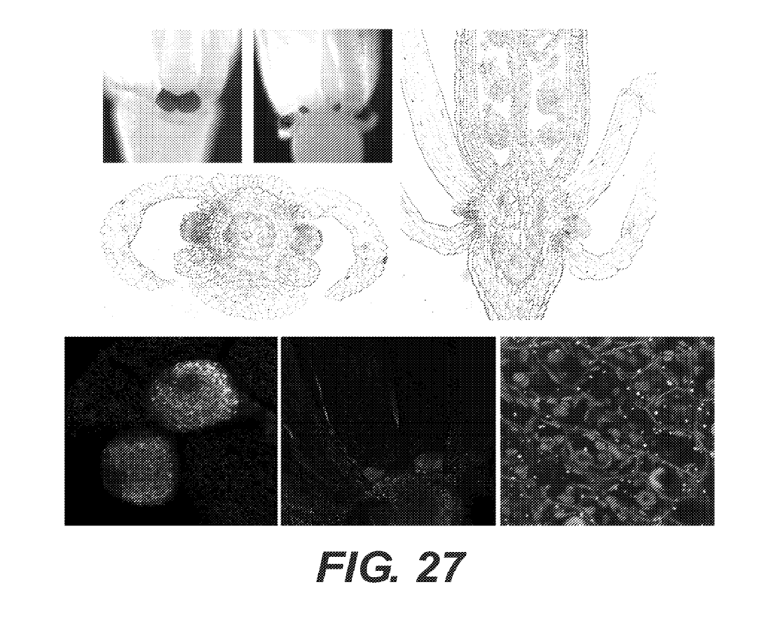

To confirm the tissue specific localization of AtSWEET9, the localization of AtSWEET9-GUS and AtSWEET9-eGFP proteins was examined in transgenic Arabidopsis containing AtSWEET9 native promoter and the complete coding region of AtSWEET9 including introns fusion GUS or enhanced GFP proteins. Both AtSWEET9-GUS and AtSWEET9-eGFP proteins are localized specifically in both lateral and medium nectaries of Arabidopsis flowers (FIG. 27). To further investigate the specific localization of cell type for AtSWEET9 in the nectary, flowers were stained and embedded into LR-White resin and sectioned using microtome. FIG. 27 shows sections of GUS-stained AtSWEET9-GUS transgenic flowers. The results demonstrate that AtSWEET9-GUS fusion proteins localize in nectaries, specifically in parenchyma but not in guard cells and most of the epidermis cells of the nectaries (FIG. 27). The AtSWEET9-GUS and eGFP fusion proteins were concentrated in the base of the nectary parenchyma cells. The signal of AtSWEET9-eGFP in the mature lateral nectaries (at anthesis, floral stage 14^{˜}15) is much stronger than the signal in the medium nectaries and immature lateral nectaries (before anthesis). The results are compatible with PhNEC1 promoter-GUS expression which showed the highest expression in the open flowers in which active secretion of nectar and starch hydrolysis had taken place. The AtSWEET9-eGFP proteins showed the subcellular localization in plasma membrane, Golgi and also as vesicles (FIG. 27). By using the FRAP technique (fluorescent recovery after photobleaching), the AtSWEET9-eGFP diffusion in the plasma membrane was monitored. The half time of recovery into the bleached region is about 80 seconds, which indicates rapid diffusion rate of AtSWEET9-eGFP in the plasma membrane. The results suggest that AtSWEET9 was constitutively sent to the plasma membrane. The vesicular localization of AtSWEET9-eGFP showed highly dynamic movement. Together, the localization results indicate that AtSWEET9 functions as transporters in plasma membrane or vesicle in the base of the nectary parenchyma.

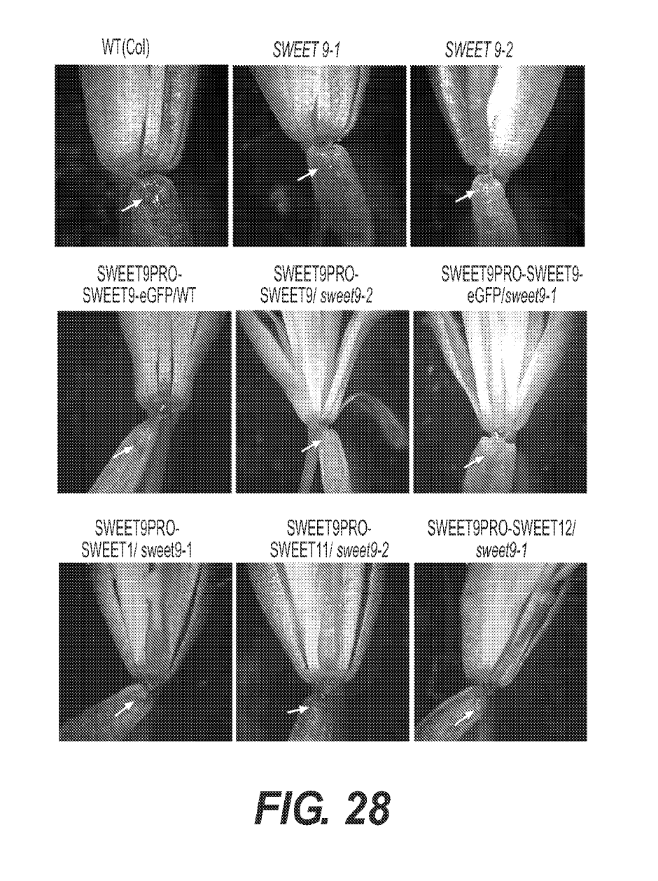

To determine whether AtSWEET9 is necessary for nectar production, two independent T-DNA insertion mutant lines were identified (sweet9-1 carries a T-DNA insertion in pos. −308 before start codon which had no detectable transcript levels; sweet9-2 pos. −940 before start codon, which had reduced transcript levels. Normally, nectar droplets accumulate inside the cups formed by sepals surrounding the lateral nectaries. FIG. 28 shows nectar droplet clinging to the inside of a sepal of a wild-type flower. Contrary to wild-type flowers, no nectar droplets were found in mutant flowers. The mutants with the exception of non-nectar phenotype, looks identical to wild-type plants. As judged by scanning electron microcopy (SEM), mutant nectaries appeared to have similar morphology to wild-type nectaries, including the shape of nectaries, indicating that the phenotype was not due to the lack of nectaries. To verify that the phenotype is instead due to loss function of AtSWEET9, complemented lines were generated by transforming constructs containing native promoter and the complete coding region of AtSWEET9, or native promoter and the complete coding region of AtSWEET9 fusion eGFP into the sweet9 mutant lines. In both complemented transgenic lines, the nectar production of nectaries can be restored. Nectar production in the transgenic lines containing native promoter and the complete coding region of AtSWEET9 fusion eGFP in wild-type background was also observed. The result showed that more nectar produced than wild-type flowers. Thus, AtSWEET9 is necessary for nectar production (FIG. 28) and more copies of AtSWEET9s are sufficient to produce more nectar. The nectar production phenotype was complemented by expression of AtSWEET1, AtSWEET11 and 12 under AtSWEET9 promoter in the sweet9 mutant (FIG. 28). Together, these data indicate that an impaired ability of the sweet9 mutants to export sugars from the nectaries. The function of AtSWEET9 can be restored by complemented the sugar efflux transporters AtSWEET11/12 and glucose efflux transporter AtSWEET1 expressing in the nectaries.

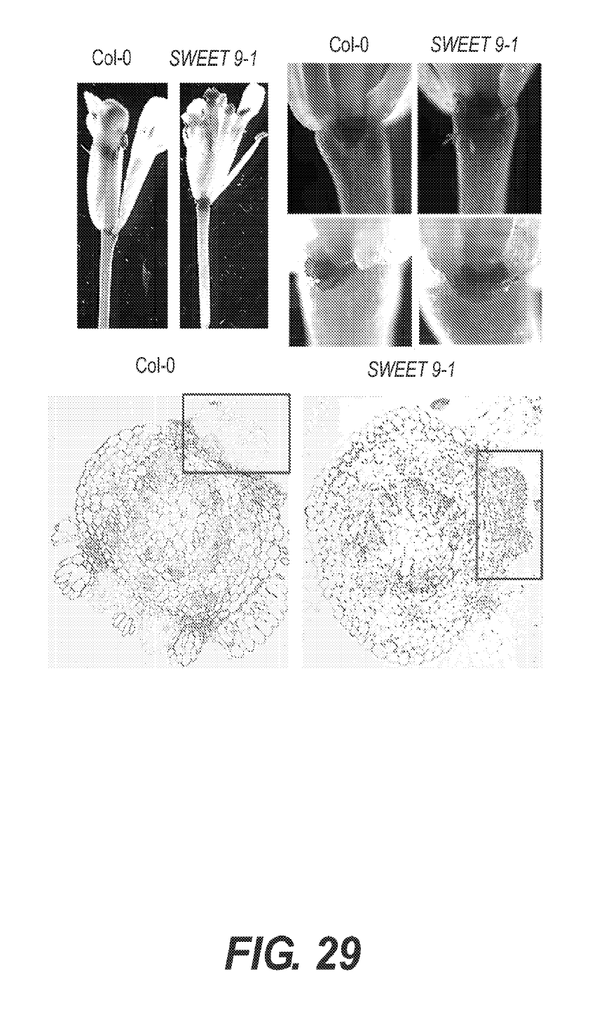

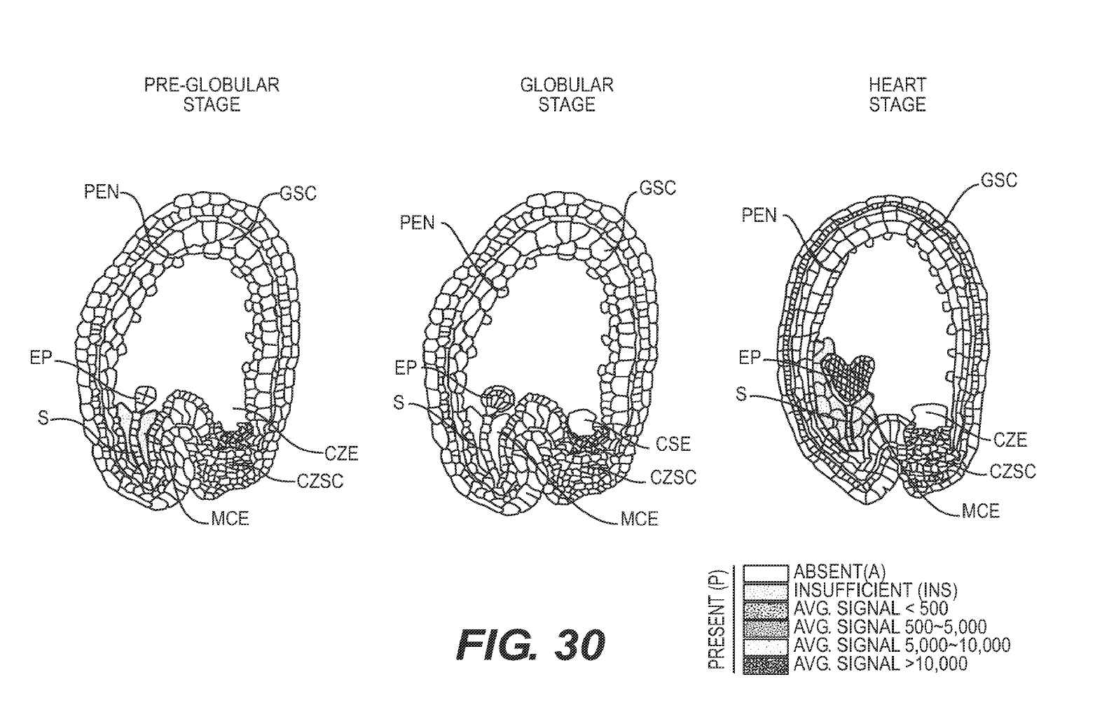

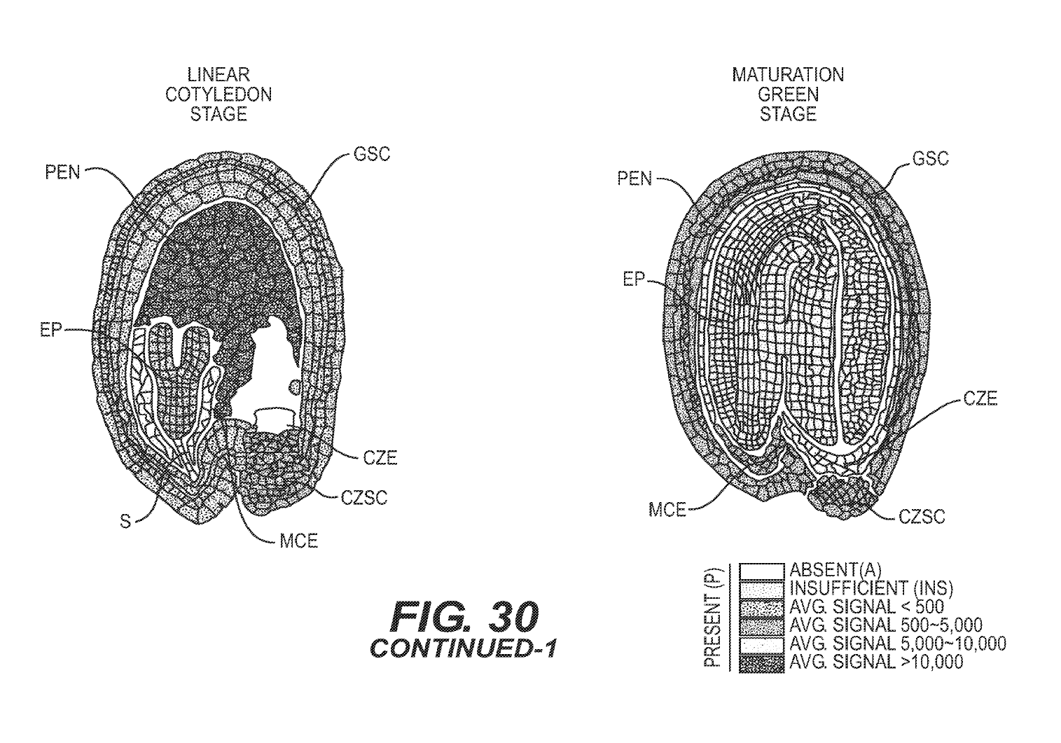

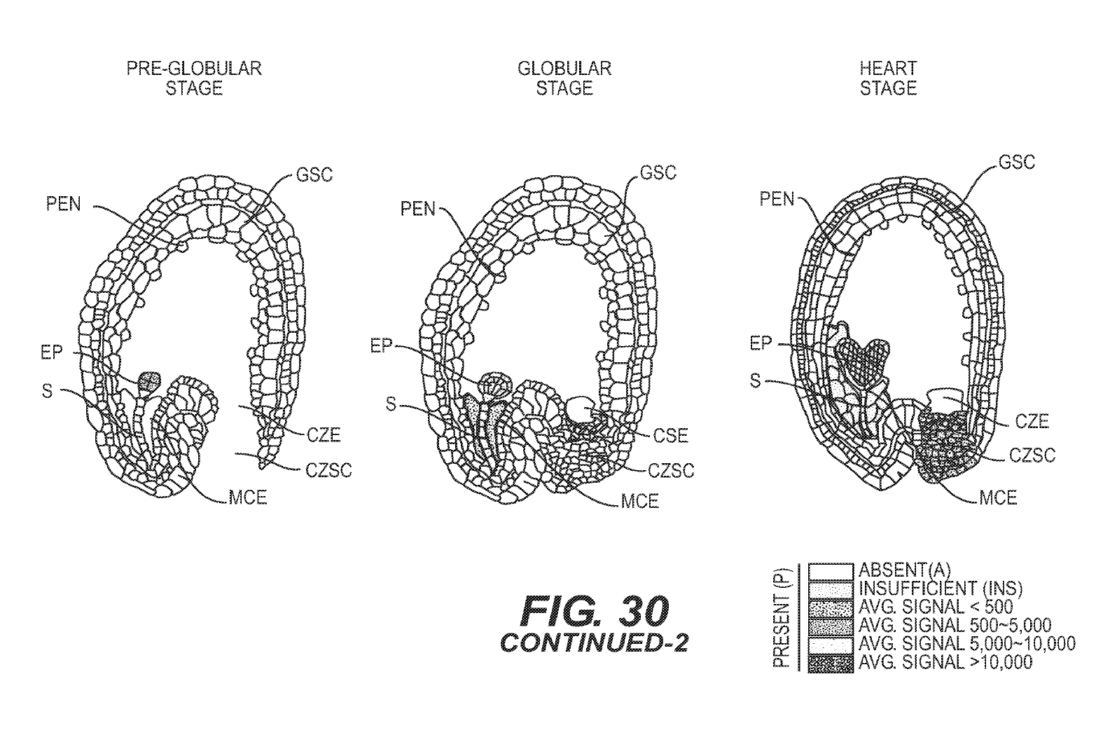

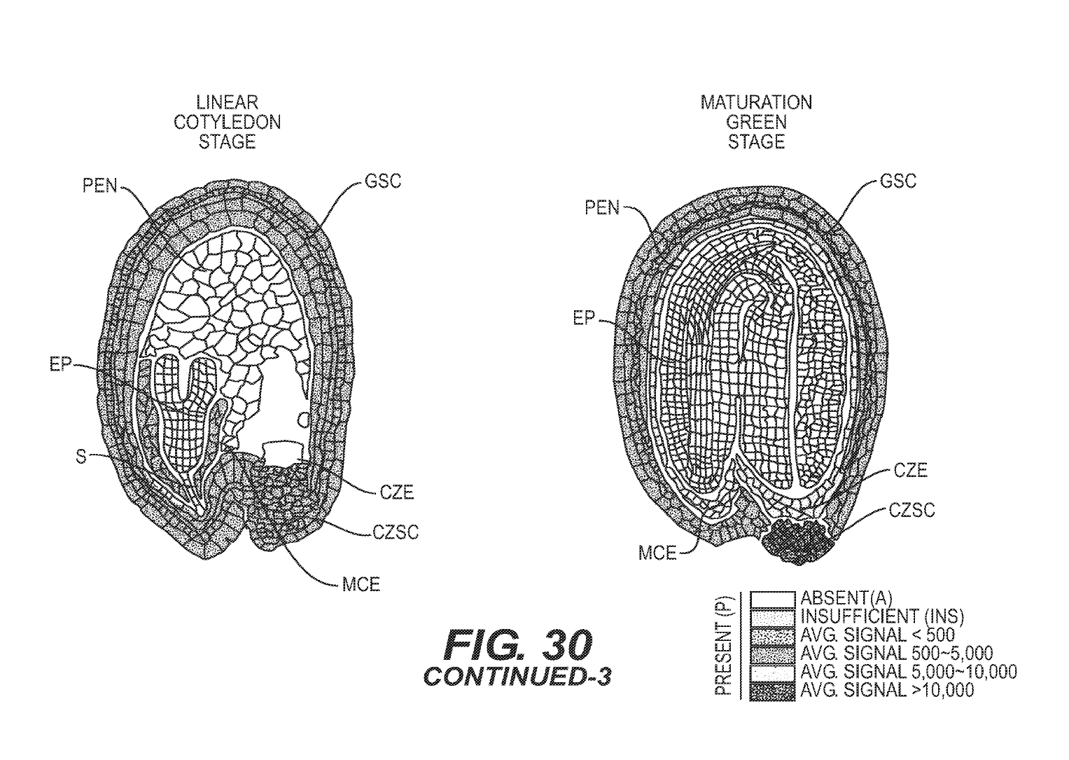

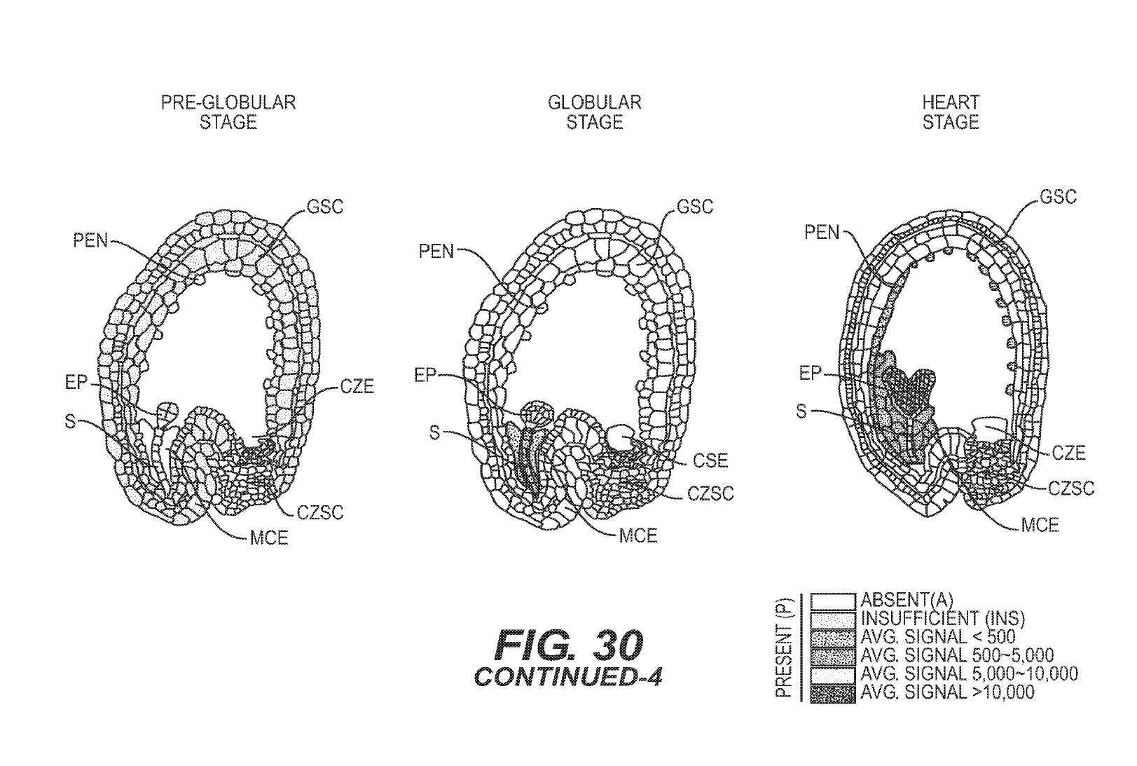

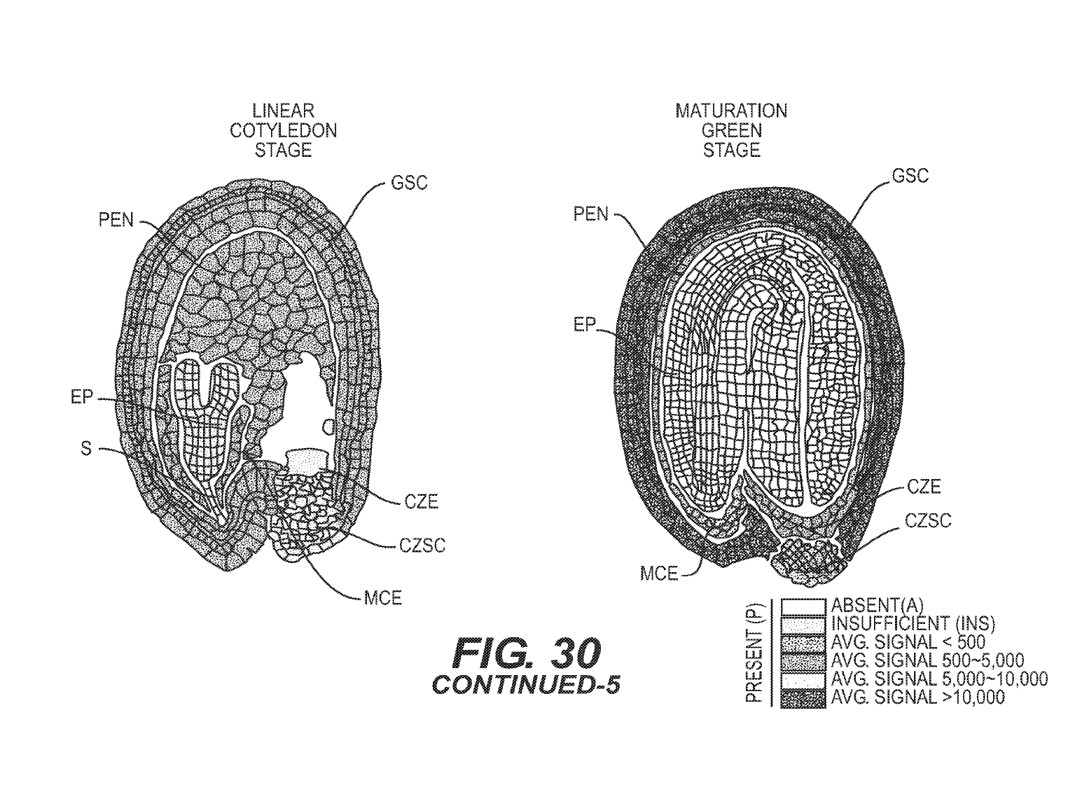

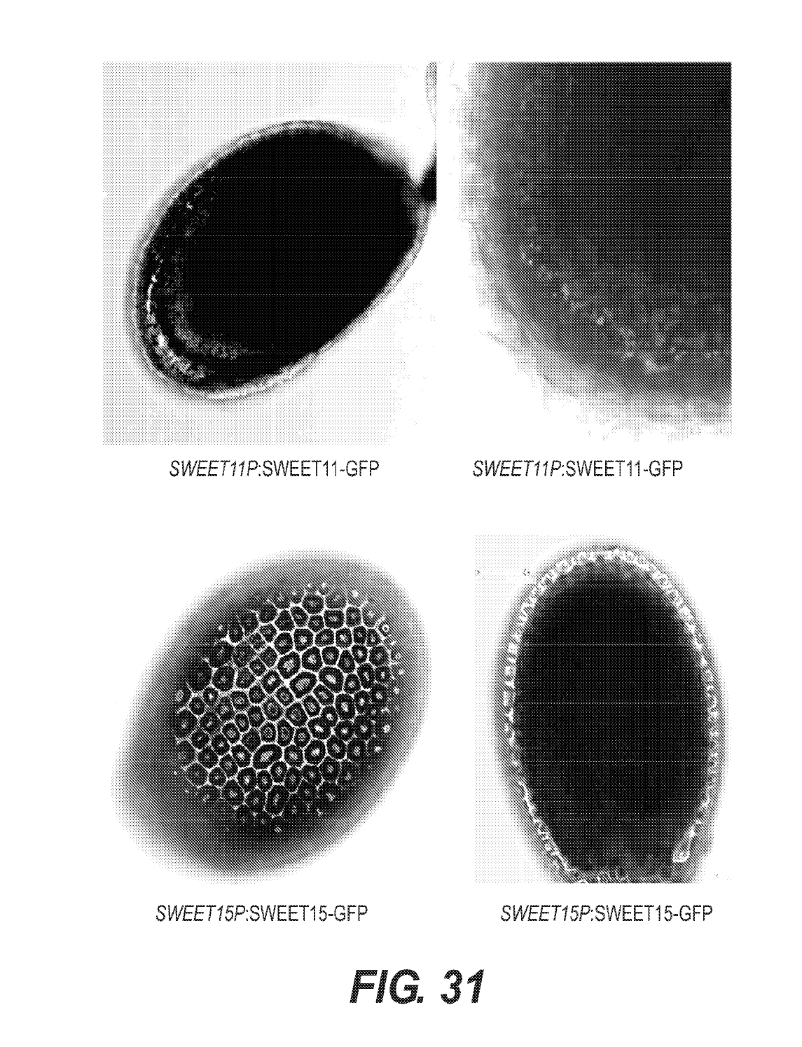

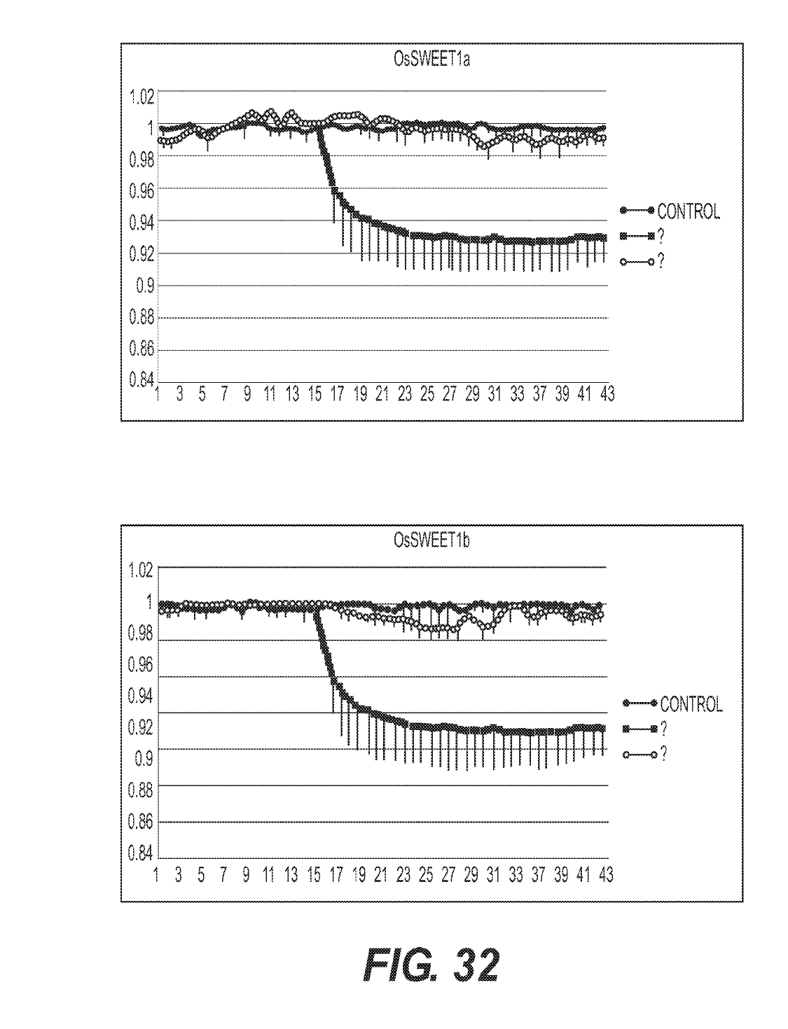

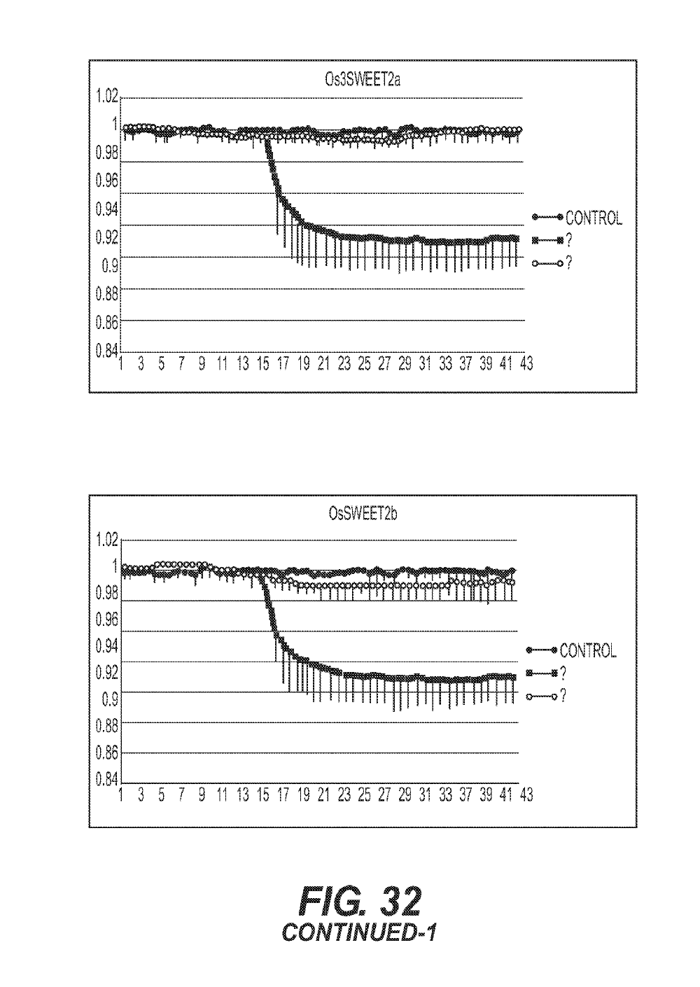

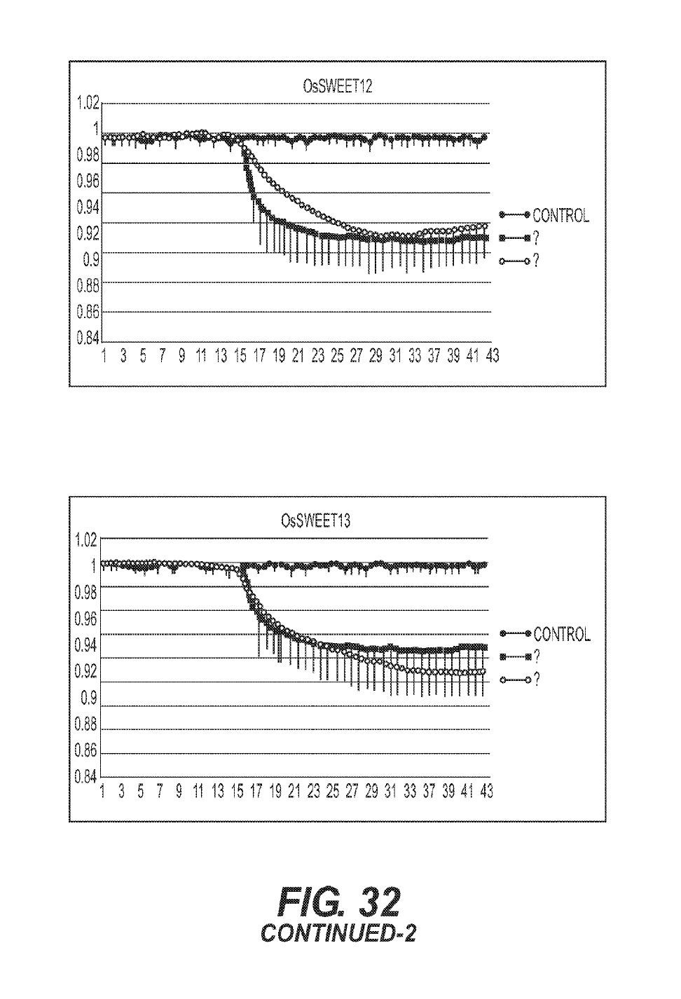

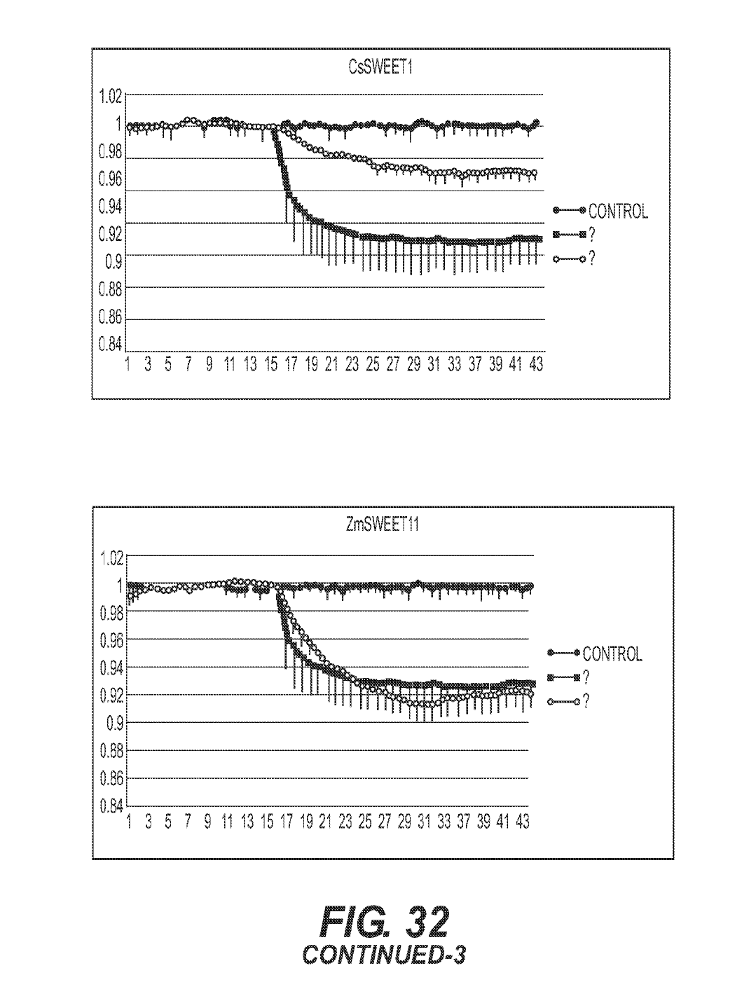

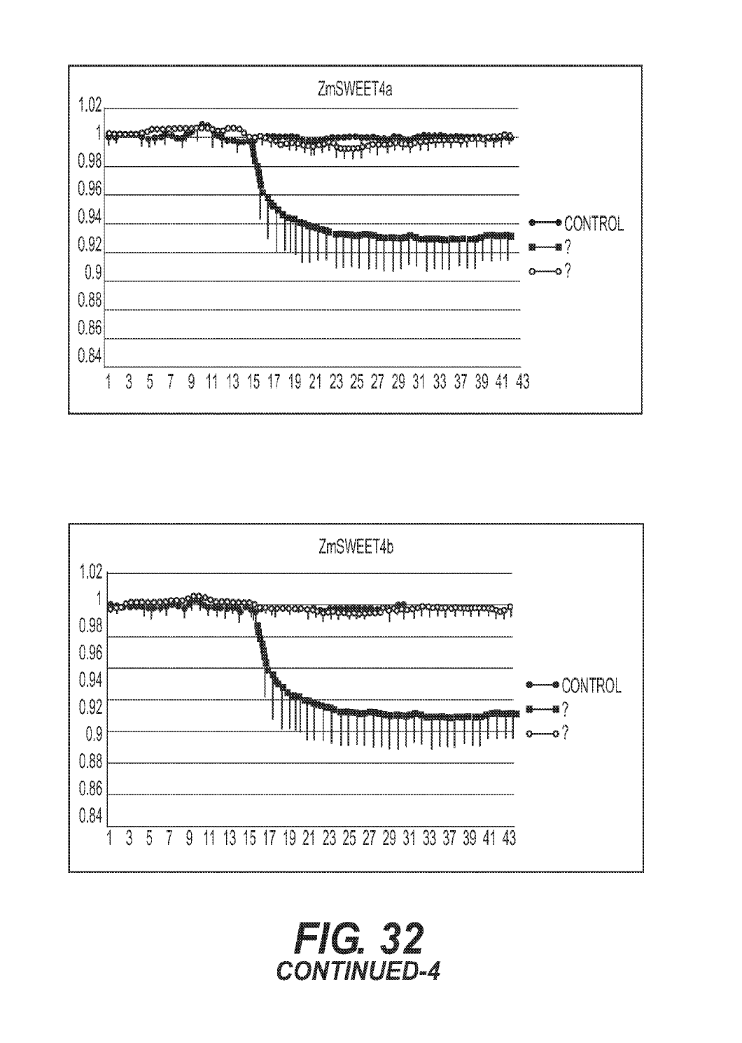

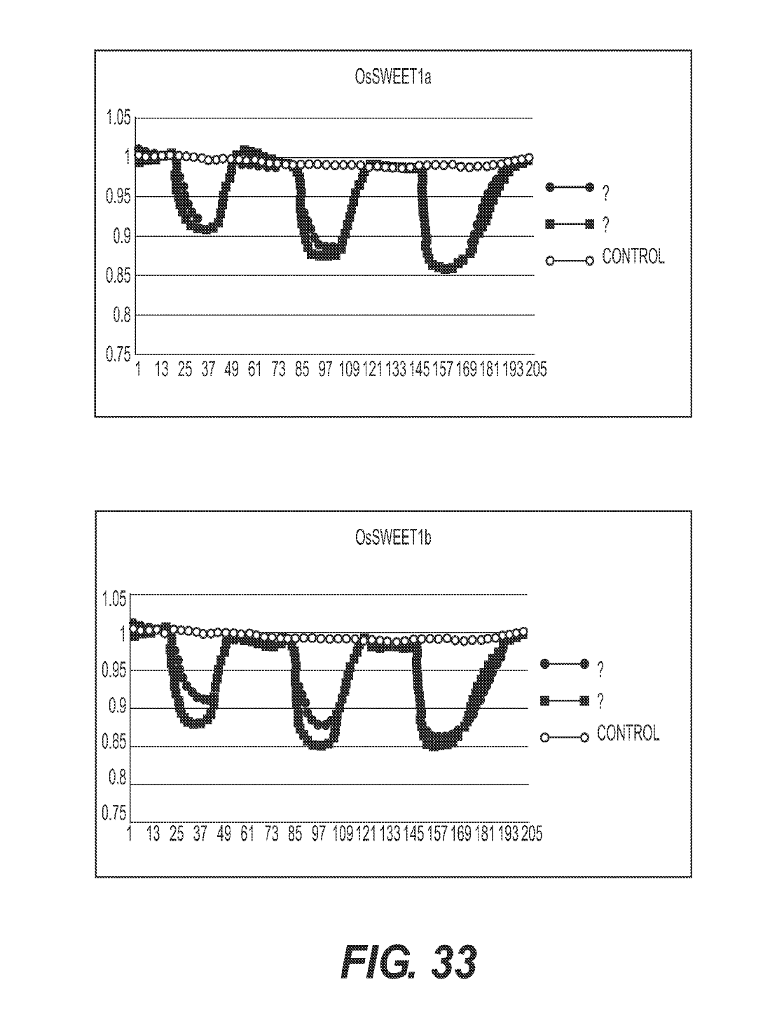

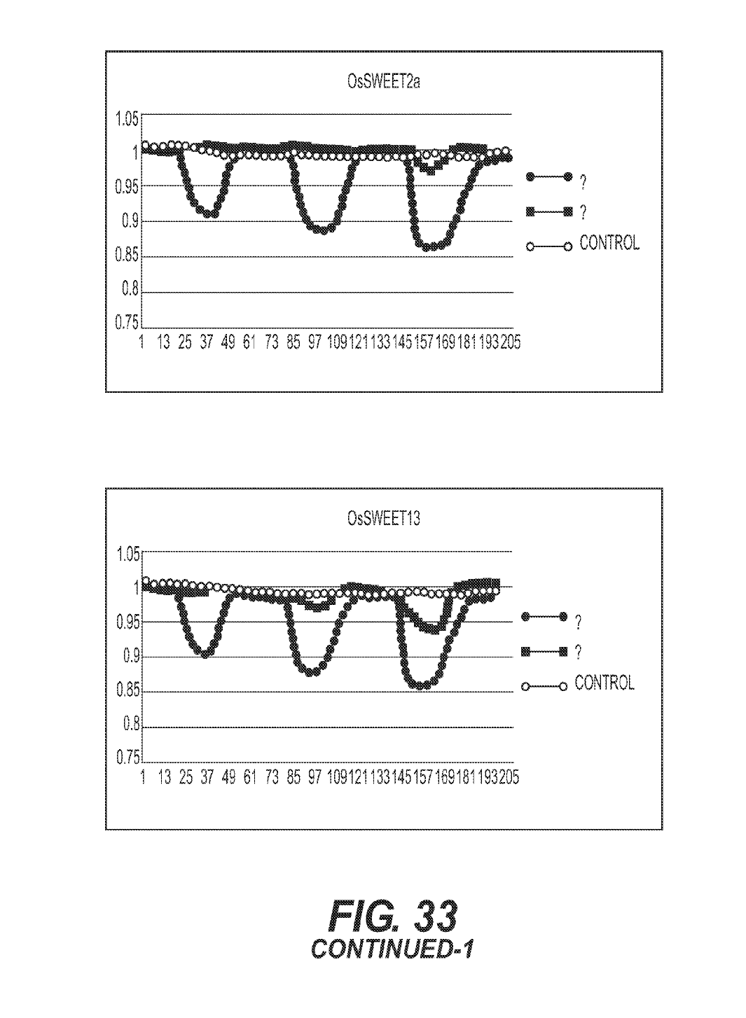

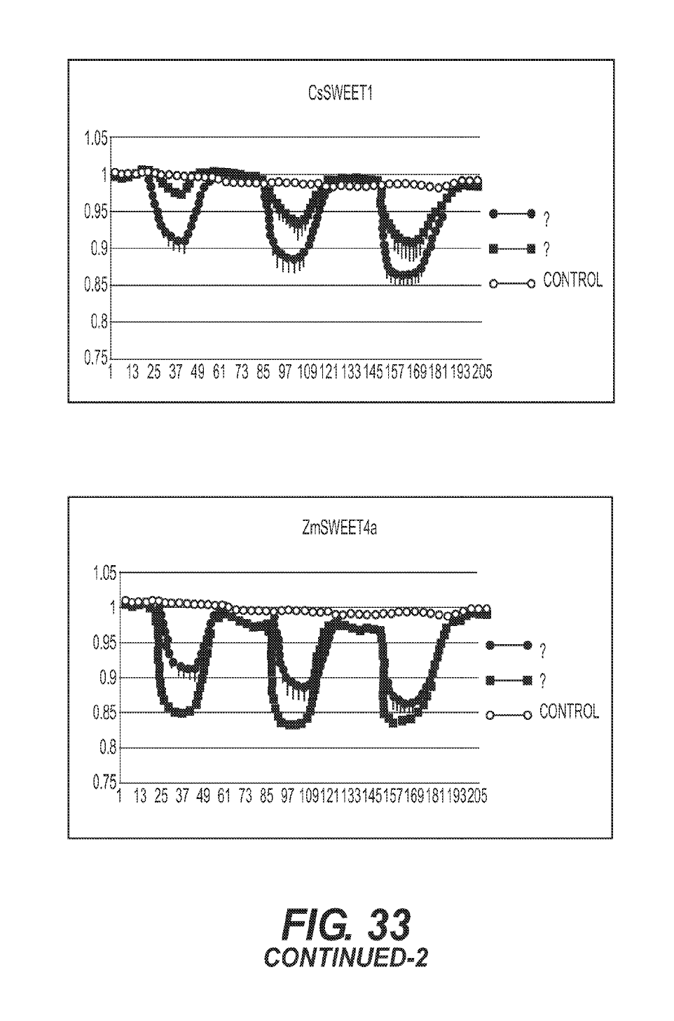

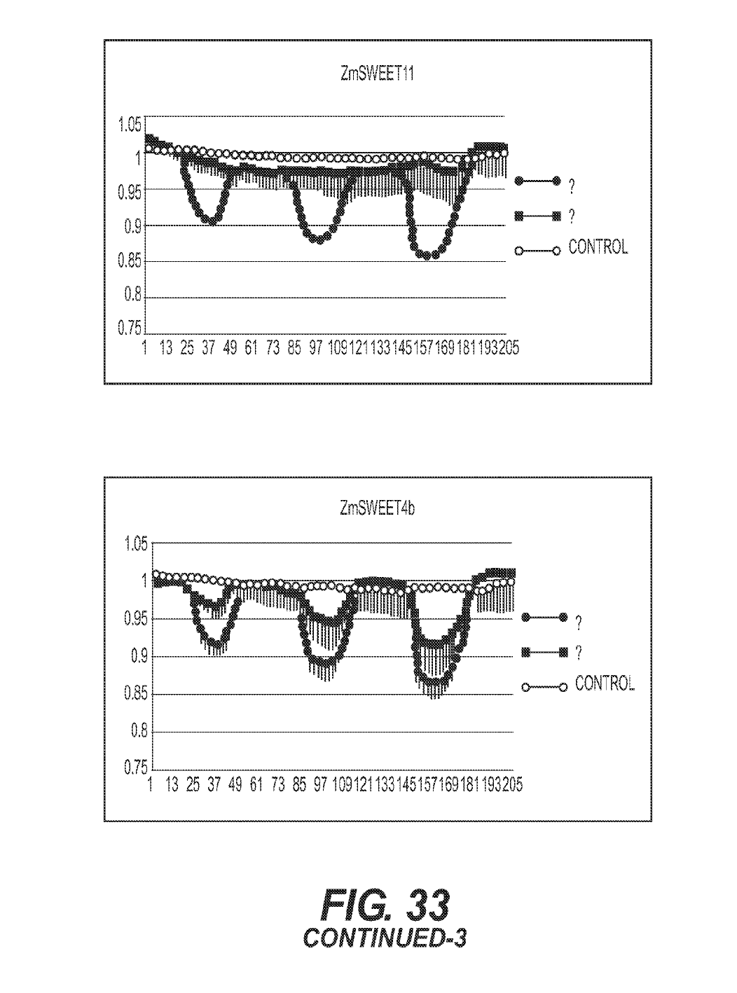

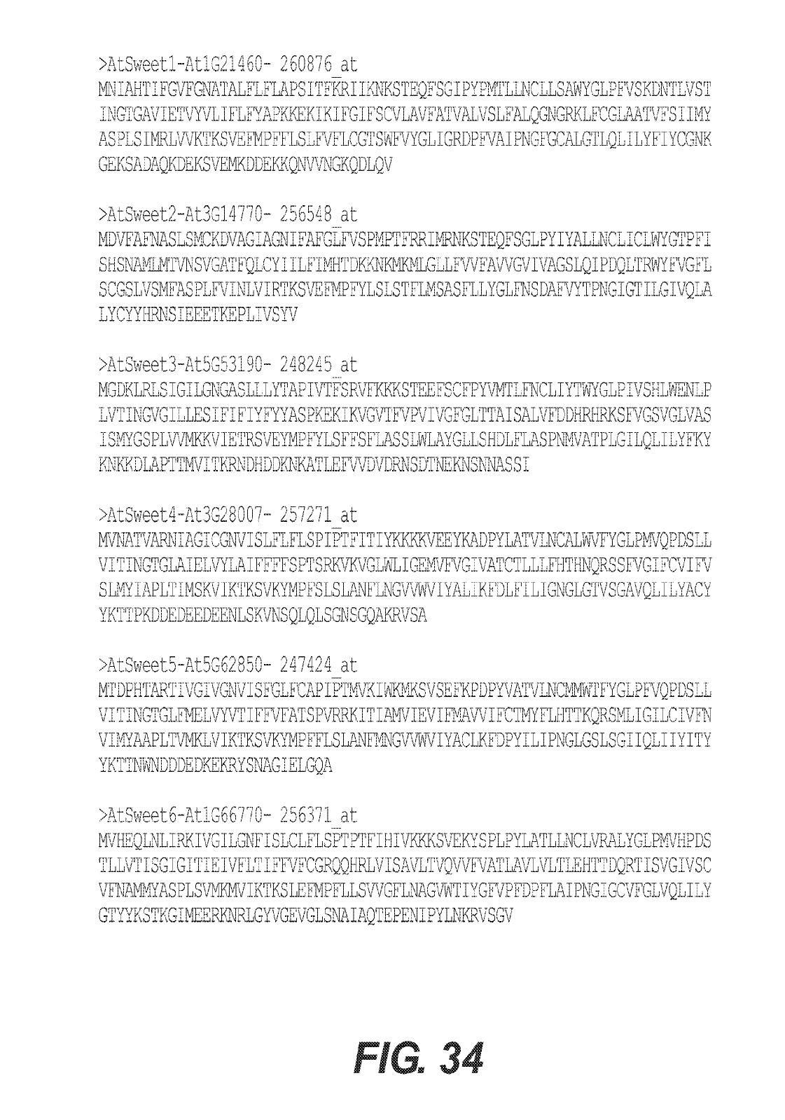

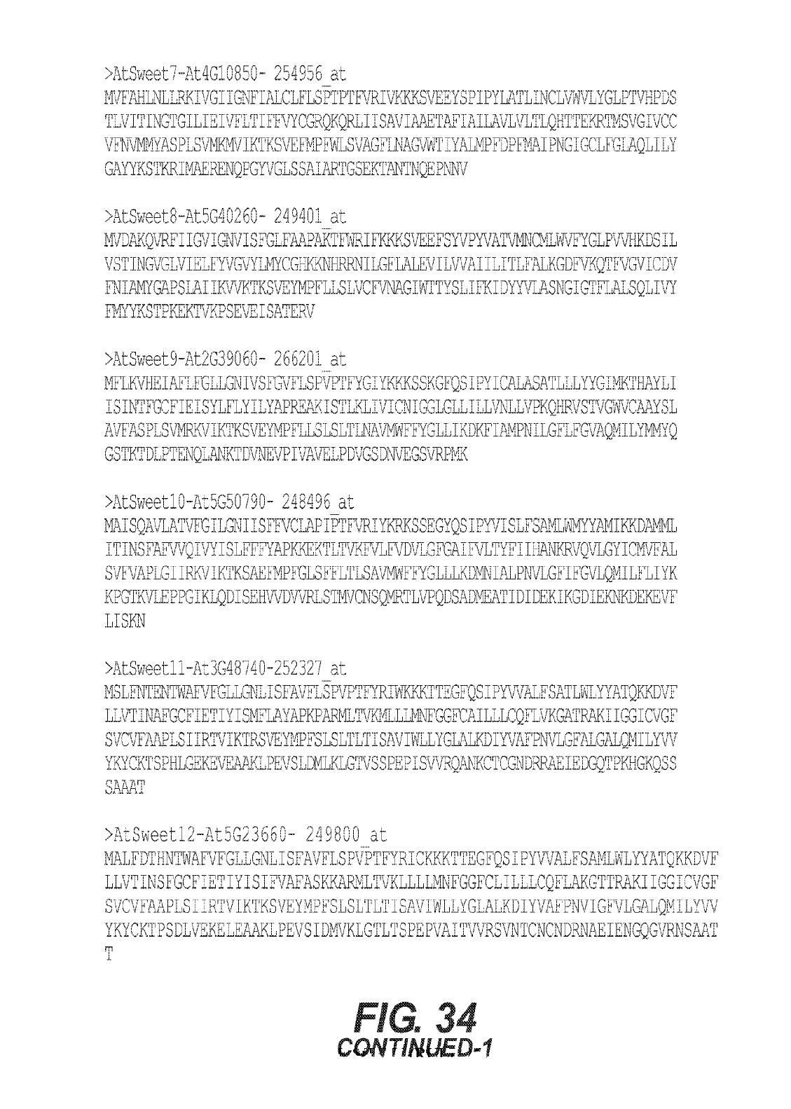

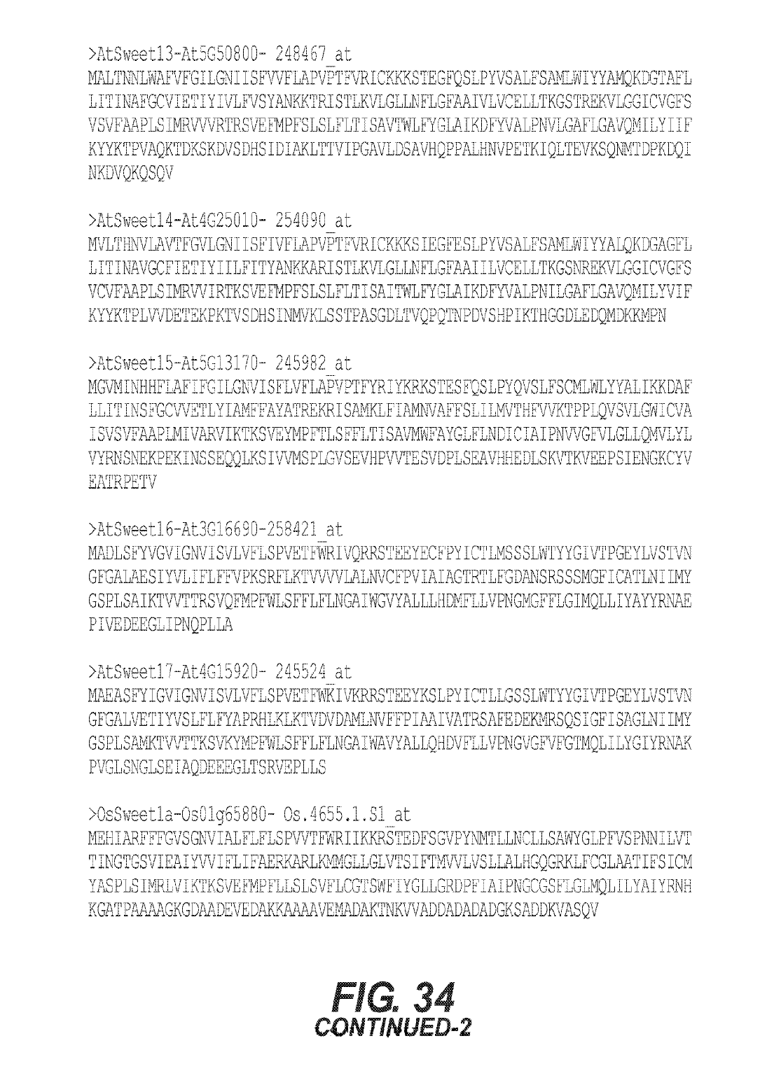

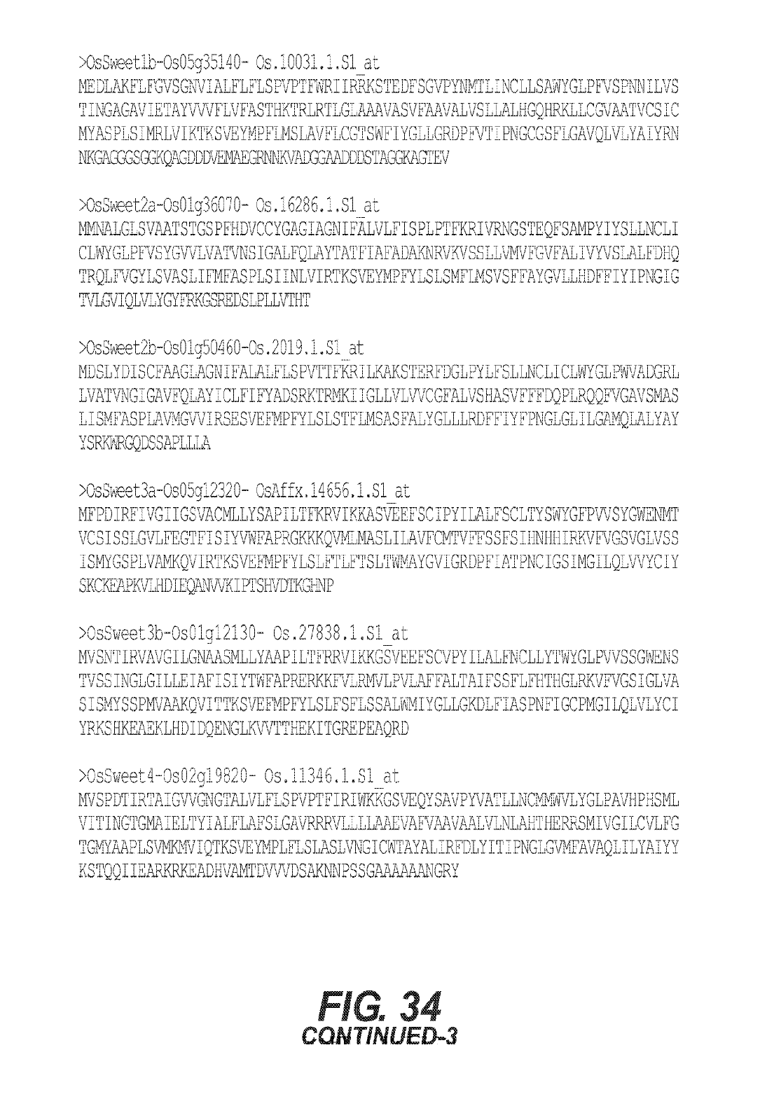

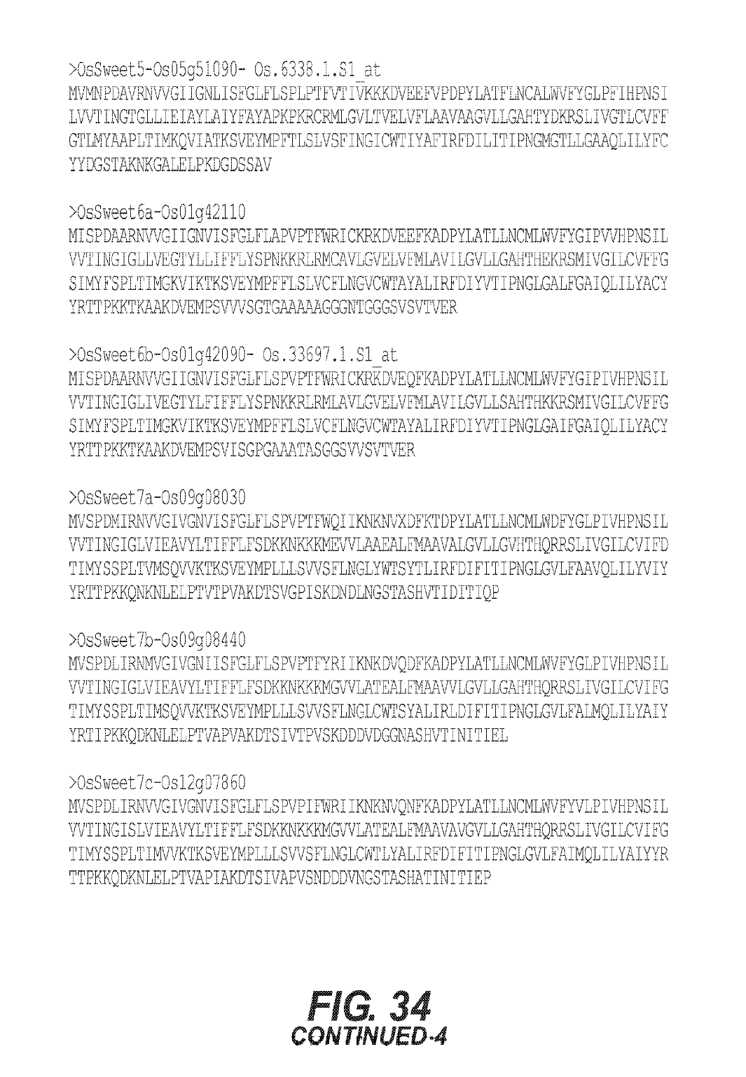

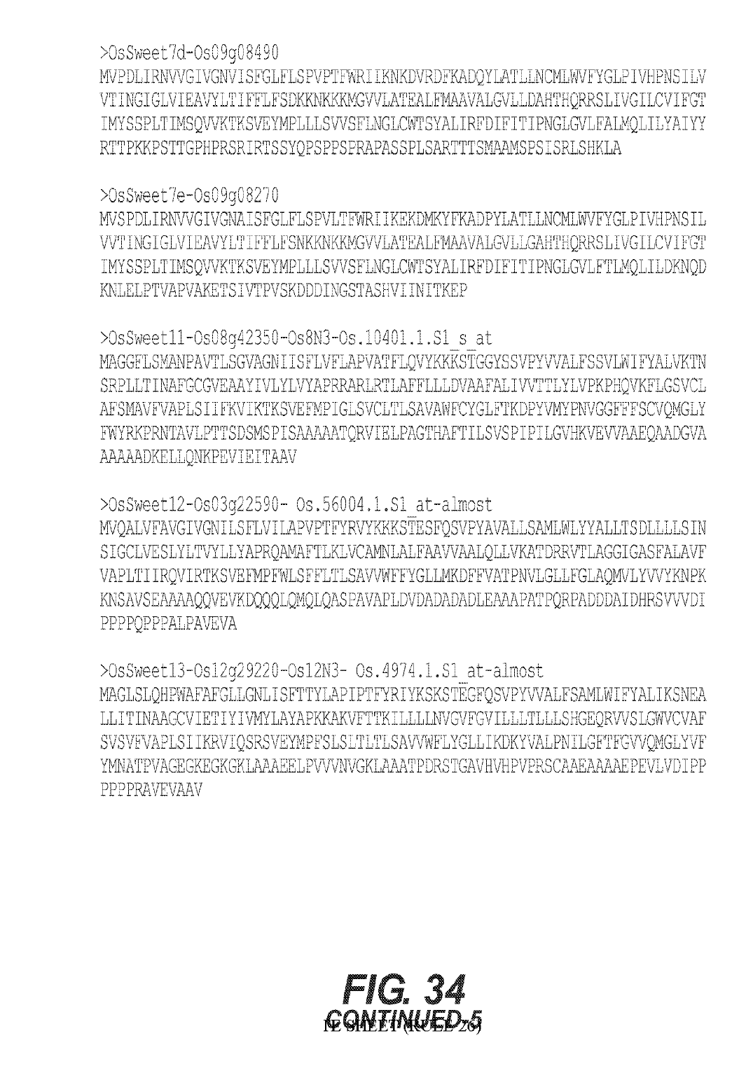

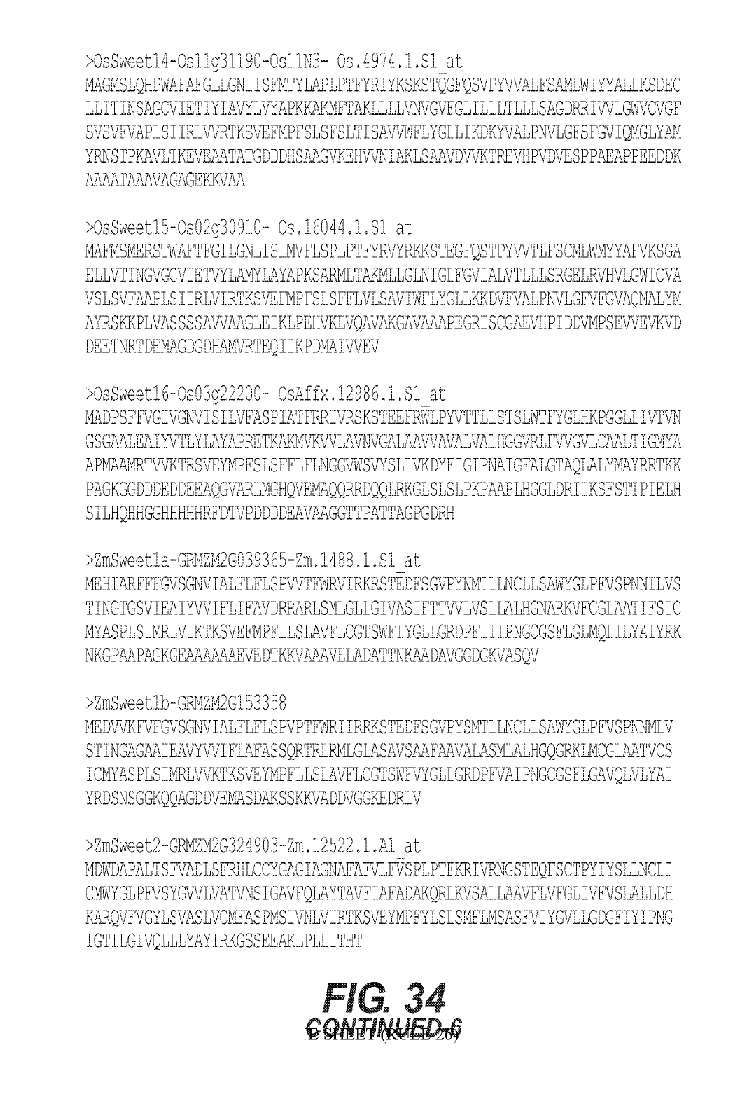

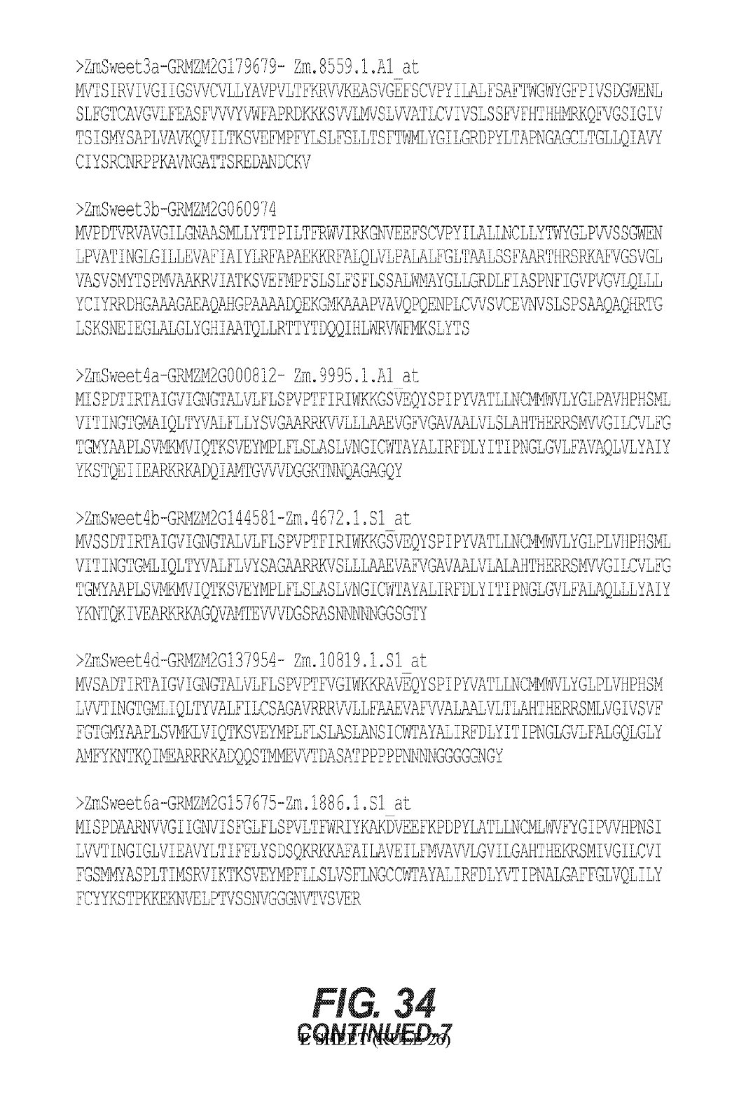

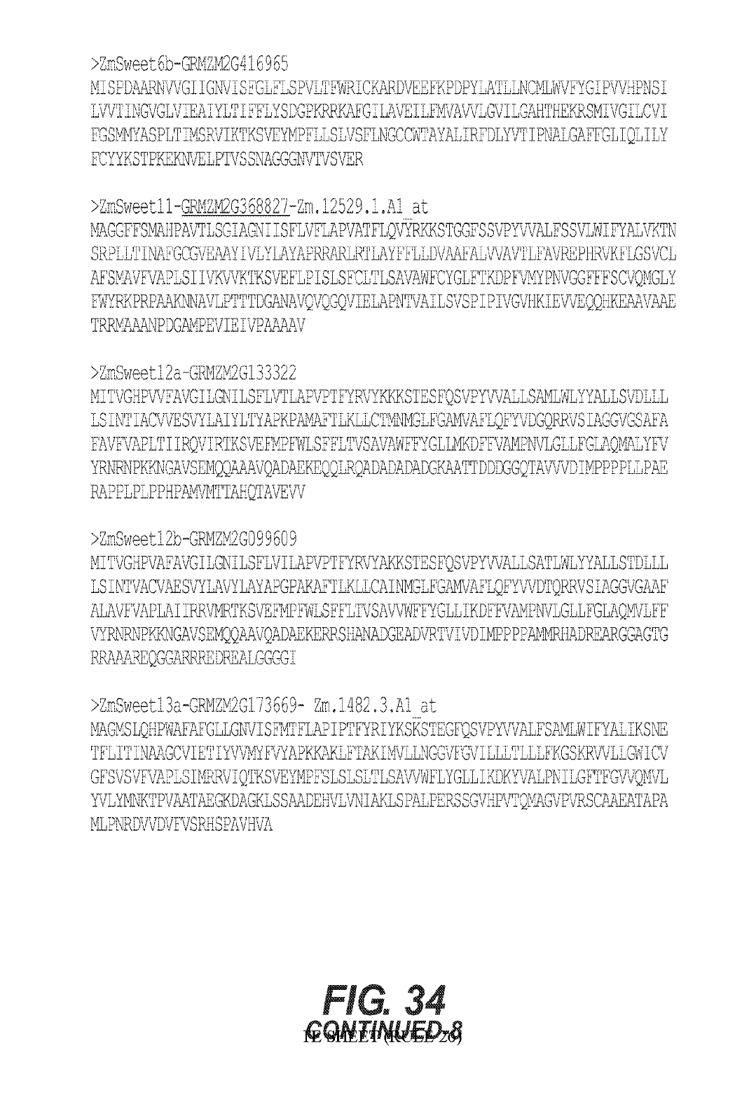

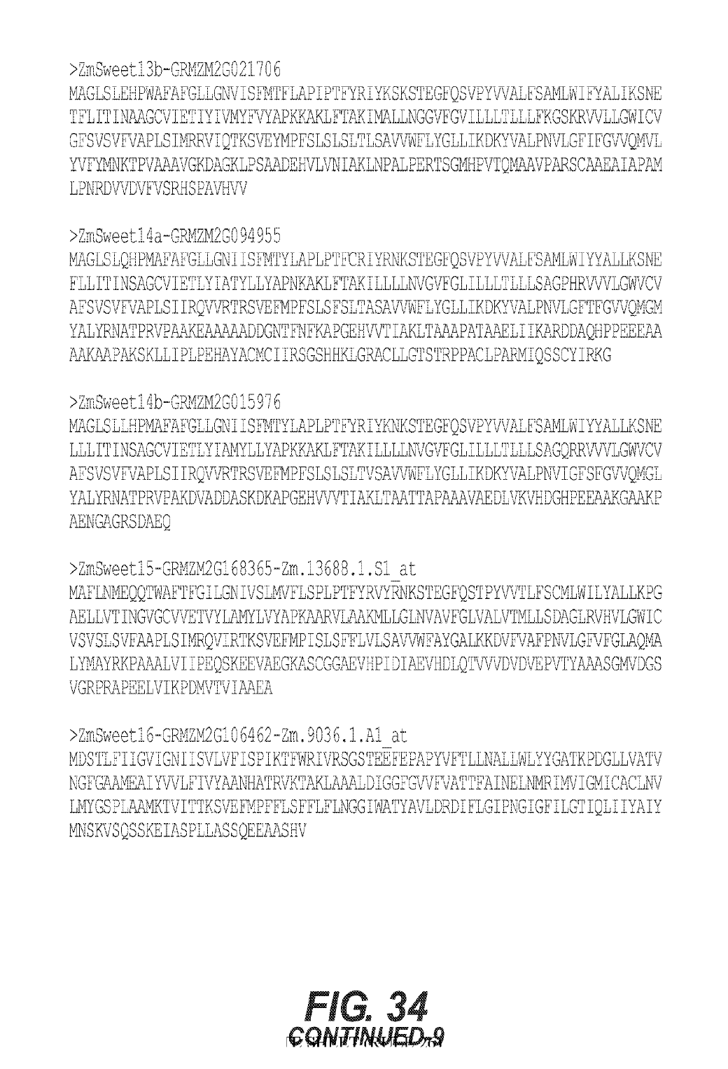

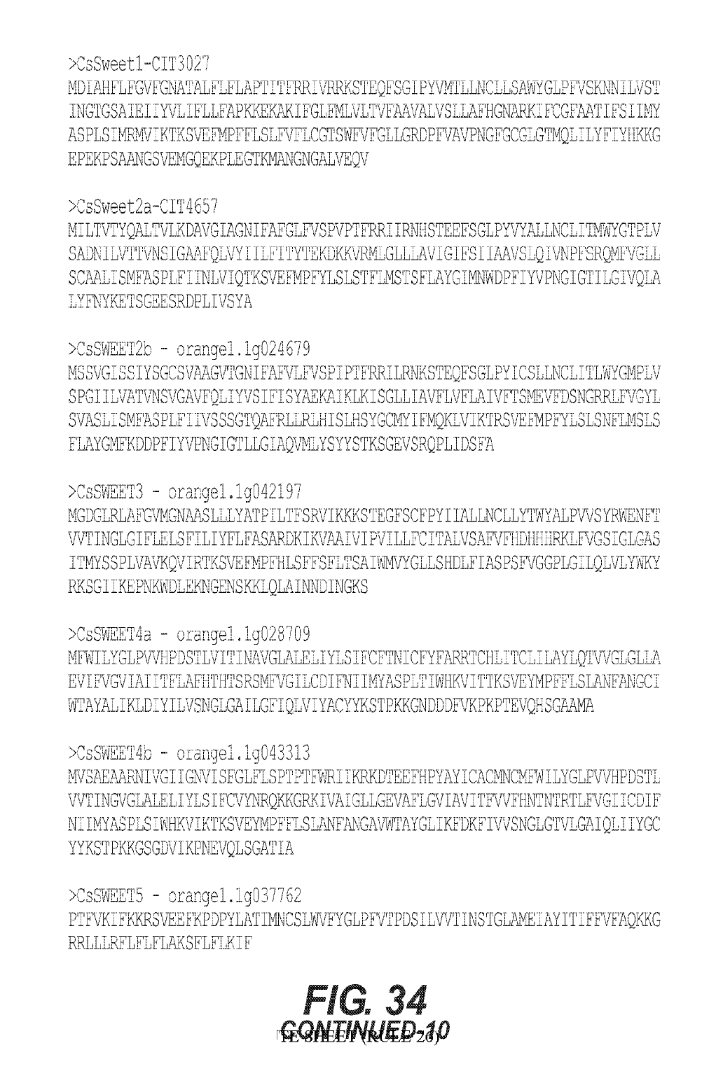

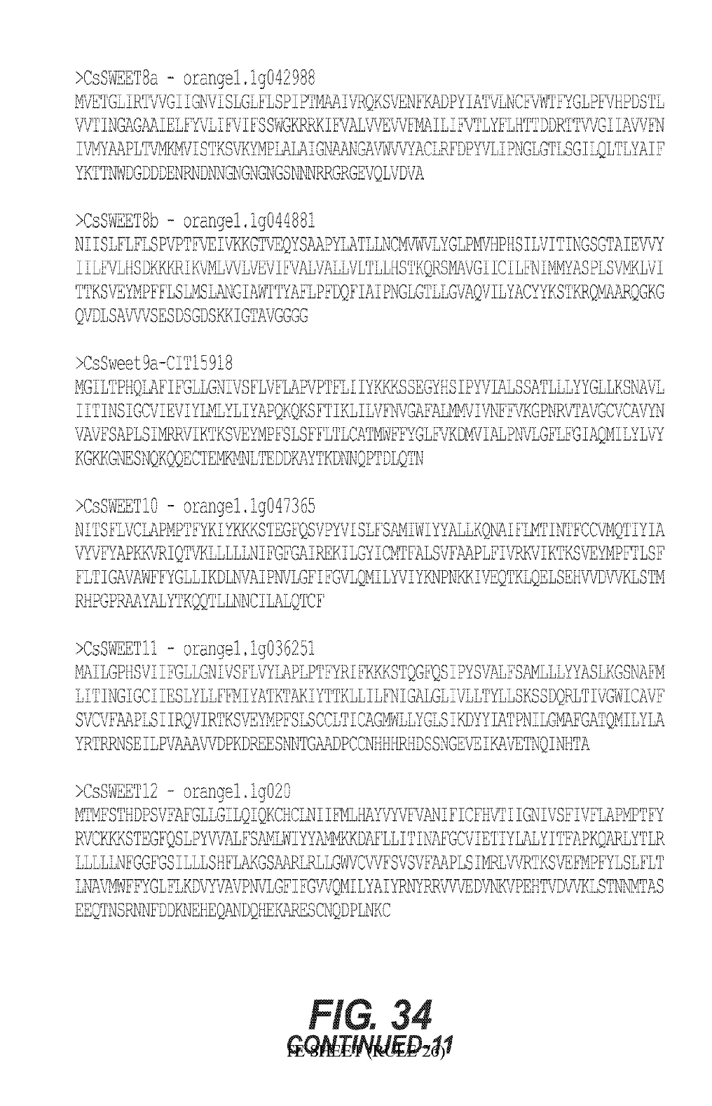

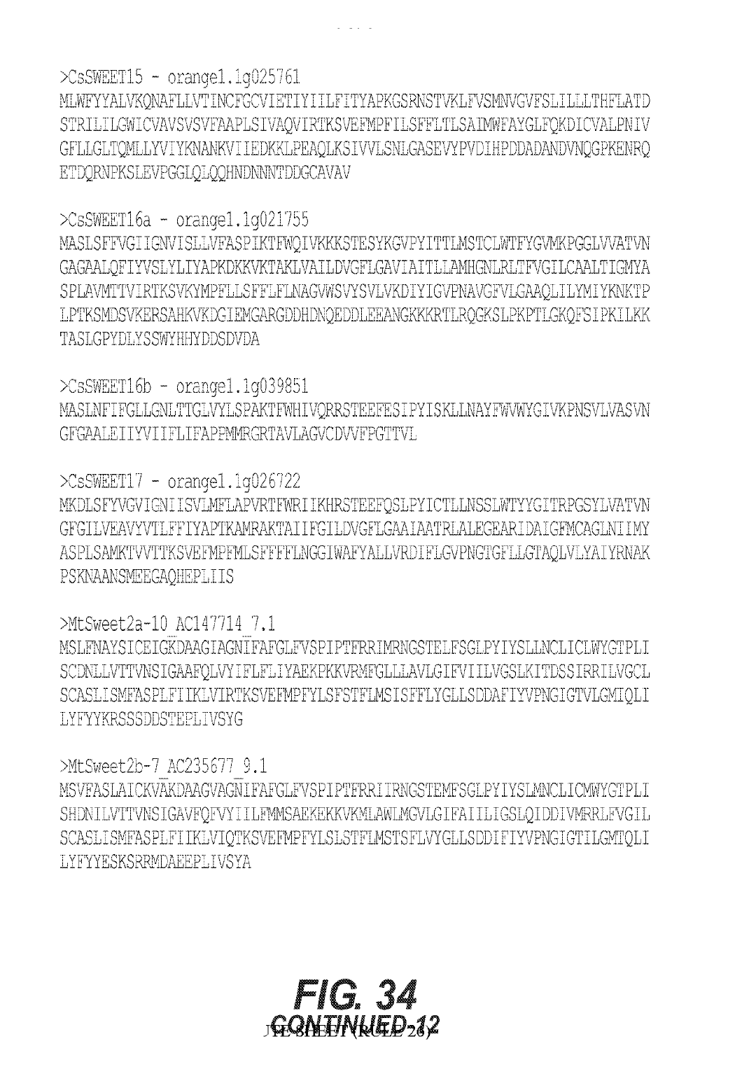

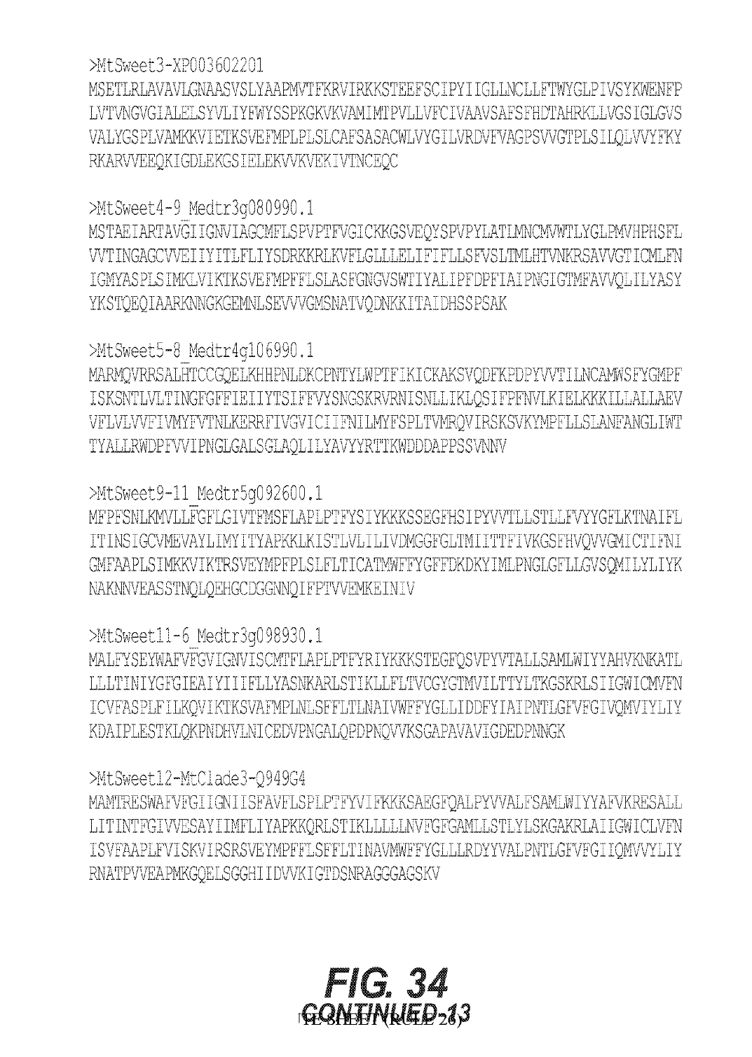

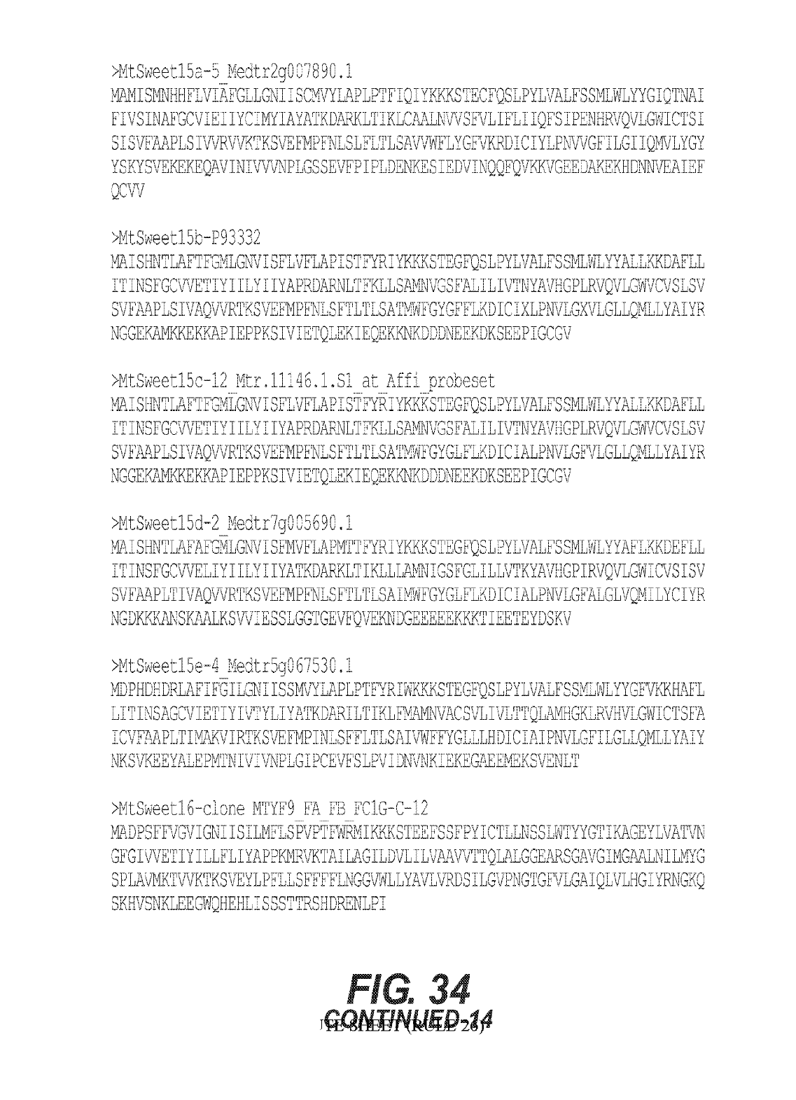

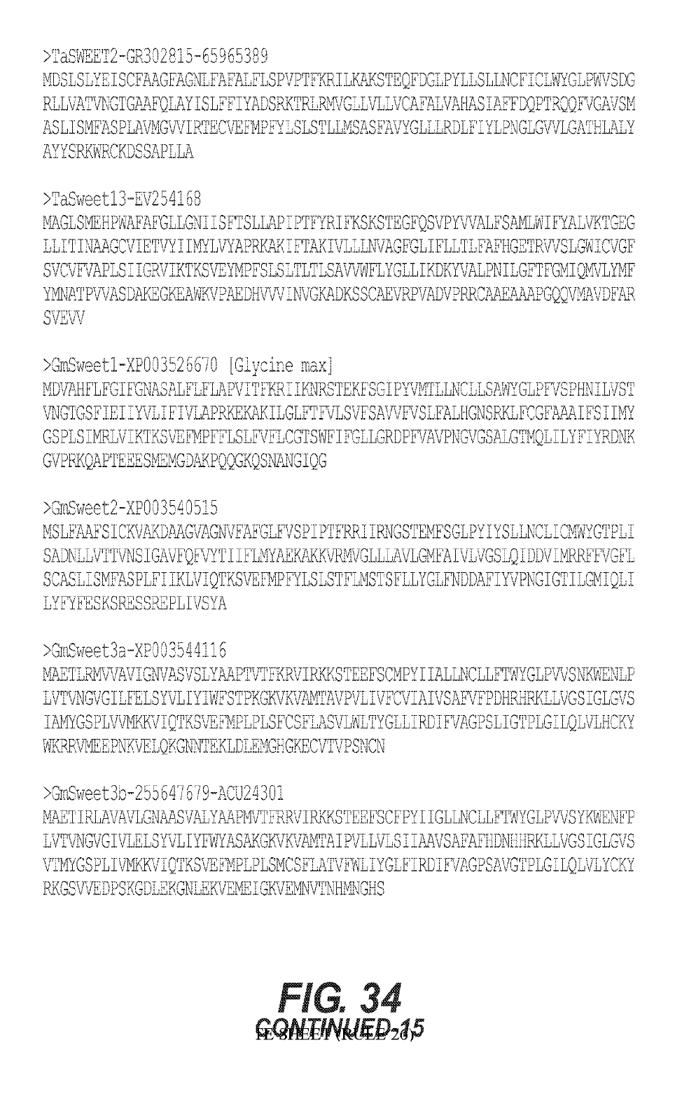

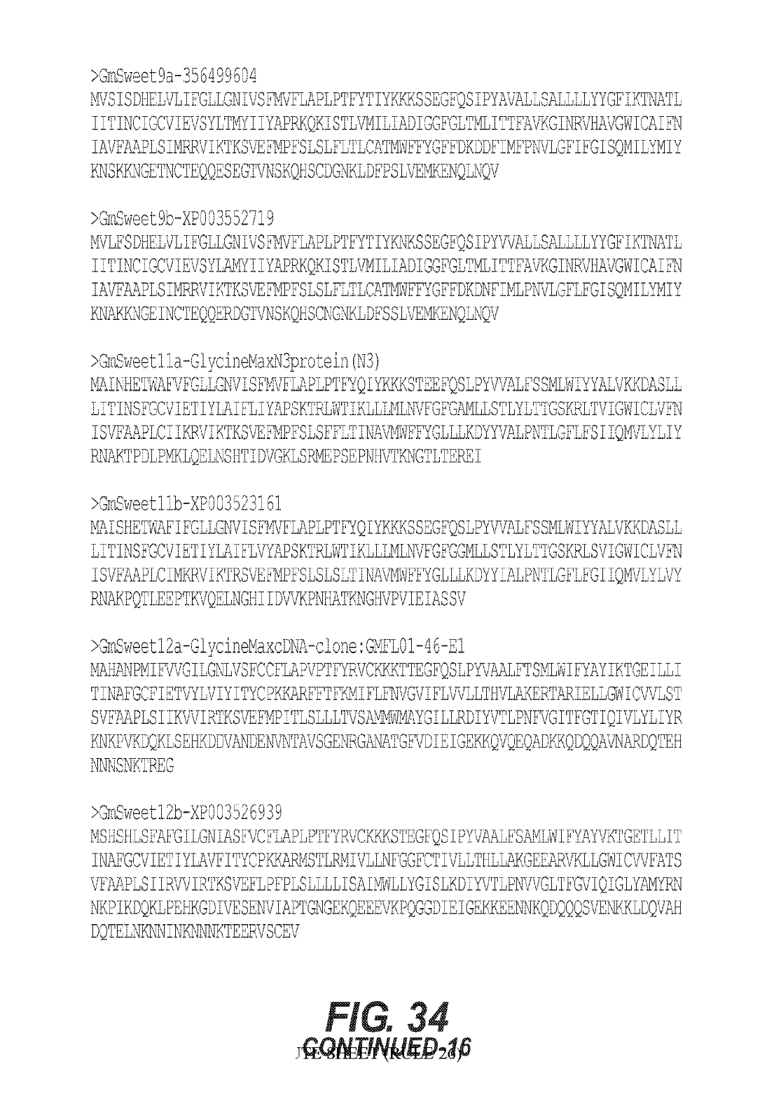

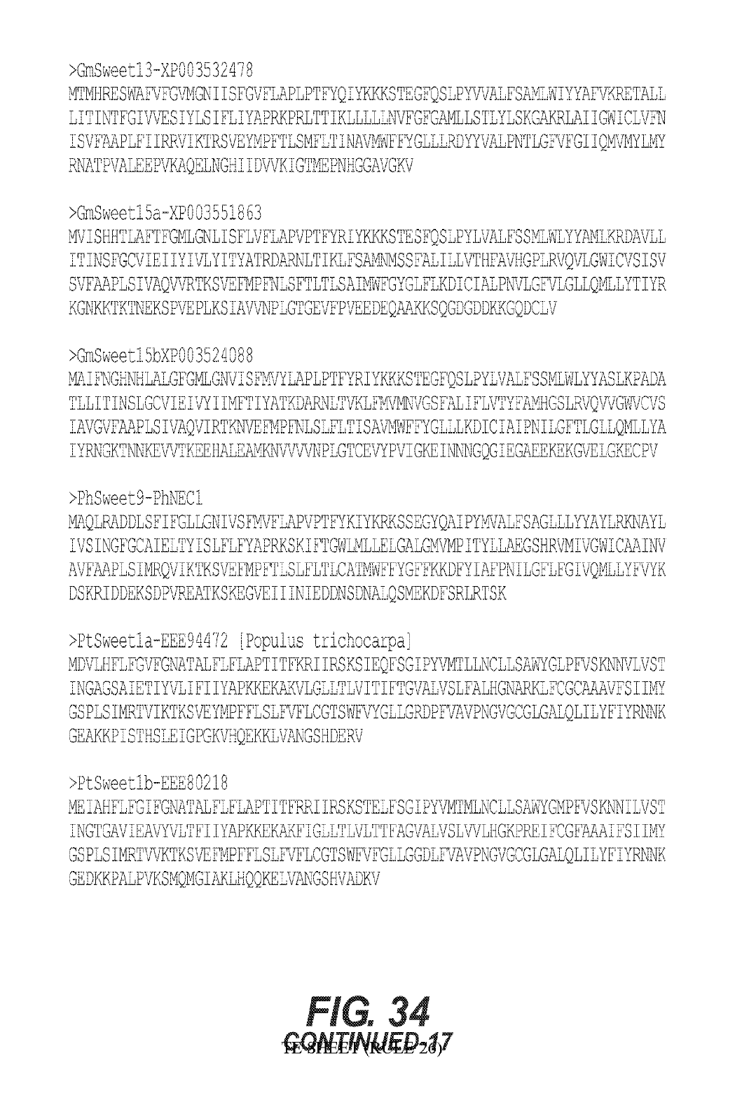

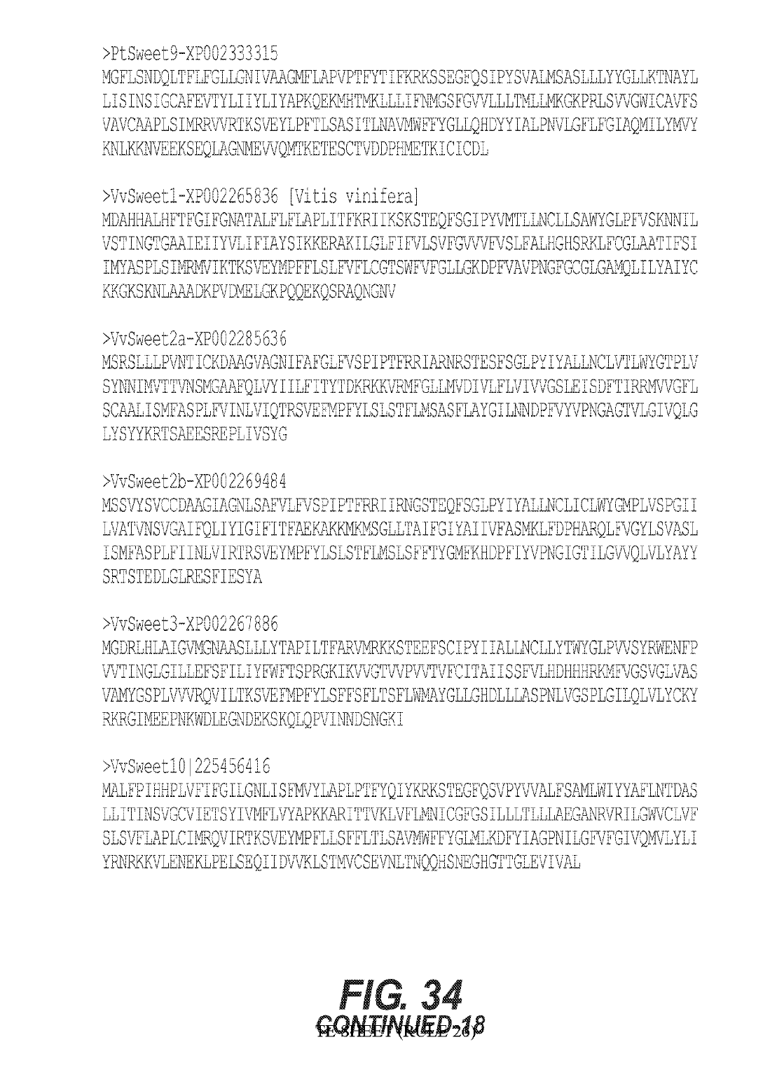

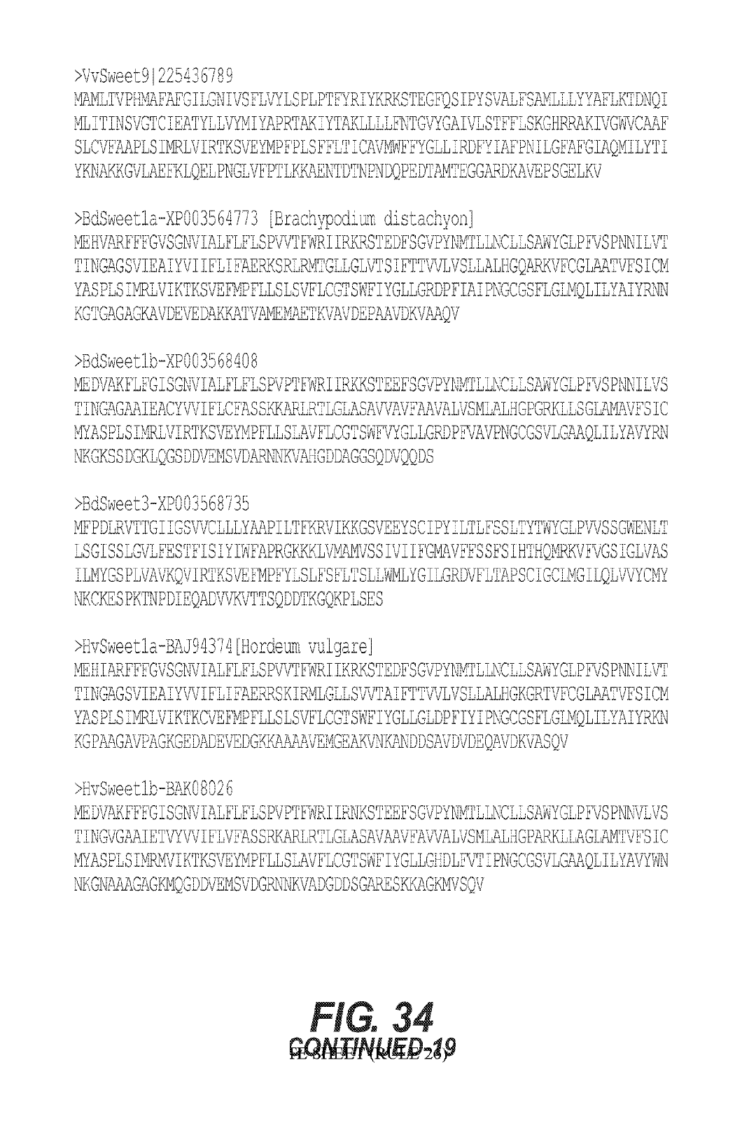

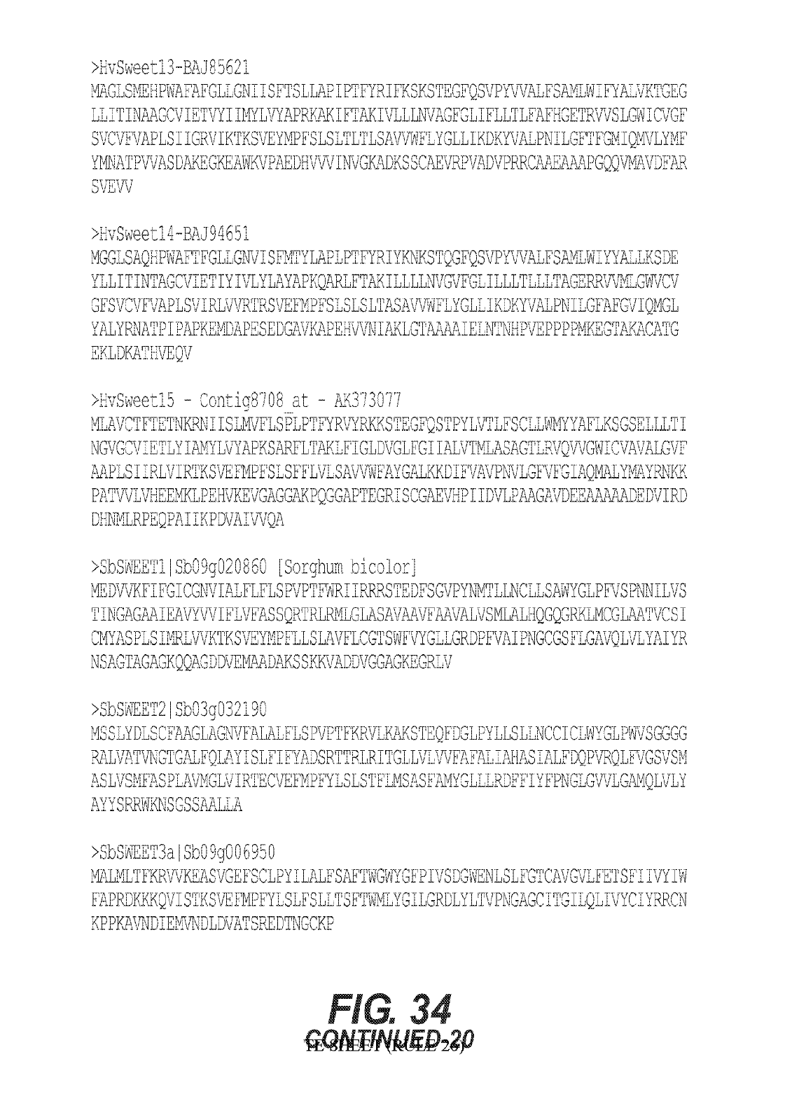

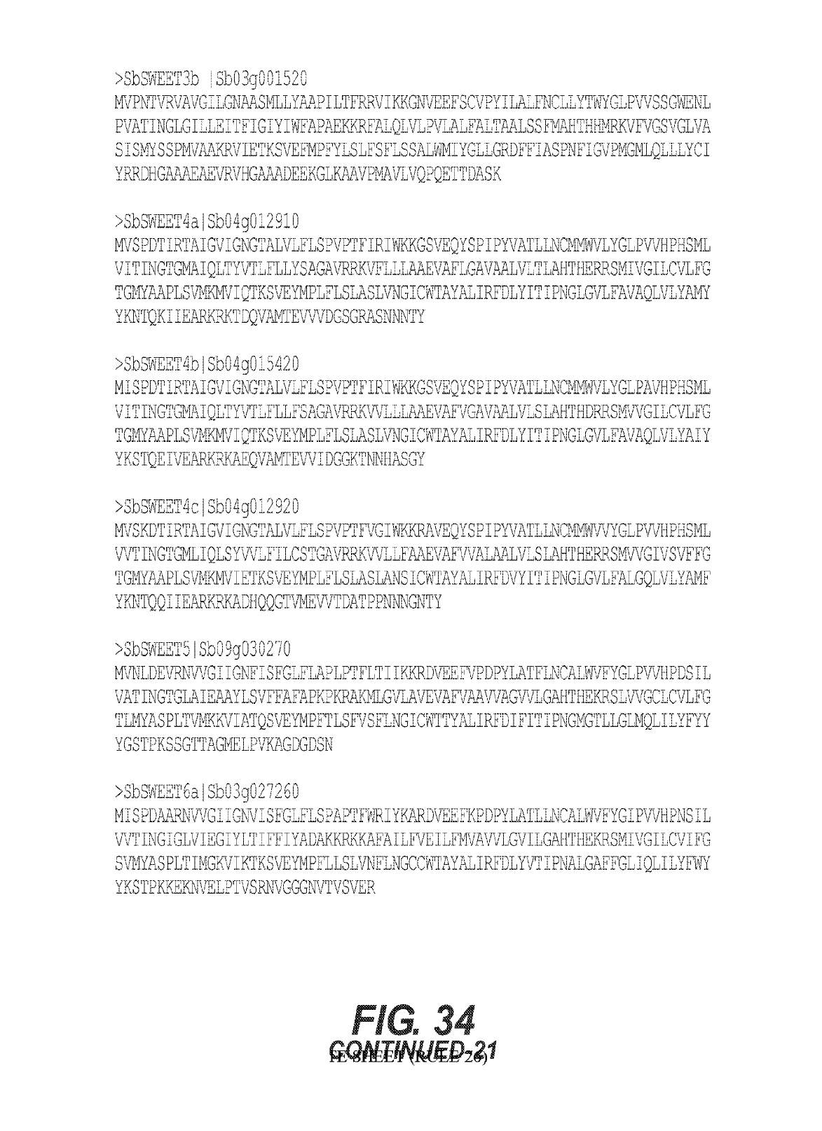

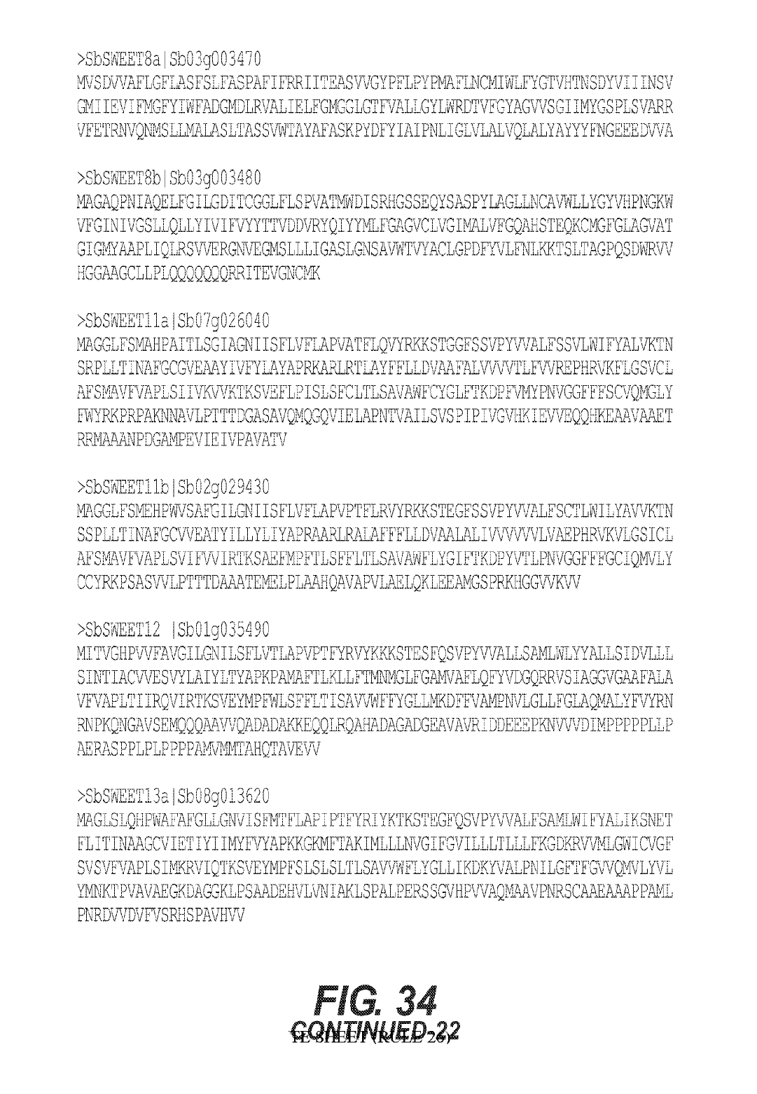

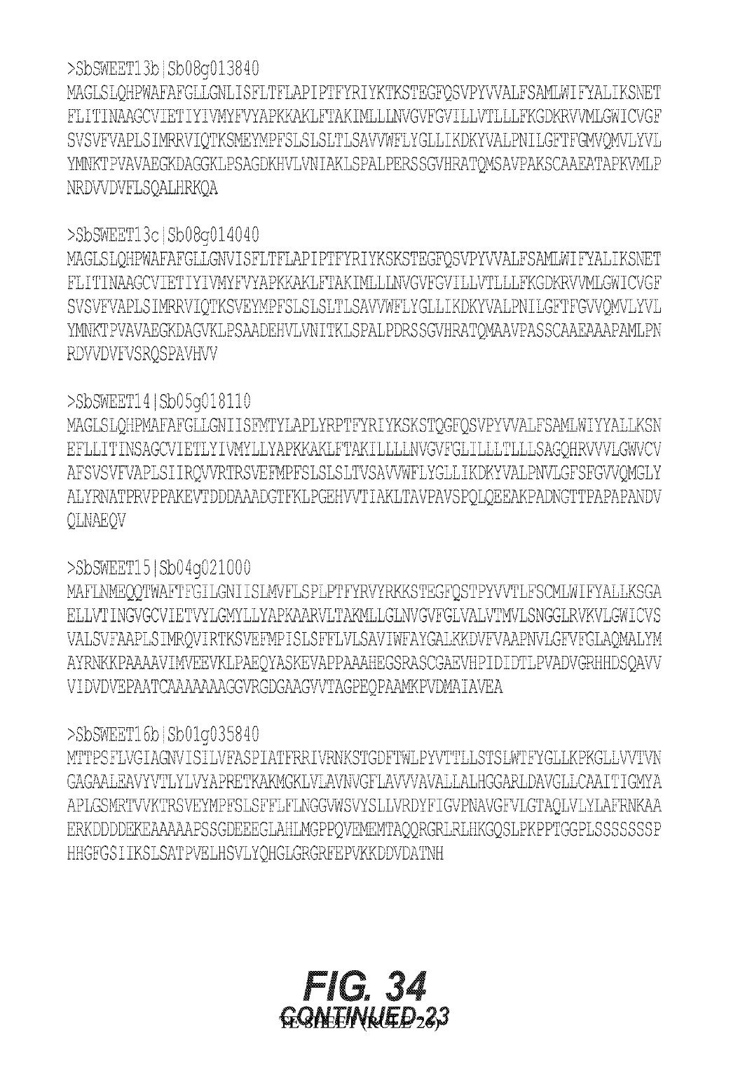

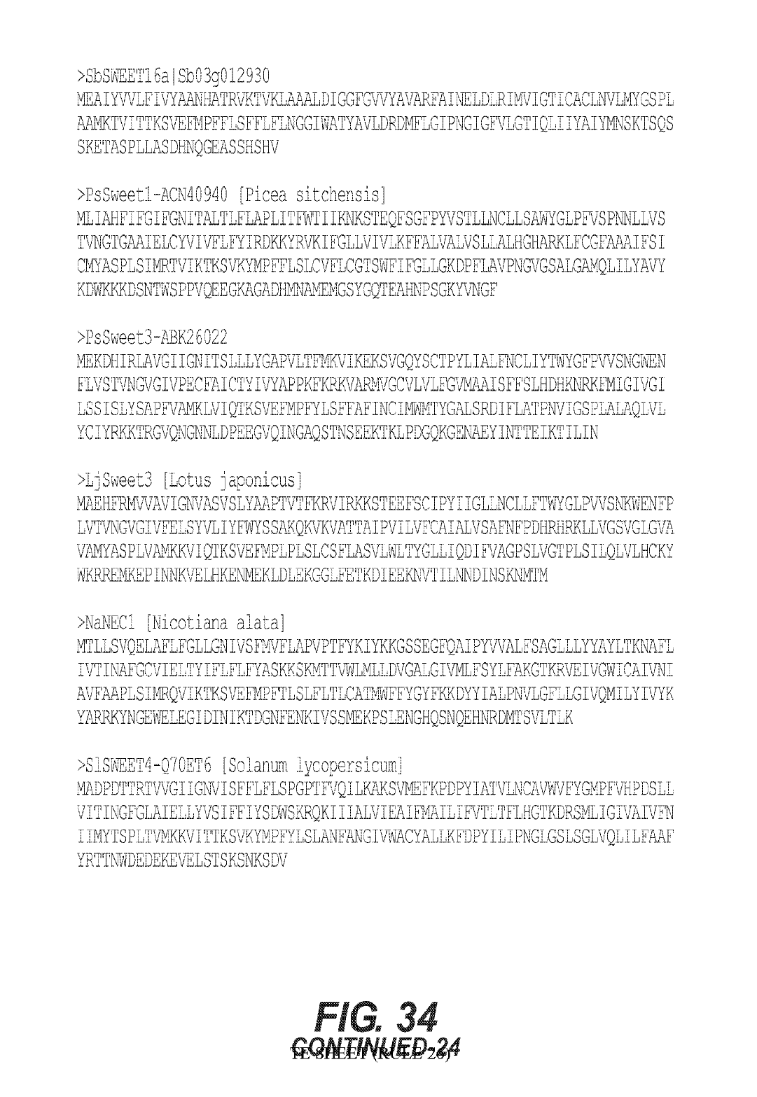

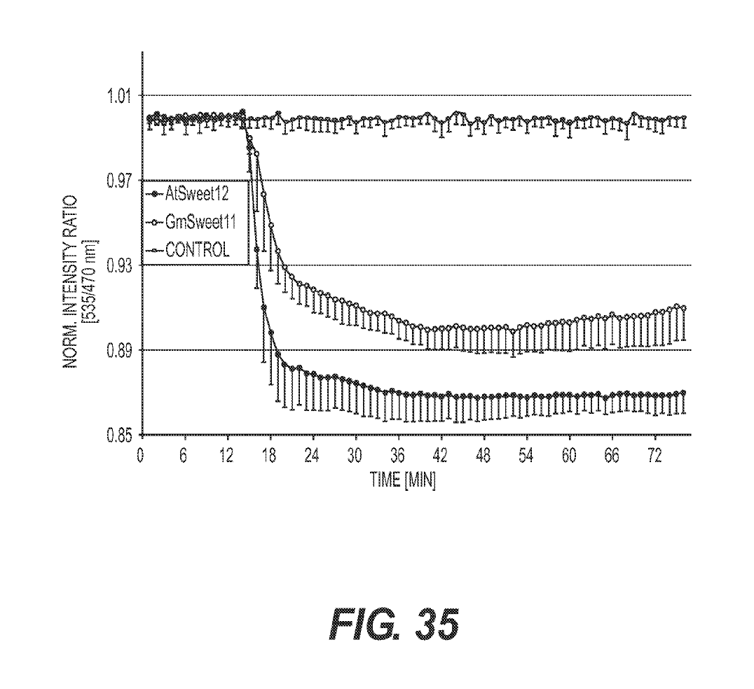

Nectary parenchyma cells may serve as a storage site for starch that is hydrolyzed to provide at least a fraction of the sugars for secretion. AtSWEET9 is localized in the parenchyma of the nectaries and shows sugar efflux function in oocytes. Therefore, it was hypothesized that in SWEET9 mutant lines, the sugar (starch) in the nectaries could not be secreted and the starch would accumulate in the nectary parenchyma at anthesis. To test the hypothesis, the starch in the nectaries of wild-type and SWEET9 mutant lines at anthesis were stained with Lugol's iodine solution and were investigated by LR white sections (sampling at the end of dark) (FIG. 29). The results show that starch accumulation in the floral stalks abundant of starch grains presented in the nectary parenchyma of SWEET9 mutant lines, but very few starch grains presented in the wild-type floral stalks and nectaries. The guard cells of the nectaries contained strong staining of starch grains in wild-type at anthesis but the starch grains were not observed in SWEET9 guard cells. According to the results, SWEET9 mutant lines accumulate the starch in the nectary parenchyma reveals its function as sugar efflux transporter; and the accumulation of starch in the guard cells in wild-type nectaries may due to reabsorption of nectar.

All publications and patent applications herein are incorporated by reference to the same extent as if each individual publication or patent application was specifically and individually indicated to be incorporated by reference. The publications discussed herein are provided solely for their disclosure prior to the filing date of the present application. Nothing herein is to be construed as an admission that the present invention is not entitled to antedate such publication by virtue of prior invention.

1. A genetically modified plant cell that has altered expression or activity of at least one sucrose efflux transporter compared to levels of expression or activity of the at least one sucrose efflux transporter in an unmodified plant cell.

2. The genetically modified plant cell of claim 1, wherein the sucrose efflux transporter is selected from the group consisting of SWEET9, SWEET10, SWEET11, SWEET12, SWEET13, SWEET14 and SWEET15.

3. The genetically modified plant cell of claim 2, wherein the genetic modification comprises the presence of at least one mutated copy of a gene encoding the sucrose efflux transporter.

4. The genetically modified plant cell of claim 3, wherein the mutated copy of the gene encoding the sucrose efflux transporter is integrated into the genome of plant cell.

5. The genetically modified plant cell of claim 3, wherein the at least one mutated copy of the at least one gene is operably linked to a tissue-specific promoter or an inducible plant promoter.

6. The genetically modified plant cell of claim 5, wherein the tissue-specific promoter promotes transcription in a leaf, flower, seed, stem or root cell.

7. The genetically modified plant cell of claim 2, wherein the genetic modification comprises the presence of at least one genetic construct encoding an antisense copy of at least one gene encoding the sucrose efflux transporter or encoding an siRNA corresponding to at least one gene encoding the sucrose efflux transporter.

8. The genetically modified plant cell of claim 7, wherein the genetic modification is integrated into the genome of the plant cell.

9. The genetically modified plant of claim 7, wherein the at least one genetic construct comprises a tissue-specific promoter or an inducible plant promoter.

10. The genetically modified plant cell of claim 9, wherein the tissue-specific promoter promotes transcription of the genetic construct in a leaf, flower, seed, stem or root cell.

11. The genetically modified plant cell of claim 1, wherein the expression or activity of more than one sucrose efflux transporter is increased or reduced.

12. The genetically modified plant cell of claim 1, wherein the genetically modified plant cell is comprised within a plant.

13. A method of producing a pathogen-resistant or pathogen-tolerant plant cell, the method comprising
(a) identifying at least one sucrose efflux transporter wherein the levels of expression or activity of the at least sucrose efflux transporter are altered in the plant cell in response to an infection of the pathogen as compared to an uninfected plant cell, and
(b) genetically modifying the plant cell to either (i) inhibit the activity or reduce the expression of the at least one identified sucrose efflux transporter in (a), or (ii) increase the activity or expression of the at least one identified sucrose efflux transporter in (a),
whereby inhibiting the activity or reducing the expression of the at least one identified sucrose efflux transporter or whereby increasing the activity or the expression of the at least one identified sucrose efflux transporter produces the pathogen-resistant or pathogen-tolerant plant cell.

14. The method of claim 13, wherein the at least one sucrose efflux transporter is selected from the group consisting of SWEET9, SWEET10, SWEET11, SWEET12, SWEET13, SWEET14 and SWEET15.

15. The method of claim 14, wherein the genetic modification comprises introducing at least one mutated copy of a gene encoding the sucrose efflux transporter.

16. The method of claim 15, wherein the genetic modification comprises introducing at least one mutated copy of the at least one gene into the genome of a plant cell.

17. The method claim 15, wherein the at least one mutated copy of the at least one gene is operably linked to a tissue-specific promoter or an inducible plant promoter.

18. The method of claim 17, wherein the tissue-specific promoter promotes transcription of the at least one mutated copy of the at least one gene in a leaf, flower, seed, stem or root cell.

19. The method of claim 14, wherein the genetic modification comprises the presence of at least one genetic construct encoding an antisense copy of at least one gene encoding the sucrose efflux transporter or encoding an siRNA corresponding to at least one gene encoding the sucrose efflux transporter.

20. The method of claim 19, wherein the genetic modification is integrated into the genome of the plant cell.

21. The method of claim 19, wherein the at least one genetic construct comprises a tissue-specific promoter or an inducible plant promoter.

22. The genetically modified plant of claim 21, wherein the tissue-specific promoter promotes transcription of the genetic construct in a leaf, flower, seed, stem or root cell.

23. The method of claim 13, wherein the genetic modification inhibits the activity or reduces the expression of more than one identified sucrose efflux transporter.

24. The method of claim 13, wherein the genetically modified plant cell is comprised within a plant.


SEQUENCE LISTING (180 sequences)

SEQ ID NO: 1 - protein, 247 aa, Arabidopsis thaliana
MNIAHTIFGVFGNATALFLFLAPSITFKRIIKNKSTEQFSGIPYPMTLLNCLLSAWYGLPFVSKDNTLVSTINGTGAVIETVYVLIFLFYAPKKEKIKIFGIFSCVLAVFATVALVSLFALQGNGRKLFCGLAATVFSIIMYASPLSIMRLVVKTKSVEFMPFFLSLFVFLCGTSWFVYGLIGRDPFVAIPNGFGCALGTLQLILYFIYCGNKGEKSADAQKDEKSVEMKDDEKKQNVVNGKQDLQV

SEQ ID NO: 2 - protein, 236 aa, Arabidopsis thaliana
MDVFAFNASLSMCKDVAGIAGNIFAFGLFVSPMPTFRRIMRNKSTEQFSGLPYIYALLNCLICLWYGTPFISHSNAMLMTVNSVGATFQLCYIILFIMHTDKKNKMKMLGLLFVVFAVVGVIVAGSLQIPDQLTRWYFVGFLSCGSLVSMFASPLFVINLVIRTKSVEFMPFYLSLSTFLMSASFLLYGLFNSDAFVYTPNGIGTILGIVQLALYCYYHRNSIEEETKEPLIVSYV

SEQ ID NO: 3 - protein, 263 aa, Arabidopsis thaliana
MGDKLRLSIGILGNGASLLLYTAPIVTFSRVFKKKSTEEFSCFPYVMTLFNCLIYTWYGLPIVSHLWENLPLVTINGVGILLESIFIFIYFYYASPKEKIKVGVTFVPVIVGFGLTTAISALVFDDHRHRKSFVGSVGLVASISMYGSPLVVMKKVIETRSVEYMPFYLSFFSFLASSLWLAYGLLSHDLFLASPNMVATPLGILQLILYFKYKNKKDLAPTTMVITKRNDHDDKNKATLEFVVDVDRNSDTNEKNSNNASSI

SEQ ID NO: 4 - protein, 251 aa, Arabidopsis thaliana
MVNATVARNIAGICGNVISLFLFLSPIPTFITIYKKKKVEEYKADPYLATVLNCALWVFYGLPMVQPDSLLVITINGTGLAIELVYLAIFFFFSPTSRKVKVGLWLIGEMVFVGIVATCTLLLFHTHNQRSSFVGIFCVIFVSLMYIAPLTIMSKVIKTKSVKYMPFSLSLANFLNGVVWVIYALIKFDLFILIGNGLGTVSGAVQLILYACYYKTTPKDDEDEEDEENLSKVNSQLQLSGNSGQAKRVSA

SEQ ID NO: 5 - protein, 240 aa, Arabidopsis thaliana
MTDPHTARTIVGIVGNVISFGLFCAPIPTMVKIWKMKSVSEFKPDPYVATVLNCMMWTFYGLPFVQPDSLLVITINGTGLFMELVYVTIFFVFATSPVRRKITIAMVIEVIFMAVVIFCTMYFLHTTKQRSMLIGILCIVFNVIMYAAPLTVMKLVIKTKSVKYMPFFLSLANFMNGVVWVIYACLKFDPYILIPNGLGSLSGIIQLIIYITYYKTTNWNDDDEDKEKRYSNAGIELGQA

SEQ ID NO: 6 - protein, 261 aa, Arabidopsis thaliana
MVHEQLNLIRKIVGILGNFISLCLFLSPTPTFIHIVKKKSVEKYSPLPYLATLLNCLVRALYGLPMVHPDSTLLVTISGIGITIEIVFLTIFFVFCGRQQHRLVISAVLTVQVVFVATLAVLVLTLEHTTDQRTISVGIVSCVFNAMMYASPLSVMKMVIKTKSLEFMPFLLSVVGFLNAGVWTIYGFVPFDPFLAIPNGIGCVFGLVQLILYGTYYKSTKGIMEERKNRLGYVGEVGLSNAIAQTEPENIPYLNKRVSGV

SEQ ID NO: 7 - protein, 258 aa, Arabidopsis thaliana
MVFAHLNLLRKIVGIIGNFIALCLFLSPTPTFVRIVKKKSVEEYSPIPYLATLINCLVWVLYGLPTVHPDSTLVITINGTGILIEIVFLTIFFVYCGRQKQRLIISAVIAAETAFIAILAVLVLTLQHTTEKRTMSVGIVCCVFNVMMYASPLSVMKMVIKTKSVEFMPFWLSVAGFLNAGVWTIYALMPFDPFMAIPNGIGCLFGLAQLILYGAYYKSTKRIMAERENQPGYVGLSSAIARTGSEKTANTNQEPNNV

SEQ ID NO: 8 - protein, 239 aa, Arabidopsis thaliana
MVDAKQVRFIIGVIGNVISFGLFAAPAKTFWRIFKKKSVEEFSYVPYVATVMNCMLWVFYGLPVVHKDSILVSTINGVGLVIELFYVGVYLMYCGHKKNHRRNILGFLALEVILVVAIILITLFALKGDFVKQTFVGVICDVFNIAMYGAPSLAIIKVVKTKSVEYMPFLLSLVCFVNAGIWTTYSLIFKIDYYVLASNGIGTFLALSQLIVYFMYYKSTPKEKTVKPSEVEISATERV

SEQ ID NO: 9 - protein, 258 aa, Arabidopsis thaliana
MFLKVHEIAFLFGLLGNIVSFGVFLSPVPTFYGIYKKKSSKGFQSIPYICALASATLLLYYGIMKTHAYLIISINTFGCFIEISYLFLYILYAPREAKISTLKLIVICNIGGLGLLILLVNLLVPKQHRVSTVGWVCAAYSLAVFASPLSVMRKVIKTKSVEYMPFLLSLSLTLNAVMWFFYGLLIKDKFIAMPNILGFLFGVAQMILYMMYQGSTKTDLPTENQLANKTDVNEVPIVAVELPDVGSDNVEGSVRPMK

SEQ ID NO: 10 - protein, 289 aa, Arabidopsis thaliana
MAISQAVLATVFGILGNIISFFVCLAPIPTFVRIYKRKSSEGYQSIPYVISLFSAMLWMYYAMIKKDAMMLITINSFAFVVQIVYISLFFFYAPKKEKTLTVKFVLFVDVLGFGAIFVLTYFIIHANKRVQVLGYICMVFALSVFVAPLGIIRKVIKTKSAEFMPFGLSFFLTLSAVMWFFYGLLLKDMNIALPNVLGFIFGVLQMILFLIYKKPGTKVLEPPGIKLQDISEHVVDVVRLSTMVCNSQMRTLVPQDSADMEATIDIDEKIKGDIEKNKDEKEVFLISKN

SEQ ID NO: 11 - protein, 289 aa, Arabidopsis thaliana
MSLFNTENTWAFVFGLLGNLISFAVFLSPVPTFYRIWKKKTTEGFQSIPYVVALFSATLWLYYATQKKDVFLLVTINAFGCFIETIYISMFLAYAPKPARMLTVKMLLLMNFGGFCAILLLCQFLVKGATRAKIIGGICVGFSVCVFAAPLSIIRTVIKTRSVEYMPFSLSLTLTISAVIWLLYGLALKDIYVAFPNVLGFALGALQMILYVVYKYCKTSPHLGEKEVEAAKLPEVSLDMLKLGTVSSPEPISVVRQANKCTCGNDRRAEIEDGQTPKHGKQSSSAAAT

SEQ ID NO: 12 - protein, 285 aa, Arabidopsis thaliana
MALFDTHNTWAFVFGLLGNLISFAVFLSPVPTFYRICKKKTTEGFQSIPYVVALFSAMLWLYYATQKKDVFLLVTINSFGCFIETIYISIFVAFASKKARMLTVKLLLLMNFGGFCLILLLCQFLAKGTTRAKIIGGICVGFSVCVFAAPLSIIRTVIKTKSVEYMPFSLSLTLTISAVIWLLYGLALKDIYVAFPNVIGFVLGALQMILYVVYKYCKTPSDLVEKELEAAKLPEVSIDMVKLGTLTSPEPVAITVVRSVNTCNCNDRNAEIENGQGVRNSAATT

SEQ ID NO: 13 - protein, 294 aa, Arabidopsis thaliana
MALTNNLWAFVFGILGNIISFVVFLAPVPTFVRICKKKSTEGFQSLPYVSALFSAMLWIYYAMQKDGTAFLLITINAFGCVIETIYIVLFVSYANKKTRISTLKVLGLLNFLGFAAIVLVCELLTKGSTREKVLGGICVGFSVSVFAAPLSIMRVVVRTRSVEFMPFSLSLFLTISAVTWLFYGLAIKDFYVALPNVLGAFLGAVQMILYIIFKYYKTPVAQKTDKSKDVSDHSIDIAKLTTVIPGAVLDSAVHQPPALHNVPETKIQLTEVKSQNMTDPKDQINKDVQKQSQV

SEQ ID NO: 14 - protein, 281 aa, Arabidopsis thaliana
MVLTHNVLAVTFGVLGNIISFIVFLAPVPTFVRICKKKSIEGFESLPYVSALFSAMLWIYYALQKDGAGFLLITINAVGCFIETIYIILFITYANKKARISTLKVLGLLNFLGFAAIILVCELLTKGSNREKVLGGICVGFSVCVFAAPLSIMRVVIRTKSVEFMPFSLSLFLTISAITWLFYGLAIKDFYVALPNILGAFLGAVQMILYVIFKYYKTPLVVDETEKPKTVSDHSINMVKLSSTPASGDLTVQPQTNPDVSHPIKTHGGDLEDQMDKKMPN

SEQ ID NO: 15 - protein, 292 aa, Arabidopsis thaliana
MGVMINHHFLAFIFGILGNVISFLVFLAPVPTFYRIYKRKSTESFQSLPYQVSLFSCMLWLYYALIKKDAFLLITINSFGCVVETLYIAMFFAYATREKRISAMKLFIAMNVAFFSLILMVTHFVVKTPPLQVSVLGWICVAISVSVFAAPLMIVARVIKTKSVEYMPFTLSFFLTISAVMWFAYGLFLNDICIAIPNVVGFVLGLLQMVLYLVYRNSNEKPEKINSSEQQLKSIVVMSPLGVSEVHPVVTESVDPLSEAVHHEDLSKVTKVEEPSIENGKCYVEATRPETV

SEQ ID NO: 16 - protein, 230 aa, Arabidopsis thaliana
MADLSFYVGVIGNVISVLVFLSPVETFWRIVQRRSTEEYECFPYICTLMSSSLWTYYGIVTPGEYLVSTVNGFGALAESIYVLIFLFFVPKSRFLKTVVVVLALNVCFPVIAIAGTRTLFGDANSRSSSMGFICATLNIIMYGSPLSAIKTVVTTRSVQFMPFWLSFFLFLNGAIWGVYALLLHDMFLLVPNGMGFFLGIMQLLIYAYYRNAEPIVEDEEGLIPNQPLLA

SEQ ID NO: 17 - protein, 241 aa, Arabidopsis thaliana
MAEASFYIGVIGNVISVLVFLSPVETFWKIVKRRSTEEYKSLPYICTLLGSSLWTYYGIVTPGEYLVSTVNGFGALVETIYVSLFLFYAPRHLKLKTVDVDAMLNVFFPIAAIVATRSAFEDEKMRSQSIGFISAGLNIIMYGSPLSAMKTVVTTKSVKYMPFWLSFFLFLNGAIWAVYALLQHDVFLLVPNGVGFVFGTMQLILYGIYRNAKPVGLSNGLSEIAQDEEEGLTSRVEPLLS

SEQ ID NO: 18 - protein, 273 aa, Oryza sativa
MEHIARFFFGVSGNVIALFLFLSPVVTFWRIIKKRSTEDFSGVPYNMTLLNCLLSAWYGLPFVSPNNILVTTINGTGSVIEAIYVVIFLIFAERKARLKMMGLLGLVTSIFTMVVLVSLLALHGQGRKLFCGLAATIFSICMYASPLSIMRLVIKTKSVEFMPFLLSLSVFLCGTSWFIYGLLGRDPFIAIPNGCGSFLGLMQLILYAIYRNHKGATPAAAAGKGDAADEVEDAKKAAAAVEMADAKTNKVVADDADADADGKSADDKVASQV

SEQ ID NO: 19 - protein, 261 aa, Oryza sativa
MEDLAKFLFGVSGNVIALFLFLSPVPTFWRIIRRKSTEDFSGVPYNMTLINCLLSAWYGLPFVSPNNILVSTINGAGAVIETAYVVVFLVFASTHKTRLRTLGLAAAVASVFAAVALVSLLALHGQHRKLLCGVAATVCSICMYASPLSIMRLVIKTKSVEYMPFLMSLAVFLCGTSWFIYGLLGRDPFVTIPNGCGSFLGAVQLVLYAIYRNNKGAGGGSGGKQAGDDDVEMAEGRNNKVADGGAADDDSTAGGKAGTEV

SEQ ID NO: 20 - protein, 243 aa, Oryza sativa
MMNALGLSVAATSTGSPFHDVCCYGAGIAGNIFALVLFISPLPTFKRIVRNGSTEQFSAMPYIYSLLNCLICLWYGLPFVSYGVVLVATVNSIGALFQLAYTATFIAFADAKNRVKVSSLLVMVFGVFALIVYVSLALFDHQTRQLFVGYLSVASLIFMFASPLSIINLVIRTKSVEYMPFYLSLSMFLMSVSFFAYGVLLHDFFIYIPNGIGTVLGVIQLVLYGYFRKGSREDSLPLLVTHT

SEQ ID NO: 21 - protein, 230 aa, Oryza sativa
MDSLYDISCFAAGLAGNIFALALFLSPVTTFKRILKAKSTERFDGLPYLFSLLNCLICLWYGLPWVADGRLLVATVNGIGAVFQLAYICLFIFYADSRKTRMKIIGLLVLVVCGFALVSHASVFFFDQPLRQQFVGAVSMASLISMFASPLAVMGVVIRSESVEFMPFYLSLSTFLMSASFALYGLLLRDFFIYFPNGLGLILGAMQLALYAYYSRKWRGQDSSAPLLLA

SEQ ID NO: 22 - protein, 246 aa, Oryza sativa
MFPDIRFIVGIIGSVACMLLYSAPILTFKRVIKKASVEEFSCIPYILALFSCLTYSWYGFPVVSYGWENMTVCSISSLGVLFEGTFISIYVWFAPRGKKKQVMLMASLILAVFCMTVFFSSFSIHNHHIRKVFVGSVGLVSSISMYGSPLVAMKQVIRTKSVEFMPFYLSLFTLFTSLTWMAYGVIGRDPFIATPNCIGSIMGILQLVVYCIYSKCKEAPKVLHDIEQANVVKIPTSHVDTKGHNP

SEQ ID NO: 23 - protein, 252 aa, Oryza sativa
MVSNTIRVAVGILGNAASMLLYAAPILTFRRVIKKGSVEEFSCVPYILALFNCLLYTWYGLPVVSSGWENSTVSSINGLGILLEIAFISIYTWFAPRERKKFVLRMVLPVLAFFALTAIFSSFLFHTHGLRKVFVGSIGLVASISMYSSPMVAAKQVITTKSVEFMPFYLSLFSFLSSALWMIYGLLGKDLFIASPNFIGCPMGILQLVLYCIYRKSHKEAEKLHDIDQENGLKVVTTHEKITGREPEAQRD

SEQ ID NO: 24 - protein, 259 aa, Oryza sativa
MVSPDTIRTAIGVVGNGTALVLFLSPVPTFIRIWKKGSVEQYSAVPYVATLLNCMMWVLYGLPAVHPHSMLVITINGTGMAIELTYIALFLAFSLGAVRRRVLLLLAAEVAFVAAVAALVLNLAHTHERRSMIVGILCVLFGTGMYAAPLSVMKMVIQTKSVEYMPLFLSLASLVNGICWTAYALIRFDLYITIPNGLGVMFAVAQLILYAIYYKSTQQIIEARKRKEADHVAMTDVVVDSAKNNPSSGAAAAAANGRY

SEQ ID NO: 25 - protein, 237 aa, Oryza sativa
MVMNPDAVRNVVGIIGNLISFGLFLSPLPTFVTIVKKKDVEEFVPDPYLATFLNCALWVFYGLPFIHPNSILVVTINGTGLLIEIAYLAIYFAYAPKPKRCRMLGVLTVELVFLAAVAAGVLLGAHTYDKRSLIVGTLCVFFGTLMYAAPLTIMKQVIATKSVEYMPFTLSLVSFINGICWTIYAFIRFDILITIPNGMGTLLGAAQLILYFCYYDGSTAKNKGALELPKDGDSSAV

SEQ ID NO: 26 - protein, 259 aa, Oryza sativa
MISPDAARNVVGIIGNVISFGLFLAPVPTFWRICKRKDVEEFKADPYLATLLNCMLWVFYGIPVVHPNSILVVTINGIGLLVEGTYLLIFFLYSPNKKRLRMCAVLGVELVFMLAVILGVLLGAHTHEKRSMIVGILCVFFGSIMYFSPLTIMGKVIKTKSVEYMPFFLSLVCFLNGVCWTAYALIRFDIYVTIPNGLGALFGAIQLILYACYYRTTPKKTKAAKDVEMPSVVVSGTGAAAAAGGGNTGGGSVSVTVER

SEQ ID NO: 27 - protein, 254 aa, Oryza sativa
MISPDAARNVVGIIGNVISFGLFLSPVPTFWRICKRKDVEQFKADPYLATLLNCMLWVFYGIPIVHPNSILVVTINGIGLIVEGTYLFIFFLYSPNKKRLRMLAVLGVELVFMLAVILGVLLSAHTHKKRSMIVGILCVFFGSIMYFSPLTIMGKVIKTKSVEYMPFFLSLVCFLNGVCWTAYALIRFDIYVTIPNGLGAIFGAIQLILYACYYRTTPKKTKAAKDVEMPSVISGPGAAATASGGSVVSVTVER

SEQ ID NO: 28 - protein, 264 aa, Oryza sativa
  misc_feature (40)..(40): Xaa can be any naturally occurring amino acid
MVSPDMIRNVVGIVGNVISFGLFLSPVPTFWQIIKNKNVXDFKTDPYLATLLNCMLWDFYGLPIVHPNSILVVTINGIGLVIEAVYLTIFFLFSDKKNKKKMEVVLAAEALFMAAVALGVLLGVHTHQRRSLIVGILCVIFDTIMYSSPLTVMSQVVKTKSVEYMPLLLSVVSFLNGLYWTSYTLIRFDIFITIPNGLGVLFAAVQLILYVIYYRTTPKKQNKNLELPTVTPVAKDTSVGPISKDNDLNGSTASHVTIDITIQP

SEQ ID NO: 29 - protein, 265 aa, Oryza sativa
MVSPDLIRNMVGIVGNIISFGLFLSPVPTFYRIIKNKDVQDFKADPYLATLLNCMLWVFYGLPIVHPNSILVVTINGIGLVIEAVYLTIFFLFSDKKNKKKMGVVLATEALFMAAVVLGVLLGAHTHQRRSLIVGILCVIFGTIMYSSPLTIMSQVVKTKSVEYMPLLLSVVSFLNGLCWTSYALIRLDIFITIPNGLGVLFALMQLILYAIYYRTIPKKQDKNLELPTVAPVAKDTSIVTPVSKDDDVDGGNASHVTINITIEL

SEQ ID NO: 30 - protein, 263 aa, Oryza sativa
MVSPDLIRNVVGIVGNVISFGLFLSPVPIFWRIIKNKNVQNFKADPYLATLLNCMLWVFYVLPIVHPNSILVVTINGISLVIEAVYLTIFFLFSDKKNKKKMGVVLATEALFMAAVAVGVLLGAHTHQRRSLIVGILCVIFGTIMYSSPLTIMVVKTKSVEYMPLLLSVVSFLNGLCWTLYALIRFDIFITIPNGLGVLFAIMQLILYAIYYRTTPKKQDKNLELPTVAPIAKDTSIVAPVSNDDDVNGSTASHATINITIEP

SEQ ID NO: 31 - protein, 274 aa, Oryza sativa
MVPDLIRNVVGIVGNVISFGLFLSPVPTFWRIIKNKDVRDFKADQYLATLLNCMLWVFYGLPIVHPNSILVVTINGIGLVIEAVYLTIFFLFSDKKNKKKMGVVLATEALFMAAVALGVLLDAHTHQRRSLIVGILCVIFGTIMYSSPLTIMSQVVKTKSVEYMPLLLSVVSFLNGLCWTSYALIRFDIFITIPNGLGVLFALMQLILYAIYYRTTPKKPSTTGPHPRSRIRTSSYQPSPPSPRAPASSPLSARTTTSMAAMSPSISRLSHKLA

SEQ ID NO: 32 - protein, 256 aa, Oryza sativa
MVSPDLIRNVVGIVGNAISFGLFLSPVLTFWRIIKEKDMKYFKADPYLATLLNCMLWVFYGLPIVHPNSILVVTINGIGLVIEAVYLTIFFLFSNKKNKKMGVVLATEALFMAAVALGVLLGAHTHQRRSLIVGILCVIFGTIMYSSPLTIMSQVVKTKSVEYMPLLLSVVSFLNGLCWTSYALIRFDIFITIPNGLGVLFTLMQLILDKNQDKNLELPTVAPVAKETSIVTPVSKDDDINGSTASHVIINITKEP

SEQ ID NO: 33 - protein, 307 aa, Oryza sativa
MAGGFLSMANPAVTLSGVAGNIISFLVFLAPVATFLQVYKKKSTGGYSSVPYVVALFSSVLWIFYALVKTNSRPLLTINAFGCGVEAAYIVLYLVYAPRRARLRTLAFFLLLDVAAFALIVVTTLYLVPKPHQVKFLGSVCLAFSMAVFVAPLSIIFKVIKTKSVEFMPIGLSVCLTLSAVAWFCYGLFTKDPYVMYPNVGGFFFSCVQMGLYFWYRKPRNTAVLPTTSDSMSPISAAAAATQRVIELPAGTHAFTILSVSPIPILGVHKVEVVAAEQAADGVAAAAAADKELLQNKPEVIEITAAV

SEQ ID NO: 34 - protein, 300 aa, Oryza sativa
MVQALVFAVGIVGNILSFLVILAPVPTFYRVYKKKSTESFQSVPYAVALLSAMLWLYYALLTSDLLLLSINSIGCLVESLYLTVYLLYAPRQAMAFTLKLVCAMNLALFAAVVAALQLLVKATDRRVTLAGGIGASFALAVFVAPLTIIRQVIRTKSVEFMPFWLSFFLTLSAVVWFFYGLLMKDFFVATPNVLGLLFGLAQMVLYVVYKNPKKNSAVSEAAAAQQVEVKDQQQLQMQLQASPAVAPLDVDADADADLEAAAPATPQRPADDDAIDHRSVVVDIPPPPQPPPALPAVEVA

SEQ ID NO: 35 - protein, 296 aa, Oryza sativa
MAGLSLQHPWAFAFGLLGNLISFTTYLAPIPTFYRIYKSKSTEGFQSVPYVVALFSAMLWIFYALIKSNEALLITINAAGCVIETIYIVMYLAYAPKKAKVFTTKILLLLNVGVFGVILLLTLLLSHGEQRVVSLGWVCVAFSVSVFVAPLSIIKRVIQSRSVEYMPFSLSLTLTLSAVVWFLYGLLIKDKYVALPNILGFTFGVVQMGLYVFYMNATPVAGEGKEGKGKLAAAEELPVVVNVGKLAAATPDRSTGAVHVHPVPRSCAAEAAAAEPEVLVDIPPPPPPRAVEVAAV

SEQ ID NO: 36 - protein, 303 aa, Oryza sativa
MAGMSLQHPWAFAFGLLGNIISFMTYLAPLPTFYRIYKSKSTQGFQSVPYVVALFSAMLWIYYALLKSDECLLITINSAGCVIETIYIAVYLVYAPKKAKMFTAKLLLLVNVGVFGLILLLTLLLSAGDRRIVVLGWVCVGFSVSVFVAPLSIIRLVVRTKSVEFMPFSLSFSLTISAVVWFLYGLLIKDKYVALPNVLGFSFGVIQMGLYAMYRNSTPKAVLTKEVEAATATGDDDHSAAGVKEHVVNIAKLSAAVDVVKTREVHPVDVESPPAEAPPEEDDKAAAATAAAVAGAGEKKVAA

SEQ ID NO: 37 - protein, 319 aa, Oryza sativa
MAFMSMERSTWAFTFGILGNLISLMVFLSPLPTFYRVYRKKSTEGFQSTPYVVTLFSCMLWMYYAFVKSGAELLVTINGVGCVIETVYLAMYLAYAPKSARMLTAKMLLGLNIGLFGVIALVTLLLSRGELRVHVLGWICVAVSLSVFAAPLSIIRLVIRTKSVEFMPFSLSFFLVLSAVIWFLYGLLKKDVFVALPNVLGFVFGVAQMALYMAYRSKKPLVASSSSAVVAAGLEIKLPEHVKEVQAVAKGAVAAAPEGRISCGAEVHPIDDVMPSEVVEVKVDDEETNRTDEMAGDGDHAMVRTEQIIKPDMAIVVEV

SEQ ID NO: 38 - protein, 328 aa, Oryza sativa
MADPSFFVGIVGNVISILVFASPIATFRRIVRSKSTEEFRWLPYVTTLLSTSLWTFYGLHKPGGLLIVTVNGSGAALEAIYVTLYLAYAPRETKAKMVKVVLAVNVGALAAVVAVALVALHGGVRLFVVGVLCAALTIGMYAAPMAAMRTVVKTRSVEYMPFSLSFFLFLNGGVWSVYSLLVKDYFIGIPNAIGFALGTAQLALYMAYRRTKKPAGKGGDDDEDDEEAQGVARLMGHQVEMAQQRRDQQLRKGLSLSLPKPAAPLHGGLDRIIKSFSTTPIELHSILHQHHGGHHHHHRFDTVPDDDDEAVAAGGTTPATTAGPGDRH

SEQ ID NO: 39 - protein, 267 aa, Zea mays
MEHIARFFFGVSGNVIALFLFLSPVVTFWRVIRKRSTEDFSGVPYNMTLLNCLLSAWYGLPFVSPNNILVSTINGTGSVIEAIYVVIFLIFAVDRRARLSMLGLLGIVASIFTTVVLVSLLALHGNARKVFCGLAATIFSICMYASPLSIMRLVIKTKSVEFMPFLLSLAVFLCGTSWFIYGLLGRDPFIIIPNGCGSFLGLMQLILYAIYRKNKGPAAPAGKGEAAAAAAEVEDTKKVAAAVELADATTNKAADAVGGDGKVASQV

SEQ ID NO: 40 - protein, 250 aa, Zea mays
MEDVVKFVFGVSGNVIALFLFLSPVPTFWRIIRRKSTEDFSGVPYSMTLLNCLLSAWYGLPFVSPNNMLVSTINGAGAAIEAVYVVIFLAFASSQRTRLRMLGLASAVSAAFAAVALASMLALHGQGRKLMCGLAATVCSICMYASPLSIMRLVVKTKSVEYMPFLLSLAVFLCGTSWFVYGLLGRDPFVAIPNGCGSFLGAVQLVLYAIYRDSNSGGKQQAGDDVEMASDAKSSKKVADDVGGKEDRLV

SEQ ID NO: 41 - protein, 243 aa, Zea mays
MDWDAPALTSFVADLSFRHLCCYGAGIAGNAFAFVLFVSPLPTFKRIVRNGSTEQFSCTPYIYSLLNCLICMWYGLPFVSYGVVLVATVNSIGAVFQLAYTAVFIAFADAKQRLKVSALLAAVFLVFGLIVFVSLALLDHKARQVFVGYLSVASLVCMFASPMSIVNLVIRTKSVEYMPFYLSLSMFLMSASFVIYGVLLGDGFIYIPNGIGTILGIVQLLLYAYIRKGSSEEAKLPLLITHT

SEQ ID NO: 42 - protein, 238 aa, Zea mays
MVTSIRVIVGIIGSVVCVLLYAVPVLTFKRVVKEASVGEFSCVPYILALFSAFTWGWYGFPIVSDGWENLSLFGTCAVGVLFEASFVVVYVWFAPRDKKKSVVLMVSLVVATLCVIVSLSSFVFHTHHMRKQFVGSIGIVTSISMYSAPLVAVKQVILTKSVEFMPFYLSLFSLLTSFTWMLYGILGRDPYLTAPNGAGCLTGLLQIAVYCIYSRCNRPPKAVNGATTSREDANDCKV

SEQ ID NO: 43 - protein, 327 aa, Zea mays
MVPDTVRVAVGILGNAASMLLYTTPILTFRWVIRKGNVEEFSCVPYILALLNCLLYTWYGLPVVSSGWENLPVATINGLGILLEVAFIAIYLRFAPAEKKRFALQLVLPALALFGLTAALSSFAARTHRSRKAFVGSVGLVASVSMYTSPMVAAKRVIATKSVEFMPFSLSLFSFLSSALWMAYGLLGRDLFIASPNFIGVPVGVLQLLLYCIYRRDHGAAAGAEAQAHGPAAAADQEKGMKAAAPVAVQPQENPLCVVSVCEVNVSLSPSAAQAQHRTGLSKSNEIEGLALGLYGHIAATQLLRTTYTDQQIHLWRVWFMKSLYTS

SEQ ID NO: 44 - protein, 252 aa, Zea mays
MISPDTIRTAIGVIGNGTALVLFLSPVPTFIRIWKKGSVEQYSPIPYVATLLNCMMWVLYGLPAVHPHSMLVITINGTGMAIQLTYVALFLLYSVGAARRKVVLLLAAEVGFVGAVAALVLSLAHTHERRSMVVGILCVLFGTGMYAAPLSVMKMVIQTKSVEYMPLFLSLASLVNGICWTAYALIRFDLYITIPNGLGVLFAVAQLVLYAIYYKSTQEIIEARKRKADQIAMTGVVVDGGKTNNQAGAGQY

SEQ ID NO: 45 - protein, 255 aa, Zea mays
MVSSDTIRTAIGVIGNGTALVLFLSPVPTFIRIWKKGSVEQYSPIPYVATLLNCMMWVLYGLPLVHPHSMLVITINGTGMLIQLTYVALFLVYSAGAARRKVSLLLAAEVAFVGAVAALVLALAHTHERRSMVVGILCVLFGTGMYAAPLSVMKMVIQTKSVEYMPLFLSLASLVNGICWTAYALIRFDLYITIPNGLGVLFALAQLLLYAIYYKNTQKIVEARKRKAGQVAMTEVVVDGSRASNNNNNGGSGTY

SEQ ID NO: 46 - protein, 261 aa, Zea mays
MVSADTIRTAIGVIGNGTALVLFLSPVPTFVGIWKKRAVEQYSPIPYVATLLNCMMWVLYGLPLVHPHSMLVVTINGTGMLIQLTYVALFILCSAGAVRRRVVLLFAAEVAFVVALAALVLTLAHTHERRSMLVGIVSVFFGTGMYAAPLSVMKLVIQTKSVEYMPLFLSLASLANSICWTAYALIRFDLYITIPNGLGVLFALGQLGLYAMFYKNTKQIMEARRRKADQQSTMMEVVTDASATPPPPPNNNNGGGGGNGY

SEQ ID NO: 47 - protein, 244 aa, Zea mays
MISPDAARNVVGIIGNVISFGLFLSPVLTFWRIYKAKDVEEFKPDPYLATLLNCMLWVFYGIPVVHPNSILVVTINGIGLVIEAVYLTIFFLYSDSQKRKKAFAILAVEILFMVAVVLGVILGAHTHEKRSMIVGILCVIFGSMMYASPLTIMSRVIKTKSVEYMPFLLSLVSFLNGCCWTAYALIRFDLYVTIPNALGAFFGLVQLILYFCYYKSTPKKEKNVELPTVSSNVGGGNVTVSVER

SEQ ID NO: 48 - protein, 243 aa, Zea mays
MISPDAARNVVGIIGNVISFGLFLSPVLTFWRICKARDVEEFKPDPYLATLLNCMLWVFYGIPVVHPNSILVVTINGVGLVIEAIYLTIFFLYSDGPKRRKAFGILAVEILFMVAVVLGVILGAHTHEKRSMIVGILCVIFGSMMYASPLTIMSRVIKTKSVEYMPFLLSLVSFLNGCCWTAYALIRFDLYVTIPNALGAFFGLIQLILYFCYYKSTPKEKNVELPTVSSNAGGGNVTVSVER

SEQ ID NO: 49 - protein, 310 aa, Zea mays
MAGGFFSMAHPAVTLSGIAGNIISFLVFLAPVATFLQVYRKKSTGGFSSVPYVVALFSSVLWIFYALVKTNSRPLLTINAFGCGVEAAYIVLYLAYAPRRARLRTLAYFFLLDVAAFALVVAVTLFAVREPHRVKFLGSVCLAFSMAVFVAPLSIIVKVVKTKSVEFLPISLSFCLTLSAVAWFCYGLFTKDPFVMYPNVGGFFFSCVQMGLYFWYRKPRPAAKNNAVLPTTTDGANAVQVQGQVIELAPNTVAILSVSPIPIVGVHKIEVVEQQHKEAAVAAETRRMAAANPDGAMPEVIEIVPAAAAV

SEQ ID NO: 50 - protein, 306 aa, Zea mays
MITVGHPVVFAVGILGNILSFLVTLAPVPTFYRVYKKKSTESFQSVPYVVALLSAMLWLYYALLSVDLLLLSINTIACVVESVYLAIYLTYAPKPAMAFTLKLLCTMNMGLFGAMVAFLQFYVDGQRRVSIAGGVGSAFAFAVFVAPLTIIRQVIRTKSVEFMPFWLSFFLTVSAVAWFFYGLLMKDFFVAMPNVLGLLFGLAQMALYFVYRNRNPKKNGAVSEMQQAAAVQADAEKEQQLRQADADADADGKAATTDDDGGQTAVVVDIMPPPPLLPAERAPPLPLPPHPAMVMTTAHQTAVEVV

SEQ ID NO: 51 - protein, 305 aa, Zea mays
MITVGHPVAFAVGILGNILSFLVILAPVPTFYRVYAKKSTESFQSVPYVVALLSATLWLYYALLSTDLLLLSINTVACVAESVYLAVYLAYAPGPAKAFTLKLLCAINMGLFGAMVAFLQFYVVDTQRRVSIAGGVGAAFALAVFVAPLAIIRRVMRTKSVEFMPFWLSFFLTVSAVVWFFYGLLIKDFFVAMPNVLGLLFGLAQMVLFFVYRNRNPKKNGAVSEMQQAAVQADAEKERRSHANADGEADVRTVIVDIMPPPPAMMRHADREARGGAGTGRRAAAREQGGARRREDREALGGGGI

SEQ ID NO: 52 - protein, 302 aa, Zea mays
MAGMSLQHPWAFAFGLLGNVISFMTFLAPIPTFYRIYKSKSTEGFQSVPYVVALFSAMLWIFYALIKSNETFLITINAAGCVIETIYVVMYFVYAPKKAKLFTAKIMVLLNGGVFGVILLLTLLLFKGSKRVVLLGWICVGFSVSVFVAPLSIMRRVIQTKSVEYMPFSLSLSLTLSAVVWFLYGLLIKDKYVALPNILGFTFGVVQMVLYVLYMNKTPVAATAEGKDAGKLSSAADEHVLVNIAKLSPALPERSSGVHPVTQMAGVPVRSCAAEATAPAMLPNRDVVDVFVSRHSPAVHVA

SEQ ID NO: 53 - protein, 301 aa, Zea mays
MAGLSLEHPWAFAFGLLGNVISFMTFLAPIPTFYRIYKSKSTEGFQSVPYVVALFSAMLWIFYALIKSNETFLITINAAGCVIETIYIVMYFVYAPKKAKLFTAKIMALLNGGVFGVILLLTLLLFKGSKRVVLLGWICVGFSVSVFVAPLSIMRRVIQTKSVEYMPFSLSLSLTLSAVVWFLYGLLIKDKYVALPNVLGFIFGVVQMVLYVFYMNKTPVAAAVGKDAGKLPSAADEHVLVNIAKLNPALPERTSGMHPVTQMAAVPARSCAAEAIAPAMLPNRDVVDVFVSRHSPAVHVV

SEQ ID NO: 54 - protein, 344 aa, Zea mays
MAGLSLQHPMAFAFGLLGNIISFMTYLAPLPTFCRIYRNKSTEGFQSVPYVVALFSAMLWIYYALLKSNEFLLITINSAGCVIETLYIATYLLYAPNKAKLFTAKILLLLNVGVFGLILLLTLLLSAGPHRVVVLGWVCVAFSVSVFVAPLSIIRQVVRTRSVEFMPFSLSFSLTASAVVWFLYGLLIKDKYVALPNVLGFTFGVVQMGMYALYRNATPRVPAAKEAAAAADDGNTFNFKAPGEHVVTIAKLTAAAPATAAELIIKARDDAQHPPEEEAAAAKAAPAKSKLLIPLPEHAYACMCIIRSGSHHKLGRACLLGTSTRPPACLPARMIQSSCYIRKG

SEQ ID NO: 55 - protein, 292 aa, Zea mays
MAGLSLLHPMAFAFGLLGNIISFMTYLAPLPTFYRIYKNKSTEGFQSVPYVVALFSAMLWIYYALLKSNELLLITINSAGCVIETLYIAMYLLYAPKKAKLFTAKILLLLNVGVFGLILLLTLLLSAGQRRVVVLGWVCVAFSVSVFVAPLSIIRQVVRTRSVEFMPFSLSLSLTVSAVVWFLYGLLIKDKYVALPNVIGFSFGVVQMGLYALYRNATPRVPAKDVADDASKDKAPGEHVVVTIAKLTAATTAPAAAVAEDLVKVHDGHPEEAAKGAAKPAENGAGRSDAEQ

SEQ ID NO: 56 - protein, 304 aa, Zea mays
MAFLNMEQQTWAFTFGILGNIVSLMVFLSPLPTFYRVYRNKSTEGFQSTPYVVTLFSCMLWILYALLKPGAELLVTINGVGCVVETVYLAMYLVYAPKAARVLAAKMLLGLNVAVFGLVALVTMLLSDAGLRVHVLGWICVSVSLSVFAAPLSIMRQVIRTKSVEFMPISLSFFLVLSAVVWFAYGALKKDVFVAFPNVLGFVFGLAQMALYMAYRKPAAALVIIPEQSKEEVAEGKASCGGAEVHPIDIAEVHDLQTVVVDVDVEPVTYAAASGMVDGSVGRPRAPEELVIKPDMVTVIAAEA

SEQ ID NO: 57 - protein, 238 aa, Zea mays
MDSTLFIIGVIGNIISVLVFISPIKTFWRIVRSGSTEEFEPAPYVFTLLNALLWLYYGATKPDGLLVATVNGFGAAMEAIYVVLFIVYAANHATRVKTAKLAAALDIGGFGVVFVATTFAINELNMRIMVIGMICACLNVLMYGSPLAAMKTVITTKSVEFMPFFLSFFLFLNGGIWATYAVLDRDIFLGIPNGIGFILGTIQLIIYAIYMNSKVSQSSKEIASPLLASSQEEAASHV

SEQ ID NO: 58 - protein, 249 aa, Citrus sinensis
MDIAHFLFGVFGNATALFLFLAPTITFRRIVRRKSTEQFSGIPYVMTLLNCLLSAWYGLPFVSKNNILVSTINGTGSAIEIIYVLIFLLFAPKKEKAKIFGLFMLVLTVFAAVALVSLLAFHGNARKIFCGFAATIFSIIMYASPLSIMRMVIKTKSVEFMPFFLSLFVFLCGTSWFVFGLLGRDPFVAVPNGFGCGLGTMQLILYFIYHKKGEPEKPSAANGSVEMGQEKPLEGTKMANGNGALVEQV

SEQ ID NO: 59 - protein, 235 aa, Citrus sinensis
MILTVTYQALTVLKDAVGIAGNIFAFGLFVSPVPTFRRIIRNHSTEEFSGLPYVYALLNCLITMWYGTPLVSADNILVTTVNSIGAAFQLVYIILFITYTEKDKKVRMLGLLLAVIGIFSIIAAVSLQIVNPFSRQMFVGLLSCAALISMFASPLFIINLVIQTKSVEFMPFYLSLSTFLMSTSFLAYGIMNWDPFIYVPNGIGTILGIVQLALYFNYKETSGEESRDPLIVSYA

SEQ ID NO: 60 - protein, 264 aa, Citrus sinensis
MSSVGISSIYSGCSVAAGVTGNIFAFVLFVSPIPTFRRILRNKSTEQFSGLPYICSLLNCLITLWYGMPLVSPGIILVATVNSVGAVFQLIYVSIFISYAEKAIKLKISGLLIAVFLVFLAIVFTSMEVFDSNGRRLFVGYLSVASLISMFASPLFIIVSSSGTQAFRLLRLHISLHSYGCMYIFMQKLVIKTRSVEFMPFYLSLSNFLMSLSFLAYGMFKDDPFIYVPNGIGTLLGIAQVMLYSYYSTKSGEVSRQPLIDSFA

SEQ ID NO: 61 - protein, 249 aa, Citrus sinensis
MGDGLRLAFGVMGNAASLLLYATPILTFSRVIKKKSTEGFSCFPYIIALLNCLLYTWYALPVVSYRWENFTVVTINGLGIFLELSFILIYFLFASARDKIKVAAIVIPVILLFCITALVSAFVFHDHHHRKLFVGSIGLGASITMYSSPLVAVKQVIRTKSVEFMPFHLSFFSFLTSAIWMVYGLLSHDLFIASPSFVGGPLGILQLVLYWKYRKSGIIKEPNKWDLEKNGENSKKLQLAINNDINGKS

SEQ ID NO: 62 - protein, 205 aa, Citrus sinensis
MFWILYGLPVVHPDSTLVITINAVGLALELIYLSIFCFTNICFYFARRTCHLITCLILAYLQTVVGLGLLAEVIFVGVIAIITFLAFHTHTSRSMFVGILCDIFNIIMYASPLTIWHKVITTKSVEYMPFFLSLANFANGCIWTAYALIKLDIYILVSNGLGAILGFIQLVIYACYYKSTPKKGNDDDFVKPKPTEVQHSGAAMA

SEQ ID NO: 63 - protein, 240 aa, Citrus sinensis
MVSAEAARNIVGIIGNVISFGLFLSPTPTFWRIIKRKDTEEFHPYAYICACMNCMFWILYGLPVVHPDSTLVVTINGVGLALELIYLSIFCVYNRQKKGRKIVAIGLLGEVAFLGVIAVITFVVFHNTNTRTLFVGIICDIFNIIMYASPLSIWHKVIKTKSVEYMPFFLSLANFANGAVWTAYGLIKFDKFIVVSNGLGTVLGAIQLIIYGCYYKSTPKKGSGDVIKPNEVQLSGATIA

SEQ ID NO: 64 - protein, 93 aa, Citrus sinensis
PTFVKIFKKRSVEEFKPDPYLATIMNCSLWVFYGLPFVTPDSILVVTINSTGLAMEIAYITIFFVFAQKKGRRLLLRFLFLFLAKSFLFLKIF

SEQ ID NO: 65 - protein, 255 aa, Citrus sinensis
MVETGLIRTVVGIIGNVISLGLFLSPIPTMAAIVRQKSVENFKADPYIATVLNCFVWTFYGLPFVHPDSTLVVTINGAGAAIELFYVLIFVIFSSWGKRRKIFVALVVEVVFMAILIFVTLYFLHTTDDRTTVVGIIAVVFNIVMYAAPLTVMKMVISTKSVKYMPLALAIGNAANGAVWVVYACLRFDPYVLIPNGLGTLSGILQLTLYAIFYKTTNWDGDDDENRNDNNGNGNGNGSNNNRRGRGEVQLVDVA

SEQ ID NO: 66 - protein, 241 aa, Citrus sinensis
NIISLFLFLSPVPTFVEIVKKGTVEQYSAAPYLATLLNCMVWVLYGLPMVHPHSILVITINGSGTAIEVVYIILFVLHSDKKKRIKVMLVVLVEVIFVALVALLVLTLLHSTKQRSMAVGIICILFNIMMYASPLSVMKLVITTKSVEYMPFFLSLMSLANGIAWTTYAFLPFDQFIAIPNGLGTLLGVAQVILYACYYKSTKRQMAARQGKGQVDLSAVVVSESDSGDSKKIGTAVGGGG

SEQ ID NO: 67 - protein, 255 aa, Citrus sinensis
MGILTPHQLAFIFGLLGNIVSFLVFLAPVPTFLIIYKKKSSEGYHSIPYVIALSSATLLLYYGLLKSNAVLIITINSIGCVIEVIYLMLYLIYAPQKQKSFTIKLILVFNVGAFALMMVIVNFFVKGPNRVTAVGCVCAVYNVAVFSAPLSIMRRVIKTKSVEYMPFSLSFFLTLCATMWFFYGLFVKDMVIALPNVLGFLFGIAQMILYLVYKGKKGNESNQKQQECTEMKMNLTEDDKAYTKDNNQPTDLQTN

SEQ ID NO: 68 - protein, 243 aa, Citrus sinensis
NITSFLVCLAPMPTFYKIYKKKSTEGFQSVPYVISLFSAMIWIYYALLKQNAIFLMTINTFCCVMQTIYIAVYVFYAPKKVRIQTVKLLLLLNIFGFGAIREKILGYICMTFALSVFAAPLFIVRKVIKTKSVEYMPFTLSFFLTIGAVAWFFYGLLIKDLNVAIPNVLGFIFGVLQMILYVIYKNPNKKIVEQTKLQELSEHVVDVVKLSTMRHPGPRAAYALYTKQQTLLNNCILALQTCF

SEQ ID NO: 69 - protein, 275 aa, Citrus sinensis
MAILGPHSVIIFGLLGNIVSFLVYLAPLPTFYRIFKKKSTQGFQSIPYSVALFSAMLLLYYASLKGSNAFMLITINGIGCIIESLYLLFFMIYATKTAKIYTTKLLILFNIGALGLIVLLTYLLSKSSDQRLTIVGWICAVFSVCVFAAPLSIIRQVIRTKSVEYMPFSLSCCLTICAGMWLLYGLSIKDYYIATPNILGMAFGATQMILYLAYRTRRNSEILPVAAAVVDPKDREESNNTGAADPCCNHHHRHDSSNGEVEIKAVETNQINHTA

SEQ ID NO: 70 - protein, 322 aa, Citrus sinensis
MTMFSTHDPSVFAFGLLGILQIQKCHCLNIIFMLHAYVYVFVANIFICFHVTIIGNIVSFIVFLAPMPTFYRVCKKKSTEGFQSLPYVVALFSAMLWIYYAMMKKDAFLLITINAFGCVIETIYLALYITFAPKQARLYTLRLLLLLNFGGFGSILLLSHFLAKGSAARLRLLGWVCVVFSVSVFAAPLSIMRLVVRTKSVEFMPFYLSLFLTLNAVMWFFYGLFLKDVYVAVPNVLGFIFGVVQMILYAIYRNYRRVVVEDVNKVPEHTVDVVKLSTNNMTASEEQTNSRNNFDDKNEHEQANDQHEKARESCNQDPLNKC

SEQ ID NO: 71 - protein, 248 aa, Citrus sinensis
MLWFYYALVKQNAFLLVTINCFGCVIETIYIILFITYAPKGSRNSTVKLFVSMNVGVFSLILLLTHFLATDSTRILILGWICVAVSVSVFAAPLSIVAQVIRTKSVEFMPFILSFFLTLSAIMWFAYGLFQKDICVALPNIVGFLLGLTQMLLYVIYKNANKVIIEDKKLPEAQLKSIVVLSNLGASEVYPVDIHPDDADANDVNQGPKENRQETDQRNPKSLEVPGGLQLQQHNDNNNTDDGCAVAV

SEQ ID NO: 72 - protein, 308 aa, Citrus sinensis
MASLSFFVGIIGNVISLLVFASPIKTFWQIVKKKSTESYKGVPYITTLMSTCLWTFYGVMKPGGLVVATVNGAGAALQFIYVSLYLIYAPKDKKVKTAKLVAILDVGFLGAVIAITLLAMHGNLRLTFVGILCAALTIGMYASPLAVMTTVIRTKSVKYMPFLLSFFLFLNAGVWSVYSVLVKDIYIGVPNAVGFVLGAAQLILYMIYKNKTPLPTKSMDSVKERSAHKVKDGIEMGARGDDHDNQEDDLEEANGKKKRTLRQGKSLPKPTLGKQFSIPKILKKTASLGPYDLYSSWYHHYDDSDVDA

SEQ ID NO: 73 - protein, 114 aa, Citrus sinensis
MASLNFIFGLLGNLTTGLVYLSPAKTFWHIVQRRSTEEFESIPYISKLLNAYFWVWYGIVKPNSVLVASVNGFGAALEIIYVIIFLIFAPPMMRGRTAVLAGVCDVVFPGTTVL

SEQ ID NO: 74 - protein, 234 aa, Citrus sinensis
MKDLSFYVGVIGNIISVLMFLAPVRTFWRIIKHRSTEEFQSLPYICTLLNSSLWTYYGITRPGSYLVATVNGFGILVEAVYVTLFFIYAPTKAMRAKTAIIFGILDVGFLGAAIAATRLALEGEARIDAIGFMCAGLNIIMYASPLSAMKTVVTTKSVEFMPFMLSFFFFLNGGIWAFYALLVRDIFLGVPNGTGFLLGTAQLVLYAIYRNAKPSKNAANSMEEGAQHEPLIIS

SEQ ID NO: 75 - protein, 235 aa, Medicago trunculata
MSLFNAYSICEIGKDAAGIAGNIFAFGLFVSPIPTFRRIMRNGSTELFSGLPYIYSLLNCLICLWYGTPLISCDNLLVTTVNSIGAAFQLVYIFLFLIYAEKPKKVRMFGLLLAVLGIFVIILVGSLKITDSSIRRILVGCLSCASLISMFASPLFIIKLVIRTKSVEFMPFYLSFSTFLMSISFFLYGLLSDDAFIYVPNGIGTVLGMIQLILYFYYKRSSSDDSTEPLIVSYG

SEQ ID NO: 76 - protein, 236 aa, Medicago trunculata
MSVFASLAICKVAKDAAGVAGNIFAFGLFVSPIPTFRRIIRNGSTEMFSGLPYIYSLMNCLICMWYGTPLISHDNILVTTVNSIGAVFQFVYIILFMMSAEKEKKVKMLAWLMGVLGIFAIILIGSLQIDDIVMRRLFVGILSCASLISMFASPLFIIKLVIQTKSVEFMPFYLSLSTFLMSTSFLVYGLLSDDIFIYVPNGIGTILGMTQLILYFYYESKSRRMDAEEPLIVSYA

SEQ ID NO: 77 - protein, 250 aa, Medicago trunculata
MSETLRLAVAVLGNAASVSLYAAPMVTFKRVIRKKSTEEFSCIPYIIGLLNCLLFTWYGLPIVSYKWENFPLVTVNGVGIALELSYVLIYFWYSSPKGKVKVAMIMTPVLLVFCIVAAVSAFSFHDTAHRKLLVGSIGLGVSVALYGSPLVAMKKVIETKSVEFMPLPLSLCAFSASACWLVYGILVRDVFVAGPSVVGTPLSILQLVVYFKYRKARVVEEQKIGDLEKGSIELEKVVKVEKIVTNCEQC

SEQ ID NO: 78 - protein, 263 aa, Medicago trunculata
MSTAEIARTAVGIIGNVIAGCMFLSPVPTFVGICKKGSVEQYSPVPYLATLMNCMVWTLYGLPMVHPHSFLVVTINGAGCVVEIIYITLFLIYSDRKKRLKVFLGLLLELIFIFLLSFVSLTMLHTVNKRSAVVGTICMLFNIGMYASPLSIMKLVIKTKSVEFMPFFLSLASFGNGVSWTIYALIPFDPFIAIPNGIGTMFAVVQLILYASYYKSTQEQIAARKNNGKGEMNLSEVVVGMSNATVQDNKKITAIDHSSPSAK

SEQ ID NO: 79 - protein, 263 aa, Medicago trunculata
MARMQVRRSALHTCCGQELKHHPNLDKCPNTYLWPTFIKICKAKSVQDFKPDPYVVTILNCAMWSFYGMPFISKSNTLVLTINGFGFFIEIIYTSIFFVYSNGSKRVRNISNLLIKLQSIFPFNVLKIELKKKILLALLAEVVFLVLVVFIVMYFVTNLKERRFIVGVICIIFNILMYFSPLTVMRQVIRSKSVKYMPFLLSLANFANGLIWTTYALLRWDPFVVIPNGLGALSGLAQLILYAVYYRTTKWDDDAPPSSVNNV

SEQ ID NO: 80 - protein, 252 aa, Medicago trunculata
MFPFSNLKMVLLFGFLGIVTFMSFLAPLPTFYSIYKKKSSEGFHSIPYVVTLLSTLLFVYYGFLKTNAIFLITINSIGCVMEVAYLIMYITYAPKKLKISTLVLILIVDMGGFGLTMIITTFIVKGSFHVQVVGMICTIFNIGMFAAPLSIMKKVIKTRSVEYMPFPLSLFLTICATMWFFYGFFDKDKYIMLPNGLGFLLGVSQMILYLIYKNAKNNVEASSTNQLQEHGCDGGNNQIFPTVVEMKEINIV

SEQ ID NO: 81 - protein, 270 aa, Medicago trunculata
MALFYSEYWAFVFGVIGNVISCMTFLAPLPTFYRIYKKKSTEGFQSVPYVTALLSAMLWIYYAHVKNKATLLLLTINIYGFGIEAIYIIIFLLYASNKARLSTIKLLFLTVCGYGTMVILTTYLTKGSKRLSIIGWICMVFNICVFASPLFILKQVIKTKSVAFMPLNLSFFLTLNAIVWFFYGLLIDDFYIAIPNTLGFVFGIVQMVIYLIYKDAIPLESTKLQKPNDHVLNICEDVPNGALQPDPNQVVKSGAPAVAVIGDEDPNNGK

SEQ ID NO: 82 - protein, 255 aa, Medicago trunculata
MAMTRESWAFVFGIIGNIISFAVFLSPLPTFYVIFKKKSAEGFQALPYVVALFSAMLWIYYAFVKRESALLLITINTFGIVVESAYIIMFLIYAPKKQRLSTIKLLLLLNVFGFGAMLLSTLYLSKGAKRLAIIGWICLVFNISVFAAPLFVISKVIRSRSVEYMPFFLSFFLTINAVMWFFYGLLLRDYYVALPNTLGFVFGIIQMVVYLIYRNATPVVEAPMKGQELSGGHIIDVVKIGTDSNRAGGGAGSKV

SEQ ID NO: 83 - protein, 288 aa, Medicago trunculata
MAMISMNHHFLVIAFGLLGNIISCMVYLAPLPTFIQIYKKKSTECFQSLPYLVALFSSMLWLYYGIQTNAIFIVSINAFGCVIEIIYCIMYIAYATKDARKLTIKLCAALNVVSFVLIFLIIQFSIPENHRVQVLGWICTSISISVFAAPLSIVVRVVKTKSVEFMPFNLSLFLTLSAVVWFLYGFVKRDICIYLPNVVGFILGIIQMVLYGYYSKYSVEKEKEQAVINIVVVNPLGSSEVFPIPLDENKESIEDVINQQFQVKKVGEEDAKEKHDNNVEAIEFQCVV

SEQ ID NO: 84 - protein, 268 aa, Medicago trunculata
  misc_feature (192)..(192): Xaa can be any naturally occurring amino acid
MAISHNTLAFTFGMLGNVISFLVFLAPISTFYRIYKKKSTEGFQSLPYLVALFSSMLWLYYALLKKDAFLLITINSFGCVVETIYIILYIIYAPRDARNLTFKLLSAMNVGSFALILIVTNYAVHGPLRVQVLGWVCVSLSVSVFAAPLSIVAQVVRTKSVEFMPFNLSFTLTLSATMWFGYGFFLKDICIXLPNVLGXVLGLLQMLLYAIYRNGGEKAMKKEKKAPIEPPKSIVIETQLEKIEQEKKNKDDDNEEKDKSEEPIGCGV

SEQ ID NO: 85 - protein, 268 aa, Medicago trunculata
MAISHNTLAFTFGMLGNVISFLVFLAPISTFYRIYKKKSTEGFQSLPYLVALFSSMLWLYYALLKKDAFLLITINSFGCVVETIYIILYIIYAPRDARNLTFKLLSAMNVGSFALILIVTNYAVHGPLRVQVLGWVCVSLSVSVFAAPLSIVAQVVRTKSVEFMPFNLSFTLTLSATMWFGYGLFLKDICIALPNVLGFVLGLLQMLLYAIYRNGGEKAMKKEKKAPIEPPKSIVIETQLEKIEQEKKNKDDDNEEKDKSEEPIGCGV

SEQ ID NO: 86 - protein, 268 aa, Medicago trunculata
MAISHNTLAFAFGMLGNVISFMVFLAPMTTFYRIYKKKSTEGFQSLPYLVALFSSMLWLYYAFLKKDEFLLITINSFGCVVELIYIILYIIYATKDARKLTIKLLLAMNIGSFGLILLVTKYAVHGPIRVQVLGWICVSISVSVFAAPLTIVAQVVRTKSVEFMPFNLSFTLTLSAIMWFGYGLFLKDICIALPNVLGFALGLVQMILYCIYRNGDKKKANSKAALKSVVIESSLGGTGEVFQVEKNDGEEEEEKKKTIEETEYDSKV

SEQ ID NO: 87 - protein, 269 aa, Medicago trunculata
MDPHDHDRLAFIFGILGNIISSMVYLAPLPTFYRIWKKKSTEGFQSLPYLVALFSSMLWLYYGFVKKHAFLLITINSAGCVIETIYIVTYLIYATKDARILTIKLFMAMNVACSVLIVLTTQLAMHGKLRVHVLGWICTSFAICVFAAPLTIMAKVIRTKSVEFMPINLSFFLTLSAIVWFFYGLLLHDICIAIPNVLGFILGLLQMLLYAIYNKSVKEEYALEPMTNIVIVNPLGIPCEVFSLPVIDNVNKIEKEGAEEMEKSVENLT

SEQ ID NO: 88 - protein, 246 aa, Medicago trunculata
MADPSFFVGVIGNIISILMFLSPVPTFWRMIKKKSTEEFSSFPYICTLLNSSLWTYYGTIKAGEYLVATVNGFGIVVETIYILLFLIYAPPKMRVKTAILAGILDVLILVAAVVTTQLALGGEARSGAVGIMGAALNILMYGSPLAVMKTVVKTKSVEYLPFLLSFFFFLNGGVWLLYAVLVRDSILGVPNGTGFVLGAIQLVLHGIYRNGKQSKHVSNKLEEGWQHEHLISSSTTRSHDRENLPI

SEQ ID NO: 89 - protein, 231 aa, Triticum aestivum
MDSLSLYEISCFAAGFAGNLFAFALFLSPVPTFKRILKAKSTEQFDGLPYLLSLLNCFICLWYGLPWVSDGRLLVATVNGTGAAFQLAYISLFFIYADSRKTRLRMVGLLVLLVCAFALVAHASIAFFDQPTRQQFVGAVSMASLISMFASPLAVMGVVIRTECVEFMPFYLSLSTLLMSASFAVYGLLLRDLFIYLPNGLGVVLGATHLALYAYYSRKWRCKDSSAPLLA

SEQ ID NO: 90 - protein, 289 aa, Triticum aestivum
MAGLSMEHPWAFAFGLLGNIISFTSLLAPIPTFYRIFKSKSTEGFQSVPYVVALFSAMLWIFYALVKTGEGLLITINAAGCVIETVYIIMYLVYAPRKAKIFTAKIVLLLNVAGFGLIFLLTLFAFHGETRVVSLGWICVGFSVCVFVAPLSIIGRVIKTKSVEYMPFSLSLTLTLSAVVWFLYGLLIKDKYVALPNILGFTFGMIQMVLYMFYMNATPVVASDAKEGKEAWKVPAEDHVVVINVGKADKSSCAEVRPVADVPRRCAAEAAAPGQQVMAVDFARSVEVV

SEQ ID NO: 91 - protein, 247 aa, Glycine max
MDVAHFLFGIFGNASALFLFLAPVITFKRIIKNRSTEKFSGIPYVMTLLNCLLSAWYGLPFVSPHNILVSTVNGTGSFIEIIYVLIFIVLAPRKEKAKILGLFTFVLSVFSAVVFVSLFALHGNSRKLFCGFAAAIFSIIMYGSPLSIMRLVIKTKSVEFMPFFLSLFVFLCGTSWFIFGLLGRDPFVAVPNGVGSALGTMQLILYFIYRDNKGVPRKQAPTEEESMEMGDAKPQQGKQSNANGIQG

SEQ ID NO: 92 - protein, 235 aa, Glycine max
MSLFAAFSICKVAKDAAGVAGNVFAFGLFVSPIPTFRRIIRNGSTEMFSGLPYIYSLLNCLICMWYGTPLISADNLLVTTVNSIGAVFQFVYTIIFLMYAEKAKKVRMVGLLLAVLGMFAIVLVGSLQIDDVIMRRFFVGFLSCASLISMFASPLFIIKLVIQTKSVEFMPFYLSLSTFLMSTSFLLYGLFNDDAFIYVPNGIGTILGMIQLILYFYFESKSRESSREPLIVSYA

SEQ ID NO: 93 - protein, 254 aa, Glycine max
MAETLRMVVAVIGNVASVSLYAAPTVTFKRVIRKKSTEEFSCMPYIIALLNCLLFTWYGLPVVSNKWENLPLVTVNGVGILFELSYVLIYIWFSTPKGKVKVAMTAVPVLIVFCVIAIVSAFVFPDHRHRKLLVGSIGLGVSIAMYGSPLVVMKKVIQTKSVEFMPLPLSFCSFLASVLWLTYGLLIRDIFVAGPSLIGTPLGILQLVLHCKYWKRRVMEEPNKVELQKGNNTEKLDLEMGHGKECVTVPSNCN

SEQ ID NO: 94 - protein, 254 aa, Glycine max
MAETIRLAVAVLGNAASVALYAAPMVTFRRVIRKKSTEEFSCFPYIIGLLNCLLFTWYGLPVVSYKWENFPLVTVNGVGIVLELSYVLIYFWYASAKGKVKVAMTAIPVLLVLSIIAAVSAFAFHDNHHRKLLVGSIGLGVSVTMYGSPLIVMKKVIQTKSVEFMPLPLSMCSFLATVFWLIYGLFIRDIFVAGPSAVGTPLGILQLVLYCKYRKGSVVEDPSKGDLEKGNLEKVEMEIGKVEMNVTNHMNGHS

SEQ ID NO: 95 - protein, 262 aa, Glycine max
MVSISDHELVLIFGLLGNIVSFMVFLAPLPTFYTIYKKKSSEGFQSIPYAVALLSALLLLYYGFIKTNATLIITINCIGCVIEVSYLTMYIIYAPRKQKISTLVMILIADIGGFGLTMLITTFAVKGINRVHAVGWICAIFNIAVFAAPLSIMRRVIKTKSVEFMPFSLSLFLTLCATMWFFYGFFDKDDFIMFPNVLGFIFGISQMILYMIYKNSKKNGETNCTEQQESEGTVNSKQHSCDGNKLDFPSLVEMKENQLNQV

SEQ ID NO: 96 - protein, 262 aa, Glycine max
MVLFSDHELVLIFGLLGNIVSFMVFLAPLPTFYTIYKNKSSEGFQSIPYVVALLSALLLLYYGFIKTNATLIITINCIGCVIEVSYLAMYIIYAPRKQKISTLVMILIADIGGFGLTMLITTFAVKGINRVHAVGWICAIFNIAVFAAPLSIMRRVIKTKSVEFMPFSLSLFLTLCATMWFFYGFFDKDNFIMLPNVLGFLFGISQMILYMIYKNAKKNGEINCTEQQERDGTVNSKQHSCNGNKLDFSSLVEMKENQLNQV

SEQ ID NO: 97 - protein, 260 aa, Glycine max
MAINHETWAFVFGLLGNVISFMVFLAPLPTFYQIYKKKSTEEFQSLPYVVALFSSMLWIYYALVKKDASLLLITINSFGCVIETIYLAIFLIYAPSKTRLWTIKLLLMLNVFGFGAMLLSTLYLTTGSKRLTVIGWICLVFNISVFAAPLCIIKRVIKTKSVEFMPFSLSFFLTINAVMWFFYGLLLKDYYVALPNTLGFLFSIIQMVLYLIYRNAKTPDLPMKLQELNSHTIDVGKLSRMEPSEPNHVTKNGTLTEREI

SEQ ID NO: 98 - protein, 258 aa, Glycine max
MAISHETWAFIFGLLGNVISFMVFLAPLPTFYQIYKKKSSEGFQSLPYVVALFSSMLWIYYALVKKDASLLLITINSFGCVIETIYLAIFLVYAPSKTRLWTIKLLLMLNVFGFGGMLLSTLYLTTGSKRLSVIGWICLVFNISVFAAPLCIMKRVIKTRSVEFMPFSLSLSLTINAVMWFFYGLLLKDYYIALPNTLGFLFGIIQMVLYLVYRNAKPQTLEEPTKVQELNGHIIDVVKPNHATKNGHVPVIEIASSV

SEQ ID NO: 99 - protein, 294 aa, Glycine max
MAHANPMIFVVGILGNLVSFCCFLAPVPTFYRVCKKKTTEGFQSLPYVAALFTSMLWIFYAYIKTGEILLITINAFGCFIETVYLVIYITYCPKKARFFTFKMIFLFNVGVIFLVVLLTHVLAKERTARIELLGWICVVLSTSVFAAPLSIIKVVIRTKSVEFMPITLSLLLTVSAMMWMAYGILLRDIYVTLPNFVGITFGTIQIVLYLIYRKNKPVKDQKLSEHKDDVANDENVNTAVSGENRGANATGFVDIEIGEKKQVQEQADKKQDQQAVNARDQTEHNNNSNKTREG

SEQ ID NO: 100 - protein, 309 aa, Glycine max
MSHSHLSFAFGILGNIASFVCFLAPLPTFYRVCKKKSTEGFQSIPYVAALFSAMLWIFYAYVKTGETLLITINAFGCVIETIYLAVFITYCPKKARMSTLRMIVLLNFGGFCTIVLLTHLLAKGEEARVKLLGWICVVFATSVFAAPLSIIRVVIRTKSVEFLPFPLSLLLLISAIMWLLYGISLKDIYVTLPNVVGLTFGVIQIGLYAMYRNNKPIKDQKLPEHKGDIVESENVIAPTGNGEKQEEEVKPQGGDIEIGEKKEENNKQDQQQSVENKKLDQVAHDQTELNKNNINKNNNKTEERVSCEV

SEQ ID NO: 101 - protein, 254 aa, Glycine max
MTMHRESWAFVFGVMGNIISFGVFLAPLPTFYQIYKKKSTEGFQSLPYVVALFSAMLWIYYAFVKRETALLLITINTFGIVVESIYLSIFLIYAPRKPRLTTIKLLLLLNVFGFGAMLLSTLYLSKGAKRLAIIGWICLVFNISVFAAPLFIIRRVIKTRSVEYMPFTLSMFLTINAVMWFFYGLLLRDYYVALPNTLGFVFGIIQMVMYLMYRNATPVALEEPVKAQELNGHIIDVVKIGTMEPNHGGAVGKV

SEQ ID NO: 102 - protein, 271 aa, Glycine max
MVISHHTLAFTFGMLGNLISFLVFLAPVPTFYRIYKKKSTESFQSLPYLVALFSSMLWLYYAMLKRDAVLLITINSFGCVIEIIYIVLYITYATRDARNLTIKLFSAMNMSSFALILLVTHFAVHGPLRVQVLGWICVSISVSVFAAPLSIVAQVVRTKSVEFMPFNLSFTLTLSAIMWFGYGLFLKDICIALPNVLGFVLGLLQMLLYTIYRKGNKKTKTNEKSPVEPLKSIAVVNPLGTGEVFPVEEDEQAAKKSQGDGDDKKGQDCLV

SEQ ID NO: 103 - protein, 283 aa, Glycine max
MAIFNGHNHLALGFGMLGNVISFMVYLAPLPTFYRIYKKKSTEGFQSLPYLVALFSSMLWLYYASLKPADATLLITINSLGCVIEIVYIIMFTIYATKDARNLTVKLFMVMNVGSFALIFLVTYFAMHGSLRVQVVGWVCVSIAVGVFAAPLSIVAQVIRTKNVEFMPFNLSLFLTISAVMWFFYGLLLKDICIAIPNILGFTLGLLQMLLYAIYRNGKTNNKEVVTKEEHALEAMKNVVVVNPLGTCEVYPVIGKEINNNGQGIEGAEEKEKGVELGKECPV

SEQ ID NO: 104 - protein, 265 aa, Petunia hybrida
MAQLRADDLSFIFGLLGNIVSFMVFLAPVPTFYKIYKRKSSEGYQAIPYMVALFSAGLLLYYAYLRKNAYLIVSINGFGCAIELTYISLFLFYAPRKSKIFTGWLMLLELGALGMVMPITYLLAEGSHRVMIVGWICAAINVAVFAAPLSIMRQVIKTKSVEFMPFTLSLFLTLCATMWFFYGFFKKDFYIAFPNILGFLFGIVQMLLYFVYKDSKRIDDEKSDPVREATKSKEGVEIIINIEDDNSDNALQSMEKDFSRLRTSK

SEQ ID NO: 105 - protein, 248 aa, Populus trichocarpa
MDVLHFLFGVFGNATALFLFLAPTITFKRIIRSKSIEQFSGIPYVMTLLNCLLSAWYGLPFVSKNNVLVSTINGAGSAIETIYVLIFIIYAPKKEKAKVLGLLTLVITIFTGVALVSLFALHGNARKLFCGCAAAVFSIIMYGSPLSIMRTVIKTKSVEYMPFFLSLFVFLCGTSWFVYGLLGRDPFVAVPNGVGCGLGALQLILYFIYRNNKGEAKKPISTHSLEIGPGKVHQEKKLVANGSHDERV

SEQ ID NO: 106 - protein, 250 aa, Populus trichocarpa
MEIAHFLFGIFGNATALFLFLAPTITFRRIIRSKSTELFSGIPYVMTMLNCLLSAWYGMPFVSKNNILVSTINGTGAVIEAVYVLTFIIYAPKKEKAKFIGLLTLVLTTFAGVALVSLVVLHGKPREIFCGFAAAIFSIIMYGSPLSIMRTVVKTKSVEFMPFFLSLFVFLCGTSWFVFGLLGGDLFVAVPNGVGCGLGALQLILYFIYRNNKGEDKKPALPVKSMQMGIAKLHQQKELVANGSHVADKV

SEQ ID NO: 107 - protein, 259 aa, Populus trichocarpa
MGFLSNDQLTFLFGLLGNIVAAGMFLAPVPTFYTIFKRKSSEGFQSIPYSVALMSASLLLYYGLLKTNAYLLISINSIGCAFEVTYLIIYLIYAPKQEKMHTMKLLLIFNMGSFGVVLLLTMLLMKGKPRLSVVGWICAVFSVAVCAAPLSIMRRVVRTKSVEYLPFTLSASITLNAVMWFFYGLLQHDYYIALPNVLGFLFGIAQMILYMVYKNLKKNVEEKSEQLAGNMEVVQMTKETESCTVDDPHMETKICICDL

SEQ ID NO: 108 - protein, 248 aa, vitis vinifera
MDAHHALHFTFGIFGNATALFLFLAPLITFKRIIKSKSTEQFSGIPYVMTLLNCLLSAWYGLPFVSKNNILVSTINGTGAAIEIIYVLIFIAYSIKKERAKILGLFIFVLSVFGVVVFVSLFALHGHSRKLFCGLAATIFSIIMYASPLSIMRMVIKTKSVEYMPFFLSLFVFLCGTSWFVFGLLGKDPFVAVPNGFGCGLGAMQLILYAIYCKKGKSKNLAAADKPVDMELGKPQQEKQSRAQNGNV

SEQ ID NO: 109 - protein, 235 aa, vitis vinifera
MSRSLLLPVNTICKDAAGVAGNIFAFGLFVSPIPTFRRIARNRSTESFSGLPYIYALLNCLVTLWYGTPLVSYNNIMVTTVNSMGAAFQLVYIILFITYTDKRKKVRMFGLLMVDIVLFLVIVVGSLEISDFTIRRMVVGFLSCAALISMFASPLFVINLVIQTRSVEFMPFYLSLSTFLMSASFLAYGILNNDPFVYVPNGAGTVLGIVQLGLYSYYKRTSAEESREPLIVSYG

SEQ ID NO: 110 - protein, 232 aa, vitis vinifera
MSSVYSVCCDAAGIAGNLSAFVLFVSPIPTFRRIIRNGSTEQFSGLPYIYALLNCLICLWYGMPLVSPGIILVATVNSVGAIFQLIYIGIFITFAEKAKKMKMSGLLTAIFGIYAIIVFASMKLFDPHARQLFVGYLSVASLISMFASPLFIINLVIRTRSVEYMPFYLSLSTFLMSLSFFTYGMFKHDPFIYVPNGIGTILGVVQLVLYAYYSRTSTEDLGLRESFIESYA

SEQ ID NO: 111 - protein, 249 aa, vitis vinifera
MGDRLHLAIGVMGNAASLLLYTAPILTFARVMRKKSTEEFSCIPYIIALLNCLLYTWYGLPVVSYRWENFPVVTINGLGILLEFSFILIYFWFTSPRGKIKVVGTVVPVVTVFCITAIISSFVLHDHHHRKMFVGSVGLVASVAMYGSPLVVVRQVILTKSVEFMPFYLSFFSFLTSFLWMAYGLLGHDLLLASPNLVGSPLGILQLVLYCKYRKRGIMEEPNKWDLEGNDEKSKQLQPVINNDSNGKI

SEQ ID NO: 112 - protein, 270 aa, vitis vinifera
MALFPIHHPLVFIFGILGNLISFMVYLAPLPTFYQIYKRKSTEGFQSVPYVVALFSAMLWIYYAFLNTDASLLITINSVGCVIETSYIVMFLVYAPKKARITTVKLVFLMNICGFGSILLLTLLLAEGANRVRILGWVCLVFSLSVFLAPLCIMRQVIRTKSVEYMPFLLSFFLTLSAVMWFFYGLMLKDFYIAGPNILGFVFGIVQMVLYLIYRNRKKVLENEKLPELSEQIIDVVKLSTMVCSEVNLTNQQHSNEGHGTTGLEVIVAL

SEQ ID NO: 113 - protein, 276 aa, vitis vinifera
MAMLTVPHMAFAFGILGNIVSFLVYLSPLPTFYRIYKRKSTEGFQSIPYSVALFSAMLLLYYAFLKTDNQIMLITINSVGTCIEATYLLVYMIYAPRTAKIYTAKLLLLFNTGVYGAIVLSTFFLSKGHRRAKIVGWVCAAFSLCVFAAPLSIMRLVIRTKSVEYMPFPLSFFLTICAVMWFFYGLLIRDFYIAFPNILGFAFGIAQMILYTIYKNAKKGVLAEFKLQELPNGLVFPTLKKAENTDTNPNDQPEDTAMTEGGARDKAVEPSGELKV

SEQ ID NO: 114 - protein, 259 aa, Brachypodium distachyon
MEHVARFFFGVSGNVIALFLFLSPVVTFWRIIRKRSTEDFSGVPYNMTLLNCLLSAWYGLPFVSPNNILVTTINGAGSVIEAIYVIIFLIFAERKSRLRMTGLLGLVTSIFTTVVLVSLLALHGQARKVFCGLAATVFSICMYASPLSIMRLVIKTKSVEFMPFLLSLSVFLCGTSWFIYGLLGRDPFIAIPNGCGSFLGLMQLILYAIYRNNKGTGAGAGKAVDEVEDAKKATVAMEMAETKVAVDEPAAVDKVAAQV

SEQ ID NO: 115 - protein, 256 aa, Brachypodium distachyon
MEDVAKFLFGISGNVIALFLFLSPVPTFWRIIRKKSTEEFSGVPYNMTLLNCLLSAWYGLPFVSPNNILVSTINGAGAAIEACYVVIFLCFASSKKARLRTLGLASAVVAVFAAVALVSMLALHGPGRKLLSGLAMAVFSICMYASPLSIMRLVIRTKSVEYMPFLLSLAVFLCGTSWFVYGLLGRDPFVAVPNGCGSVLGAAQLILYAVYRNNKGKSSDGKLQGSDDVEMSVDARNNKVAHGDDAGGSQDVQQDS

SEQ ID NO: 116 - protein, 250 aa, Brachypodium distachyon
MFPDLRVTTGIIGSVVCLLLYAAPILTFKRVIKKGSVEEYSCIPYILTLFSSLTYTWYGLPVVSSGWENLTLSGISSLGVLFESTFISIYIWFAPRGKKKLVMAMVSSIVIIFGMAVFFSSFSIHTHQMRKVFVGSIGLVASILMYGSPLVAVKQVIRTKSVEFMPFYLSLFSFLTSLLWMLYGILGRDVFLTAPSCIGCLMGILQLVVYCMYNKCKESPKTNPDIEQADVVKVTTSQDDTKGQKPLSES

SEQ ID NO: 117 - protein, 272 aa, Hordeum vulgare
MEHIARFFFGVSGNVIALFLFLSPVVTFWRIIKRKSTEDFSGVPYNMTLLNCLLSAWYGLPFVSPNNILVTTINGAGSVIEAIYVVIFLIFAERRSKIRMLGLLSVVTAIFTTVVLVSLLALHGKGRTVFCGLAATVFSICMYASPLSIMRLVIKTKCVEFMPFLLSLSVFLCGTSWFIYGLLGLDPFIYIPNGCGSFLGLMQLILYAIYRKNKGPAAGAVPAGKGEDADEVEDGKKAAAAVEMGEAKVNKANDDSAVDVDEQAVDKVASQV

SEQ ID NO: 118 - protein, 262 aa, Hordeum vulgare
MEDVAKFFFGISGNVIALFLFLSPVPTFWRIIRNKSTEEFSGVPYNMTLLNCLLSAWYGLPFVSPNNVLVSTINGVGAAIETVYVVIFLVFASSRKARLRTLGLASAVAAVFAVVALVSMLALHGPARKLLAGLAMTVFSICMYASPLSIMRMVIKTKSVEYMPFLLSLAVFLCGTSWFIYGLLGHDLFVTIPNGCGSVLGAAQLILYAVYWNNKGNAAAGAGKMQGDDVEMSVDGRNNKVADGDDSGARESKKAGKMVSQV

SEQ ID NO: 119 - protein, 289 aa, Hordeum vulgare; identical to SEQ ID NO: 90

SEQ ID NO: 120 - protein, 292 aa, Hordeum vulgare
MGGLSAQHPWAFTFGLLGNVISFMTYLAPLPTFYRIYKNKSTQGFQSVPYVVALFSAMLWIYYALLKSDEYLLITINTAGCVIETIYIVLYLAYAPKQARLFTAKILLLLNVGVFGLILLLTLLLTAGERRVVMLGWVCVGFSVCVFVAPLSVIRLVVRTRSVEFMPFSLSLSLTASAVVWFLYGLLIKDKYVALPNILGFAFGVIQMGLYALYRNATPIPAPKEMDAPESEDGAVKAPEHVVNIAKLGTAAAAIELNTNHPVEPPPPMKEGTAKACATGEKLDKATHVEQV

SEQ ID NO: 121 - protein, 307 aa, Hordeum vulgare
MLAVCTFTETNKRNIISLMVFLSPLPTFYRVYRKKSTEGFQSTPYLVTLFSCLLWMYYAFLKSGSELLLTINGVGCVIETLYIAMYLVYAPKSARFLTAKLFIGLDVGLFGIIALVTMLASAGTLRVQVVGWICVAVALGVFAAPLSIIRLVIRTKSVEFMPFSLSFFLVLSAVVWFAYGALKKDIFVAVPNVLGFVFGIAQMALYMAYRNKKPATVVLVHEEMKLPEHVKEVGAGGAKPQGGAPTEGRISCGAEVHPIIDVLPAAGAVDEEAAAAADEDVIRDDHNMLRPEQPAIIKPDVAIVVQA

SEQ ID NO: 122 - protein, 256 aa, Sorghum bicolor
MEDVVKFIFGICGNVIALFLFLSPVPTFWRIIRRRSTEDFSGVPYNMTLLNCLLSAWYGLPFVSPNNILVSTINGAGAAIEAVYVVIFLVFASSQRTRLRMLGLASAVAAVFAAVALVSMLALHQGQGRKLMCGLAATVCSICMYASPLSIMRLVVKTKSVEYMPFLLSLAVFLCGTSWFVYGLLGRDPFVAIPNGCGSFLGAVQLVLYAIYRNSAGTAGAGKQQAGDDVEMAADAKSSKKVADDVGGAGKEGRLV

SEQ ID NO: 123 - protein, 231 aa, Sorghum bicolor
MSSLYDLSCFAAGLAGNVFALALFLSPVPTFKRVLKAKSTEQFDGLPYLLSLLNCCICLWYGLPWVSGGGGRALVATVNGTGALFQLAYISLFIFYADSRTTRLRITGLLVLVVFAFALIAHASIALFDQPVRQLFVGSVSMASLVSMFASPLAVMGLVIRTECVEFMPFYLSLSTFLMSASFAMYGLLLRDFFIYFPNGLGVVLGAMQLVLYAYYSRRWKNSGSSAALLA

SEQ ID NO: 124 - protein, 171 aa, Sorghum bicolor
MALMLTFKRVVKEASVGEFSCLPYILALFSAFTWGWYGFPIVSDGWENLSLFGTCAVGVLFETSFIIVYIWFAPRDKKKQVISTKSVEFMPFYLSLFSLLTSFTWMLYGILGRDLYLTVPNGAGCITGILQLIVYCIYRRCNKPPKAVNDIEMVNDLDVATSREDTNGCKP

SEQ ID NO: 125 - protein, 259 aa, Sorghum bicolor
MVPNTVRVAVGILGNAASMLLYAAPILTFRRVIKKGNVEEFSCVPYILALFNCLLYTWYGLPVVSSGWENLPVATINGLGILLEITFIGIYIWFAPAEKKRFALQLVLPVLALFALTAALSSFMAHTHHMRKVFVGSVGLVASISMYSSPMVAAKRVIETKSVEFMPFYLSLFSFLSSALWMIYGLLGRDFFIASPNFIGVPMGMLQLLLYCIYRRDHGAAAEAEVRVHGAAADEEKGLKAAVPMAVLVQPQETTDASK

SEQ ID NO: 126 - protein, 250 aa, Sorghum bicolor
MVSPDTIRTAIGVIGNGTALVLFLSPVPTFIRIWKKGSVEQYSPIPYVATLLNCMMWVLYGLPVVHPHSMLVITINGTGMAIQLTYVTLFLLYSAGAVRRKVFLLLAAEVAFLGAVAALVLTLAHTHERRSMIVGILCVLFGTGMYAAPLSVMKMVIQTKSVEYMPLFLSLASLVNGICWTAYALIRFDLYITIPNGLGVLFAVAQLVLYAMYYKNTQKIIEARKRKTDQVAMTEVVVDGSGRASNNNTY

SEQ ID NO: 127 - protein, 250 aa, Sorghum bicolor
MISPDTIRTAIGVIGNGTALVLFLSPVPTFIRIWKKGSVEQYSPIPYVATLLNCMMWVLYGLPAVHPHSMLVITINGTGMAIQLTYVTLFLLFSAGAVRRKVVLLLAAEVAFVGAVAALVLSLAHTHDRRSMVVGILCVLFGTGMYAAPLSVMKMVIQTKSVEYMPLFLSLASLVNGICWTAYALIRFDLYITIPNGLGVLFAVAQLVLYAIYYKSTQEIVEARKRKAEQVAMTEVVIDGGKTNNHASGY

SEQ ID NO: 128 - protein, 252 aa, Sorghum bicolor
MVSKDTIRTAIGVIGNGTALVLFLSPVPTFVGIWKKRAVEQYSPIPYVATLLNCMMWVVYGLPVVHPHSMLVVTINGTGMLIQLSYVVLFILCSTGAVRRKVVLLFAAEVAFVVALAALVLSLAHTHERRSMVVGIVSVFFGTGMYAAPLSVMKMVIETKSVEYMPLFLSLASLANSICWTAYALIRFDVYITIPNGLGVLFALGQLVLYAMFYKNTQQIIEARKRKADHQQGTVMEVVTDATPPNNNGNTY

SEQ ID NO: 129 - protein, 239 aa, Sorghum bicolor
MVNLDEVRNVVGIIGNFISFGLFLAPLPTFLTIIKKRDVEEFVPDPYLATFLNCALWVFYGLPVVHPDSILVATINGTGLAIEAAYLSVFFAFAPKPKRAKMLGVLAVEVAFVAAVVAGVVLGAHTHEKRSLVVGCLCVLFGTLMYASPLTVMKKVIATQSVEYMPFTLSFVSFLNGICWTTYALIRFDIFITIPNGMGTLLGLMQLILYFYYYGSTPKSSGTTAGMELPVKAGDGDSN

SEQ ID NO: 130 - protein, 244 aa, Sorghum bicolor
MISPDAARNVVGIIGNVISFGLFLSPAPTFWRIYKARDVEEFKPDPYLATLLNCALWVFYGIPVVHPNSILVVTINGIGLVIEGIYLTIFFIYADAKKRKKAFAILFVEILFMVAVVLGVILGAHTHEKRSMIVGILCVIFGSVMYASPLTIMGKVIKTKSVEYMPFLLSLVNFLNGCCWTAYALIRFDLYVTIPNALGAFFGLIQLILYFWYYKSTPKKEKNVELPTVSRNVGGGNVTVSVER

SEQ ID NO: 131 - protein, 213 aa, Sorghum bicolor
MVSDVVAFLGFLASFSLFASPAFIFRRIITEASVVGYPFLPYPMAFLNCMIWLFYGTVHTNSDYVIIINSVGMIIEVIFMGFYIWFADGMDLRVALIELFGMGGLGTFVALLGYLWRDTVFGYAGVVSGIIMYGSPLSVARRVFETRNVQNMSLLMALASLTASSVWTAYAFASKPYDFYIAIPNLIGLVLALVQLALYAYYYFNGEEEDVVA

SEQ ID NO: 132 - protein, 242 aa, Sorghum bicolor
MAGAQPNIAQELFGILGDITCGGLFLSPVATMWDISRHGSSEQYSASPYLAGLLNCAVWLLYGYVHPNGKWVFGINIVGSLLQLLYIVIFVYYTTVDDVRYQIYYMLFGAGVCLVGIMALVFGQAHSTEQKCMGFGLAGVATGIGMYAAPLIQLRSVVERGNVEGMSLLLIGASLGNSAVWTVYACLGPDFYVLFNLKKTSLTAGPQSDWRVVHGGAAGCLLPLQQQQQQQRRITEVGNCMK

SEQ ID NO: 133 - protein, 309 aa, Sorghum bicolor
MAGGLFSMAHPAITLSGIAGNIISFLVFLAPVATFLQVYRKKSTGGFSSVPYVVALFSSVLWIFYALVKTNSRPLLTINAFGCGVEAAYIVFYLAYAPRKARLRTLAYFFLLDVAAFALVVVVTLFVVREPHRVKFLGSVCLAFSMAVFVAPLSIIVKVVKTKSVEFLPISLSFCLTLSAVAWFCYGLFTKDPFVMYPNVGGFFFSCVQMGLYFWYRKPRPAKNNAVLPTTTDGASAVQMQGQVIELAPNTVAILSVSPIPIVGVHKIEVVEQQHKEAAVAAETRRMAAANPDGAMPEVIEIVPAVATV

SEQ ID NO: 134 - protein, 273 aa, Sorghum bicolor
MAGGLFSMEHPWVSAFGILGNIISFLVFLAPVPTFLRVYRKKSTEGFSSVPYVVALFSCTLWILYAVVKTNSSPLLTINAFGCVVEATYILLYLIYAPRAARLRALAFFFLLDVAALALIVVVVVVLVAEPHRVKVLGSICLAFSMAVFVAPLSVIFVVIRTKSAEFMPFTLSFFLTLSAVAWFLYGIFTKDPYVTLPNVGGFFFGCIQMVLYCCYRKPSASVVLPTTTDAAATEMELPLAAHQAVAPVLAELQKLEEAMGSPRKHGGVVKVV

SEQ ID NO: 135 - protein, 313 aa, Sorghum bicolor
MITVGHPVVFAVGILGNILSFLVTLAPVPTFYRVYKKKSTESFQSVPYVVALLSAMLWLYYALLSIDVLLLSINTIACVVESVYLAIYLTYAPKPAMAFTLKLLFTMNMGLFGAMVAFLQFYVDGQRRVSIAGGVGAAFALAVFVAPLTIIRQVIRTKSVEYMPFWLSFFLTISAVVWFFYGLLMKDFFVAMPNVLGLLFGLAQMALYFVYRNRNPKQNGAVSEMQQQAAVVQADADAKKEQQLRQAHADAGADGEAVAVRIDDEEEPKNVVVDIMPPPPPLLPAERASPPLPLPPPPAMVMMTAHQTAVEVV

SEQ ID NO: 136 - protein, 304 aa, Sorghum bicolor
MAGLSLQHPWAFAFGLLGNVISFMTFLAPIPTFYRIYKTKSTEGFQSVPYVVALFSAMLWIFYALIKSNETFLITINAAGCVIETIYIIMYFVYAPKKGKMFTAKIMLLLNVGIFGVILLLTLLLFKGDKRVVMLGWICVGFSVSVFVAPLSIMKRVIQTKSVEYMPFSLSLSLTLSAVVWFLYGLLIKDKYVALPNILGFTFGVVQMVLYVLYMNKTPVAVAEGKDAGGKLPSAADEHVLVNIAKLSPALPERSSGVHPVVAQMAAVPNRSCAAEAAAPPAMLPNRDVVDVFVSRHSPAVHVV

SEQ ID NO: 137 - protein, 302 aa, Sorghum bicolor
MAGLSLQHPWAFAFGLLGNLISFLTFLAPIPTFYRIYKTKSTEGFQSVPYVVALFSAMLWIFYALIKSNETFLITINAAGCVIETIYIVMYFVYAPKKAKLFTAKIMLLLNVGVFGVILLVTLLLFKGDKRVVMLGWICVGFSVSVFVAPLSIMRRVIQTKSMEYMPFSLSLSLTLSAVVWFLYGLLIKDKYVALPNILGFTFGMVQMVLYVLYMNKTPVAVAEGKDAGGKLPSAGDKHVLVNIAKLSPALPERSSGVHRATQMSAVPAKSCAAEATAPKVMLPNRDVVDVFLSQALHRKQA

SEQ ID NO: 138 - protein, 302 aa, Sorghum bicolor
MAGLSLQHPWAFAFGLLGNVISFLTFLAPIPTFYRIYKSKSTEGFQSVPYVVALFSAMLWIFYALIKSNETFLITINAAGCVIETIYIVMYFVYAPKKAKLFTAKIMLLLNVGVFGVILLVTLLLFKGDKRVVMLGWICVGFSVSVFVAPLSIMRRVIQTKSVEYMPFSLSLSLTLSAVVWFLYGLLIKDKYVALPNILGFTFGVVQMVLYVLYMNKTPVAVAEGKDAGVKLPSAADEHVLVNITKLSPALPDRSSGVHRATQMAAVPASSCAAEAAAPAMLPNRDVVDVFVSRQSPAVHVV

SEQ ID NO: 139 - protein, 291 aa, Sorghum bicolor
MAGLSLQHPMAFAFGLLGNIISFMTYLAPLYRPTFYRIYKSKSTQGFQSVPYVVALFSAMLWIYYALLKSNEFLLITINSAGCVIETLYIVMYLLYAPKKAKLFTAKILLLLNVGVFGLILLLTLLLSAGQHRVVVLGWVCVAFSVSVFVAPLSIIRQVVRTRSVEFMPFSLSLSLTVSAVVWFLYGLLIKDKYVALPNVLGFSFGVVQMGLYALYRNATPRVPPAKEVTDDDAAADGTFKLPGEHVVTIAKLTAVPAVSPQLQEEAKPADNGTTPAPAPANDVQLNAEQV

SEQ ID NO: 140 - protein, 336 aa, Sorghum bicolor
MAFLNMEQQTWAFTFGILGNIISLMVFLSPLPTFYRVYRKKSTEGFQSTPYVVTLFSCMLWIFYALLKSGAELLVTINGVGCVIETVYLGMYLLYAPKAARVLTAKMLLGLNVGVFGLVALVTMVLSNGGLRVKVLGWICVSVALSVFAAPLSIMRQVIRTKSVEFMPISLSFFLVLSAVIWFAYGALKKDVFVAAPNVLGFVFGLAQMALYMAYRNKKPAAAAVIMVEEVKLPAEQYASKEVAPPAAAHEGSRASCGAEVHPIDIDTLPVADVGRHHDSQAVVVIDVDVEPAATCAAAAAAAGGVRGDGAAGVVTAGPEQPAAMKPVDMAIAVEA

SEQ ID NO: 141 - protein, 329 aa, Sorghum bicolor
MTTPSFLVGIAGNVISILVFASPIATFRRIVRNKSTGDFTWLPYVTTLLSTSLWTFYGLLKPKGLLVVTVNGAGAALEAVYVTLYLVYAPRETKAKMGKLVLAVNVGFLAVVVAVALLALHGGARLDAVGLLCAAITIGMYAAPLGSMRTVVKTRSVEYMPFSLSFFLFLNGGVWSVYSLLVRDYFIGVPNAVGFVLGTAQLVLYLAFRNKAAERKDDDDEKEAAAAAPSSGDEEEGLAHLMGPPQVEMEMTAQQRGRLRLHKGQSLPKPPTGGPLSSSSSSSPHHGFGSIIKSLSATPVELHSVLYQHGLGRGRFEPVKKDDVDATNH

SEQ ID NO: 142 - protein, 166 aa, Sorghum bicolor
MEAIYVVLFIVYAANHATRVKTVKLAAALDIGGFGVVYAVARFAINELDLRIMVIGTICACLNVLMYGSPLAAMKTVITTKSVEFMPFFLSFFLFLNGGIWATYAVLDRDMFLGIPNGIGFVLGTIQLIIYAIYMNSKTSQSSKETASPLLASDHNQGEASSHSHV

SEQ ID NO: 143 - protein, 260 aa, Picea sitchensis
MLIAHFIFGIFGNITALTLFLAPLITFWTIIKNKSTEQFSGFPYVSTLLNCLLSAWYGLPFVSPNNLLVSTVNGTGAAIELCYVIVFLFYIRDKKYRVKIFGLLVIVLKFFALVALVSLLALHGHARKLFCGFAAAIFSICMYASPLSIMRTVIKTKSVKYMPFFLSLCVFLCGTSWFIFGLLGKDPFLAVPNGVGSALGAMQLILYAVYKDWKKKDSNTWSPPVQEEGKAGADHMNAMEMGSYGQTEAHNPSGKYVNGF

SEQ ID NO: 144 - protein, 272 aa, Picea sitchensis
MEKDHIRLAVGIIGNITSLLLYGAPVLTFMKVIKEKSVGQYSCTPYLIALFNCLIYTWYGFPVVSNGWENFLVSTVNGVGIVPECFAICTYIVYAPPKFKRKVARMVGCVLVLFGVMAAISFFSLHDHKNRKFMIGIVGILSSISLYSAPFVAMKLVIQTKSVEFMPFYLSFFAFINCIMWMTYGALSRDIFLATPNVIGSPLALAQLVLYCIYRKKTRGVQNGNNLDPEEGVQINGAQSTNSEEKTKLPDGQKGENAEYINTTEIKTILIN

SEQ ID NO: 145 - protein, 269 aa, Lotus japonicus
MAEHFRMVVAVIGNVASVSLYAAPTVTFKRVIRKKSTEEFSCIPYIIGLLNCLLFTWYGLPVVSNKWENFPLVTVNGVGIVFELSYVLIYFWYSSAKQKVKVATTAIPVILVFCAIALVSAFNFPDHRHRKLLVGSVGLGVAVAMYASPLVAMKKVIQTKSVEFMPLPLSLCSFLASVLWLTYGLLIQDIFVAGPSLVGTPLSILQLVLHCKYWKRREMKEPINNKVELHKENMEKLDLEKGGLFETKDIEEKNVTILNNDINSKNMTM

SEQ ID NO: 146 - protein, 272 aa, Nicotiana alata
MTLLSVQELAFLFGLLGNIVSFMVFLAPVPTFYKIYKKGSSEGFQAIPYVVALFSAGLLLYYAYLTKNAFLIVTINAFGCVIELTYIFLFLFYASKKSKMTTVWLMLLDVGALGIVMLFSYLFAKGTKRVEIVGWICAIVNIAVFAAPLSIMRQVIKTKSVEFMPFTLSLFLTLCATMWFFYGYFKKDYYIALPNVLGFLLGIVQMILYIVYKYARRKYNGEWELEGIDINIKTDGNFENKIVSSMEKPSLENGHQSNQEHNRDMTSVLTLK

SEQ ID NO: 147 - protein, 238 aa, Solanum lycopersicum
MADPDTTRTVVGIIGNVISFFLFLSPGPTFVQILKAKSVMEFKPDPYIATVLNCAVWVFYGMPFVHPDSLLVITINGFGLAIELLYVSIFFIYSDWSKRQKIIIALVIEAIFMAILIFVTLTFLHGTKDRSMLIGIVAIVFNIIMYTSPLTVMKKVITTKSVKYMPFYLSLANFANGIVWACYALLKFDPYILIPNGLGSLSGLVQLILFAAFYRTTNWDEDEKEVELSTSKSNKSDV

SEQ ID NO: 148 - DNA, 45 bp, Artificial sequence
  Primer
gctttcccgaatgtgcttggttgagctctcggtgcactccaaatg

SEQ ID NO: 149 - DNA, 45 bp, Artificial sequence
  Primer
catttggagtgcaccgagagctcaaccaagcacattcgggaaagc

SEQ ID NO: 150 - DNA, 47 bp, Artificial sequence
  Primer
gcagtcctcttccgcagcagctacatagccagctttcttgtacaaag

SEQ ID NO: 151 - DNA, 47 bp, Artificial sequence
  Primer
ctttgtacaagaaagctggctatgtagctgctgcggaagaggactgc

SEQ ID NO: 152 - DNA, 21 bp, Artificial sequence
  Primer
ccgaagagtaatgtgaccacg

SEQ ID NO: 153 - DNA, 21 bp, Artificial sequence
  Primer
tgaagtgggtgcttttgtttc

SEQ ID NO: 154 - DNA, 21 bp, Artificial sequence
  Primer
atgcaggccaacgttctatag

SEQ ID NO: 155 - DNA, 22 bp, Artificial sequence
  Primer
tcaaaggccaaagcaatatacc

SEQ ID NO: 156 - DNA, 25 bp, Artificial sequence
  Primer
ggggacaagtttgtacaaaaaagca

SEQ ID NO: 157 - DNA, 26 bp, Artificial sequence
  Primer
ggcttacacacgcatcggatcggaga

SEQ ID NO: 158 - DNA, 25 bp, Artificial sequence
  Primer
ggggaccactttgtacaagaaagct

SEQ ID NO: 159 - DNA, 25 bp, Artificial sequence
  Primer
gggtatgtagctgctgcggaagagg

SEQ ID NO: 160 - DNA, 30 bp, Artificial sequence
  Primer
ggggggtacccacacgcatcggatcggaga

SEQ ID NO: 161 - DNA, 31 bp, Artificial sequence
  Primer
ggggctgcagctgtagctgctgcggaagagg

SEQ ID NO: 162 - DNA, 56 bp, Artificial sequence
  Primer
ggggacaagtttgtacaaaaaagcaggcttcaaatggtgaacaatctcgtcgttat

SEQ ID NO: 163 - DNA, 52 bp, Artificial sequence
  Primer
ggggaccactttgtacaagaaagctgggtaagtagttgcagcactgtttcta

SEQ ID NO: 164 - DNA, 25 bp, Artificial sequence
  Primer
ggggacaagtttgtacaaaaaagca

SEQ ID NO: 165 - DNA, 30 bp, Artificial sequence
  Primer
ggcttaatgagtctcttcaacactgaaaac

SEQ ID NO: 166 - DNA, 25 bp, Artificial sequence
  Primer
ggggaccactttgtacaagaaagct

SEQ ID NO: 167 - DNA, 25 bp, Artificial sequence
  Primer
gggtatgtagctgctgcggaagagg

SEQ ID NO: 168 - DNA, 56 bp, Artificial sequence; identical to SEQ ID NO: 162
  Primer

SEQ ID NO: 169 - DNA, 52 bp, Artificial sequence; identical to SEQ ID NO: 163
  Primer

SEQ ID NO: 170 - DNA, 20 bp, Artificial sequence
  Primer
tccaagctgttctctccttg

SEQ ID NO: 171 - DNA, 20 bp, Artificial sequence
  Primer
gagggctggaacaagacttc

SEQ ID NO: 172 - DNA, 22 bp, Artificial sequence
  Primer
gccaatctcagtggttcgtcaa

SEQ ID NO: 173 - DNA, 21 bp, Artificial sequence
  Primer
gaagaggactgcttgccatgt

SEQ ID NO: 174 - DNA, 28 bp, Artificial sequence
  Primer
tccttctcctaacaacttatataccatg

SEQ ID NO: 175 - DNA, 23 bp, Artificial sequence
  Primer
tcctatagaacgttggcacagga

SEQ ID NO: 176 - DNA, 23 bp, Artificial sequence
  Primer
ctcacatctcctgaaccagtagc

SEQ ID NO: 177 - DNA, 22 bp, Artificial sequence
  Primer
tgcagcactgtttctaactccc

SEQ ID NO: 178 - DNA, 29 bp, Artificial sequence
  Primer
aaagctgatatctttcttactacttcgaa

SEQ ID NO: 179 - DNA, 27 bp, Artificial sequence
  Primer
cttacaaatcctatagaacgttggcac

SEQ ID NO: 180 - DNA, 23 bp, Artificial sequence
  Primer
cttctacgttgcccttccaaatg